SEQUENCE LISTING (128 sequences)

SEQ ID NO: 1 - DNA, 1847 bp, Artificial Sequence
  XL.D0-AAV9
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggtggctctgcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 2 - DNA, 1829 bp, Artificial Sequence
  XL.D1-AAV9
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggtggctctgcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 3 - DNA, 1841 bp, Artificial Sequence
  XL.D1b-AAV9
aggaaaacagcaagcgctggaacccggagatccagtacacttccaactattacaagtctaataatgttgaatttgctgttaatactgaaggtgtatatagtgaaccccgccccattggcaccagatacctgactcgtaatctggagaatctttatttccagggcgcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 4 - DNA, 2163 bp, Artificial Sequence
  XL.D1b-AAV2
gcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctggagaatctttatttccagggcgctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttttgcccggaatggtctggcaggatcgagatgtatacctccagggtccgatttgggctaaaatccctcacaccgatggacacttccatccttcccctttgatgggtgggttcggtctgaagcatccccctcctcagattctcataaaaaacacaccagtccctgctaatcccggcagtttcgcatcttttatcactcagtattccaccggccaggtaagtgtggagatcgagtgggaattgcaaaaggagaactcaaagaggtggaaccctgagatacaatacacctcaaactataacaaatctgtgaacgtcgatttcaccgttgacactaacggcgtttactccgagcctcgcccaatcggcacacgatatcttaccagaaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggag

SEQ ID NO: 5 - DNA, 1791 bp, Artificial Sequence
  XL.D1b-AAV5
agaaggaaaactccaagaggtggaacccagagatccagtacacaaacaactacaacgacccccagtttgtggactttgccccggacagcaccggggaatacagaaccaccagacctatcggaacccgataccttacccgaccccttgagaatctttatttccagggctctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcggcagtgtcagcagcttcatcacccagtacagcaccgggcaggtcaccgtggagatggagtgggagctcaagaaagaaaacagtaaaaggtggaaccccgaaattcagtacaccaacaattacaatgatccacaattcgttgacttcgcaccagattcaacaggtgagtatcgaactactcggcctataggtacacggtatctgacacggcctctttaacccattcatgtcgcataccctcaataaaccggttaattcgtgt

SEQ ID NO: 6 - DNA, 1785 bp, Artificial Sequence
  XL.D1b-AAVDJ
aggaaaacagcaagcgctggaaccccgagatccagtacacctccaactactacaaatctacaagtgtggactttgctgttaatacagaaggcgtgtactctgaaccccgccccattggcacccgttacctcacccgtaatctggagaatctttatttccagggcgctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctggcagtctgaactctttcatcacccagtattctactggccaagtcagcgtggagatcgagtgggagctgcagaaggagaactctaagcggtggaaccctgagatacaatacacatccaattactataaatccacatctgttgattttgctgtcaacacggagggcgtttactccgagccgagacccatcggtaccaggtatctgacgcggaatctgtaattgcctgttaatcaataaaccggttgattcgttt

SEQ ID NO: 7 - DNA, 1847 bp, Artificial Sequence
  XL.D1c-AAV9
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggaggtgagaatctttatttccagggcgcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 8 - DNA, 1845 bp, Artificial Sequence
  XL-D1c-AAVDJ
aaggaaaacagcaagcgctggaaccccgagatccagtacacctccaactactacaaatctacaagtgtggactttgctgttaatacagaaggcgtgtactctgaaccccgccccattggcacccgttacctcacccgtaatctgggaggtgagaatctttatttccagggcgctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctggcagtctgaactctttcatcacccagtattctactggccaagtcagcgtggagatcgagtgggagctgcagaaggagaactctaagcggtggaaccctgagatacaatacacatccaattactataaatccacatctgttgattttgctgtcaacacggagggcgtttactccgagccgagacccatcggtaccaggtatctgacgcggaatctgtaattgcctgttaatcaataaaccggttgattcgtttcagttgaactttggtctctgcgaagggcgaattcgtttaaacctgcaggacta

SEQ ID NO: 9 - DNA, 3431 bp, Artificial Sequence
  XL.T1-AAV9
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggtgggggcggatctgcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaactccaaacgctggaacccggagatccagtacacttccaactattacaagtctaataatgttgaatttgctgttaatactgaaggtgtatatagtgaaccccgccccattggcaccagatacctgactcgtaatctgggaggtggaggctctgcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 10 - DNA, 5039 bp, Artificial Sequence
  XL.Q1-AAV9
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggtgggggcggatctgcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaactccaaacgctggaacccggagatccagtacacttccaactattacaagtctaataatgttgaatttgctgttaatactgaaggtgtatatagtgaaccccgccccattggcaccagatacctgactcgtaatctgggaggtggaggctctgcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaactccaaacgctggaacccggagatccagtacacttccaactattacaagtctaataatgttgaatttgctgttaatactgaaggtgtatatagtgaaccccgccccattggcaccagatacctgactcgtaatctgggaggcgggagtggcggagggggatcagcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 11 - DNA, 280 bp, Artificial Sequence
  XL.N0-AAV9
ttcggatccgcggccgccatggcaatggttaaacgaaacaacaatactgggatgatccccgtcagtaccaagcagctcctggccctcggagccgctgctggggctaccgcacttcagggatttgtcaaaaacaatggcatggccattgttgagggcgcggtggatctgaccaagagggcgtataaagctgttcggagaagggggggtaaaaaacaacaaatgattaaccacgtaggtggtactggtggagcaatcatggcacctgtgggtagttcctcgg

SEQ ID NO: 12 - DNA, 627 bp, Artificial Sequence
  XL.N1-AAV9
agagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatgattaaccacgtaggtggtactggtggagcaatcatggcacctgtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtc

SEQ ID NO: 13 - protein, 7 aa, Artificial Sequence
  TEV linker
ENLYFQG

SEQ ID NO: 14 - protein, 9 aa, Artificial Sequence
  Extended TEV linker
GGENLYFQG

SEQ ID NO: 15 - DNA, 3305 bp, Artificial Sequence
  rAAV genome 1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggcccaacggtaccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 16 - DNA, 4923 bp, Artificial Sequence
  rAAV genome 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatcccctagctaagggctagcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggcccaacggtaccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 17 - DNA, 6957 bp, Artificial Sequence
  rAAV genome 3
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtaccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcaagcttataaataattcaataaagatgtcgttttgctagtatacgtctaggcgtcacccgccatctctgtgcaggtgggccgacgagacattgtccctgatttctccactactaatagcacacacggggcaataccagcacaagctagtctcgcgggaacgctcgtcagcatacgaaagagcttaaggcacgccaattcgcactgtcagggtcacttgggtgttttgcactaccgtcaggtacgctagtatgcgttcttccttccagaggtatgtggctgcgtggtcaaaagtgcggcattcgtatttgctcctcgtgtttactctcacaaacttgacctggagatcaaggagatgcttcttgtggaactggacaacgcatcaacgcaacggatctacgttacagcgtgcatagtgaaaacggagttgctgacgacgaaagcgacattgggatctgtctgttgtcattcgcggaaaacatccgttcacgaggcggacactgattgacacggttttgcagaaggttaggggaataggttaaattgagtggcttaaaaatgctatgtctgggattaaagtgtagtaaactgtgattaacggagacggttttaagacaggagttcgcaaaatcaagcggggtcattacaacggttattcctggtggtttaggcgtacaatgtcctgaagaatatttaagaaaaaagcacccctcgtcgcctagaattacctaccgcggtcgaccataccttcgattatcgcgcccactctcccattagtcggcagaggtggttgtgttgcgatagcccagtatgatattctaaggcgttacgctgatgaatattctacggaattgccataggcgttgaacgctacacggacgatacgaatttatgtatagagcgggtcatcgaaaggttatactcttgtagttaacatgtagcccggccctattagtacagcagtgccttgaatgacgaattcgtttaaacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcatatgatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgcgccctgctcgacgcccttgccatcgagatgctggacaggcatcatacccacttctgccccctggaaggcgagtcatggcaagactttctgcggaacaacgccaagtcattccgctgtgctctcctctcacatagagacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagccattgagctgttcgaccggcagggagccgaacctgccttccttttcggcctggaactaatcatctgtggcctggagaaacagctaaagtgcgaaagcggcgggccagcagatgccctcgatgactttgatcttgacatgctccccgccgacgcactggatgattttgaccttgatatgctgcctgctgacgctcttgacgattttgaccttgacatgctccctggggctagcggatccgagggcagaggaagccttctaacatgcggtgacgtggaggagaatcccggcccttccgggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatatccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 18 - DNA, 6863 bp, Artificial Sequence
  rAAV genome 4
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggacggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 19 - DNA, 5183 bp, Artificial Sequence
  rAAV genome 5
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacactagaattccctaggccggccacgcgttttttttctcgttttactcacaaatacttaagtatgtaagtagatacacttcacccacataatagagaatactctatacatgtattcatgggtcaaaggggtccctgagatacacacaattcatactcatccctctgaacactgcaagcgtcgtgtggagccatttgctttcccacacactcgctttctgccgcagcaaggctggaccctgctggagtggtgatgtgcaggaactgcctcatagctcaggtggtaaagttatttggctgggaactgagtctctccccacatgctgtcacacccttcccggccctgacaaacacaaagagatgcatcccattgagtcacccacttggctgaaccgtgtacctggtaacctccttgggaggttctttggagggtatttttctcctctacacaaagcatgcctgcgaagtaagctgagaatgacaaaaacaggaaagcccaatcttgcagagacagaggggccaaggcctgcatgataatgcggcccaaattcatagctcagacccaaaagaaaacaaatgttccattctttctgtgtccacctctaaactaagcaatacagtgagggcagtaaattgcaattacaagtaagaactttagcagttgctgatcataaaatgaaaagggacaaatatgtttatccttaacagggtcacaaataattccacagctgttaattttggcagccacgactctgcaggcagtatgggctcatggtatactatcttcattgtcaccttggacaaagatcctgttggccaaaggcacacaggcatcttcaggaaaactagatttaaatggtaagattacctatgcttatgtttttaagctccaaagactactggttctgggaaggactgggtcactatgtgaccttgtgtaggtctcttttggccctgagtccaggagggctctgtgactccaggttgtggctgcgtctggcagctaacaattgtttagtttacagtgtccttcctcttcatcttgtttgcattcagttgcccatgcctgcctctggggggaggtgggaaggaatgtggctcctgggaggagcccagctccatgtgccattctgaggagccaggctgcacgcccctgggccatagctgctggtggatggatgagtacactggcagaggcactgggaagggaagacagaattgttggccaatagtccccttttactgattttcttgtcatttttgtgtttgttttattataaagataacagctgtatttgagaaagaaagctcaaactataaagaaaatttaaaattgccctgtcttactccaactccattccctgactaggtgagccagacgctggccttatctcatttaccatctcagagccatctgaagggggagaagggaaccgggccccaggagggagaaagtcatcaaacctcccacatctgtgaccagcctcagtgccatactttttctatggagggccctgtccaatggaactgagcacagaccagataaaagaactgggcacccagtggcctcagtccagggcctggagttcaaactttactggaaacaaaggggccgagagagactggggaaagaactactagaaaggggctggtgcccccatggggctgtgggttttggagccgcgtgcccccacctgagcctcagggggcccggagtgtccacaccagtggacctttcgagaaatggctgggccattgtgcagaagaatgcccggaaatcccgcgcctccctcctccagcaaggatgggggctcttcctcctggccaggaaactccaagttggcttccggagggtggcctgggggctggggtgccagggacaccatcgccactggtgggagggcagggcacagcccctccgtgtccctttgtctctcctgtctgaaggccagagcaggctgctaggcctggggccaccactgcccctgggtgctacacccagtgtgctgggtcactgggaacttcctgaagtggtgtcacctgaactggacccccaaggatggggtgcgggcagtaccgcaggaagaggagcagcccctgtgaagattgagaggtctgggaagcccctgcggcttgggagagtgggggtcgccaggcagggggaaagcccctgtgccaccgctttttgccagagactcaggctccagagaggcagtgagtggcatggggggtgaggctggggccctgggcctgacctccacacgcctgcctggcctctctgtttgccatgggatgagagagacagtgctgggactcagagcggggctggagagtgagagtgcgagaaagggcctgggtggggcttggaccccggggcgggctttctggagagcccccctacgagggcctctacggcggtgacggggtggggggcttctgcaaaccttggtcagggaagtggagctggctcgagtggaagagaccacccggctcagtcggggatgtgggagtggactgggtggtgcagactgggggtcgagcgccttctgaagtgacggggccgggacgcgcagggaggcggcccaagaagcgcgccctaggccagcccagaatgcgctcggccgcgactaggacaacggcgggtggggctgggggcggctgccgggcggggagcggtcccgcgccctcagctacccctcaagagccgttgtttccctaacttcagctgccagaggctctgtgattggctgcggcacgatgacccgcgcacggattggctgcttcgggccggggggccgggcccgggggacagaatccgcccccgaaccttcaaagagggtaccccccggcaggagctggcagacccaggaggtgcgacagacccgcggggcaaacggactggggccaagagccgggagcgcgggcgcaaaggcaccagggcccgcccagggcgccgcgcagcacggccttgggggttctgcgggccttcgggtggcgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 3765 bp, Artificial Sequence
  rAAV genome 6
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtaccggatccaccatgcccaagaagaagaggaaggtgtccaatctcctgactgttcaccagaacctccctgcgctgccagtagatgccactagcgatgaggtcaggaaaaatctcatggatatgtttagggatagacaggcgttttctgaacacacctggaaaatgctgcttagcgtgtgccgatcctgggcagcctggtgtaagctgaacaatcgcaaatggttccccgccgagccggaggacgtgcgcgattacctgctgtatctccaggcaagagggctggctgtcaagactatccagcagcacttgggccaactgaatatgctgcatcgacgcagcgggctcccccggcctagcgattcaaacgcagtctcccttgttatgaggagaattagaaaggaaaacgtagatgcgggtgagagggctaagcaggctctcgcttttgagcggactgatttcgaccaggtcagatccctgatggagaacagcgatcggtgccaggacatcaggaacctcgcatttctgggaattgcatataacacacttctgcgcatagctgagatcgcccggatcagagtgaaagacatcagtcgaacggacggcggccggatgcttattcatattggacgcacaaagacattggtcagcaccgctggcgttgaaaaggccttgtccctgggcgtaacgaagctggtggaaagatggatctcagtgtccggcgtggctgacgaccctaataattacttgttctgtcgagtgagaaaaaacggagtcgccgcgccctctgccaccagccaattgagtacacgggcccttgaagggatctttgaggcaacccaccgactcatatacggagccaaggatgacagtggccagaggtatctcgcctggtcaggtcattctgctagggtgggggccgcacgagacatggcgcgggcaggagtctccataccagagattatgcaagctggaggttggacaaatgtgaacatcgttatgaactatatccgcaatcttgactctgaaaccggggccatggtgagactgctcgaagatggtgactgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - DNA, 8212 bp, Artificial Sequence
  rAAV genome 7
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtaccggatccgttaacaccatgttcgtattccttgtcctcctccccctcgtcagctcccagtgtgtcaatctgacaactcgaactcagctcccacccgcatacaccaatagttttacccgaggagtctattatcccgacaaagttttcaggtctagtgtgttgcatagcacgcaggacctgttcttgccgtttttttcaaatgtgacatggttccatgccatccatgtctctggtacgaacgggaccaaaaggtttgataaccccgttcttccatttaacgacggggtttatttcgcctctactgaaaagtctaatataatacgaggttggatttttgggacaacactggactctaaaacgcaatctctcctcatagtaaataatgcgacgaatgtcgtaatcaaagtatgtgagtttcagttttgcaacgacccttttcttggtgtatactatcacaagaacaacaagtcttggatggaaagtgaatttagagtctacagctccgcaaacaactgtacctttgagtacgtcagtcagcccttccttatggacctcgaaggcaaacaggggaacttcaagaatctgcgcgagttcgtgttcaagaatatcgacggctatttcaagatatattcaaaacatacaccaatcaatttggtacgcgatttgccgcagggcttctctgctcttgagcctctcgtagatttgcctatagggatcaatattactcgctttcaaactctccttgcccttcatcggtcctacctgacaccaggcgacagctccagtgggtggacagctggtgcagcggcttactacgtcggctacctccaacccaggacttttttgctgaaatataatgagaatggtacgattactgacgccgtcgattgtgccctcgacccattgagcgaaactaaatgcaccctgaaaagctttacggttgagaaagggatatatcagacatctaattttcgcgttcaacccacggaatccattgtacgatttcccaacattaccaatttgtgtccttttggtgaggtatttaatgcaaccagattcgcgagtgtgtatgcctggaataggaaaagaatttctaactgtgtagcagactacagcgttctgtataatagtgcgtcctttagtacgttcaagtgctacggagtaagcccgacaaagttgaacgatttgtgtttcactaacgtctatgccgatagcttcgtcatcaggggagacgaagtaaggcaaattgcaccaggccagaccggaaaaatagccgactacaactataagttgccggacgatttcaccgggtgtgtcatcgcgtggaatagtaataacctggactccaaggtgggcggcaactacaactacctgtaccgattgttccgcaagtctaacctcaagccctttgaacgagacatatccaccgagatataccaagcgggaagcactccctgtaatggcgttgaaggtttcaactgctattttcctctccagtcctatggcttccaacctaccaatggagtaggctatcaaccgtatcgagtcgtggtgctgagtttcgagctcctgcacgctccagcgaccgtttgtggtcctaagaaaagtacaaacctcgtaaaaaataagtgcgtaaactttaactttaacggtctcacaggaacgggagttcttacagagtccaataagaagttcctgccctttcagcagtttgggcgcgacatcgcggacaccacagatgcagtcagggacccgcagactcttgagatcctggacatcaccccatgctcttttggcggtgtgagtgtcattacacccgggaccaacaccagcaatcaggttgctgtgctctatcaagacgtcaactgtacggaagtcccagttgcaatacacgcggaccagttgacgccgacgtggcgggtttattctaccggctcaaacgtctttcaaactagagccggttgtttgatcggggcggagcatgtgaacaattcatacgaatgtgatattcccataggagccggaatatgtgcttcttaccagacccaaaccaactctccgagacgagcccggtccgtagccagtcaaagcataattgcgtacaccatgagcctcggtgcagaaaactcagttgcgtactcaaataactccattgccatcccaacaaattttaccatatcagtgaccactgaaatccttccagtgagcatgactaaaacaagtgtagattgcacaatgtatatttgcggagactcaactgagtgctctaacctcctgttgcagtacggttcattctgtacccagctcaatcgcgcacttacgggaatagcagtagaacaagataagaatactcaggaagtcttcgctcaagtaaagcaaatctacaagacgccccccattaaggactttgggggttttaattttagccaaatactcccggaccccagtaaaccctctaagaggtcatttatagaagacctgttgtttaataaagttactctggccgatgctggctttattaaacaatacggtgattgtttgggcgacatcgcggcgcgggatctgatatgcgcccaaaaatttaacggtctcactgtgttgccgcctttgctcacagacgagatgatagctcagtacacatcagcgttgttggcgggtaccataacgtctggatggacattcggagcaggggcggccttgcaaataccattcgcaatgcaaatggcatacaggttcaacgggataggtgttacacagaatgtactttacgagaaccagaaattgattgcaaaccagtttaactctgcgattggcaaaattcaggatagcctcagctccaccgcgtctgcactgggaaaactccaagatgttgtgaaccaaaatgcccaagccctcaatacacttgtgaagcaactctccagcaatttcggtgcaataagtagtgtgctgaacgacatactttctcggttggacaaagtagaggcggaggtgcaaatcgacagactcataacaggtcgactgcaatcattgcaaacgtatgttactcagcaattgatacgggcggcggagattcgagcgtctgccaacctcgccgcgactaaaatgtctgagtgtgtgctcggtcagtcaaaacgcgtggacttctgtggaaagggataccatctcatgtccttccctcagtctgcccctcacggagtagtctttcttcatgtgacctatgttccagcacaggagaaaaattttaccacagcgcccgcgatatgccacgatggcaaagctcattttccgcgagagggagtatttgtctctaacggcacccattggtttgtaacccagcgcaatttttacgaacctcagataatcaccactgacaacacctttgttagtggtaattgtgatgtagtgatcgggatagtgaacaatacagtgtacgaccccctgcaacctgagctggactcctttaaagaagaactcgacaagtacttcaaaaaccacacttccccggatgtcgatttgggtgatatatctggcatcaatgcgagtgtagtcaacatacagaaggaaatcgacaggctcaatgaagtagcaaaaaacctgaatgaatccctcatagacctccaagagctgggcaaatatgagcagtacattaaatggccctggtacatttggctgggcttcatcgctggcctgatagcgatagtgatggttactattatgctctgctgtatgacgagttgttgcagctgcctcaaagggtgttgttcttgcggctcctgttgtaagttcgacgaggatgattcagagcccgttcttaagggtgtaaaactccattacactggaggaggaggttacccatacgatgttccagattacgcttagggcgcgcccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgcccaagaagaagaggaaggtgtccaatctcctgactgttcaccagaacctccctgcgctgccagtagatgccactagcgatgaggtcaggaaaaatctcatggatatgtttagggatagacaggcgttttctgaacacacctggaaaatgctgcttagcgtgtgccgatcctgggcagcctggtgtaagctgaacaatcgcaaatggttccccgccgagccggaggacgtgcgcgattacctgctgtatctccaggcaagagggctggctgtcaagactatccagcagcacttgggccaactgaatatgctgcatcgacgcagcgggctcccccggcctagcgattcaaacgcagtctcccttgttatgaggagaattagaaaggaaaacgtagatgcgggtgagagggctaagcaggctctcgcttttgagcggactgatttcgaccaggtcagatccctgatggagaacagcgatcggtgccaggacatcaggaacctcgcatttctgggaattgcatataacacacttctgcgcatagctgagatcgcccggatcagagtgaaagacatcagtcgaacggacggcggccggatgcttattcatattggacgcacaaagacattggtcagcaccgctggcgttgaaaaggccttgtccctgggcgtaacgaagctggtggaaagatggatctcagtgtccggcgtggctgacgaccctaataattacttgttctgtcgagtgagaaaaaacggagtcgccgcgccctctgccaccagccaattgagtacacgggcccttgaagggatctttgaggcaacccaccgactcatatacggagccaaggatgacagtggccagaggtatctcgcctggtcaggtcattctgctagggtgggggccgcacgagacatggcgcgggcaggagtctccataccagagattatgcaagctggaggttggacaaatgtgaacatcgttatgaactatatccgcaatcttgactctgaaaccggggccatggtgagactgctcgaagatggtgactgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 22 - DNA, 6089 bp, Artificial Sequence
  rAAV genome 8
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtaccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcaagcttcggtcaccagccaacaatgtgcggatggcgttacgacttactgggcctgatctcaccgctttagataccgcacactgggcaatacgaggtaaagccagtcgaattcgtttaaacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcatatgatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgcgccctgctcgacgcccttgccatcgagatgctggacaggcatcatacccacttctgccccctggaaggcgagtcatggcaagactttctgcggaacaacgccaagtcattccgctgtgctctcctctcacatagagacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagccattgagctgttcgaccggcagggagccgaacctgccttccttttcggcctggaactaatcatctgtggcctggagaaacagctaaagtgcgaaagcggcgggccagcagatgccctcgatgactttgatcttgacatgctccccgccgacgcactggatgattttgaccttgatatgctgcctgctgacgctcttgacgattttgaccttgacatgctccctggggctagcggatccgagggcagaggaagccttctaacatgcggtgacgtggaggagaatcccggcccttccgggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatatccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 23 - DNA, 7460 bp, Artificial Sequence
  rAAV genome 9
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtaccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcaagcttcattcaccagcttgcgaagtctacattagtaatatgaatgcgacctagaagagggcgcttaaaattgggagtggttgatgctctatactccatttggttttttcgtgcatcaccgcgataggctgacaagggtttaacattgaatagcaaggcacttccggtctcaatgaagggccgggaaaggtacgcgcgtggtatgggaggatcaaggggccaatagaaaggcttctccctcactcgctaggaggcaaatgcagaacaatggttactacattgatacgtgaaacatgtccaacggttgcccaaagtgttaagtgtctatcacccatagggccgtttcccggatataaacgccaggttgaatccgcatttgaagctaccatggatgagtctgggtcgagcgcgcctcatttattgcgtgagtagggtcgaccaagaaccgctagatgcgtcgctgtacaaatagttgtcgacagaccgtcgagtttagaaaatggtaccagcattttcggggaatctcaatcaagtatggattacggtgtttacactgtcctgcggctacccatggcctgaaatccagctcgtgtcaagccattgcctctccgggacgccgcatgaagtaatacatataccttgcacgggttcactgcggtccgttcagagtcgaccaaggacacaatcgagttccgatccgtatgctcgactaacttgtacccaacccccggagcttggcagctcctggggtatcatggagcctgtggttcatcccgtctatatcaaacttcgtcttgataaagccccccgctcgggagtaccagagaagatgtctactgagttgtgcgatccctgcacttcagctaaggaagctaccaatatttagtttctgagtctcacgacagacctcgcgcgtagattgccatgcgtagagctaacgagccagcggaaagcgtgaggcgcttttaagcatggcgagtaagtgatccaacgcttcggatatgactatatacttaggttcgatctcgtcccgagaatctaagcctcaacatctatgagttatgaggttagccgaaaaagcacgtggtggcgcccaccgactgttcccagactgtagctctttgttctgtcaaggcccgaccttcatcgcggccgattccttctgcggaccataccgtcctgatactttggtcatgtttccgttgtaggagtgaacccacttgcctttgcgtcttaataccaatgaaaaacctatgcactttgtacagggtaccatcgggattctgaaccctcagatagtggggatcccgggtatagacctttatctgcggtccaacttaggcataaacctccatgctaccttgtcagacccaccctgcacgaggtaaatatggtacgacgtccgacctggctcctggcgttctacgccgccacgtgttcgttgactgttgattggtagcacaaaagtaataccatggaattcgtttaaacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcatatgatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgcgccctgctcgacgcccttgccatcgagatgctggacaggcatcatacccacttctgccccctggaaggcgagtcatggcaagactttctgcggaacaacgccaagtcattccgctgtgctctcctctcacatagagacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagccattgagctgttcgaccggcagggagccgaacctgccttccttttcggcctggaactaatcatctgtggcctggagaaacagctaaagtgcgaaagcggcgggccagcagatgccctcgatgactttgatcttgacatgctccccgccgacgcactggatgattttgaccttgatatgctgcctgctgacgctcttgacgattttgaccttgacatgctccctggggctagcggatccgagggcagaggaagccttctaacatgcggtgacgtggaggagaatcccggcccttccgggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatatccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 24 - DNA, 8457 bp, Artificial Sequence
  rAAV genome 10
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtaccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcaagcttcattcaccagcttgcgaagtctacattagtaatatgaatgcgacctagaagagggcgcttaaaattgggagtggttgatgctctatactccatttggttttttcgtgcatcaccgcgataggctgacaagggtttaacattgaatagcaaggcacttccggtctcaatgaagggccgggaaaggtacgcgcgtggtatgggaggatcaaggggccaatagaaaggcttctccctcactcgctaggaggcaaatgcagaacaatggttactacattgatacgtgaaacatgtccaacggttgcccaaagtgttaagtgtctatcacccatagggccgtttcccggatataaacgccaggttgaatccgcatttgaagctaccatggatgagtctgggtcgagcgcgcctcatttattgcgtgagtagggtcgaccaagaaccgctagatgcgtcgctgtacaaatagttgtcgacagaccgtcgagtttagaaaatggtaccagcattttcggggaatctcaatcaagtatggattacggtgtttacactgtcctgcggctacccatggcctgaaatccagctcgtgtcaagccattgcctctccgggacgccgcatgaagtaatacatataccttgcacgggttcactgcggtccgttcagagtcgaccaaggacacaatcgagttccgatccgtatgctcgactaacttgtacccaacccccggagcttggcagctcctggggtatcatggagcctgtggttcatcccgtctatatcaaacttcgtcttgataaagccccccgctcgggagtaccagagaagatgtctactgagttgtgcgatccctgcacttcagctaaggaagctaccaatatttagtttctgagtctcacgacagacctcgcgcgtagattgccatgcgtagagctaacgagccagcggaaagcgtgaggcgcttttaagcatggcgagtaagtgatccaacgcttcggatatgactatatacttaggttcgatctcgtcccgagaatctaagcctcaacatctatgagttatgaggttagccgaaaaagcacgtggtggcgcccaccgactgttcccagactgtagctctttgttctgtcaaggcccgaccttcatcgcggccgattccttctgcggaccataccgtcctgatactttggtcatgtttccgttgtaggagtgaacccacttgcctttgcgtcttaataccaatgaaaaacctatgcactttgtacagggtaccatcgggattctgaaccctcagatagtggggatcccgggtatagacctttatctgcggtccaacttaggcataaacctccatgctaccttgtcagacccaccctgcacgaggtaaatatggtacgacgtccgacctggctcctggcgttctacgccgccacgtgttcgttgactgttgattggtagcacaaaagtaataccatggtccttgaaattcggctcagttagttcgagcgtaatgtcacaaatggcgcagaacggcaatgagtgtttgacactaggtggtgttcagttcggtaactgagagactgtgcggcatacttaattatacatttgaaacgcgcccaagtgacgctaggcaagtcagagcaggttcccgtgttagcttaatggtaaacatacaagtcgattgaagatgggtagggggcttcaattcgtccagcactctacggtacctccgagagcaagtagggcaccctgtagttcgaagcggaactatttcgtggggcgagcccacatcgtctcttctgcggatgacttaacacgttagggaggtggagttgattcgaacgatggttataaatcaaaaaaacggaacgctgtctggaggatgaatctaacggtgcgtaactcgatcactcactcgctattcgaactgcgcgaaagttcccagtgctcatacacttggttccgaggcctgtcctgatatatgaacccaaactagagcggggctgttgacgtttggagttgaaaaaatctaatattccaatcggcttcaacgtgcaccaccgcaggcggctgacgaggggctcacaccgagaaagtagactgttgcgcgttgggggtagcgccggctaacaaagacgcctggtacagcaggagtatcaaacccgtacaaaggcaacatcctcacttcggtgaatcgaagcgcggcatcagggttactttttggatacctgaaacaaaacccatcgtagtccttagacttggcacacttacacctgcagcgcgcgcatctggaaatagaggccaagttcgatccgtactccgactacgatgcaacagtgtggatgtgacgagcttcatttatacccttcgcgcgccggaccggcctccgcaaggcgcggcggtgcacaagcaattgacaactaaccaccgtgtattcgttatggcatcaggcagtttaagtcgagacaatagggaattcgtttaaacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcatatgatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgcgccctgctcgacgcccttgccatcgagatgctggacaggcatcatacccacttctgccccctggaaggcgagtcatggcaagactttctgcggaacaacgccaagtcattccgctgtgctctcctctcacatagagacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagccattgagctgttcgaccggcagggagccgaacctgccttccttttcggcctggaactaatcatctgtggcctggagaaacagctaaagtgcgaaagcggcgggccagcagatgccctcgatgactttgatcttgacatgctccccgccgacgcactggatgattttgaccttgatatgctgcctgctgacgctcttgacgattttgaccttgacatgctccctggggctagcggatccgagggcagaggaagccttctaacatgcggtgacgtggaggagaatcccggcccttccgggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatatccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 25 - DNA, 5902 bp, Artificial Sequence
  rAAV genome 1 vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggcccaacggtaccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 26 - DNA, 7520 bp, Artificial Sequence
  rAAV genome 2 vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatcccctagctaagggctagcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggcccaacggtaccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 27 - DNA, 9554 bp, Artificial Sequence
  rAAV genome 3 vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtaccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcaagcttataaataattcaataaagatgtcgttttgctagtatacgtctaggcgtcacccgccatctctgtgcaggtgggccgacgagacattgtccctgatttctccactactaatagcacacacggggcaataccagcacaagctagtctcgcgggaacgctcgtcagcatacgaaagagcttaaggcacgccaattcgcactgtcagggtcacttgggtgttttgcactaccgtcaggtacgctagtatgcgttcttccttccagaggtatgtggctgcgtggtcaaaagtgcggcattcgtatttgctcctcgtgtttactctcacaaacttgacctggagatcaaggagatgcttcttgtggaactggacaacgcatcaacgcaacggatctacgttacagcgtgcatagtgaaaacggagttgctgacgacgaaagcgacattgggatctgtctgttgtcattcgcggaaaacatccgttcacgaggcggacactgattgacacggttttgcagaaggttaggggaataggttaaattgagtggcttaaaaatgctatgtctgggattaaagtgtagtaaactgtgattaacggagacggttttaagacaggagttcgcaaaatcaagcggggtcattacaacggttattcctggtggtttaggcgtacaatgtcctgaagaatatttaagaaaaaagcacccctcgtcgcctagaattacctaccgcggtcgaccataccttcgattatcgcgcccactctcccattagtcggcagaggtggttgtgttgcgatagcccagtatgatattctaaggcgttacgctgatgaatattctacggaattgccataggcgttgaacgctacacggacgatacgaatttatgtatagagcgggtcatcgaaaggttatactcttgtagttaacatgtagcccggccctattagtacagcagtgccttgaatgacgaattcgtttaaacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcatatgatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgcgccctgctcgacgcccttgccatcgagatgctggacaggcatcatacccacttctgccccctggaaggcgagtcatggcaagactttctgcggaacaacgccaagtcattccgctgtgctctcctctcacatagagacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagccattgagctgttcgaccggcagggagccgaacctgccttccttttcggcctggaactaatcatctgtggcctggagaaacagctaaagtgcgaaagcggcgggccagcagatgccctcgatgactttgatcttgacatgctccccgccgacgcactggatgattttgaccttgatatgctgcctgctgacgctcttgacgattttgaccttgacatgctccctggggctagcggatccgagggcagaggaagccttctaacatgcggtgacgtggaggagaatcccggcccttccgggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatatccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 28 - DNA, 9460 bp, Artificial Sequence
  rAAV genome 4 vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggacggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 29 - DNA, 7780 bp, Artificial Sequence
  rAAV genome 5 vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaacactagaattccctaggccggccacgcgttttttttctcgttttactcacaaatacttaagtatgtaagtagatacacttcacccacataatagagaatactctatacatgtattcatgggtcaaaggggtccctgagatacacacaattcatactcatccctctgaacactgcaagcgtcgtgtggagccatttgctttcccacacactcgctttctgccgcagcaaggctggaccctgctggagtggtgatgtgcaggaactgcctcatagctcaggtggtaaagttatttggctgggaactgagtctctccccacatgctgtcacacccttcccggccctgacaaacacaaagagatgcatcccattgagtcacccacttggctgaaccgtgtacctggtaacctccttgggaggttctttggagggtatttttctcctctacacaaagcatgcctgcgaagtaagctgagaatgacaaaaacaggaaagcccaatcttgcagagacagaggggccaaggcctgcatgataatgcggcccaaattcatagctcagacccaaaagaaaacaaatgttccattctttctgtgtccacctctaaactaagcaatacagtgagggcagtaaattgcaattacaagtaagaactttagcagttgctgatcataaaatgaaaagggacaaatatgtttatccttaacagggtcacaaataattccacagctgttaattttggcagccacgactctgcaggcagtatgggctcatggtatactatcttcattgtcaccttggacaaagatcctgttggccaaaggcacacaggcatcttcaggaaaactagatttaaatggtaagattacctatgcttatgtttttaagctccaaagactactggttctgggaaggactgggtcactatgtgaccttgtgtaggtctcttttggccctgagtccaggagggctctgtgactccaggttgtggctgcgtctggcagctaacaattgtttagtttacagtgtccttcctcttcatcttgtttgcattcagttgcccatgcctgcctctggggggaggtgggaaggaatgtggctcctgggaggagcccagctccatgtgccattctgaggagccaggctgcacgcccctgggccatagctgctggtggatggatgagtacactggcagaggcactgggaagggaagacagaattgttggccaatagtccccttttactgattttcttgtcatttttgtgtttgttttattataaagataacagctgtatttgagaaagaaagctcaaactataaagaaaatttaaaattgccctgtcttactccaactccattccctgactaggtgagccagacgctggccttatctcatttaccatctcagagccatctgaagggggagaagggaaccgggccccaggagggagaaagtcatcaaacctcccacatctgtgaccagcctcagtgccatactttttctatggagggccctgtccaatggaactgagcacagaccagataaaagaactgggcacccagtggcctcagtccagggcctggagttcaaactttactggaaacaaaggggccgagagagactggggaaagaactactagaaaggggctggtgcccccatggggctgtgggttttggagccgcgtgcccccacctgagcctcagggggcccggagtgtccacaccagtggacctttcgagaaatggctgggccattgtgcagaagaatgcccggaaatcccgcgcctccctcctccagcaaggatgggggctcttcctcctggccaggaaactccaagttggcttccggagggtggcctgggggctggggtgccagggacaccatcgccactggtgggagggcagggcacagcccctccgtgtccctttgtctctcctgtctgaaggccagagcaggctgctaggcctggggccaccactgcccctgggtgctacacccagtgtgctgggtcactgggaacttcctgaagtggtgtcacctgaactggacccccaaggatggggtgcgggcagtaccgcaggaagaggagcagcccctgtgaagattgagaggtctgggaagcccctgcggcttgggagagtgggggtcgccaggcagggggaaagcccctgtgccaccgctttttgccagagactcaggctccagagaggcagtgagtggcatggggggtgaggctggggccctgggcctgacctccacacgcctgcctggcctctctgtttgccatgggatgagagagacagtgctgggactcagagcggggctggagagtgagagtgcgagaaagggcctgggtggggcttggaccccggggcgggctttctggagagcccccctacgagggcctctacggcggtgacggggtggggggcttctgcaaaccttggtcagggaagtggagctggctcgagtggaagagaccacccggctcagtcggggatgtgggagtggactgggtggtgcagactgggggtcgagcgccttctgaagtgacggggccgggacgcgcagggaggcggcccaagaagcgcgccctaggccagcccagaatgcgctcggccgcgactaggacaacggcgggtggggctgggggcggctgccgggcggggagcggtcccgcgccctcagctacccctcaagagccgttgtttccctaacttcagctgccagaggctctgtgattggctgcggcacgatgacccgcgcacggattggctgcttcgggccggggggccgggcccgggggacagaatccgcccccgaaccttcaaagagggtaccccccggcaggagctggcagacccaggaggtgcgacagacccgcggggcaaacggactggggccaagagccgggagcgcgggcgcaaaggcaccagggcccgcccagggcgccgcgcagcacggccttgggggttctgcgggccttcgggtggcgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 30 - DNA, 6373 bp, Artificial Sequence
  rAAV genome 6 vector
gggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtaccggatccaccatgcccaagaagaagaggaaggtgtccaatctcctgactgttcaccagaacctccctgcgctgccagtagatgccactagcgatgaggtcaggaaaaatctcatggatatgtttagggatagacaggcgttttctgaacacacctggaaaatgctgcttagcgtgtgccgatcctgggcagcctggtgtaagctgaacaatcgcaaatggttccccgccgagccggaggacgtgcgcgattacctgctgtatctccaggcaagagggctggctgtcaagactatccagcagcacttgggccaactgaatatgctgcatcgacgcagcgggctcccccggcctagcgattcaaacgcagtctcccttgttatgaggagaattagaaaggaaaacgtagatgcgggtgagagggctaagcaggctctcgcttttgagcggactgatttcgaccaggtcagatccctgatggagaacagcgatcggtgccaggacatcaggaacctcgcatttctgggaattgcatataacacacttctgcgcatagctgagatcgcccggatcagagtgaaagacatcagtcgaacggacggcggccggatgcttattcatattggacgcacaaagacattggtcagcaccgctggcgttgaaaaggccttgtccctgggcgtaacgaagctggtggaaagatggatctcagtgtccggcgtggctgacgaccctaataattacttgttctgtcgagtgagaaaaaacggagtcgccgcgccctctgccaccagccaattgagtacacgggcccttgaagggatctttgaggcaacccaccgactcatatacggagccaaggatgacagtggccagaggtatctcgcctggtcaggtcattctgctagggtgggggccgcacgagacatggcgcgggcaggagtctccataccagagattatgcaagctggaggttggacaaatgtgaacatcgttatgaactatatccgcaatcttgactctgaaaccggggccatggtgagactgctcgaagatggtgactgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggg

SEQ ID NO: 31 - DNA, 10820 bp, Artificial Sequence
  rAAV genome 7 vector
ttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtaccggatccgttaacaccatgttcgtattccttgtcctcctccccctcgtcagctcccagtgtgtcaatctgacaactcgaactcagctcccacccgcatacaccaatagttttacccgaggagtctattatcccgacaaagttttcaggtctagtgtgttgcatagcacgcaggacctgttcttgccgtttttttcaaatgtgacatggttccatgccatccatgtctctggtacgaacgggaccaaaaggtttgataaccccgttcttccatttaacgacggggtttatttcgcctctactgaaaagtctaatataatacgaggttggatttttgggacaacactggactctaaaacgcaatctctcctcatagtaaataatgcgacgaatgtcgtaatcaaagtatgtgagtttcagttttgcaacgacccttttcttggtgtatactatcacaagaacaacaagtcttggatggaaagtgaatttagagtctacagctccgcaaacaactgtacctttgagtacgtcagtcagcccttccttatggacctcgaaggcaaacaggggaacttcaagaatctgcgcgagttcgtgttcaagaatatcgacggctatttcaagatatattcaaaacatacaccaatcaatttggtacgcgatttgccgcagggcttctctgctcttgagcctctcgtagatttgcctatagggatcaatattactcgctttcaaactctccttgcccttcatcggtcctacctgacaccaggcgacagctccagtgggtggacagctggtgcagcggcttactacgtcggctacctccaacccaggacttttttgctgaaatataatgagaatggtacgattactgacgccgtcgattgtgccctcgacccattgagcgaaactaaatgcaccctgaaaagctttacggttgagaaagggatatatcagacatctaattttcgcgttcaacccacggaatccattgtacgatttcccaacattaccaatttgtgtccttttggtgaggtatttaatgcaaccagattcgcgagtgtgtatgcctggaataggaaaagaatttctaactgtgtagcagactacagcgttctgtataatagtgcgtcctttagtacgttcaagtgctacggagtaagcccgacaaagttgaacgatttgtgtttcactaacgtctatgccgatagcttcgtcatcaggggagacgaagtaaggcaaattgcaccaggccagaccggaaaaatagccgactacaactataagttgccggacgatttcaccgggtgtgtcatcgcgtggaatagtaataacctggactccaaggtgggcggcaactacaactacctgtaccgattgttccgcaagtctaacctcaagccctttgaacgagacatatccaccgagatataccaagcgggaagcactccctgtaatggcgttgaaggtttcaactgctattttcctctccagtcctatggcttccaacctaccaatggagtaggctatcaaccgtatcgagtcgtggtgctgagtttcgagctcctgcacgctccagcgaccgtttgtggtcctaagaaaagtacaaacctcgtaaaaaataagtgcgtaaactttaactttaacggtctcacaggaacgggagttcttacagagtccaataagaagttcctgccctttcagcagtttgggcgcgacatcgcggacaccacagatgcagtcagggacccgcagactcttgagatcctggacatcaccccatgctcttttggcggtgtgagtgtcattacacccgggaccaacaccagcaatcaggttgctgtgctctatcaagacgtcaactgtacggaagtcccagttgcaatacacgcggaccagttgacgccgacgtggcgggtttattctaccggctcaaacgtctttcaaactagagccggttgtttgatcggggcggagcatgtgaacaattcatacgaatgtgatattcccataggagccggaatatgtgcttcttaccagacccaaaccaactctccgagacgagcccggtccgtagccagtcaaagcataattgcgtacaccatgagcctcggtgcagaaaactcagttgcgtactcaaataactccattgccatcccaacaaattttaccatatcagtgaccactgaaatccttccagtgagcatgactaaaacaagtgtagattgcacaatgtatatttgcggagactcaactgagtgctctaacctcctgttgcagtacggttcattctgtacccagctcaatcgcgcacttacgggaatagcagtagaacaagataagaatactcaggaagtcttcgctcaagtaaagcaaatctacaagacgccccccattaaggactttgggggttttaattttagccaaatactcccggaccccagtaaaccctctaagaggtcatttatagaagacctgttgtttaataaagttactctggccgatgctggctttattaaacaatacggtgattgtttgggcgacatcgcggcgcgggatctgatatgcgcccaaaaatttaacggtctcactgtgttgccgcctttgctcacagacgagatgatagctcagtacacatcagcgttgttggcgggtaccataacgtctggatggacattcggagcaggggcggccttgcaaataccattcgcaatgcaaatggcatacaggttcaacgggataggtgttacacagaatgtactttacgagaaccagaaattgattgcaaaccagtttaactctgcgattggcaaaattcaggatagcctcagctccaccgcgtctgcactgggaaaactccaagatgttgtgaaccaaaatgcccaagccctcaatacacttgtgaagcaactctccagcaatttcggtgcaataagtagtgtgctgaacgacatactttctcggttggacaaagtagaggcggaggtgcaaatcgacagactcataacaggtcgactgcaatcattgcaaacgtatgttactcagcaattgatacgggcggcggagattcgagcgtctgccaacctcgccgcgactaaaatgtctgagtgtgtgctcggtcagtcaaaacgcgtggacttctgtggaaagggataccatctcatgtccttccctcagtctgcccctcacggagtagtctttcttcatgtgacctatgttccagcacaggagaaaaattttaccacagcgcccgcgatatgccacgatggcaaagctcattttccgcgagagggagtatttgtctctaacggcacccattggtttgtaacccagcgcaatttttacgaacctcagataatcaccactgacaacacctttgttagtggtaattgtgatgtagtgatcgggatagtgaacaatacagtgtacgaccccctgcaacctgagctggactcctttaaagaagaactcgacaagtacttcaaaaaccacacttccccggatgtcgatttgggtgatatatctggcatcaatgcgagtgtagtcaacatacagaaggaaatcgacaggctcaatgaagtagcaaaaaacctgaatgaatccctcatagacctccaagagctgggcaaatatgagcagtacattaaatggccctggtacatttggctgggcttcatcgctggcctgatagcgatagtgatggttactattatgctctgctgtatgacgagttgttgcagctgcctcaaagggtgttgttcttgcggctcctgttgtaagttcgacgaggatgattcagagcccgttcttaagggtgtaaaactccattacactggaggaggaggttacccatacgatgttccagattacgcttagggcgcgcccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgcccaagaagaagaggaaggtgtccaatctcctgactgttcaccagaacctccctgcgctgccagtagatgccactagcgatgaggtcaggaaaaatctcatggatatgtttagggatagacaggcgttttctgaacacacctggaaaatgctgcttagcgtgtgccgatcctgggcagcctggtgtaagctgaacaatcgcaaatggttccccgccgagccggaggacgtgcgcgattacctgctgtatctccaggcaagagggctggctgtcaagactatccagcagcacttgggccaactgaatatgctgcatcgacgcagcgggctcccccggcctagcgattcaaacgcagtctcccttgttatgaggagaattagaaaggaaaacgtagatgcgggtgagagggctaagcaggctctcgcttttgagcggactgatttcgaccaggtcagatccctgatggagaacagcgatcggtgccaggacatcaggaacctcgcatttctgggaattgcatataacacacttctgcgcatagctgagatcgcccggatcagagtgaaagacatcagtcgaacggacggcggccggatgcttattcatattggacgcacaaagacattggtcagcaccgctggcgttgaaaaggccttgtccctgggcgtaacgaagctggtggaaagatggatctcagtgtccggcgtggctgacgaccctaataattacttgttctgtcgagtgagaaaaaacggagtcgccgcgccctctgccaccagccaattgagtacacgggcccttgaagggatctttgaggcaacccaccgactcatatacggagccaaggatgacagtggccagaggtatctcgcctggtcaggtcattctgctagggtgggggccgcacgagacatggcgcgggcaggagtctccataccagagattatgcaagctggaggttggacaaatgtgaacatcgttatgaactatatccgcaatcttgactctgaaaccggggccatggtgagactgctcgaagatggtgactgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggcctt

SEQ ID NO: 32 - DNA, 8686 bp, Artificial Sequence
  rAAV genome 8 vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtaccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcaagcttcggtcaccagccaacaatgtgcggatggcgttacgacttactgggcctgatctcaccgctttagataccgcacactgggcaatacgaggtaaagccagtcgaattcgtttaaacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcatatgatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgcgccctgctcgacgcccttgccatcgagatgctggacaggcatcatacccacttctgccccctggaaggcgagtcatggcaagactttctgcggaacaacgccaagtcattccgctgtgctctcctctcacatagagacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagccattgagctgttcgaccggcagggagccgaacctgccttccttttcggcctggaactaatcatctgtggcctggagaaacagctaaagtgcgaaagcggcgggccagcagatgccctcgatgactttgatcttgacatgctccccgccgacgcactggatgattttgaccttgatatgctgcctgctgacgctcttgacgattttgaccttgacatgctccctggggctagcggatccgagggcagaggaagccttctaacatgcggtgacgtggaggagaatcccggcccttccgggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatatccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 33 - DNA, 10057 bp, Artificial Sequence
  rAAV genome 9 vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtaccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcaagcttcattcaccagcttgcgaagtctacattagtaatatgaatgcgacctagaagagggcgcttaaaattgggagtggttgatgctctatactccatttggttttttcgtgcatcaccgcgataggctgacaagggtttaacattgaatagcaaggcacttccggtctcaatgaagggccgggaaaggtacgcgcgtggtatgggaggatcaaggggccaatagaaaggcttctccctcactcgctaggaggcaaatgcagaacaatggttactacattgatacgtgaaacatgtccaacggttgcccaaagtgttaagtgtctatcacccatagggccgtttcccggatataaacgccaggttgaatccgcatttgaagctaccatggatgagtctgggtcgagcgcgcctcatttattgcgtgagtagggtcgaccaagaaccgctagatgcgtcgctgtacaaatagttgtcgacagaccgtcgagtttagaaaatggtaccagcattttcggggaatctcaatcaagtatggattacggtgtttacactgtcctgcggctacccatggcctgaaatccagctcgtgtcaagccattgcctctccgggacgccgcatgaagtaatacatataccttgcacgggttcactgcggtccgttcagagtcgaccaaggacacaatcgagttccgatccgtatgctcgactaacttgtacccaacccccggagcttggcagctcctggggtatcatggagcctgtggttcatcccgtctatatcaaacttcgtcttgataaagccccccgctcgggagtaccagagaagatgtctactgagttgtgcgatccctgcacttcagctaaggaagctaccaatatttagtttctgagtctcacgacagacctcgcgcgtagattgccatgcgtagagctaacgagccagcggaaagcgtgaggcgcttttaagcatggcgagtaagtgatccaacgcttcggatatgactatatacttaggttcgatctcgtcccgagaatctaagcctcaacatctatgagttatgaggttagccgaaaaagcacgtggtggcgcccaccgactgttcccagactgtagctctttgttctgtcaaggcccgaccttcatcgcggccgattccttctgcggaccataccgtcctgatactttggtcatgtttccgttgtaggagtgaacccacttgcctttgcgtcttaataccaatgaaaaacctatgcactttgtacagggtaccatcgggattctgaaccctcagatagtggggatcccgggtatagacctttatctgcggtccaacttaggcataaacctccatgctaccttgtcagacccaccctgcacgaggtaaatatggtacgacgtccgacctggctcctggcgttctacgccgccacgtgttcgttgactgttgattggtagcacaaaagtaataccatggaattcgtttaaacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcatatgatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgcgccctgctcgacgcccttgccatcgagatgctggacaggcatcatacccacttctgccccctggaaggcgagtcatggcaagactttctgcggaacaacgccaagtcattccgctgtgctctcctctcacatagagacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagccattgagctgttcgaccggcagggagccgaacctgccttccttttcggcctggaactaatcatctgtggcctggagaaacagctaaagtgcgaaagcggcgggccagcagatgccctcgatgactttgatcttgacatgctccccgccgacgcactggatgattttgaccttgatatgctgcctgctgacgctcttgacgattttgaccttgacatgctccctggggctagcggatccgagggcagaggaagccttctaacatgcggtgacgtggaggagaatcccggcccttccgggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatatccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 34 - DNA, 11054 bp, Artificial Sequence
  rAAV genome 10 vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtaccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcaagcttcattcaccagcttgcgaagtctacattagtaatatgaatgcgacctagaagagggcgcttaaaattgggagtggttgatgctctatactccatttggttttttcgtgcatcaccgcgataggctgacaagggtttaacattgaatagcaaggcacttccggtctcaatgaagggccgggaaaggtacgcgcgtggtatgggaggatcaaggggccaatagaaaggcttctccctcactcgctaggaggcaaatgcagaacaatggttactacattgatacgtgaaacatgtccaacggttgcccaaagtgttaagtgtctatcacccatagggccgtttcccggatataaacgccaggttgaatccgcatttgaagctaccatggatgagtctgggtcgagcgcgcctcatttattgcgtgagtagggtcgaccaagaaccgctagatgcgtcgctgtacaaatagttgtcgacagaccgtcgagtttagaaaatggtaccagcattttcggggaatctcaatcaagtatggattacggtgtttacactgtcctgcggctacccatggcctgaaatccagctcgtgtcaagccattgcctctccgggacgccgcatgaagtaatacatataccttgcacgggttcactgcggtccgttcagagtcgaccaaggacacaatcgagttccgatccgtatgctcgactaacttgtacccaacccccggagcttggcagctcctggggtatcatggagcctgtggttcatcccgtctatatcaaacttcgtcttgataaagccccccgctcgggagtaccagagaagatgtctactgagttgtgcgatccctgcacttcagctaaggaagctaccaatatttagtttctgagtctcacgacagacctcgcgcgtagattgccatgcgtagagctaacgagccagcggaaagcgtgaggcgcttttaagcatggcgagtaagtgatccaacgcttcggatatgactatatacttaggttcgatctcgtcccgagaatctaagcctcaacatctatgagttatgaggttagccgaaaaagcacgtggtggcgcccaccgactgttcccagactgtagctctttgttctgtcaaggcccgaccttcatcgcggccgattccttctgcggaccataccgtcctgatactttggtcatgtttccgttgtaggagtgaacccacttgcctttgcgtcttaataccaatgaaaaacctatgcactttgtacagggtaccatcgggattctgaaccctcagatagtggggatcccgggtatagacctttatctgcggtccaacttaggcataaacctccatgctaccttgtcagacccaccctgcacgaggtaaatatggtacgacgtccgacctggctcctggcgttctacgccgccacgtgttcgttgactgttgattggtagcacaaaagtaataccatggtccttgaaattcggctcagttagttcgagcgtaatgtcacaaatggcgcagaacggcaatgagtgtttgacactaggtggtgttcagttcggtaactgagagactgtgcggcatacttaattatacatttgaaacgcgcccaagtgacgctaggcaagtcagagcaggttcccgtgttagcttaatggtaaacatacaagtcgattgaagatgggtagggggcttcaattcgtccagcactctacggtacctccgagagcaagtagggcaccctgtagttcgaagcggaactatttcgtggggcgagcccacatcgtctcttctgcggatgacttaacacgttagggaggtggagttgattcgaacgatggttataaatcaaaaaaacggaacgctgtctggaggatgaatctaacggtgcgtaactcgatcactcactcgctattcgaactgcgcgaaagttcccagtgctcatacacttggttccgaggcctgtcctgatatatgaacccaaactagagcggggctgttgacgtttggagttgaaaaaatctaatattccaatcggcttcaacgtgcaccaccgcaggcggctgacgaggggctcacaccgagaaagtagactgttgcgcgttgggggtagcgccggctaacaaagacgcctggtacagcaggagtatcaaacccgtacaaaggcaacatcctcacttcggtgaatcgaagcgcggcatcagggttactttttggatacctgaaacaaaacccatcgtagtccttagacttggcacacttacacctgcagcgcgcgcatctggaaatagaggccaagttcgatccgtactccgactacgatgcaacagtgtggatgtgacgagcttcatttatacccttcgcgcgccggaccggcctccgcaaggcgcggcggtgcacaagcaattgacaactaaccaccgtgtattcgttatggcatcaggcagtttaagtcgagacaatagggaattcgtttaaacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcatatgatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgcgccctgctcgacgcccttgccatcgagatgctggacaggcatcatacccacttctgccccctggaaggcgagtcatggcaagactttctgcggaacaacgccaagtcattccgctgtgctctcctctcacatagagacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagccattgagctgttcgaccggcagggagccgaacctgccttccttttcggcctggaactaatcatctgtggcctggagaaacagctaaagtgcgaaagcggcgggccagcagatgccctcgatgactttgatcttgacatgctccccgccgacgcactggatgattttgaccttgatatgctgcctgctgacgctcttgacgattttgaccttgacatgctccctggggctagcggatccgagggcagaggaagccttctaacatgcggtgacgtggaggagaatcccggcccttccgggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatatccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 35 - DNA, 20 bp, Artificial Sequence
  genome 1 forward
acgtccaggagcgcaccatc

SEQ ID NO: 36 - DNA, 20 bp, Artificial Sequence
  genome 1 reverse
tgcggttcaccagggtgtcg

SEQ ID NO: 37 - DNA, 20 bp, Artificial Sequence
  genome 2 forward
acgtccaggagcgcaccatc

SEQ ID NO: 38 - DNA, 20 bp, Artificial Sequence
  genome 2 reverse
tgcggttcaccagggtgtcg

SEQ ID NO: 39 - DNA, 20 bp, Artificial Sequence
  genome 3 forward
acgtccaggagcgcaccatc

SEQ ID NO: 40 - DNA, 20 bp, Artificial Sequence
  genome 3 reverse
tgcggttcaccagggtgtcg

SEQ ID NO: 41 - DNA, 20 bp, Artificial Sequence
  genome 4 forward
acgtccaggagcgcaccatc

SEQ ID NO: 42 - DNA, 20 bp, Artificial Sequence
  genome 4 reverse
tgcggttcaccagggtgtcg

SEQ ID NO: 43 - DNA, 20 bp, Artificial Sequence
  genome 5 forward
acgtccaggagcgcaccatc

SEQ ID NO: 44 - DNA, 20 bp, Artificial Sequence
  genome 5 reverse
tgcggttcaccagggtgtcg

SEQ ID NO: 45 - DNA, 20 bp, Artificial Sequence
  genome 6 forward
ggctgttgggcactgacaat

SEQ ID NO: 46 - DNA, 20 bp, Artificial Sequence
  genome 6 reverse
ccgaagggacgtagcagaag

SEQ ID NO: 47 - DNA, 20 bp, Artificial Sequence
  genome 7 forward
ggctgttgggcactgacaat

SEQ ID NO: 48 - DNA, 20 bp, Artificial Sequence
  genome 7 reverse
ccgaagggacgtagcagaag

SEQ ID NO: 49 - DNA, 20 bp, Artificial Sequence
  genome 8 forward
acgtccaggagcgcaccatc

SEQ ID NO: 50 - DNA, 20 bp, Artificial Sequence
  genome 8 reverse
tgcggttcaccagggtgtcg

SEQ ID NO: 51 - DNA, 20 bp, Artificial Sequence
  genome 9 forward
acgtccaggagcgcaccatc

SEQ ID NO: 52 - DNA, 20 bp, Artificial Sequence
  genome 9 reverse
tgcggttcaccagggtgtcg

SEQ ID NO: 53 - DNA, 20 bp, Artificial Sequence
  genome 10 forward
acgtccaggagcgcaccatc

SEQ ID NO: 54 - DNA, 20 bp, Artificial Sequence
  genome 10 reverse
tgcggttcaccagggtgtcg

SEQ ID NO: 55 - DNA, 500 bp, Artificial Sequence
  AAV9d3
cacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcgggcagctctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaagtttaaacctgcaggacta

SEQ ID NO: 56 - DNA, 515 bp, Artificial Sequence
  AAV9d6
cacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggacta

SEQ ID NO: 57 - DNA, 1847 bp, Artificial Sequence; identical to SEQ ID NO: 1
  djrAAV9 [d6-(G/S)3-wt]

SEQ ID NO: 58 - DNA, 1829 bp, Artificial Sequence; identical to SEQ ID NO: 2
  djrAAV9 [d6-(G/S)3-d6]

SEQ ID NO: 59 - DNA, 1847 bp, Artificial Sequence
  djrAAV9 [wt-(G/S)9-d6]
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggaggcgggagtggcggagggggatcagcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 60 - DNA, 1841 bp, Artificial Sequence
  djrAAV9 [wt-(G/S)7-d6]
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggaggttcaggtggcgggtcagcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 61 - DNA, 1835 bp, Artificial Sequence
  djrAAV9 [wt-(G/S)5-d6]
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggtgggggcggatctgcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 62 - DNA, 1823 bp, Artificial Sequence
  djrAAV9 [wt-(G/S)3-d6]
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggtggctctggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 63 - DNA, 1817 bp, Artificial Sequence
  djrAAV9 [wt-(G/S)3-d6]
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggtggctctggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 64 - DNA, 1811 bp, Artificial Sequence
  djrAAV9 [wt-(G/S)3-d6]
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggtggctctccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 65 - DNA, 1805 bp, Artificial Sequence
  djrAAV9 [wt-(G/S)3-d6]
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggtggctctgcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 66 - DNA, 1799 bp, Artificial Sequence
  djrAAV9 [wt-(G/S)3-d6]
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggtggctctaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 67 - DNA, 1904 bp, Artificial Sequence
  djrAAV9 [wt-(G)-cpVP3d6]
aggaaaacagcaagcgctggaacccggagatccagtacacttccaactattacaagtctaataatgttgaatttgctgttaatactgaaggtgtatatagtgaaccccgccccattggcaccagatacctgactcgtaatctgggtcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctggggcatggcaccggcagcaccggcagcggcagctccgggcacggaactgggtctacgggttctggttctagtcccgggggagcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattccttaaacgcgtttgcttgttaatcaataaaccgtttaattcgtttcagttgaactttggtctctgcgaagggcgaattcgtttaaacctgcaggacta

SEQ ID NO: 68 - DNA, 1874 bp, Artificial Sequence
  djrAAV9 [wt-(T2A)-d6]
aggaaaacagcaagcgctggaacccggagatccagtacacttccaactattacaagtctaataatgttgaatttgctgttaatactgaaggtgtatatagtgaaccccgccccattggcaccagatacctgactcgtaatctggagggcagaggaagccttctaacatgcggtgacgtggaggagaatcccggccctgcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctggcagcctgaactctttcatcacccagtattctactggccaagtcagcgtggagatcgagtgggagctgcagaaggaaaatagtaaacgctggaatccagagatccagtacacctctaactactataagtcaaataatgtagagttcgcagtgaatacggagggagtctactctgaaccacgcccaatcggcactcgctatctgaccaggaatctgtaaacgcgtttgcttgttaatcaataaaccgtttaattcgtttcagttgaactttggtctctgcgaagggcgaattcgtttaaacctgcaggacta

SEQ ID NO: 69 - DNA, 262 bp, Artificial Sequence
  TBSV_2-82i-VP3
ttcggatccgcggccgccatggcaatggtgaaacgcaataacaatactggaatgatccccgtgtccacgaaacaacttctggctctgggtgcagcggcaggggcaactgcactgcaagggtttgttaagaacaatggtatggctatcgtagaaggggcagtagacctgactaaacgagcctataaagcagtccggcgcaggggcggtaagaaacagcagatgattaaccacgtaggcggcactggggtgggtagttcctcgg

SEQ ID NO: 70 - DNA, 336 bp, Artificial Sequence
  TBSV_2-101i-VP3
ttcggatccgcggccgccatggctatggtcaagagaaacaacaacaccggcatgataccagtatcaacgaaacaacttctcgcgctcggagccgcagcgggagcgacggccctccaaggcttcgtaaaaaacaatgggatggcaatagttgagggggcggttgatcttacgaaacgcgcctacaaggcggttagacggaggggcggaaagaagcaacaaatgattaatcatgtaggcggaactggtggcgcgatcatggcacctgtcgccgtgacccgccaacttgtaggtagcaaacccaagttcacagggcggactagcggggacagagtcatc

SEQ ID NO: 71 - DNA, 660 bp, Artificial Sequence
  TBSV_74-82i-CAP
agagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggtactggtggagcaatcatggcacctgtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtc

SEQ ID NO: 72 - DNA, 627 bp, Artificial Sequence
  TBSV-74-82s-CAP
agagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcggtactggtggagcaatcatggcacctgtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtc

SEQ ID NO: 73 - DNA, 651 bp, Artificial Sequence
  TBSV-77-82i-CAP
agagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagcaatcatggcacctgtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtc

SEQ ID NO: 74 - DNA, 678 bp, Artificial Sequence
  TBSV_69-82i-CAP
agagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatatgattaaccacgtaggtggtactggtggagcaatcatggcacctgtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtc

SEQ ID NO: 75 - DNA, 681 bp, Artificial Sequence
  TBSV_69-83i-CAP
agagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatatgattaaccacgtaggtggtactggtggagcaatcatggcacctgtggtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtc

SEQ ID NO: 76 - DNA, 732 bp, Artificial Sequence
  TBSV_50-82i-CAP
agagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatctgacgaagcgagcttacaaggccgttagacgacgaggtggaaagaaacagcaaatgattaaccatgtcgggggtacaggaggagctataatggcgcctgtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtc

SEQ ID NO: 77 - DNA, 681 bp, Artificial Sequence
  TBSV-50-82s-CAP
agagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacactgacgaagcgagcttacaaggccgttagacgacgaggtggaaagaaacagcaaatgattaaccatgtcgggggtacaggaggagctataatggcgcctgtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtc

SEQ ID NO: 78 - DNA, 627 bp, Artificial Sequence; identical to SEQ ID NO: 12
  TBSV-69-82s-CAPd6

SEQ ID NO: 79 - DNA, 660 bp, Artificial Sequence; identical to SEQ ID NO: 71
  TBSV_74-82i-CAPd6

SEQ ID NO: 80 - DNA, 627 bp, Artificial Sequence; identical to SEQ ID NO: 72
  TBSV-74-82s-CAPd6

SEQ ID NO: 81 - DNA, 651 bp, Artificial Sequence; identical to SEQ ID NO: 73
  TBSV-77-82i-CAPd6

SEQ ID NO: 82 - DNA, 678 bp, Artificial Sequence; identical to SEQ ID NO: 74
  TBSV_69-82i-CAPd6

SEQ ID NO: 83 - DNA, 681 bp, Artificial Sequence; identical to SEQ ID NO: 75
  TBSV_69-83i-CAPd6

SEQ ID NO: 84 - DNA, 732 bp, Artificial Sequence; identical to SEQ ID NO: 76
  TBSV_50-82i-CAPd6

SEQ ID NO: 85 - DNA, 681 bp, Artificial Sequence; identical to SEQ ID NO: 77
  TBSV-50-82s-CAPd6

SEQ ID NO: 86 - protein, 29 aa, Artificial Sequence
  linker in XL.D0-AAV9
YLTRNLGGSASGGGAPVADNNEGADGVGS

SEQ ID NO: 87 - DNA, 3596 bp, Artificial Sequence
  XD.AAV40
aaaaataaacaaataggggttccgcgcacatttccccgaaaagtgccacctgacgtctaagaaaccattattatcatgacattaacctataaaaataggcgtatcacgaggccctttcgttgtaaaacgacggccagtcgaaccacgcaatgcgtctcgatccgcagtgtcttgcgtctcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgccaccgaattcggatccgcggccgccatggctacgcaaagccaaagccaaactttgaatcagagcgaaaatttgcctcaaccaccccaggtttgggacttgctgcagtggctccaagtagtagctcatcaatggcaaacgattactaaagtcccaatggaatgggtggtcccaagagagataggaatagctatcccgaacggttggggcacggagagtagccctcctgccccagagccaggaccttgtccgcccactactataacttccacttcaaagagtcccacagctcaccttgaagacctgcagatgactacaccaaccagtgccactgcgccgcccggagggattctgacaagtactgacagtactgctacatcacaccatgtcaccgggtccgactctagtactaccaccggcgattccggtctctcagatagcacaagttcaagtagcaccttccgaagcaaaaggcttcgcactaccatggaatccaggccatcaccaataacactcccggctcgaagccgaagcagtagaacccaaacaatttctagtaggacttgtagtggaagattgactcgcgccgcttctaggagaagtcaaagaactttcagcgactacaaagacgatgacgacaagtgagtttaaacaccggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaagagacggagtcactgccaaccgagacggtcatagctgtttcctgtgtgccgcttcctcgctcactgactcgctgcgctcggtcgttcggctgcggcgagcggtatcagctcactcaaaggcggtaatacggttacccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaataataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtatcacgctcgtcgtttggtatggcttcattcagctccggttcccaacgatcaaggcgagttacatgatcccccatgttgcgcaaaaaagcggttagctccttcggtcctccgatcgttgtcagaagtaagttggccgccgtgttatcactcatggttatggcagcactacataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttag

SEQ ID NO: 88 - DNA, 5743 bp, Artificial Sequence
  XD.AAV54
gtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtgccgccatggctacgcaaagccaaagccaaactttgaatcagagcgaaaatttgcctcaaccaccccaggtttgggacttgctgcagtggctccaagtagtagctcatcaatggcaaacgattactaaagtcccaatggaatgggtggtcccaagagagataggaatagctatcccgaacggttggggcacggagagtagccctcctgccccagagccaggaccttgtccgcccactactataacttccacttcaaagagtcccacagctcaccttgaagacctgcagatgactacaccaaccagtgccactgcgccgcccggagggattctgacaagtactgacagtactgctacatcacaccatgtcaccgggtccgactctagtactaccaccggcgattccggtctctcagatagcacaagttcaagtagcaccttccgaagcaaaaggcttcgcactaccatggaatccaggccatcaccaataacactcccggctcgaagccgaagcagtagaacccaaacaatttctagtaggacttgtagtggaagattgactcgcgccgcttctaggagaagtcaaagaactttcagcgactacaaagacgatgacgacaagtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgag

SEQ ID NO: 89 - protein, 70 aa, Artificial Sequence
  A1_Cucumber necrosis virus
MALVSRNNNMRTLAKLAAPLATAGTRTIVDNKEAIWNGVKWIWGKLPKGKKGKNGNGALIAHPQAFPGAI

SEQ ID NO: 90 - protein, 84 aa, Artificial Sequence
  A2-TBSV
MAMVKRNNNTGMIPVSTKQLLALGAAAGATALQGFVKNNGMAIVEGAVDLTKRAYKAVRRRGGKKQQMINHVGGTGGAIMAPVA

SEQ ID NO: 91 - protein, 77 aa, Artificial Sequence
  A3-SMV
MAKRLSKQQLAKAIANTLETPPQPKAGRRRNRRRQRSAVQQLQPTQAGISMAPSAQGAMVRIRNPAVSSSRGGITVL

SEQ ID NO: 92 - protein, 73 aa, Artificial Sequence
  A4-SBMV
MAKRLTKQQLAKAIANTLEAPATQSRRPRNRRRRRSAARQPQSIQAGASMAPIAQGAMVRLREPSLRTAGGVT

SEQ ID NO: 93 - protein, 86 aa, Artificial Sequence
  A5-SCMV
MSGLFHHRTKPREIRAFVMATRLTKKQLAQAIQNTLPNPPRRKRRAKRRAAQVPKPTQAGVSMAPIAQGTMVKLRPPMLRSSMDVT

SEQ ID NO: 94 - protein, 47 aa, Artificial Sequence
  A6-Brome MV
MSTSGTGKMTRAQRRAAARRNRWTARVQPVIVEPLAAGQGKAIKAIA

SEQ ID NO: 95 - protein, 54 aa, Artificial Sequence
  A7-RHDV
MEGKARTAPQGEAAGTATTASVPGTTTDGMDPGVVAATSVVTAENSSASVATAG

SEQ ID NO: 96 - protein, 16 aa, Artificial Sequence
  B1-CNV
GNGALIAHPQAFPGAI

SEQ ID NO: 97 - protein, 16 aa, Artificial Sequence
  B2-TBSV
MINHVGGTGGAIMAPV

SEQ ID NO: 98 - protein, 12 aa, Artificial Sequence
  B3-SMV
NPAVSSSRGGIT

SEQ ID NO: 99 - protein, 13 aa, Artificial Sequence
  B4-SBMV
VRLREPSLRTAGG

SEQ ID NO: 100 - protein, 12 aa, Artificial Sequence
  B5-SCMV
KLRPPMLRSSMD

SEQ ID NO: 101 - protein, 13 aa, Artificial Sequence
  B6-Brome MV
EPLAAGQGKAIKA

SEQ ID NO: 102 - protein, 15 aa, Artificial Sequence
  B7-RHDV
TSVVTAENSSASVAT

SEQ ID NO: 103 - protein, 380 aa, Artificial Sequence
  Cucumber necrosis virus capsid protein
MALVSRNNNMRTLAKLAAPLATAGTRTIVDNKEAIWNGVKWIWGKLPKGKKGKNGNGALIAHPQAFPGAIAAPISYAYAVKGRKPRFQTAKGSVRITHREYVSVLSGTNGEFLRNNGTGPNNDFSINPLNPFLFPWLVNIAANFDQYKFNSLRFEYVPLVNTTTNGRVALYFDKDSEDPGPDDRAALANYAHLSEISPWAITKLTVPTDNVKRFISDTSSGDPKLINLGQFGWVAYSGPTAELGDIFVEYTVDLFEAQPTSPLLESLFRESASSVQTRMGLPYFSLEVASATDLVWQARVPGTYVVTIIFNSTVGGLTPSISGGGTINSSFSVSTAGSSAYVANITIRVNANLSLSGLTGATNAQLFAVRAITENAVQVV

SEQ ID NO: 104 - protein, 388 aa, Artificial Sequence
  Tomato bushy stunt virus capsid protein
MAMVKRNNNTGMIPVSTKQLLALGAAAGATALQGFVKNNGMAIVEGAVDLTKRAYKAVRRRGGKKQQMINHVGGTGGAIMAPVAVTRQLVGSKPKFTGRTSGSVTVTHREYLSQVNNSTGFQVNGGIVGNLLQLNPLNGTLFSWLPAIASNFDQYTFNSVVLHYVPLCSTTEVGRVAIYFDKDSEDPEPADRVELANYSVLKETAPWAEAMLRVPTDKIKRFCDDSSTSDHKLIDLGQLGIATYGGAGTNAVGDIFISYSVTLYFPQPTNTLLSTRRLDLAGALVTASGPGYLLVSRTATVLTMTFRATGTFVISGTYRCLTATTLGLAGGVNVNSITVVDNIGTDSAFFINCTVSNLPSVVTFTSTGITSATVHCVRATRQNDVSLI

SEQ ID NO: 105 - protein, 268 aa, Artificial Sequence
  Sesbania mosaic virus capsid protein
MAKRLSKQQLAKAIANTLETPPQPKAGRRRNRRRQRSAVQQLQPTQAGISMAPSAQGAMVRIRNPAVSSSRGGITVLTHSELSAEIGVTDSIVVSSELVMPYTVGTWLRGVAANWSKYSWLSVRYTYIPSCPSSTAGSIHMGFQYDMADTVPVSVNQLSNLRGYVSGQVWSGSAGLCFINGTRCSDTSTAISTTLDVSKLGKKWYPYKTSADYATAVGVDVNIATPLVPARLVIALLDGSSSTAVAAGRIYCTYTIQMIEPTASALNN

SEQ ID NO: 106 - protein, 266 aa, Artificial Sequence
  Southern bean mosaic virus capsid protein
MAKRLTKQQLAKAIANTLEAPATQSRRPRNRRRRRSAARQPQSIQAGASMAPIAQGAMVRLREPSLRTAGGVTVLTHSELSTELAVTNAIVVSSELVMPFTMGTWLRGVAANWSKYSLFSVRYTYLPSCPSTTSGSIHMGFQYDMADTLPVSVNQLSNLRGYVSGQVWSGSSGLCYINGTRCSDTANAITTTLDVAKLGKKWYPFKTSTDFTAAVGVIVNIATPLVPARLVIAMLDGSSSTAVSTGRLYVSYTVQLIEPTALALNN

SEQ ID NO: 107 - protein, 279 aa, Artificial Sequence
  Southern cowpea mosaic virus capsid protein
MSGLFHHRTKPREIRAFVMATRLTKKQLAQAIQNTLPNPPRRKRRAKRRAAQVPKPTQAGVSMAPIAQGTMVKLRPPMLRSSMDVTILSHCELSTELAVTVTIVVTSELVMPFTVGTWLRGVAQNWSKYAWVAIRYTYLPSCPTTTSGAIHMGFQYDMADTLPVSVNQLSNLKGYVTGPVWEGQSGLCFVNNTKCPDTSRAITIALDTNEVSEKRYPFKTATDYATAVGVNANIGNILVPARLVTAMEGGSSKTAVNTGRLYASYTIRLIEPIAAALNL

SEQ ID NO: 108 - protein, 189 aa, Artificial Sequence
  Brome mosaic virus capsid protein
MSTSGTGKMTRAQRRAAARRNRWTARVQPVIVEPLAAGQGKAIKAIAGYSISKWEASSDAITAKATNAMSITLPHELSSEKNKELKVGRVLLWLGLLPSVAGRIKACVAEKQAQAEAAFQVALAVADSSKEVVAAMYTDAFRGATLGDLLNLQIYLYASEAVPAKAVVVHLEVEHVRPTFDDFFTPVYR

SEQ ID NO: 109 - protein, 579 aa, Artificial Sequence
  Rabbit hemorrhagic disease virus capsid protein
MEGKARTAPQGEAAGTATTASVPGTTTDGMDPGVVAATSVVTAENSSASVATAGIGGPPQQVDQQETWRTNFYYNDVFTWSVADAPGSILYTVQHSPQNNPFTAVLSQMYAGWAGGMQFRFIVAGSGVFGGRLVAAVIPPGIEIGPGLEVRQFPHVVIDARSLEPVTITMPDLRPNMYHPTGDPGLVPTLVLSVYNNLINPFGGSTNAIQVTVETRPSDDFEFVMIRAPSSKTVDSISPAGLLTTPVLTGVGNDNRWNGQIVGLQPVPGGFSTCNRHWNLNGSTYGWSSPRFADIDHRRGSASFAGNNSTNVLQFWYANAGSAIDNPISQVAPDGFPDMSFVPFNSPNIPTAGWVGFGGIWNSNNGAPAATTVQAYELGFATGAPNNLQPTTNTSGAQTVAKSIYAVVTGTNQNPTGLFVMASGVISTPNANAVTYTPQPDRIVTTPGTPAAAPVGKNTPIMFASVVKRTGDVNAAAGSTNGTQYGTGSQPLPVTIGLSLNNYSSALMPGQFFVWQLTFASGFMEIGLSVDGYFYAGTGASTTLIDLTELIDVRPVGPRPSKSTLVFNLGGATNGFSYV

SEQ ID NO: 110 - DNA, 24 bp, Artificial Sequence
  XP.AAV183_VP3_V221_f
gtgggtagttcctcgggaaattgg

SEQ ID NO: 111 - DNA, 45 bp, Artificial Sequence
  XP.AAV77_9E-VP1_PmeI_r
ccatcaccggtgtttaaaccagattacgagtcaggtatctggtgc

SEQ ID NO: 112 - DNA, 23 bp, Artificial Sequence
  XP.AAV184_VP3_G236_f
ggggacagagtcatcaccaccag

SEQ ID NO: 113 - protein, 29 aa, Artificial Sequence
  vp3(portion)-inserted linker-vp3(portion)
YLTRNLGGSASGGGAPVADNNEGADGVGS

SEQ ID NO: 114 - protein, 730 aa, Artificial Sequence
  AAV9-d6 PROTEIN SEQUENCE
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPGSLNSFITQYSTGQVSVEIEWELQKENSKRWNPEIQYTSNYYKSNNVEFAVNTEGVYSEPRPIGTRYLTRNL

SEQ ID NO: 115 - protein, 726 aa, Artificial Sequence
  AAV9-d3 PROTEIN SEQUENCE
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPAGSSFITQYSTGQVSVEIEWELQKENSKRWNPEIQYTSNYYKSNNVEFAVNTEGVYSEPRPIGTRYLTRNL

SEQ ID NO: 116 - protein, 1272 aa, Artificial Sequence
  XL.D0-AAV9 PROTEIN SEQUENCE
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLGGSASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 117 - protein, 1266 aa, Artificial Sequence
  XL.D1a-AAV9 PROTEIN SEQUENCE
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLGGSASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPGSLNSFITQYSTGQVSVEIEWELQKENSKRWNPEIQYTSNYYKSNNVEFAVNTEGVYSEPRPIGTRYLTRNL

SEQ ID NO: 118 - protein, 1270 aa, Artificial Sequence
  XL.D1b-AAV9 PROTEIN SEQUENCE
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLENLYFQGASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPGSLNSFITQYSTGQVSVEIEWELQKENSKRWNPEIQYTSNYYKSNNVEFAVNTEGVYSEPRPIGTRYLTRNL

SEQ ID NO: 119 - protein, 1272 aa, Artificial Sequence
  XL.D1c-AAV9 PROTEIN SEQUENCE
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLGGENLYFQGASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPGSLNSFITQYSTGQVSVEIEWELQKENSKRWNPEIQYTSNYYKSNNVEFAVNTEGVYSEPRPIGTRYLTRNL

SEQ ID NO: 120 - protein, 1800 aa, Artificial Sequence
  XL.T1a-AAV9 PROTEIN SEQUENCE
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLGGGGSASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPGSLNSFITQYSTGQVSVEIEWELQKENSKRWNPEIQYTSNYYKSNNVEFAVNTEGVYSEPRPIGTRYLTRNLGGGGSASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPGSLNSFITQYSTGQVSVEIEWELQKENSKRWNPEIQYTSNYYKSNNVEFAVNTEGVYSEPRPIGTRYLTRNL

SEQ ID NO: 121 - protein, 2336 aa, Artificial Sequence
  XL.Q1a-AAV9 PROTEIN SEQUENCE
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLGGGGSASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPGSLNSFITQYSTGQVSVEIEWELQKENSKRWNPEIQYTSNYYKSNNVEFAVNTEGVYSEPRPIGTRYLTRNLGGGGSASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPGSLNSFITQYSTGQVSVEIEWELQKENSKRWNPEIQYTSNYYKSNNVEFAVNTEGVYSEPRPIGTRYLTRNLGGGSGGGGSASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPGSLNSFITQYSTGQVSVEIEWELQKENSKRWNPEIQYTSNYYKSNNVEFAVNTEGVYSEPRPIGTRYLTRNL

SEQ ID NO: 122 - protein, 598 aa, Artificial Sequence
  XL.N0-AAV9 PROTEIN SEQUENCE (VP3 only)
MAMVKRNNNTGMIPVNTKQLLALGAAAGATALQGFVKNNGMAIVEGAVDLTKRAYKAVRRRGGKKQQMINHVGGTGGAIMAPVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 123 - protein, 733 aa, Artificial Sequence
  XL.N1-AAV9 PROTEIN SEQUENCE
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMINHVGGTGGAIMAPVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 124 - protein, 1272 aa, Artificial Sequence
  XL.D1b-AAV-DJ PROTEIN SEQUENCE
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNLENLYFQGAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPGSLNSFITQYSTGQVSVEIEWELQKENSKRWNPEIQYTSNYYKSTSVDFAVNTEGVYSEPRPIGTRYLTRNL

SEQ ID NO: 125 - protein, 1274 aa, Artificial Sequence
  XL.D1c-AAV-DJ PROTEIN SEQUENCE
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNLGGENLYFQGAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPGSLNSFITQYSTGQVSVEIEWELQKENSKRWNPEIQYTSNYYKSTSVDFAVNTEGVYSEPRPIGTRYLTRNL

SEQ ID NO: 126 - protein, 736 aa, Artificial Sequence
  wild-type AAV9 VP1 PROTEIN SEQUENCE
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 127 - protein, 12 aa, Artificial Sequence
  HI variant 657-668
DPPTAFNKDKLN

SEQ ID NO: 128 - protein, 8 aa, Artificial Sequence
  HI variant 659-666
PTAFNKDK

(19) United States
(12) Patent Application Publication
(10) Pub. No.: US 2021/0284699 A1
(43) Pub. Date: Sep. 16, 2021

(54) ADENO-ASSOCIATED VIRAL CAPSIDS WITH EXPANDED SIZES

(71) Applicant: California Institute of Technology, Pasadena, CA (US)

(72) Inventors: Viviana Gradinaru, Pasadena, CA (US); Xiaozhe Ding, Pasadena, CA (US)

(21) Appl. No.: 17/199,293

(22) Filed: Mar. 11, 2021

Related U.S. Application Data
(60) Provisional application No. 62/988,884, filed on Mar. 12, 2020.
(60) Provisional application No. 62/990,351, filed on Mar. 16, 2020.

(51) Int. Cl. C07K 14/015; C12N 7/00

(52) U.S. Cl. CPC C07K 14/015; A61K 38/00; C12N 2750/14133; C12N 7/00

(57) ABSTRACT

Disclosed herein include methods, compositions, and kits comprising variant AAV capsids. Variant capsid proteins, including guided variant capsid proteins, tandem multimers, and/or HI loop variant capsid proteins, are provided in some embodiments. The variant capsid proteins disclosed herein are capable of assembling into a variant AAV capsid with an expanded size (e.g., diameter) and/or genetic cargo capacity. Methods generating recombinant AAV (rAAV) with expanded capsids are provided. Methods of treating diseases and disorders using said rAAV are also disclosed.

### RELATED APPLICATIONS

The present application claims priority under 35 U.S.C. § 119(e) to U.S. Provisional Application No. 62/988,884, filed Mar. 12, 2020; and U.S. Provisional Application No. 62/990,351, filed Mar. 16, 2020. The entire contents of these applications are hereby expressly incorporated by reference in their entireties.

### STATEMENT REGARDING FEDERALLY SPONSORED R&D

This invention was made with government support under Grant No(s). NS111369 & NS087949 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ 302426 US, created Mar. 11, 2021, which is 448 kilobytes in size. The information in the electronic format of the Sequence Listing is incorporated herein by reference in its entirety.

### BACKGROUND

### Field

The present disclosure relates generally to the field of gene delivery. More particularly, the application relates to engineered adeno-associated viruses (AAV) of expanded size capable of packaging oversized cargoes.

### Description of the Related Art

The adeno-associated viral (AAV) capsid, a 25-nm protein nanoparticle, has been widely applied as an in vivo gene delivery vector because of its low immunogenicity, engineerable tropism, and excellent safety profile. However, the capsid's small physical volume imposes a modest upper limit of ˜5 kb (kilobases) on its cargo capacity; oversized cargoes are truncated to fit this limit when packaged. This restriction precludes single-vector packaging of many important genetic cargoes, including a number of CRISPR-based tools, many cell-type-specific promoters and enhancers, and at least 6% of human cDNAs including many disease-related genes. Because the icosahedral capsid has an evolutionarily conserved geometry, altering the size of the AAV capsid has been challenging. There is a need for AAV capsids of expanded size to enable larger genetic cargos.

### SUMMARY

Disclosed herein include variant AAV capsids. In some embodiments, the variant AAV capsid has a diameter of at least 30 nm. In some embodiments, the variant AAV capsid has a diameter of about 30 nm to about 35 nm, of about 30 nm to about 60 nm, of about 40 nm to about 60 nm, of about 45 nm to about 60 nm, of about 50 nm to about 65 nm, or of about 55 nm to about 60 nm. In some embodiments, diameter is calculated as the mean of the major axis length and the minor axis length. In some embodiments, the diameter is measured by transmission electron microscopy (TEM). In some embodiments, the diameter is hydrodynamic diameter. In some embodiments, the hydrodynamic diameter is measured by dynamic light scattering (DLS).

In some embodiments, the variant AAV capsid has a genetic cargo capacity of at least 5.2 kb. In some embodiments, about 5.2 kb to about 8.5 kb. In some embodiments, the variant AAV capsid has a genetic cargo capacity of about 5.2 kb to about 5.5 kb, about 5.5 kb to about 6.0 kb, about 6.0 kb to about 6.5 kb, about 6.5 kb to about 7.0 kb, about 7.0 kb to about 7.5 kb, about 7.5 kb to about 8.0 kb, or about 8.0 kb to about 8.5 kb. In some embodiments, the genetic cargo capacity is: (i) the maximum length of a single-stranded DNA molecule that the variant AAV capsid is capable of protecting from DNAse I digestion; and/or (ii) the maximum length of a double-stranded DNA molecule that the variant AAV capsid is capable of protecting from DNAse I digestion. In some embodiments, the single-stranded DNA molecule is capable of self-hybridizing to form a double-stranded region. In some embodiments, the single-stranded DNA molecule comprises a self-complementary AAV (scAAV) vector. In some embodiments, the variant AAV capsid comprises VP1, VP2, and/or VP3. In some embodiments, the variant AAV capsid comprises an about 1:1:10 ratio of VP1:VP2:VP3.

In some embodiments, the variant AAV capsid comprises a plurality of tandem multimers. In some embodiments, a tandem multimer comprises two or more AAV capsid proteins, wherein the tandem multimer comprises one or more linkers connecting the two or more AAV capsid proteins. In some embodiments, said two or more AAV capsid proteins comprise VP1, VP2, VP3, derivatives thereof, or any combination thereof. In some embodiments, the variant AAV capsid comprises two or more tandem multimers that differ with respect to the capsid protein isoforms that compose said tandem multimers. In some embodiments, one or more of said two or more AAV capsid proteins comprise a HI loop variant capsid protein. In some embodiments, said two or more AAV capsid proteins comprise two or more parental AAV capsid proteins, or derivatives thereof. In some embodiments, a plurality of tandem multimers are capable of assembling into the variant AAV capsid.

In some embodiments, the variant AAV capsid comprises a plurality of HI loop variant capsid proteins. In some embodiments, a HI loop variant capsid protein comprises a HI loop variant of a parental AAV capsid protein, and wherein a HI loop variant capsid protein comprises a removal of one or more amino acids in the capsid protein HI loop relative to a corresponding parental AAV capsid protein. In some embodiments, said HI loop variant capsid protein comprises VP1, VP2, and/or VP3. In some embodiments, a plurality of HI loop variant capsid proteins are capable of assembling into the variant AAV capsid. In some embodiments, the variant AAV capsid comprises a plurality of guided variant capsid proteins. In some embodiments, the guided variant capsid protein comprises an insertion of a guide peptide relative to a corresponding parental AAV capsid protein. In some embodiments, said guided variant capsid protein comprises VP1, VP2, and/or VP3. In some embodiments, a plurality of guided variant capsid proteins are capable of assembling into the variant AAV capsid.

In some embodiments, a plurality of parental AAV capsid proteins are capable of assembling into a corresponding parental AAV capsid. In some embodiments, the variant AAV capsid comprises a larger diameter and/or genetic cargo capacity as compared to a corresponding parental AAV capsid assembled from said parental AAV capsid proteins. In some embodiments, the parental AAV capsid proteins comprises VP1, VP2, and/or VP3. In some embodiments, the corresponding parental AAV capsid does not comprise: (i) a tandem multimer; (ii) a HI loop variant capsid protein; and/or (iii) a guided variant capsid protein. In some embodiments, the corresponding parental AAV capsid comprises a genetic cargo capacity of less than about 4.8 kb, of less than about 4.9 kb, of less than about 5.0 kb, of less than about 5.1 kb, or of less than about 5.2 kb. In some embodiments, the corresponding parental AAV capsid comprises a diameter of less than about 25 nm, of less than about 26 nm, of less than about 27 nm, or of less than about 28 nm. In some embodiments, the variant AAV capsid comprises at least about 10% greater molecular weight as compared to the corresponding parental AAV capsid. In some embodiments, the variant AAV capsid comprises at least about 10% more capsid subunits as compared to the corresponding parental AAV capsid. In some embodiments, the variant AAV capsid comprises at least about 10% larger diameter and/or genetic cargo capacity as compared to the corresponding parental AAV capsid. In some embodiments, the packaging efficiency of the variant AAV capsid is at least about 1%, at least about 5%, at least about 10%, at least about 20%, at least about 30%, at least about 40%, or at least about 50%, of the packaging efficiency of the corresponding parental AAV capsid. In some embodiments, the transduction efficiency of the variant AAV capsid is at least about 0.5%, at least about 1%, at least about 5%, at least about 10%, at least about 20%, at least about 30%, at least about 40%, or at least about 50%, of the transduction efficiency of the corresponding parental AAV capsid. In some embodiments, the structure of the variant AAV capsid retains at least one surface epitope present on the corresponding parental AAV capsid. In some embodiments, the at least one surface epitope is responsible for targeting the variant AAV capsid to one or more cell types. In some embodiments, the variant AAV capsid is capable of being purified with an antigen-binding fragment versus the corresponding parental AAV capsid. In some embodiments, the variant AAV capsid and/or the corresponding parental AAV capsid comprises an icosahedral geometry.

In some embodiments, the one or more linkers are situated at the termini of the two or more AAV capsid proteins. In some embodiments, at least one linker of the one or more linkers comprise the amino acid sequence of a protease cleavage site. In some embodiments, at least one linker of the one or more linkers comprise the amino acid sequence of [GGS]. In some embodiments, the tandem multimer comprises an amino acid sequence that is at least 80%, 85%, 90%, 95%, 98%, 99%, or 100% identical to SEQ ID NO: 116. In some embodiments, at least one linker of the one or more linkers comprise the amino acid sequence of SEQ ID NO: 13. In some embodiments, the tandem multimer comprises an amino acid sequence that is at least 80%, 85%, 90%, 95%, 98%, 99%, or 100% identical to SEQ ID NO: 118. In some embodiments, at least one linker of the one or more linkers comprise the amino acid sequence of SEQ ID NO: 14. In some embodiments, the tandem multimer comprises an amino acid sequence that is at least 80%, 85%, 90%, 95%, 98%, 99%, or 100% identical to SEQ ID NO: 119. In some embodiments, at least one linker of the one or more linkers comprise a flexible peptide linker. In some embodiments, a flexible peptide linker comprises about 1 to about 18 flexible amino acid residues. In some embodiments, the flexible amino acid residues comprise glycine, serine, or a combination thereof.

In some embodiments, the tandem multimer comprises a tandem dimer of a first capsid protein and a second capsid protein. In some embodiments, the tandem dimer comprises a first linker. In some embodiments, a tandem dimer of two AAV capsid proteins comprises a stronger intramolecular 2-fold interaction as compared to the first capsid protein and the second capsid protein not connected by the first linker. In some embodiments, the first linker enhances the apparent association rate and/or decreases the apparent dissociation rate of the dimeric interaction between the first capsid protein and the second capsid protein. In some embodiments, the tandem multimer comprises a tandem trimer of a first capsid protein, a second capsid protein, and a third capsid protein. In some embodiments, the tandem trimer comprises a first linker and a second linker. In some embodiments, the first linker and the second linker comprise about 5 flexible amino acid residues. In some embodiments, the tandem multimer comprises an amino acid sequence that is at least 80%, 85%, 90%, 95%, 98%, 99%, or 100% identical to SEQ ID NO: 120. In some embodiments, the tandem multimer comprises a tandem tetramer of a first capsid protein, a second capsid protein, a third capsid protein, and a fourth capsid protein. In some embodiments, the tandem tetramer comprises a first linker, a second linker, and a third linker. In some embodiments, the first linker and the second linker comprise about 5 flexible amino acid residues, and wherein the third linker comprises about 9 flexible amino acid residues In some embodiments, the tandem multimer comprises an amino acid sequence that is at least 80%, 85%, 90%, 95%, 98%, 99%, or 100% identical to SEQ ID NO: 121. In some embodiments, the first capsid protein comprises VP1, VP2, VP3, or a HI loop variant thereof. In some embodiments, the second capsid protein comprises VP1, VP2, VP3, or a HI loop variant thereof. In some embodiments, the third capsid protein comprises VP1, VP2, VP3, or a HI loop variant thereof. In some embodiments, the fourth capsid protein comprises VP1, VP2, VP3, or a HI loop variant thereof. In some embodiments, the first capsid protein, second capsid protein, third capsid protein, and/or fourth capsid protein comprises a HI loop variant capsid protein. In some embodiments, the HI loop variant capsid protein comprises VP1, VP2, and/or VP3.

In some embodiments, the removal of one or more amino acids in the capsid protein HI loop further comprises an insertion of a flexible peptide linker in the HI loop, wherein the insertion of a flexible peptide linker replaces a contiguous stretch of from 2 amino acids to 18 amino acids of the parental AAV capsid protein. In some embodiments, a flexible peptide linker comprises about 1 to about 18 flexible amino acid residues. In some embodiments, the flexible amino acid residues comprise glycine, serine, or a combination thereof. In some embodiments, a HI loop variant capsid protein comprises a HI loop truncated by 1, 2, 3, 4, 5, 6, 7, 8, 9, 10, 11, 12, 13, 14, 15, 16, 17, or 18 residues relative to a corresponding parental AAV capsid protein. In some embodiments, the HI loop of the corresponding parental AAV capsid protein comprises about 18 amino acids. In some embodiments, the HI loop is located between amino acid V654 and amino acid 1671 of VP1 of AAV9, or the corresponding position in the capsid protein of another AAV serotype. In some embodiments, the HI loop variant capsid protein comprises the removal of 1, 2, 3, 4, 5, 6, 7, 8, 9, 10, 11, or 12 amino acids of the stretch of HI Loop amino acid residues between amino acid D657 and amino acid N668 (DPPTAFNKDKLN; SEQ ID NO: 127) of VP1 of AAV9, or the corresponding amino acids in the capsid protein of another AAV serotype. In some embodiments, the HI loop variant capsid protein comprises the removal of 1, 2, 3, 4, 5, 6, 7, or 8 amino acids of the stretch of HI Loop amino acid residues between amino acid P659 and amino acid K666 (PTAFNKDK; SEQ ID NO: 128) of VP1 of AAV9, or the corresponding amino acids in the capsid protein of another AAV serotype. In some embodiments, the HI loop variant capsid protein comprises an amino acid sequence that is at least 80%, 85%, 90%, 95%, 98%, 99%, or 100% identical to SEQ ID NO: 114 or to SEQ ID NO: 115. In some embodiments, the tandem multimer comprises an amino acid sequence that is at least 80%, 85%, 90%, 95%, 98%, 99%, or 100% identical to SEQ ID NO: 117.

In some embodiments, the guide peptide comprises about 3 amino acids to about 100 amino acids. In some embodiments, the guide peptide comprises a contiguous stretch of from about 2 amino acids to about 100 amino acids from the N-terminal region of a capsid protein of a larger virus species. In some embodiments, the N-terminal region of a capsid protein of a larger virus species comprises: (i) the first 100 amino acids of said capsid protein; and/or (ii) the amino acid sequence of the first 25% of full-length amino acid sequence of said capsid protein. In some embodiments, the insertion of a guide peptide is at a structurally analogous turn in the parental AAV capsid protein relative to the capsid protein of the larger virus species. In some embodiments, said larger virus species forms a capsid comprising a larger diameter and/or genetic cargo capacity than the corresponding parental AAV capsid. In some embodiments, the capsid of the larger virus species comprises a triangulation (T) number of greater than 1. In some embodiments, the larger virus species comprises Tomato bushy stunt virus (TBSV), Sesbania mosaic virus (SMV), Norwalk virus, variants thereof, or any combination thereof. In some embodiments, the larger virus species comprises Cucumber necrosis virus, Tomato bushy stunt virus (cherry strain), Tomato bushy stunt virus (BS-3 strain), Turnip crinkle virus, Carrot mottle virus, Carnation ringspot virus, Sesbania mosaic virus, Southern bean mosaic virus, Southern cowpea mosaic virus, Brome mosaic virus, Rabbit hemorrhagic disease virus, Feline Calicivirus, Nowalk virus, variants thereof, or any combination thereof. In some embodiments, the larger virus species comprises a species of Orthornavirae. In some embodiments, the guide peptide comprises a contiguous stretch of at least about 10 amino acids of any one of the sequences of SEQ ID NOS: 89-102 or of a sequence comprising one mismatch or two mismatches relative to any one of the sequences of SEQ ID NOS: 89-102. In some embodiments, the guide peptide comprises a contiguous stretch of from about 2 amino acids to about 100 amino acids of any one of the sequences of SEQ ID NOS: 103-109. In some embodiments, the capsid protein of the larger virus comprises a similar core fold as an AAV capsid protein. In some embodiments, the capsid protein of the larger virus comprises a core jelly-roll protein fold. In some embodiments, a removal of the guide peptide from the capsid protein of the larger virus species causes a shrinkage of the capsid of the larger virus species.

In some embodiments, the insertion of a guide peptide is between any one of amino acid 1 to amino acid 240 of VP1 of AAV9, or the corresponding position in the capsid protein of another AAV serotype. In some embodiments, the insertion of a guide peptide replaces a contiguous stretch of from about 2 amino acids to about 200 amino acids of the parental AAV capsid protein. In some embodiments, the insertion of a guide peptide replaces a contiguous stretch of from about 2 amino acids to about 200 amino acids of the parental AAV capsid protein following the VP1 start codon, VP2 start codon, and/or VP3 start codon. In some embodiments, the insertion of a guide peptide replaces a contiguous stretch of from about 2 amino acids to about 200 amino acids of the parental AAV capsid protein following the VP2 start codon. In some embodiments, the insertion of a guide peptide replaces a contiguous stretch of from about 2 amino acids to about 50 amino acids following the VP3 start codon. In some embodiments, the insertion of a guide peptide replaces amino acid A204 to amino acid G220 of VP1 of AAV9, or the corresponding position in the capsid protein of another AAV serotype. In some embodiments, the guided variant capsid protein comprises an amino acid sequence that is at least 80%, 85%, 90%, 95%, 98%, 99%, or 100% identical to SEQ ID NO: 122. In some embodiments, the guide peptide comprises amino acid A2 to amino acid G76 of TBSV (cherry strain) coat protein (CP). In some embodiments, the guide peptide comprises amino acid A2 to amino acid P82 of TBSV (cherry strain) CP. In some embodiments, the guide peptide comprises amino acid A2 to amino acid 5101 of TBSV (cherry strain) CP. In some embodiments, the tandem multimer comprises an amino acid sequence that is at least 80%, 85%, 90%, 95%, 98%, 99%, or 100% identical to SEQ ID NO: 124 or to SEQ ID NO: 125. In some embodiments, the guide peptide comprises amino acid 169 to amino acid P82 of TBSV (cherry strain) CP. In some embodiments, the insertion of the guide peptide replaces amino acid A204 to amino acid G220 of VP1 of AAV9, or the corresponding position in the capsid protein of another AAV serotype. In some embodiments, the guided variant capsid protein comprises an amino acid sequence that is at least 80%, 85%, 90%, 95%, 98%, 99%, or 100% identical to SEQ ID NO: 123.

In some embodiments, the guide peptide is conditionally structured. In some embodiments, the guide peptide is capable of forming a β-annulus structure. In some embodiments, the β-annulus structure is capable of stabilizing the pseudo-6-fold interface. In some embodiments, the β-annulus structure is capable of forming a guided variant trimer, wherein the guided variant trimer comprises three guided variant capsids interacting via the β-annulus structure of each guided variant capsid protein. In some embodiments, a plurality of guided variant trimers are capable of assembling into the variant AAV capsid. In some embodiments, the a variant AAV capsid comprises a pseudo-6-fold axis of symmetry. In some embodiments, the guide peptide involved in an intertwined β-annulus structure at a pseudo-6-fold symmetrical interface of the variant AAV capsid. In some embodiments, the guide peptide enables the formation of additional 3-fold interactions on the interior side of the variant AAV capsid comprises at least about 10% greater molecular weight as compared to the corresponding parental AAV capsid. In some embodiments, the corresponding parental AAV capsid comprises capsid proteins comprising an intact HI loop. In some embodiments, the corresponding parental AAV capsid comprises 2-fold symmetrical interfaces, 3-fold symmetrical interfaces, and 5-fold symmetrical interfaces, and wherein the corresponding parental AAV capsid does not comprise a pseudo-6-fold symmetrical interface. In some embodiments, the variant AAV capsid comprises a plurality of pseudo-6-fold symmetrical interfaces. In some embodiments, the plurality of pseudo-6-fold symmetrical interfaces enables the incorporation of additional AAV capsid proteins in the variant AAV capsid as compared to the corresponding parental AAV capsid. In some embodiments, the HI loop of the corresponding parental AAV capsid sterically hinders the formation of a planar hexamer, and wherein the HI loop variant capsid protein does not sterically hinder the formation of a planar hexamer. In some embodiments, the HI loop variant capsid protein can be adapted into a planar hexamer without apparent steric hindrance. In some embodiments, the assembly of the variant AAV capsid comprises a larger critical nucleus as compared to the critical nucleus formed during the assembly of the corresponding parental AAV capsid. In some embodiments, the critical nucleus comprises a pentameric critical nucleus. In some embodiments, the larger critical nucleus comprises extra space between curved pentamers. In some embodiments, said extra space between curved pentamers is eventually filled in by hexamers during assembly of the variant AAV capsid. In some embodiments, assembly of the variant AAV capsid comprises a prolonged lifetime of an intermediate scaffold with a larger radii of curvature as compared to the intermediate scaffolds formed during the assembly of the corresponding parental AAV capsid.

In some embodiments, one or more of the variant AAV capsid, the corresponding parental AAV capsid, the parental AAV capsid protein, the tandem multimer, the HI loop variant capsid protein, the guided variant capsid protein, VP1, VP2, and/or VP3 have an AAV serotype selected from the group comprising AAV9, AAV9 K449R (or K449R AAV9), AAV1, AAVrhlO, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B 3 (PHP.B 3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2 Al 5/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/rl 1.64, AAV4-9/rh.54, AAV4-19/rh.55, AAV5-3/rh.57, AAV5-22/rh.58, AAV7.3/hu.7, AAV16.8/hu.lO, AAV16.12/hu.1 1, AAV29.3/bb.1, AAV29.5/bb.2, AAV106. 1/hu.37, AAV1 14.3/hu.40, AAV127.2/hu.41, AAV127.5/hu.42, AAV128.3/hu.44, AAV130.4/hu.48, AAV145. 1/hu.53, AAV145.5/hu.54, AAV145.6/hu.55, AAV161. 10/hu.60, AAV161.6/hu.61, AAV33. 12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu. 12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu. 1, AAVhu.2, AAVhu.3, AAVhu.4, AAVhu.5, AAVhu.6, AAVhu.7, AAVhu.9, AAVhu.lO, AAVhu. 1, AAVhu. 13, AAVhu.15, AAVhu.16, AAVhu. 1 7, AAVhu.1 8, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu. 1 4/9, AAVhu.t 19, AAVrh.2, AAVrh.2R, AAVrh.8, AAVrh.8R, AAVrh. 10, AAVrh.12, AAVrh. 13, AAVrh.13R, AAVrh. 14, AAVrh.17, AAVrh. 18, AAVrh.19, AAVrh.20, AAVrh.21, AAVrh.22, AAVrh.23, AAVrh.24, AAVrh.25, AAVrh.3 1, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533 A mutant, AAAV, BAAV, caprine AAV, bovine AAV, AAVhE1.1, AAVhEr1.5, AAVhER1. 14, AAVhEr1.8, AAVhEr1. 16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.3 1, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-lOl, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu. 11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128. 1/hu.43, true type AAV (ttAAV), EGRENN AAV 10, Japanese AAV 10 serotypes, AAV CBr-7.1, AAV CBr-7. 10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6. 1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-1 3, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-1 1, AAV CSp-2, AAV CSp-3, AAV CSp-4, AAV CSp-6, AAV CSp-7, AAV CSp-8, AAV CSp-8. 10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, variants thereof, a hybrid or chimera of any of the foregoing AAV serotypes, or any combination thereof. In some embodiments, the VP1 of AAV9 comprises an amino acid sequence that is at least 80%, 85%, 90%, 95%, 98%, 99%, or 100% identical to SEQ ID NO: 126.

Disclosed herein include recombinant AAV (rAAV). In some embodiments, the recombinant AAV (rAAV) comprises: a) a variant AAV capsid provided herein; and b) a heterologous nucleic acid. In some embodiments, the heterologous nucleic acid comprises a polynucleotide encoding a payload.

In some embodiments, the length of the heterologous nucleic acid is about 70%, about 80%, about 90%, about 95%, or about 100%, of the genetic cargo capacity of the variant AAV capsid. In some embodiments, the length of the heterologous nucleic acid is about 5.1 kb to about 5.5 kb, about 5.5 kb to about 6.0 kb, about 6.0 kb to about 6.5 kb, about 6.5 kb to about 7.0 kb, about 7.0 kb to about 7.5 kb, about 7.5 kb to about 8.0 kb, or about 8.0 kb to about 8.5 kb. In some embodiments, the heterologous nucleic acid comprises a 5′ inverted terminal repeat (ITR) and a 3′ ITR. In some embodiments, the payload comprises a payload RNA agent. In some embodiments, the payload comprises a payload protein. In some embodiments, the heterologous nucleic acid further comprises a polynucleotide encoding one or more secondary proteins, wherein the payload protein and the one or more secondary proteins comprise a synthetic protein circuit. In some embodiments, the heterologous nucleic acid comprises a single-stranded AAV (ssAAV) vector or a self-complementary AAV (scAAV) vector.

In some embodiments, the heterologous nucleic acid comprises a promoter operably linked to the polynucleotide encoding a payload. In some embodiments, the promoter is capable of inducing the transcription of the polynucleotide. In some embodiments, the heterologous nucleic acid comprises one or more of a 5′ UTR, 3′ UTR, a minipromoter, an enhancer, a splicing signal, a polyadenylation signal, a terminator, one or more silencer effector binding sequences, a protein degradation signal, and an internal ribosome-entry element (IRES). In some embodiments, the silencer effector comprises a microRNA (miRNA), a precursor microRNA (pre-miRNA), a small interfering RNA (siRNA), a short-hairpin RNA (shRNA), precursors thereof, derivatives thereof, or a combination thereof. In some embodiments, said silencer effector is capable of binding the one or more silencer effector binding sequences, thereby reducing the stability of the payload transcript and/or reducing the translation of the payload transcript. In some embodiments, the polynucleotide further comprises a transcript stabilization element. In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof. In some embodiments, the promoter comprises a ubiquitous promoter. In some embodiments, the ubiquitous promoter is selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof. In some embodiments, the promoter is an inducible promoter. In some embodiments, the inducible promoter is a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, or an RU-486 responsive promoter. In some embodiments, the promoter comprises a tissue-specific promoter and/or a lineage-specific promoter. In some embodiments, the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. In some embodiments, the tissue specific promoter is a neuron-specific promoter. In some embodiments, the neuron-specific promoter comprises a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. In some embodiments, the tissue specific promoter is a muscle-specific promoter. In some embodiments, the muscle-specific promoter comprises a creatine kinase (MCK) promoter. In some embodiments, the promoter comprises an intronic sequence. In some embodiments, the promoter comprises a bidirectional promoter and/or an enhancer. In some embodiments, the enhancer is a CMV enhancer. In some embodiments, one or more cells of a subject comprise an endogenous version of the payload, and wherein the promoter comprises or is derived from the promoter of the endogenous version. In some embodiments, one or more cells of a subject comprise an endogenous version of the payload, and wherein the payload is not truncated relative to the endogenous version.

In some embodiments, the payload RNA agent comprises one or more of dsRNA, siRNA, shRNA, pre-miRNA, pri-miRNA, miRNA, stRNA, lncRNA, piRNA, and snoRNA. In some embodiments, the payload RNA agent inhibits or suppresses the expression of a gene of interest in a cell. In some embodiments, the gene of interest is selected from the group comprising SOD1, MAPT, APOE, HTT, C90RF72, TDP-43, APP, BACE, SNCA, ATXN1, ATXN2, ATXN3, ATXN7, SCN1A-SCN5A, and SCN8A-SCN11A. In some embodiments, the payload protein comprises aromatic L-amino acid decarboxylase (AADC), survival motor neuron 1 (SMN1), frataxin (FXN), Cystic Fibrosis Transmembrane Conductance Regulator (CFTR), Factor X (FIX), RPE65, Retinoid Isomerohydrolase (RPE65), Sarcoglycan Alpha (SGCA), and sarco/endoplasmic reticulum Ca2+-ATPase (SERCA2a), ApoE2, GBA1, GRN, ASP A, CLN2, GLB1, SGSH, NAGLU, IDS, NPC1, GAN, CFTR, GDE, OTOF, DYSF, MYO7A, ABCA4, F8, CEP290, CDH23, DMD, ALMS1, or any combination thereof. In some embodiments, the payload protein comprises a disease-associated protein. In some embodiments, the level of expression of the disease-associated protein correlates with the occurrence and/or progression of the disease. In some embodiments, the payload protein comprises methyl CpG binding protein 2 (MeCP2), DRK1A, KAT6A, NIPBL, HDAC4, UBE3A, EHMT1, one or more genes encoded on chromosome 9q34.3, NPHP1, LIMK1 one or more genes encoded on chromosome 7q11.23, P53, TPI1, FGFR1 and related genes, RA1, SHANK3, CLN3, NF-1, TP53, PFK, CD40L, CYP19A1, PGRN, CHRNA7, PMP22, CD40LG, derivatives thereof, or any combination thereof. In some embodiments, the payload protein comprises fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. In some embodiments, the payload protein comprises nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. In some embodiments, the payload protein comprises a CRE recombinase, GCaMP, a cell therapy component, a knock-down gene therapy component, a cell-surface exposed epitope, or any combination thereof. In some embodiments, the payload protein comprises a chimeric antigen receptor. In some embodiments, the payload protein comprises a diagnostic agent. In some embodiments, wherein the diagnostic agent comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof. In some embodiments, the payload protein comprises a nuclear localization signal (NLS) or a nuclear export signal (NES).

In some embodiments, the payload protein comprises a programmable nuclease. In some embodiments, the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. In some embodiments, the programmable nuclease comprises a zinc finger nuclease (ZFN) and/or transcription activator-like effector nuclease (TALEN). In some embodiments, the programmable nuclease comprises Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cash, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. In some embodiments, the heterologous nucleic acid further comprises a polynucleotide encoding (i) a targeting molecule and/or (ii) a donor nucleic acid. In some embodiments, the targeting molecule is capable of associating with the programmable nuclease. In some embodiments, wherein the targeting molecule comprises single strand DNA or single strand RNA. In some embodiments, wherein the targeting molecule comprises a single guide RNA (sgRNA).

Disclosed herein include pharmaceutical compositions. In some embodiments, the pharmaceutical composition comprises: a rAAV provided herein, and a pharmaceutical excipient. In some embodiments, the pharmaceutical composition is for intraventricular, intraperitoneal, intraocular, intravenous, intraarterial, intranasal, intrathecal, intracistemae magna, or subcutaneous injection, and/or for direct injection to any tissue in the body. The pharmaceutical composition can comprise: a therapeutic agent. The pharmaceutical composition can comprise: (i) a targeting molecule or a nucleic acid encoding the targeting molecule and/or (ii) a donor nucleic acid or a nucleic acid encoding the donor nucleic acid. In some embodiments, the targeting molecule is capable of associating with the programmable nuclease. In some embodiments, wherein the targeting molecule comprises single strand DNA or single strand RNA. In some embodiments, wherein the targeting molecule comprises a single guide RNA (sgRNA).

Disclosed herein include methods of treating a disease or disorder in a subject. In some embodiments, the method comprises: administering to the subject a composition comprising a variant AAV capsid provided herein, or a therapeutically effective amount of an rAAV provided herein, or a pharmaceutical composition provided herein.

In some embodiments, the administering comprises systemic administration. In some embodiments, the systemic administration is intravenous, intramuscular, intraperitoneal, or intraarticular. In some embodiments, administering comprises intrathecal administration, intracranial injection, aerosol delivery, nasal delivery, vaginal delivery, direct injection to any tissue in the body, intraventricular delivery, intraocular delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. In some embodiments, administering comprises an injection into a brain region. In some embodiments, administering comprises direct administration to the brain parenchyma. In some embodiments, the brain region comprises the Lateral parabrachial nucleus, brainstem, Medulla oblongata, Medullary pyramids, Olivary body, Inferior olivary nucleus, Rostral ventrolateral medulla, Respiratory center, Dorsal respiratory group, Ventral respiratory group, Pre-Botzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Subparabrachial nucleus (Kölliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyms, Cornu ammonis, Cornu ammonis area 1, Cornu ammonis area 2, Cornu ammonis area 3, Cornu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyms, Middle frontal gyms, Inferior frontal gyms, Brodmann areas (4, 6, 8, 9, 10, 11, 12, 24, 25, 32, 33, 44, 45, 46, and/or 47), Parietal lobe, Parietal cortex, Primary somatosensory cortex (51), Secondary somatosensory cortex (S2), Posterior parietal cortex, postcentral gyms, precuneus, Brodmann areas (1, 2, 3 (Primary somesthetic area), 5, 7, 23, 26, 29, 31, 39, and/or 40), Occipital lobe, Primary visual cortex (V1), V2, V3, V4, V5/MT, Lateral occipital gyms, Cuneus, Brodmann areas (17 (V1, primary visual cortex), 18, and/or 19), temporal lobe, Primary auditory cortex (A1), secondary auditory cortex (A2), Inferior temporal cortex, Posterior inferior temporal cortex, Superior temporal gyms, Middle temporal gyms, Inferior temporal gyms, Entorhinal Cortex, Perirhinal Cortex, Parahippocampal gyms, Fusiform gyms, Brodmann areas (9, 20, 21, 22, 27, 34, 35, 36, 37, 38, 41, and/or 42), Medial superior temporal area (MST), insular cortex, cingulate cortex, Anterior cingulate, Posterior cingulate, dorsal cingulate, Retrosplenial cortex, Indusium griseum, Subgenual area 25, Brodmann areas (23, 24; 26, 29, 30 (retrosplenial areas), 31, and/or 32), cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), or any combination thereof. In some embodiments, wherein the brain region comprises neural pathways Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract, or any combination thereof.

In some embodiments, administering comprises delivery to dorsal root ganglia, visceral organs, astrocytes, neurons, or a combination thereof of the subject. In some embodiments, the variant AAV capsid comprises tropism for a target tissue or a target cell. In some embodiments, the target tissue or the target cell comprises a tissue or a cell of a central nervous system (CNS) or a peripheral nervous system (PNS), or a combination thereof. In some embodiments, the target cell is a neuronal cell, a neural stem cell, an astrocytes, a tumor cell, a hematopoietic stem cell, an insulin producing beta cell, a lung epithelium, a skeletal cell, or a cardiac muscle cell. In some embodiments, the target cell is located in a brain or spinal cord. In some embodiments, the target cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

The method can comprise: administering an inducer of the inducible promoter to the one or more cells. In some embodiments, the inducer comprises doxycycline. In some embodiments, administering comprises contacting one or more cells from the subject with a composition comprising a variant AAV capsid provided herein, or a therapeutically effective amount of a rAAV provided herein, or a pharmaceutical composition provided herein. In some embodiments, administering comprises: (i) isolating one or more cells from the subject; (ii) contacting said one or more cells with a composition comprising a variant AAV capsid provided herein, or a therapeutically effective amount of a rAAV provided herein, or a pharmaceutical composition provided herein; and (iii) administering the one or more cells into a subject after the contacting step. In some embodiments, the contacting is performed in vivo, in vitro, and/or ex vivo. In some embodiments, the contacting comprises calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof.

In some embodiments, the disease or disorder is selected from the group consisting of pulmonary fibrosis, surfactant protein disorders, peroxisome biogenesis disorders, or chronic obstructive pulmonary disease (COPD). In some embodiments, the disease or disorder comprises a central nervous system (CNS) disorder or peripheral nervous system (PNS) disorder. In some embodiments, the subject is a subject suffering from or at a risk to develop one or more of chronic pain, cardiac failure, cardiac arrhythmias, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, and lysosomal storage disorders that involve cells within the CNS. In some embodiments, the lysosomal storage disorder is Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease. In some embodiments, the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof. In some embodiments, the disease or disorder comprises a neurological disease or disorder. In some embodiments, the neurological disease or disorder comprises epilepsy, Dravet Syndrome, Lennox Gastaut Syndrome, mycolonic seizures, juvenile mycolonic epilepsy, refractory epilepsy, schizophrenia, juvenile spasms, West syndrome, infantile spasms, refractory infantile spasms, Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic clerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, or any combination thereof.

Disclosed herein include recombinant vectors. In some embodiments, the recombinant vector comprises a nucleic acid encoding a variant AAV capsid provided herein.

Disclosed herein include methods of manufacturing a recombinant AAV from the variant AAV capsids provided herein. In some embodiments, the method comprises: a) introducing into a cell: (i) a first nucleic acid comprising a heterologous nucleic acid comprising a polynucleotide encoding a payload; (ii) a second nucleic acid encoding a variant AAV capsid provided herein; and (iii) a third nucleic acid encoding an AAV helper virus genome; and b) assembling the recombinant AAV, the recombinant AAV comprising the variant AAV capsid encapsulating a heterologous nucleic acid.

Disclosed herein include kits. In some embodiments, the kit comprises: a) a recombinant vector comprising a nucleic acid encoding a variant AAV capsid provided herein; b) a helper vector encoding a helper virus protein; and c) a payload vector comprising a heterologous nucleic acid, wherein said heterologous nucleic acid comprises a polynucleotide encoding a payload.

Disclosed herein include methods of purifying recombinant AAV (rAAV). In some embodiments, the method comprises: (a) generating a viral particle extract comprising a plurality of rAAV provided herein, wherein the viral particle extract comprises the supernatant of lysed producer cells, or a derivative thereof; (b) contacting the viral particle extract with an ionic detergent to generate a first mixture; (c) contacting the first mixture with an acid to generate a second mixture; (d) centrifuging the second mixture to generate a supernatant; (e) filtering the supernatant with one or more filters to generate a filtrate; (f) performing one or more cycles of buffer exchange of the filtrate to a final storage buffer.

The method can comprise: prior to step (b), contacting the viral particle extract with DNase I and/or MspI. In some embodiments, the ionic detergent comprises sodium deoxycholate. In some embodiments, the ionic detergent comprises 5% (m/v) sodium deoxycholate. In some embodiments, the quantity of ionic detergent contacted with the viral particle extract is about 10% of the volume of the viral particle extract. In some embodiments, the acid comprises citric acid. In some embodiments, the acid comprises 1 M citric acid. In some embodiments, the quantity of the acid contacted with first mixture is about 4% of the volume of the first mixture. In some embodiments, the one or more filters comprises an about 0.45 μm filter. In some embodiments, step (d) comprises centrifugation at about 5000 g for 5 about minutes. In some embodiments, step (f) comprises at least about 5 cycles of buffer exchange. In some embodiments, each cycle of buffer exchange comprises performing centrifugation of concentrator tubes. In some embodiments, said centrifugation comprises about 1000 g for about 20 minutes. In some embodiments, step (f) comprises buffer exchange with a 100 kD MWCO centrifugal concentrator. In some embodiments, step (f) comprises an at least about 5-fold reduction in solution volume. In some embodiments, the final storage buffer comprises PBS, additional NaCl, and/or Pluronic F-68. In some embodiments, the Pluronic F-68 is 0.001% Pluronic F-68. In some embodiments, the additional NaCl is 300 mM additional NaCl.

Disclosed herein include populations of recombinant AAV (rAAV). In some embodiments, the average diameter of the viral capsids of the population of rAAV range is about 30 nm to about 60 nm, from about 30 nm to about 50 nm, from about 30 nm to about 40 nm, or from about 30 nm to about 35 nm. In some embodiments, the diameter of the viral capsids of the population of rAAV ranges from 25 nm to about 60 nm. In some embodiments, the average diameter of the viral capsids of the population of rAAV is about 30 nm, about 32 nm, about 34 nm, about 36 nm, about 38 nm, about 40 nm, about 42 nm, about 44 nm, about 46 nm, about 48 nm, about 50 nm, about 52 nm, about 54 nm, about 56 nm, about 58 nm, or about 60 nm. In some embodiments, the average is the mean, median or mode. In some embodiments, the mean is the arithmetic mean, geometric mean, and/or harmonic mean. In some embodiments, the capsids of the rAAV have a minimum diameter of about 30 nm, about 32 nm, about 34 nm, about 36 nm, about 38 nm, about 40 nm, about 42 nm, about 44 nm, about 46 nm, about 48 nm, about 50 nm, about 52 nm, about 54 nm, about 56 nm, about 58 nm, or about 60 nm. In some embodiments, the capsids of the rAAV have a maximum diameter of about 30 nm, about 32 nm, about 34 nm, about 36 nm, about 38 nm, about 40 nm, about 42 nm, about 44 nm, about 46 nm, about 48 nm, about 50 nm, about 52 nm, about 54 nm, about 56 nm, about 58 nm, or about 60 nm.

### BRIEF DESCRIPTION OF THE DRAWINGS

The patent or application file contains at least one drawing executed in color. Copies of this patent or patent application publication with drawing(s) will be provided by the Office upon request and payment of the necessary fee.

FIGS. 1A-C depict non-limiting exemplary considerations for the design of XL-AAVs. FIG. 1A depicts non-limiting exemplary architectures of icosahedral capsids. Each icosahedral capsid is made of 60×T subunits. Representative symmetrical interactions between the subunits involved in each type of capsid are shown in the small spheres. Among the symmetrical interactions, the 5-fold interaction (black curves in the sectional view) is the major source of curvature in an icosahedral capsid. (Pseudo-)6-fold interaction (orange lines in the sectional view), which only appears in T≥3 capsids, results in planar hexamers that connect the 5-fold vertices. These extra subunits between the 5-fold vertices contribute to the expansion of the capsid's size. FIGS. 1B-1C show non-limiting exemplary comparisons of a theoretical assembly pathway for a T=1 capsid (FIG. 1B) vs. two other assembly pathways for natural T=3 icosahedral capsids (FIG. 1C). Without being bound by any particular theory, in some embodiments, the addition of building blocks around the 5-fold vertex is initially energetically unfavorable until a “critical nucleus” is formed, and further addition of building blocks to the critical nucleus then becomes energetically favorable. Compared to T=1 capsids (FIG. 1B), the larger capsids are assembled via more complicated pathways (FIG. 1C), in which sub-assembled structural units with either a rigid interface (FIG. 1C, top) or a flexible peptide (FIG. 1C, bottom) serve as the basic building blocks. These multi-subunit building blocks may extend the outer edge (highlighted by dashed orange line) of the pentameric critical nucleus, resulting in extra space between curved 5-fold vertices (highlighted by black curves). The space is eventually filled in with more subunits to form hexamers in canonical T=3 capsids. Strengthened interactions within the multi-subunit subassemblies may also help stabilize intermediate scaffolds with larger radii of curvature. Modifications in XL-AAVs aim to bias the AAV capsid subunits to form similar sub-assembled structural units. Double arrows indicate a reversible reaction, with the thicker arrow marking the favored direction. A dashed line within an arrow indicates that the stage is skipped. Structures used for visualizations throughout this figure are low-resolution models of a T=1 deletion mutant of Sesbania mosaic viral capsid (PDB ID: 1VAK) and the T=3 wild-type capsid of the same species (PDB ID: 1SMV). Blue, green, and white colors represent three minorly different conformations taken by the chemically identical subunits in a T=3 capsid.

FIGS. 2A-2I depict non-limiting exemplary embodiments and data related to tandem dimerization, which, in some embodiments, yields larger capsids that can protect DNA. FIG. 2A depicts the dimer structure of TBSV coat protein (PDB ID: 2TBV), showing the large, flat interface between the two subunits. FIG. 2B depicts the dimer structure of AAV9 capsid protein (PDB ID: 3UX1). Compared to TBSV dimers, the AAV9 dimeric interface only buries a small surface area. However, the interaction might be strengthened by tandem-dimerization, since the C-terminus of one subunit is close to the N-terminus (red) of its 2-fold (43 A) interaction counterpart. FIG. 2C depicts the design and TEM morphology of the tandem homodimer XL.D0-AAV9. The triangular arrows underneath the plasmid map denote the translational start sites that lead to the expression of VP1, VP2, and VP3 proteins, respectively. Scale bar, 100 nm. FIG. 2D depicts a comparison of the pentameric interface and a (hypothetical) hexameric interface in TBSV, AAV9, and engineered AAV9-d6 capsid proteins. Left: Structure of TBSV coat protein subunits (PDB ID: 2TBV) in the pentameric interface and the hexameric interface. Middle: Structure of AAV9 capsid subunits (PDB ID: 3UX1) placed in a pentamer or a qualitatively docked planar hexamer, with an arrow highlighting steric hindrance by the HI loop (orange). Right: Modeled structure of the HI-loop-shortened (green) AAV9-d6 capsid subunits placed in a pentamer or a qualitatively docked planar hexamer, where the shortened HI loop (orange) no longer hinders the formation of the hexamer. FIG. 2E depicts the design and TEM morphology of tandem heterodimers XL.D1a/b/c-AAV9. These variants are composed of an unmodified VP1/2/3 subunit (VP1/2/3 wt) and a HI-loop-shortened VP3 subunit (VP3 d6). XL.D1a-AAV9 uses the same GGS flexible linker as XL.D0-AAV9, XL.D1b-AAV9 uses an ENLYFQG (TEV protease cleavage site; SEQ ID NO: 13) linker, and XL.D1c-AAV9 uses a further extended GGENLYFQG linker (SEQ ID NO: 14). TEM samples were produced with a 7 kb rAAV genome (genome #3, Table 6) and were purified with a precipitation-based method. Scale bar, 100 nm. FIG. 2F depicts quantification of properties of particles in the TEM micrographs. The TEM images were segmented to generate a mask with pixels assigned to individual particles or the background, and shape parameters of each particle were measured. For each particle, the diameter was calculated as the mean of the major axis length and the minor axis length, and the roundness was defined as R=4 nA/c² (A: area; c: perimeter). Statistical significance was determined with a two-tailed Student's t-test (*P<0.05, **P<0.01, ***P<0.001). FIG. 2G depicts qPCR titers of DNasel-protected viral genomes of tandem-dimer variants in producer cell lysates. Genomes #2 (5 kb, Table 6) and #3 (7 kb, Table 6) were packaged, respectively. The qPCR amplicon was a 100 bp sequence within the GFP coding gene, and the titer may not reflect the copy number of fully protected genomes. N=3 biological replicates. All bars are mean±s.e.m. FIG. 2H depicts qPCR titers of DNasel-protected viral genomes in fractions separated by a size-exclusion column. N=3 technical replicates. Data shown are mean±s.e.m. FIG. 2I depicts the hydrodynamic size distributions of AAV9, XL.D0-AAV9, and XL.D1b-AAV9, as measured with DLS. The hydrodynamic diameter measured with DLS is larger than that measured with TEM as the diameter measured by the former includes the solvent layer influenced by the capsid. Correlation functions of the DLS measurements are included in FIG. 15.

FIGS. 3A-3F depict non-limiting exemplary embodiments and data related to peptide grafts from TBSV coat protein, which, in some embodiments, increase the size of AAV9. FIG. 3A depicts the top view (top) and side view (bottom) of a hexamer from a T=3 tomato bushy stunt virus (TBSV) capsid (PDB ID: 2TBV), highlighting the guide peptide (red) which forms additional interactions at the pseudo-6-fold interface. The guide peptide (red) can be either disordered or ordered depending on its relative position. In its ordered conformation (shown here with white subunits), the peptide forms a β-annulus (red triangle) that stabilizes the pseudo-6-fold interface. FIG. 3B depicts structural alignments demonstrating the similarity between the jelly-roll fold of a TBSV coat protein subunit type C (green, PDB ID: 2TBV) and the jelly-roll fold of an AAV9 capsid subunit (blue, PDB ID: 3UX1). Highlighted are the guide peptide (red) within the TBSV coat protein and the native AAV9 peptide (magenta) that is replaced in XL.N0-AAV9 and XL.N1-AAV9. The conventional names of the jelly-roll secondary structures are labeled on the AAV9 structure. The alignment was performed with the CE algorithm within PyMOL software. FIG. 3C depicts the design and TEM morphology of XL.N0-AAV9, in which the N-terminal sequence of AAV9 was replaced by an N-terminal sequence (A2-P82) from TBSV CP. The triangular arrow (black) underneath the plasmid map denotes the only translational start site. TEM samples were produced with a 7 kb rAAV genome (genome #3, Table 6) and were purified with a precipitation-based method. Scale bar, 100 nm. FIG. 3D depicts the design and TEM morphology of XL.N1-AAV9, in which a 14aa peptide (I69-P82) from TBSV CP protein was inserted after the start codon of VP3, replacing the native 17 mer peptide A204-G220. The triangular arrows underneath the plasmid map denote translational start sites for the expression of VP1 (gray), VP2 (gray), and VP3 (black), respectively. TEM samples were produced with a 7 kb rAAV genome (genome #3, Table 6) and were purified with a precipitation-based method. Scale bar, 100 nm. FIG. 3E depicts hydrodynamic diameter distribution of AAV9, XL.N0-AAV9, and XL.N1-AAV9 as measured by DLS. Data were collected in the same experiment as FIG. 2I. Correlation functions of the DLS measurements are included in FIG. 15. FIG. 3F shows quantification of properties of particles in the TEM micrographs. The TEM images were segmented to generate a mask with pixels assigned to individual particles or the background, and shape parameters of each particle were measured. The distributions shown for AAV9 are the same as the distributions plotted in FIG. 2F. For each particle, the diameter was calculated as the mean of the major axis length and the minor axis length, and the roundness was defined as R=4 nA/c² (A: area; c: perimeter). Statistical significance was determined with a two-tailed Student's t-test (*P<0.05, **P<0.01, ***P<0.001).

FIGS. 4A-4G depict non-limiting exemplary embodiments and data related to the ability of XL-AAV capsid to protect and transduce full-length oversized genome. FIG. 4A depicts yields of purified XL.D1c-AAV-DJ from each 15-cm dish compared to those of wild-type AAV-DJ. N=3 biological replicates. All bars are mean±s.e.m. FIG. 4B shows TEM micrographs of XL.D1c-AAV-DJ. XL.D1c-AAV-DJ forms compact, 35-50 nm particles. Scale bars, 100 nm. FIG. 4C shows southern blot on alkaline gel electrophoresis with regular-sized or oversized (>5 kb) genomes protected by XL.D1b-AAV-capsids. 3e9 copies of DNasel-protected genomes (genomes #2, #8, #9, #10, Table 6) were extracted and blotted using a probe against a GFP coding gene within the genomes. Full-length genomes as long as 8.5 kb can be detected. FIGS. 4D, 4F depict embodiments and data related to an infectivity assay showing that XL.D1c-AAV-DJ particles carrying either a 3.8 kb (genome #6, Table 6) or an 8.2 kb genome (genome #7, Table 6) can transduce HEK293T cells and induce Cre-dependent reporter expression (arrows point to a few examples). Both genomes encode a Cre recombinase expression cassette. In the 8.2 kb rAAV genome, the promoter and the coding gene for Cre recombinase were separated by a 3.8 kb gene and a 0.6 kb IRES sequence to ensure that capsids with regular cargo capacity would not package a truncated fragment containing both the promoter and the Cre coding gene. Purified capsids were applied to HEK293T cells pre-transfected with Cre-dependent GFP expression (MOI=10,000:1); images were taken seven days after infection. The green signal indicates infected cells, whereas the red nuclear-localized signal indicates density of transfected cells. Insets are zoom-in images with contrast adjusted individually to show the morphology of a representative fluorescent cell optimally. Scale bars, 100 μm (overview) or 50 μm (inset). FIGS. 4E, 4G depict embodiments and data related to an infectivity assay showing that XL.D1c-AAV-DJ particles carrying either a 3.8 kb or an 8.2 kb genome can infect mouse primary cortical neurons. Cultured primary neurons were co-transduced by AAV-DJ or XL.D1c-AAV-DJ carrying a genome expressing Cre recombinase (MOI=20,000:1) along with AAV9 carrying a Cre-dependent GFP reporter (MOI=100,000:1); green signal indicates co-transduced cells; images were taken three days after infection. Insets are zoom-in images with contrast adjusted individually to show the morphology of a representative fluorescent cell optimally. Scale bars, 100 μm (overview) or 50 μm (inset).

FIG. 5 depicts a non-limiting exemplary summary of XL-AAV designs by tandem-multimerization and guide peptide grafting. Names, design pedigrees, and TEM morphologies of XL-AAV designs presented herein. Scale bars, 100 nm.

FIGS. 6A-6B depicts characteristic structural features found in T=3 or T=4 capsids that may facilitate ordered assembly of the capsids. FIG. 6A depicts the dimeric interface of many T=3 or T=4 capsids is simple and buries a large portion of the surface area of each subunit. Without being bound by any particular theory, this may promote fast sub-assembly of multi-subunit (particularly dimeric) structural units with strong, rigid interfaces. FIG. 6B depicts a class of T=3 capsids form 3-fold interactions on the interior side of the protein shell using a flexible peptide with interchangeable conformations.

FIGS. 7A-7B depicts exemplary tandem-multimerized subunits most likely form 2-fold or 3-fold interactions. FIG. 7A depicts maximal stretched length of the flexible peptide (including the inserted linker and the disordered region of the second subunit) between the structured parts of two subunits in XL.D0-AAV9. The contour length of the peptide was calculated on the basis of 0.36 nm per residue. FIG. 7B depicts measurements of C-terminus-to-N-terminus distances between symmetrical interaction partners in the crystal structure of wild-type AAV9 (PDB ID: 3ux1). Distance between the structured C-terminus (black dot) of a subunit and the structured N-terminus (red dot) of a neighboring subunit (C-to-N distance) with a 2-fold (bottom left), counter-clockwise 3-fold (top left), clockwise 3-fold (top right), or clockwise 5-fold or counter-clockwise 5-fold (bottom right) binding partner. For each symmetric interaction, the two subunits being measured are highlighted in blue, and the other subunits participating in the interaction are colored grey.

FIGS. 8A-8B depict non-limiting exemplary embodiments and data related to morphology of affinity-purified tandem-dimer AAV9 capsids. FIG. 8A depicts electron micrographs of negatively stained wt-wt homodimer AAV9 packaging a 4.9 kb rAAV genome (genome #2, Table 6) or a 7.0 kb rAAV genome (genome #3, Table 6). FIG. 8B depicts Wt-d6 heterodimer AAV9 packaging the same genomes as in FIG. 8A. Heterogeneous, size-expanded capsids with 40-60 nm diameters were observed for both capsids when packaging an oversized genome; additional ˜25 nm particles were observed when a 4.9 kb genome was being packaged. Scale bars, 50 nm.

FIGS. 9A-9G depict non-limiting exemplary embodiments and data related to computationally aided screening for a HI-loop-shortened AAV capsid subunit. FIG. 9A depicts comparisons of the 5-fold axis structure in trimers of AAVs, other parvoviruses, and plant ssRNA viruses. The 18 mer HI loop present in AAVs is probably a recently evolved structural feature. FIG. 9B depicts design scores and accepted counts of poses for 12 different HI-loop truncation designs, as evaluated by RosettaRemodel. FIG. 9C depicts capsid-production-based screening of the 6 HI-loop-shortened variants with the highest predicted stability in 8 different plasmid backbones with a 3.4 kb rAAV genome (genome #1, Table 6). FIG. 9D depicts the same capsid-production-based screening as in FIG. 9C but with a 5.2 kb rAAV genome (genome #5, Table 6). Both genomes are within the packaging capacity of wild-type AAV capsids, and the purpose of screening with both genomes was to evaluate all candidates with two different cargo loading levels. FIG. 9E depicts a second round of screening with the highest-packaging variants from FIGS. 9C-9D. Candidates were sorted by their titer with the 5.2 kb genome. N=3 technical replicates. All bars are mean±s.e.m. FIG. 9F depicts crystal structure of AAV9 (PDB ID:3UX1) and qualitatively docked structures of AAV9d3 and AAV9d6, the two highest-packaging hits in FIG. 9E, highlighting reduced steric hindrance at the 6-fold interface. FIG. 9G shows negative-staining TEM images of AAV9 wt, AAV9 d3, and AAV9 d6. All three capsid variants form uniformly 25-nm particles that resemble canonical T=1 icosahedral capsids. Scale bars, 100 nm.

FIGS. 10A-10K depict non-limiting exemplary embodiments and data related to linker engineering with AAV9 heterodimer (wt-d6) capsids. FIG. 10A shows titers of wt-d6 heterodimeric variants with flexible linkers of different lengths. The titer was measured in cell lysates lysed with three cycles of freeze-thawing. The variants have different numbers of glycine and serine residues (denoted as the (G/S)#) and different numbers of residues truncated in the N-terminus of VP3 (A204-A218). N=3 technical replicates. All bars are mean±s.e.m. Statistical significance was determined with a two-tailed Student's t-test (*P<0.05, **P<0.01). FIG. 10B shows titer of wt-d6 heterodimeric variants with linkers containing a TEV protease recognition site. N=6 technical replicates. All bars are mean±s.e.m. Statistical significance was determined with a two-tailed Student's t-test (*P<0.05, **P<0.01). FIGS. 10C-10H depict negative-staining TEM morphology of AAV9 and wt-d6 capsids. Note that in the AAV9 images, most of the capsids are stained in the interior, indicating empty capsids. TEM samples were produced with a 7 kb rAAV genome (genome #3, Table 6) and were purified with a precipitation-based method. Scale bars, 100 nm. FIGS. 10I-10K depict special designs showing that the presence of a linker in post-translational assembly is necessary for the formation of size-expanded capsids. Each panel shows a representative negative-staining electron micrograph; insets show a schematic of the variant design. FIG. 10I depicts a heterodimer with a circularly permuted VP3 d6 with peptide chain termini at the 3-fold interface, which would presumably force the two monomers to be 3-fold interaction partners. FIG. 10J depicts a heterodimer with a T2A self-cleavage linker that is cleaved during ribosomal translation. FIG. 10K depicts a heterodimer with a TEV protease recognition site linker co-expressed with TEV protease. All samples were produced in 6-well tissue culture plates and purified with a precipitation-based method. All linker variants form size-expanded capsids sized between 35 nm-60 nm. Scale bars, 100 nm.

FIGS. 11A-11H depict non-limiting exemplary embodiments and data related to capsid size expansion via tandem-multimerization across AAV serotypes. FIG. 11A shows western blot of capsid proteins in viral particle extracts. The bands were detected with anti-VP3 antibody (clone B1). XL.D1c-AAV showed increased molecular weight and a similar ratio of VP1:VP2:VP3 to wild-type AAV9. The extra band between bands corresponding to VP2 and VP3 is possibly a result of a different level of post-translational modifications. FIG. 11B depicts TEM morphologies of XL.D1b-AAV variants with different serotypes. Scale bars, 100 nm. FIG. 11C shows QPCR titers of DNasel-protected viral genomes of tandem-multimer XL-AAV9 variants in producer cell lysates. N=3 technical replicates, all bars are mean±s.e.m. QPCR titers of DNasel-protected viral genomes of different AAV serotypes and corresponding XL.D1b-AAV variants. The AAV and XL.D1b-AAV capsid genes of the same serotype were in the same Rep-Cap plasmid backbone. N=3 technical replicates; all bars are mean±s.e.m. FIG. 11D depicts design and TEM morphology of tandem-heterotrimeric XL.T1a-AAV9. Scale bar, 100 nm. FIG. 11E depicts the design and TEM morphology of tandem-heterotetrameric XL.Q1a-AAV9. Scale bar, 100 nm. FIG. 11F shows quantification of properties of particles in the TEM micrographs. The TEM images were segmented to generate a mask with pixels assigned to individual particles or the background, and shape parameters of each particle were measured. For each particle, the diameter was calculated as the mean of the major axis length and the minor axis length, and the roundness was defined as R=4 nA/c² (A: area; c: perimeter). Distributions of XL.T1a-AAV9 and XL.Q1a-AAV9 particles were tested against the distribution of XL.D1b-AAV9 with a two-tailed Student's t-test (*P<0.05, **P<0.01, ***P<0.001). FIG. 11G depicts qPCR titers of DNasel-protected viral genomes of different tandem-multimer variants in producer cell lysates. Statistical analyses were performed with a two-tailed Student's t-test (*P<0.05, **P<0.01, ***P<0.001). FIG. 11H shows hydrodynamic size distributions of AAV9, XL.D1b-AAV9, XL.T1a-AAV9, and XL.Q1a-AAV9, as measured with DLS. The hydrodynamic diameter measured with DLS is larger than that measured with TEM as the former is influenced by the solvent layer associated with the capsid. Correlation functions for the DLS measurements are included in FIG. 15. TEM and DLS samples were produced with a 7 kb rAAV genome (genome #3, Table 6) and purified with a precipitation-based method.

FIGS. 12A-12F depict non-limiting exemplary embodiments and data related to different N-terminal grafts from TBSV CP to AAV VP3, which, in some embodiments, lead to expanded virus-like particles with different morphologies. FIG. 12A depicts the structure of the VP3 AAV9 monomer (left). The structure of the 6-fold axis of the TBSV capsid (center, green, PDB ID:2tbv) and the 5-fold axis of the AAV9 capsid (right, blue, PDB ID:3ux1) viewed from the interior of the capsid. FIGS. 12B-12D depict qualitatively modeled structures of an AAV9 monomer with peptide grafts (left). Structures of TBSV CP (center) and AAV9 VP3 (right), as in FIG. 12A but with different added N-end grafts shown in red (indicating the structured part of the grafted peptide) and with the graft sites shown in cyan. FIG. 12E shows qPCR titration of the grafting variants in wild-type AAV9 VP3 backbone. N=3 technical replicates. Statistical analyses were performed with a two-tailed Student's t-test (*P<0.05, **P<0.01, ***P<0.001). All bars are mean±s.e.m. FIG. 12F shows representative TEM morphology of some size-expanded capsids. TEM samples were produced with a 7 kb rAAV genome (genome #3, Table 6), and were purified with a precipitation-based method. Scale bars, 100 nm.

FIGS. 13A-13G depicts screening for a 14mer N-terminal peptide graft from TBSV CP for grafting to AAV capsids. FIG. 13A depicts qPCR titration of a series of AAV variants with semi-rationally designed peptide insertion/replacements produced in 6-well plates. N=3 technical replicates. All bars are mean±s.e.m. FIG. 13B depicts that in side-by-side production in 15-cm dishes, the 14mer peptide variants show a qualitatively comparable DNasel-protected titer to domain insertion variants. N=3 technical replicates. All bars are mean±s.e.m. FIG. 13C depicts western blot with XL.N1-AAV9 (TBSV_69-82s-CAP) viral particle extracts, showing a similar ratio of VP1, VP2, VP3 in XL.N1-AAV9 compared to AAV9. FIG. 13D depicts TEM images of the 81 aa peptide graft variant TBSV_2-82i-VP3. FIG. 13E depicts TEM images of the 14 aa-peptide-replacement variant TBSV_69-82s-CAP, which forms capsids with similar morphology as TBSV_2-82i-VP3. FIGS. 13F-13G depicts the same peptide insertion/replacements as in FIGS. 13D-13E also produce size-expanded capsids when the backbones had a shortened HI loop (AAV9-d6). Scale bars, 100 nm.

FIGS. 14A-14C depict non-limiting exemplary embodiments and data related to the ability of XL-AAV to infect cells from wild-type and transgenic mice and from human sources. FIG. 14A depicts an infectivity assay showing that DNasel-treated crude XL.D1b-AAV-DJ viral extracts can transduce primary cultured neurons. Cortical neurons were prepared at E16 from the Ai14 strain, which produces Cre-dependent fluorescent expression of the Rosa26::CAG::FRT::loxP-STOP-loxP::tdTomato::WPRE::polyA cassette in the genome. Five days after the neuron prep, cells were incubated with DNasel-treated viral extracts at MOI of ˜1e6:1 for a 2 hr period, and the media was refreshed after the incubation. Fluorescent images were taken three days after infection. Scale bars, 100 μm. FIG. 14B depicts an infectivity assay showing that XL.D1c-AAV-DJ can transduce HEK293T cells. The same experiment as described in FIGS. 4D, 4F was repeated, with more zoomed-in images around the fluorescent cells. Insets are zoom-in images with contrast adjusted individually to show the morphology of a representative fluorescent cell optimally. Scale bars, 100 μm (overview) or 50 μm (inset). FIG. 14C depicts the same infectivity assay as in FIGS. 4E, 4G, but the fluorescent images were taken five days after infection (arrows point to a few examples). Reporter GFP expression by AAV-DJ packaging an 8.2 kb genome is a result of intracellular reassembly of co-transduced vectors with truncated genomes from both ends. Insets are zoom-in images with contrast adjusted individually to show the morphology of a representative fluorescent cell optimally. Scale bars, 100 μm (overview) or 50 μm (inset).

FIGS. 15A-15G depict data related to the correlation functions for the DLS measurements shown in FIG. 2I, FIG. 3E, and FIG. 11H.

FIGS. 16A-16B depict data related to optimized AAV purification workflows. FIG. 16A depicts data related to the optimization of the storage conditions of XL.D1c-AAVDJ. Purified XL.D1c-AAVDJ was incubated in buffers with different ionic strengths (columns 1-6) or additives (columns 7-8) at 4 C. All buffers include the salt components indicated in the figure and an additional 0.001% Pluronic F-68. The remaining titers in the solutions were quantified with qPCR after 10 days of incubation. Titers of XL.D1c-AAVDJ are most robust at an ionic strength of ˜600 mM, and particles incubated with glycine amide (GlyNH2) shows improved stability of compared to particles incubated with NaCl at the same concentration. FIG. 16B depicts data related to the optimization of harvest times of XL.D1c-AAVDJ. XL.D1c-AAVDJ was produced following the protocol described in Example 7, and cell pellets were harvested and extracted at different time points after transfection. DNasel-protected titer in the extract was quantified with qPCR. The titer in the extract peaks at around 96 hours after transfection.

FIG. 17 depicts a non-limiting exemplary virus purification workflow. FIG. 17 shows electron micrographs of samples of XL.D1-AAV9 producer cells after different purification steps (scale bar: 100 nm).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include variant AAV capsids. In some embodiments, the variant AAV capsid has a diameter of at least 30 nm. In some embodiments, the variant AAV capsid has a diameter of about 30 nm to about 35 nm, of about 30 nm to about 60 nm, of about 40 nm to about 60 nm, of about 45 nm to about 60 nm, of about 50 nm to about 65 nm, or of about 55 nm to about 60 nm.

Disclosed herein include recombinant AAV (rAAV). In some embodiments, the recombinant AAV (rAAV) comprises: a) a variant AAV capsid provided herein; and b) a heterologous nucleic acid. In some embodiments, the heterologous nucleic acid comprises a polynucleotide encoding a payload.

Disclosed herein include pharmaceutical compositions. In some embodiments, the pharmaceutical composition comprises: a rAAV provided herein, and a pharmaceutical excipient. In some embodiments, the pharmaceutical composition is for intraventricular, intraperitoneal, intraocular, intravenous, intraarterial, intranasal, intrathecal, intracistemae magna, or subcutaneous injection, and/or for direct injection to any tissue in the body. The pharmaceutical composition can comprise: a therapeutic agent.

Disclosed herein include methods of treating a disease or disorder in a subject. In some embodiments, the method comprises: administering to the subject a composition comprising a variant AAV capsid provided herein, or a therapeutically effective amount of an rAAV provided herein, or a pharmaceutical composition provided herein.

Disclosed herein include recombinant vectors. In some embodiments, the recombinant vector comprises a nucleic acid encoding a variant AAV capsid provided herein.

Disclosed herein include methods of manufacturing a recombinant AAV from the variant AAV capsids provided herein. In some embodiments, the method comprises: a) introducing into a cell: (i) a first nucleic acid comprising a heterologous nucleic acid comprising a polynucleotide encoding a payload; (ii) a second nucleic acid encoding a variant AAV capsid provided herein; and (iii) a third nucleic acid encoding an AAV helper virus genome; and b) assembling the recombinant AAV, the recombinant AAV comprising the variant AAV capsid encapsulating a heterologous nucleic acid.

Disclosed herein include kits. In some embodiments, the kit comprises: a) a recombinant vector comprising a nucleic acid encoding a variant AAV capsid provided herein; b) a helper vector encoding a helper virus protein; and c) a payload vector comprising a heterologous nucleic acid, wherein said heterologous nucleic acid comprises a polynucleotide encoding a payload.

Disclosed herein include methods of purifying recombinant AAV (rAAV). In some embodiments, the method comprises: (a) generating a viral particle extract comprising a plurality of rAAV provided herein, wherein the viral particle extract comprises the supernatant of lysed producer cells, or a derivative thereof; (b) contacting the viral particle extract with an ionic detergent to generate a first mixture; (c) contacting the first mixture with an acid to generate a second mixture; (d) centrifuging the second mixture to generate a supernatant; (e) filtering the supernatant with one or more filters to generate a filtrate; (f) performing one or more cycles of buffer exchange of the filtrate to a final storage buffer.

Disclosed herein include populations of recombinant AAV (rAAV). In some embodiments, the average diameter of the viral capsids of the population of rAAV range is about 30 nm to about 60 nm, from about 30 nm to about 50 nm, from about 30 nm to about 40 nm, or from about 30 nm to about 35 nm.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, refers to a recombinant nucleic acid that has been generated for the purpose of the expression of a specific nucleotide sequence(s), or that is to be used in the construction of other recombinant nucleotide sequences.

As used herein, the term “plasmid” refers to a nucleic acid that can be used to replicate recombinant DNA sequences within a host organism. The sequence can be a double stranded DNA.

The term “virus genome” refers to a nucleic acid sequence that is flanked by cis acting nucleic acid sequences that mediate the packaging of the nucleic acid into a viral capsid. For AAVs and parvoviruses, for example it is known that the “inverted terminal repeats” (ITRs) that are located at the 5′ and 3′ end of the viral genome have this function and that the ITRs can mediate the packaging of heterologous, for example, non-wildtype virus genomes, into a viral capsid.

The term “element” refers to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The term “construct,” as used herein, refers to a recombinant nucleic acid that has been generated for the purpose of the expression of a specific nucleotide sequence(s), or that is to be used in the construction of other recombinant nucleotide sequences.

As used herein, the term “variant” refers to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The term “AAV” or “adeno-associated virus” refers to a Dependoparvovirus within the Parvoviridae genus of viruses. For example, the AAV can be an AAV derived from a naturally occurring “wild-type” virus, an AAV derived from a rAAV genome packaged into a capsid derived from capsid proteins encoded by a naturally occurring cap gene and/or a rAAV genome packaged into a capsid derived from capsid proteins encoded by a non-natural capsid cap gene, for example, XL.D1c-AAV9 and XL.N1-AAV9. Non-limited examples of AAV include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), AAV type DJ (AAV-DJ), avian AAV, bovine AAV, canine AAV, equine AAV, primate AAV, non-primate AAV, and ovine AAV. In some instances, the AAV is described as a “Primate AAV,” which refers to AAV that infect primates. Likewise an AAV may infect bovine animals (e.g., “bovine AAV”, and the like). In some instances, the AAV is wildtype, or naturally occurring. In some instances the AAV is recombinant.

The term “AAV capsid” as used herein refers to a capsid protein or peptide of an adeno-associated virus. In some instances, the AAV capsid protein is configured to encapsidate genetic information (e.g., a heterologous nucleic acid, a transgene, therapeutic nucleic acid, viral genome). In some instances, the AAV capsid of the instant disclosure is a variant AAV capsid, which means in some instances that it is a parental AAV capsid that has been modified in an amino acid sequence of the parental AAV capsid protein.

The term “AAV genome” as used herein refers to nucleic acid polynucleotide encoding genetic information related to the virus. The genome, in some instances, comprises a nucleic acid sequence flanked by AAV inverted terminal repeat (ITR) sequences. The AAV genome may be a recombinant AAV genome generated using recombinatorial genetics methods, and which can include a heterologous nucleic acid (e.g., transgene) that comprises and/or is flanked by the ITR sequences.

The term “rAAV” refers to a “recombinant AAV”. In some embodiments, a recombinant AAV has an AAV genome in which part or all of the rep and cap genes have been replaced with heterologous sequences. The term “AAV particle”, “AAV nanoparticle”, or an “AAV vector” as used interchangeably herein refers to an AAV virus or virion comprising an AAV capsid within which is packaged a heterologous DNA polynucleotide, or “genome”, comprising nucleic acid sequence flanked by AAV inverted terminal repeat (ITR) sequences. In some cases, the AAV particle is modified relative to a parental AAV particle.

The term “cap gene” refers to the nucleic acid sequences that encode capsid proteins that form, or contribute to the formation of, the capsid, or protein shell, of the virus. In the case of AAV, the capsid protein may be VP1, VP2, or VP3. For other parvoviruses, the names and numbers of the capsid proteins can differ.

The term “rep gene” refers to the nucleic acid sequences that encode the non-structural proteins (rep78, rep68, rep52 and rep40) required for the replication and production of virus.

As used herein, “native” refers to the form of a polynucleotide, gene or polypeptide as found in nature with its own regulatory sequences, if present.

As used herein, “endogenous” refers to the native form of a polynucleotide, gene or polypeptide in its natural location in the organism or in the genome of an organism. “Endogenous polynucleotide” includes a native polynucleotide in its natural location in the genome of an organism. “Endogenous gene” includes a native gene in its natural location in the genome of an organism. “Endogenous polypeptide” includes a native polypeptide in its natural location in the organism.

As used herein, “heterologous” refers to a polynucleotide, gene or polypeptide not normally found in the host organism but that is introduced into the host organism. “Heterologous polynucleotide” includes a native coding region, or portion thereof, that is reintroduced into the source organism in a form that is different from the corresponding native polynucleotide. “Heterologous gene” includes a native coding region, or portion thereof, that is reintroduced into the source organism in a form that is different from the corresponding native gene. For example, a heterologous gene may include a native coding region that is a portion of a chimeric gene including non-native regulatory regions that is reintroduced into the native host. “Heterologous polypeptide” includes a native polypeptide that is reintroduced into the source organism in a form that is different from the corresponding native polypeptide. The subject genes and proteins can be fused to other genes and proteins to produce chimeric or fusion proteins. The genes and proteins useful in accordance with embodiments of the subject disclosure include not only the specifically exemplified full-length sequences, but also portions, segments and/or fragments (including contiguous fragments and internal and/or terminal deletions compared to the full-length molecules) of these sequences, variants, mutants, chimerics, and fusions thereof.

The term “exogenous” gene as used herein is meant to encompass all genes that do not naturally occur within the genome of an individual. For example, a miRNA could be introduced exogenously by a virus, e.g. an AAV nanoparticle.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™ Auxiliary, stabilizer, emulsifier, lubricant, binder, pH adjustor controller, isotonic agent and other conventional additives may also be added to the carriers.

Rational Design of AAV Capsid Nanoparticles with Expanded Sizes

Recombinant AAV (rAAV) mediated gene delivery leverages the AAV mechanism of viral transduction for nuclear expression of an episomal heterologous nucleic acid (e.g., a transgene, therapeutic nucleic acid). Upon delivery to a host in vivo environment, a rAAV will (1) bind or attach to cellular surface receptors on the target cell, (2) endocytose, (3) traffic to the nucleus, (4) uncoat the virus to release the encapsidated heterologous nucleic acid, (5) convert of the heterologous nucleic acid from single-stranded to double-stranded DNA as a template for transcription in the nucleus, and/or (6) transcribe of the episomal heterologous nucleic acid in the nucleus of the host cell (“transduction”). rAAVs engineered to have an expanded size, and therefore, capable of packaging oversized genomes, are desirable for gene therapy applications.

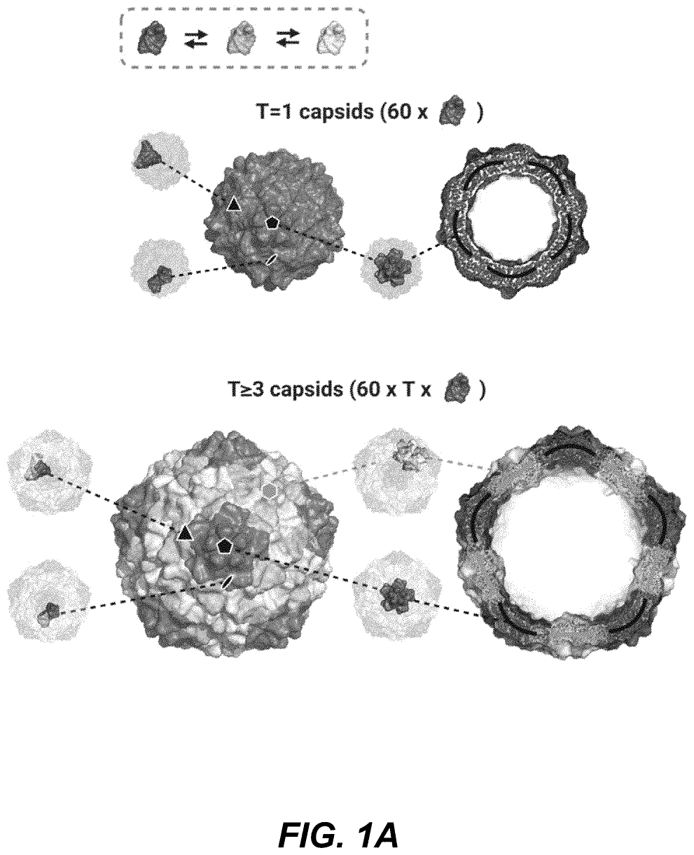

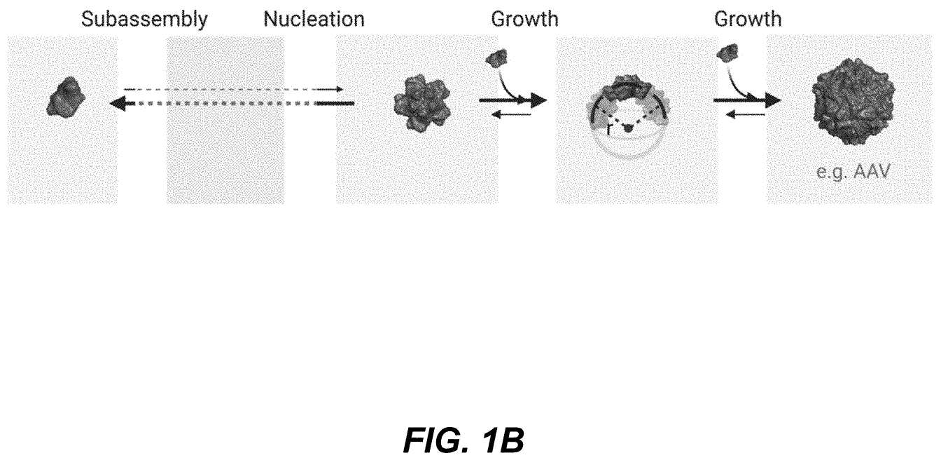

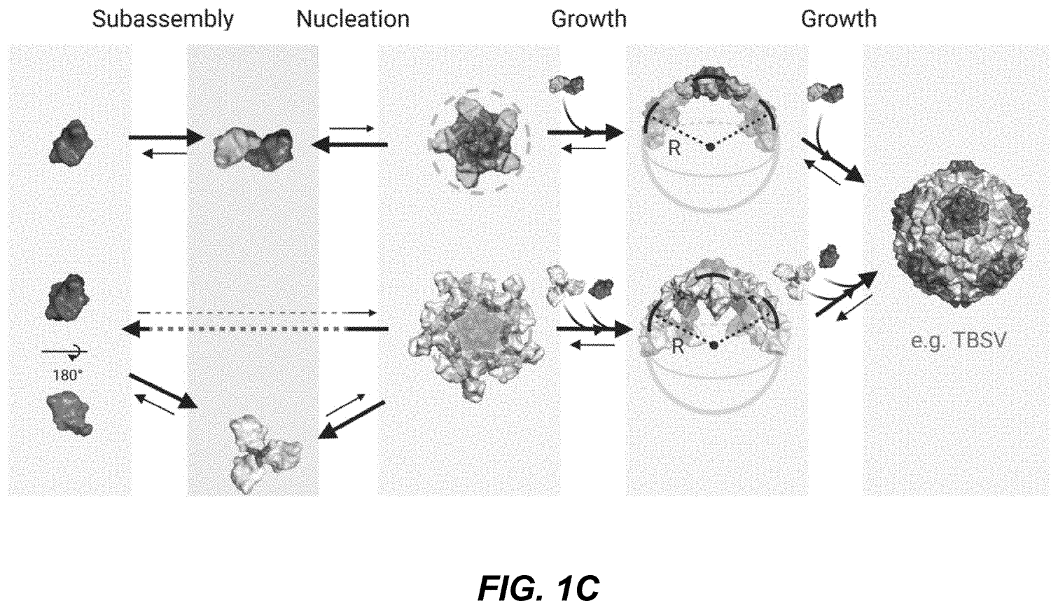

The application of the adeno-associated viral (AAV) capsid as a gene delivery vehicle is limited by its modest packaging capacity of ˜5 kb. This limitation is imposed by the capsid's 25-nm diameter, dictated by its conserved icosahedral geometry. The geometry of an icosahedral viral capsid is described by its triangulation (T) number. By this definition, a viral capsid is built from 60T subunits with symmetrical interactions. For example, a T=1 capsid like wild-type AAV comprises 60 subunits, whereas a T=3 capsid would comprise 180 subunits (FIG. 1A). Some icosahedral capsids, notably a number of ssRNA plant viral capsids that share similar jelly-roll protein folds as AAV capsids, can form spherical particles of different sizes (T numbers) with only minor sequence changes. The larger forms of these capsids are produced by organizing a greater number of identical subunits into more-complicated icosahedral geometries.

Provided herein are modified adeno-associated (AAV) virus capsid proteins and AAV nanoparticle compositions useful for integrating a transgene comprising an oversized cargo (e.g. a nucleic acid >5 kb) into a target cell or environment (e.g., a cell-type or tissue) in a subject when they are administered to the subject. Disclosed herein include variant AAV capsids. The variant AAV capsid can have a diameter of at least 30 nm. The variant AAV capsid can comprise a plurality of tandem multimers. The variant AAV capsid can comprise a plurality of HI loop variant capsid proteins. The variant AAV capsid can comprise a plurality of guided variant capsid proteins. The diameter and/or genetic cargo capacity of the variant AAV capsids can be greater than the corresponding parental AAV capsid. The modified AAV capsid proteins of the present disclosure can comprise at least one insertion or substitution of one or more amino acids in a corresponding parental AAV capsid protein that confers expanded size and cargo capacity to AAV nanoparticles as compared to a reference/parental AAV capsid. The rAAVs described herein are useful for a wide range of applications, including but not limited to the treatment of disorders and disease. The expanded size and cargo capacity of the AAV nanoparticles comprising the variant capsid proteins disclosed herein can permit delivery of nucleic acids (e.g., viral genomes, heterologous nucleic acids) comprising large therapeutic transgenes (e.g. CFTR).

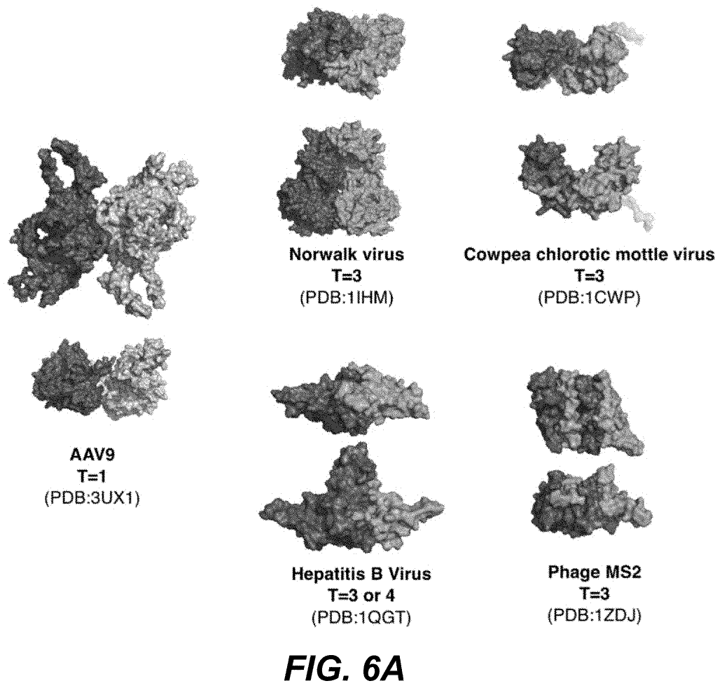

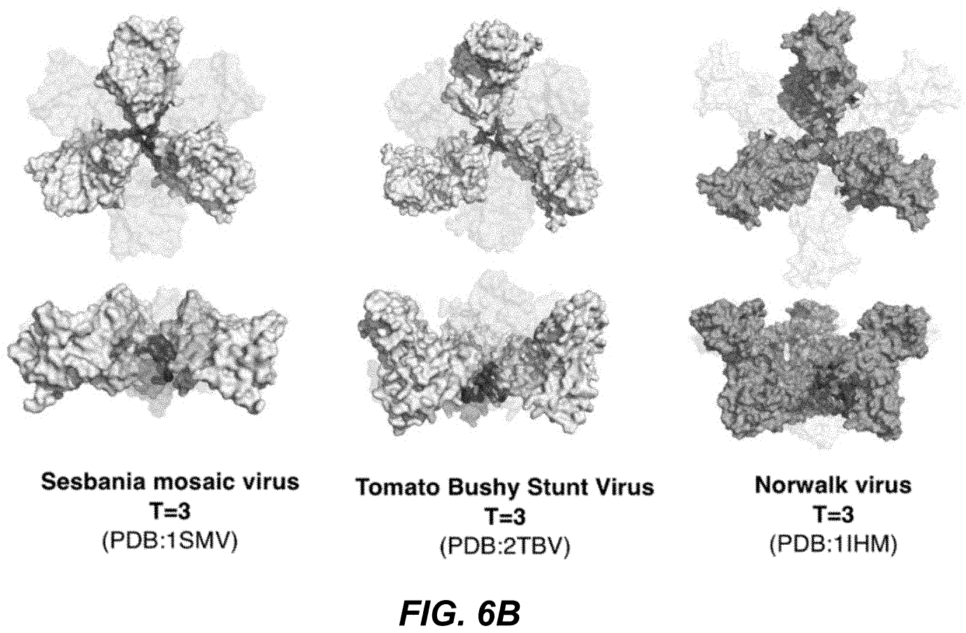

Without being bound by any particular theory, two common features of the naturally T=3 or T=4 capsids may facilitate the assembly and organization of subunits into expanded geometries (FIGS. 1B-1C). The first common feature is the fast sub-assembly of multi-subunit, particularly dimeric, structural units (FIG. 1C, top), enabled by a simple interface that buries significant surface area of each subunit (FIG. 6A). Example capsids that use such strong dimeric structural units include phage R17, cowpea chlorotic mottle virus (CCMV), norovirus, and hepatitis B virus capsids. By contrast, the dimeric interface between AAV subunits is weak and dynamic (FIG. 6A, left). The second common feature is the formation of additional 3-fold interactions on the interior side of the protein shell via a flexible peptide with interchangeable conformations (FIGS. 1B-1C), demonstrated in studies with ssRNA plant viral capsids such as tomato bushy stunt virus (TBSV) or CCMV capsids. Both features may help establish ordered assembly pathways that use multi-subunit structural units as building blocks for nucleation and growth. These sub-assembled building blocks may help extend the pentameric critical nucleus's outer edge and create extra space between two neighboring curved pentamers (FIG. 1C). In addition, strengthened interactions within the multi-subunit structural units may also help stabilize intermediate scaffolds with larger radii of curvature. Disclosed herein are methods and compositions exploiting these two features to modify AAV capsid subunits and promote the formation of larger capsids.

Disclosed herein include variant AAV capsids. The diameter of the variant capsids can vary. In some embodiments, the variant AAV capsid has a diameter of at least 30 nm. The variant AAV capsid can have a diameter of about 30 nm to about 35 nm, of about 30 nm to about 60 nm, of about 40 nm to about 60 nm, of about 45 nm to about 60 nm, of about 50 nm to about 65 nm, or of about 55 nm to about 60 nm, of about 20 nm to about 100 nm, of about 20 nm to about 80 nm, of about 20 nm to about 60 nm, of about 20 nm to about 40 nm, of about 30 nm to about 100 nm, of about 30 nm to about 80 nm, of about 30 nm to about 60 nm, of about 20 nm to about 40 nm, of about 40 nm to about 100 nm, of about 40 nm to about 80 nm, of about 40 nm to about 60 nm, of about 50 nm to about 100 nm, of about 50 nm to about 80 nm, of about 50 nm to about 60 nm, of about 60 nm to about 100 nm, or of about 60 nm to about 80 nm, or a number or a range between any two of these values. The diameter of a variant capsid can be, or can be about, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, or a number or a range between any two of these values. In some embodiments, the capsid particles demonstrate a variation in size. In some embodiments, a small population of the particles are smaller than 35 nm. In some embodiments, the relatively smaller capsids of the population of capsids are more spherical and/or more compact. In some embodiments, the target diameter of a spherical capsid is about 35 nm. In some embodiments, and without being bound by any particular theory, the particles in the smaller side of the spectrum are more functionally useful given that the nuclear pore complex has a diameter of 40 nm. The diameter can be calculated as the mean of the major axis length and the minor axis length. The diameter can be measured by transmission electron microscopy (TEM). The diameter can be hydrodynamic diameter. The hydrodynamic diameter can be measured by dynamic light scattering (DLS).

The genetic cargo capacity of the variant AAV capsids provided herein can vary. The genetic cargo capacity of the variant AAV capsids can be, or can be about, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10.0 kb, or a number or a range between any two of these values. The genetic cargo capacity of the variant AAV capsids can be at least, or can be at most, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, or 10.0 kb. The genetic cargo capacity can be: (i) the maximum length of a single-stranded DNA molecule that the variant AAV capsid is capable of protecting from DNAse I digestion; and/or (ii) the maximum length of a double-stranded DNA molecule that the variant AAV capsid is capable of protecting from DNAse I digestion. The single-stranded DNA molecule can be capable of self-hybridizing to form a double-stranded region. The single-stranded DNA molecule can comprise a self-complementary AAV (scAAV) vector.

The variant AAV capsid can comprise a plurality of tandem multimers. A tandem multimer can comprise two or more AAV capsid proteins, and the tandem multimer can comprise one or more linkers connecting the two or more AAV capsid proteins. The two or more AAV capsid proteins can comprise VP1, VP2, VP3, derivatives thereof, or any combination thereof. The variant AAV capsid can comprise two or more tandem multimers that differ with respect to the capsid protein isoforms that compose said tandem multimers. One or more of said two or more AAV capsid proteins can comprise a HI loop variant capsid protein. The two or more AAV capsid proteins can comprise two or more parental AAV capsid proteins, or derivatives thereof. A plurality of tandem multimers can be capable of assembling into the variant AAV capsid.

The variant AAV capsid can comprise a plurality of HI loop variant capsid proteins. A HI loop variant capsid protein can comprise a HI loop variant of a parental AAV capsid protein. A HI loop variant capsid protein can comprise a removal of one or more amino acids in the capsid protein HI loop relative to a corresponding parental AAV capsid protein. The HI loop variant capsid protein can comprise VP1, VP2, and/or VP3. A plurality of HI loop variant capsid proteins can be capable of assembling into the variant AAV capsid. In some embodiments, the variant AAV capsid comprises a plurality of HI loop variant capsid proteins. In some embodiments, the variant AAV capsid comprises monomeric HI loop variant capsid proteins. In some embodiments, said variant AAV capsid does not comprise tandem multimers and/or guided variant capsid proteins. In some embodiments, said variant AAV capsid is not size-expanded and/or capacity-increased capsids. In some embodiments, said variant AAV capsid is an about 25-nm particle.

The variant AAV capsid can comprise a plurality of guided variant capsid proteins. The guided variant capsid protein can comprise an insertion of a guide peptide relative to a corresponding parental AAV capsid protein. The guided variant capsid protein can comprise VP1, VP2, and/or VP3. A plurality of guided variant capsid proteins can be capable of assembling into the variant AAV capsid. A plurality of parental AAV capsid proteins can be capable of assembling into a corresponding parental AAV capsid. The variant AAV capsid can comprise a larger diameter and/or genetic cargo capacity as compared to a corresponding parental AAV capsid assembled from said parental AAV capsid proteins,

The parental AAV capsid proteins can comprise VP1, VP2, and/or VP3. In some embodiments, the corresponding parental AAV capsid does not comprise: (i) a tandem multimer; (ii) a HI loop variant capsid protein; and/or (iii) a guided variant capsid protein. The corresponding parental AAV capsid can comprise a genetic cargo capacity of less than about 4.8 kb, of less than about 4.9 kb, of less than about 5.0 kb, of less than about 5.1 kb, or of less than about 5.2 kb. The corresponding parental AAV capsid can comprise a diameter of less than about 25 nm, of less than about 26 nm, of less than about 27 nm, or of less than about 28 nm. The variant AAV capsid can comprise at least about 10% (e.g., 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900%, 1000%, or a number or a range between any two of these values) greater molecular weight as compared to the corresponding parental AAV capsid.

The variant AAV capsid can comprise at least about 10% (e.g., 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900%, 1000%, or a number or a range between any two of these values) more capsid subunits as compared to the corresponding parental AAV capsid. The variant AAV capsid can comprise a triangulation number of 1, 2, 3, 4, or 5. The variant AAV capsid can comprise at least about 60 (e.g., 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, or a number or a range between any two of these values) subunits. Subunits can be monomeric or multimers.

The variant AAV capsid can comprise at least about 10% (e.g., 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900%, 1000%, or a number or a range between any two of these values) larger diameter and/or genetic cargo capacity as compared to the corresponding parental AAV capsid.

The packaging efficiency of the variant AAV capsid can be at least about 0.1% (e.g., 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of the packaging efficiency of the corresponding parental AAV capsid.

The transduction efficiency of the variant AAV capsid can be at least about 0.1% (e.g., 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of the transduction efficiency of the corresponding parental AAV capsid.

The one or more linkers can be situated at the termini of the two or more AAV capsid proteins. At least one linker of the one or more linkers can comprise the amino acid sequence of a protease cleavage site. At least one linker of the one or more linkers can comprise the amino acid sequence of [GGS]. The tandem multimer can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 116. At least one linker of the one or more linkers can comprise the amino acid sequence of SEQ ID NO: 13. The tandem multimer can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 118. At least one linker of the one or more linkers can comprise the amino acid sequence of SEQ ID NO: 14. The tandem multimer can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 119. The tandem multimer can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 124 or to SEQ ID NO: 125.

At least one linker of the one or more linkers can comprise a flexible peptide linker. A flexible peptide linker can comprise about 1 to about 18 (e.g., 1, 2, 3, 4, 5, 6, 7, 8, 9, 10, 11, 12, 13, 14, 15, 16, 17, or 18) flexible amino acid residues. The flexible amino acid residues can comprise glycine, serine, or a combination thereof.

The tandem multimer can comprise a tandem dimer of a first capsid protein and a second capsid protein. The tandem dimer can comprise a first linker. In some embodiments, a tandem dimer of two AAV capsid proteins comprises a stronger intramolecular 2-fold interaction as compared to the first capsid protein and the second capsid protein not connected by the first linker. In some embodiments, the first linker enhances the apparent association rate and/or decreases the apparent dissociation rate of the dimeric interaction between the first capsid protein and the second capsid protein. The tandem multimer can comprise a tandem trimer of a first capsid protein, a second capsid protein, and a third capsid protein. The tandem trimer can comprise a first linker and a second linker. The first linker and the second linker can comprise about 5 flexible amino acid residues. The tandem multimer can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 120. The tandem multimer can comprise a tandem tetramer of a first capsid protein, a second capsid protein, a third capsid protein, and a fourth capsid protein. The tandem tetramer can comprise a first linker, a second linker, and a third linker. The first linker and the second linker can comprise about 5 flexible amino acid residues, and the third linker can comprise about 9 flexible amino acid residues. The tandem multimer can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 121.

The first capsid protein, the second capsid protein, the third capsid protein, and/or the fourth capsid protein can comprise VP1, VP2, VP3, a HI loop variant thereof, or any combination thereof. The first capsid protein, second capsid protein, third capsid protein, and/or fourth capsid protein can comprise a HI loop variant capsid protein. The HI loop variant capsid protein can comprise VP1, VP2, and/or VP3. The removal of one or more amino acids in the capsid protein HI loop further can comprise an insertion of a flexible peptide linker in the HI loop, and the insertion of a flexible peptide linker can replace a contiguous stretch of from 2 amino acids to 18 amino acids (e.g., 2, 3, 4, 5, 6, 7, 8, 9, 10, 11, 12, 13, 14, 15, 16, 17, or 18 residues) of the parental AAV capsid protein. A flexible peptide linker can comprise about 1 to about 18 (e.g., 1, 2, 3, 4, 5, 6, 7, 8, 9, 10, 11, 12, 13, 14, 15, 16, 17, or 18) flexible amino acid residues. The flexible amino acid residues can comprise glycine, serine, or a combination thereof.

A HI loop variant capsid protein can comprise a HI loop truncated by 1, 2, 3, 4, 5, 6, 7, 8, 9, 10, 11, 12, 13, 14, 15, 16, 17, or 18 residues relative to a corresponding parental AAV capsid protein. The HI loop of the corresponding parental AAV capsid protein can comprise about 18 amino acids. The HI loop can be located between amino acid V654 and amino acid 1671 of VP1 of AAV9, or the corresponding position in the capsid protein of another AAV serotype. The HI loop variant capsid protein can comprise the removal of 1, 2, 3, 4, 5, 6, 7, 8, 9, 10, 11, or 12 amino acids of the stretch of HI Loop amino acid residues between amino acid D657 and amino acid N668 (DPPTAFNKDKLN; SEQ ID NO: 127) of VP1 of AAV9, or the corresponding amino acids in the capsid protein of another AAV serotype. The HI loop variant capsid protein can comprise the removal of 1, 2, 3, 4, 5, 6, 7, or 8 amino acids of the stretch of HI Loop amino acid residues between amino acid P659 and amino acid K666 (PTAFNKDK; SEQ ID NO: 128) of VP1 of AAV9, or the corresponding amino acids in the capsid protein of another AAV serotype. The HI loop variant capsid protein can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 114 or to SEQ ID NO: 115. The tandem multimer can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 117.

The guide peptide can comprise about 3 amino acids to about 500 amino acids (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, or a number or a range between any two of these values). The guide peptide can comprise a contiguous stretch of from about 2 amino acids to about 100 amino acids (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values) from the N-terminal region of a capsid protein of a larger virus species. The N-terminal region of a capsid protein of a larger virus species can comprise: (i) the first 100 amino acids of said capsid protein; and/or (ii) the amino acid sequence of the first 25% of full-length amino acid sequence of said capsid protein. The insertion of a guide peptide can be at a structurally analogous turn in the parental AAV capsid protein relative to the capsid protein of the larger virus species. In some embodiments, said larger virus species forms a capsid comprising a larger diameter and/or genetic cargo capacity than the corresponding parental AAV capsid. The capsid of the larger virus species can comprise a triangulation (T) number of greater than 1. The larger virus species can comprise Tomato bushy stunt virus (TBSV), Sesbania mosaic virus (SMV), Norwalk virus, variants thereof, or any combination thereof. The larger virus species can comprise Cucumber necrosis virus, Tomato bushy stunt virus (cherry strain), Tomato bushy stunt virus (BS-3 strain), Turnip crinkle virus, Carrot mottle virus, Carnation ringspot virus, Sesbania mosaic virus, Southern bean mosaic virus, Southern cowpea mosaic virus, Brome mosaic virus, Rabbit hemorrhagic disease virus, Feline Calicivirus, Nowalk virus, variants thereof, or any combination thereof. The larger virus species can comprise a species of Orthornavirae. Exemplary larger virus species are depicted in Table 8. Exemplary capsid sequences of larger virus species to be employed for the generation of guide peptides are depicted in Tables 9-10.

The guide peptide can comprise a contiguous stretch of at least about 10 (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, or a number or a range between any two of these values) amino acids of any one of the sequences of SEQ ID NOS: 89-102 or of a sequence comprising one mismatch or two mismatches relative to any one of the sequences of SEQ ID NOS: 89-102. The guide peptide can comprise a contiguous stretch of from about 2 amino acids to about 100 amino acids (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values) of any one of the sequences of SEQ ID NOS: 103-109. The capsid protein of the larger virus can comprise a similar core fold as an AAV capsid protein. The capsid protein of the larger virus can comprise a core jelly-roll protein fold. In some embodiments, a removal of the guide peptide from the capsid protein of the larger virus species causes a shrinkage of the capsid of the larger virus species.

The insertion of a guide peptide can be between any one of amino acid 1 to amino acid 240 of VP1 of AAV9, or the corresponding position in the capsid protein of another AAV serotype. In some embodiments, the insertion of a guide peptide replaces a contiguous stretch of from about 2 amino acids to about 200 amino acids (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, or a number or a range between any two of these values) of the parental AAV capsid protein. In some embodiments, the insertion of a guide peptide replaces a contiguous stretch of from about 2 amino acids to about 200 amino acids (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, or a number or a range between any two of these values) of the parental AAV capsid protein following the VP1 start codon, VP2 start codon, and/or VP3 start codon. In some embodiments, the insertion of a guide peptide replaces a contiguous stretch of from about 2 amino acids to about 200 amino acids (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, or a number or a range between any two of these values) of the parental AAV capsid protein following the VP2 start codon. In some embodiments, the insertion of a guide peptide replaces a contiguous stretch of from about 2 amino acids to about 50 amino acids (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or a number or a range between any two of these values) following the VP3 start codon.

In some embodiments, the insertion of a guide peptide replaces amino acid A204 to amino acid G220 of VP1 of AAV9, or the corresponding position in the capsid protein of another AAV serotype. The guided variant capsid protein can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 122. The guide peptide can comprise amino acid A2 to amino acid G76 of TBSV (cherry strain) coat protein (CP). The guide peptide can comprise amino acid A2 to amino acid P82 of TBSV (cherry strain) CP. The guide peptide can comprise amino acid A2 to amino acid 5101 of TBSV (cherry strain) CP. The guide peptide can comprise amino acid 169 to amino acid P82 of TBSV (cherry strain) CP. In some embodiments, the insertion of the guide peptide replaces amino acid A204 to amino acid G220 of VP1 of AAV9, or the corresponding position in the capsid protein of another AAV serotype. The guided variant capsid protein can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 123.

The guide peptide can be conditionally structured. The guide peptide can be capable of forming a β-annulus structure. The β-annulus structure can be capable of stabilizing the pseudo-6-fold interface. The β-annulus structure can be capable of forming a guided variant trimer. The guided variant trimer can comprise three guided variant capsids interacting via the β-annulus structure of each guided variant capsid protein. A plurality of guided variant trimers can be capable of assembling into the variant AAV capsid. The variant AAV capsid can comprise a pseudo-6-fold axis of symmetry. In some embodiments, the guide peptide involved in an intertwined β-annulus structure at a pseudo-6-fold symmetrical interface of the variant AAV capsid. In some embodiments, the guide peptide enables the formation of additional 3-fold interactions on the interior side of the variant AAV capsid as compared to the corresponding parental AAV capsid. The corresponding parental AAV capsid can comprise capsid proteins comprising an intact HI loop. In some embodiments, the corresponding parental AAV capsid comprises 2-fold symmetrical interfaces, 3-fold symmetrical interfaces, and 5-fold symmetrical interfaces, and wherein the corresponding parental AAV capsid does not comprise a pseudo-6-fold symmetrical interface. The variant AAV capsid can comprise a plurality of pseudo-6-fold symmetrical interfaces. In some embodiments, the plurality of pseudo-6-fold symmetrical interfaces enables the incorporation of additional AAV capsid proteins in the variant AAV capsid as compared to the corresponding parental AAV capsid. In some embodiments, the HI loop of the corresponding parental AAV capsid sterically hinders the formation of a planar hexamer, and the HI loop variant capsid protein does not sterically hinder the formation of a planar hexamer. In some embodiments, the HI loop variant capsid protein can be adapted into a planar hexamer without apparent steric hindrance. The assembly of the variant AAV capsid can comprise a larger critical nucleus as compared to the critical nucleus formed during the assembly of the corresponding parental AAV capsid. The critical nucleus can comprise a pentameric critical nucleus. The larger critical nucleus can comprise extra space between curved pentamers. The extra space between curved pentamers can be eventually filled in by hexamers during assembly of the variant AAV capsid. Assembly of the variant AAV capsid can comprise a prolonged lifetime of an intermediate scaffold with a larger radii of curvature as compared to the intermediate scaffolds formed during the assembly of the corresponding parental AAV capsid.

TABLE 1
CAPSID VARIANT PROTEIN SEQUENCES

| NAME | SEO ID NO |
|---|---|
| AAV9-d6 | 114 |
| AAV9-d3 | 115 |
| XL.D0-AAV9 | 116 |
| XL.D1a-AAV9 | 117 |
| XL.D1b-AAV9 | 118 |
| XL.D1c-AAV9 | 119 |
| XL.T1a-AAV9 | 120 |
| XL.Q1a-AAV9 | 121 |
| XL.N0-AAV9 | 122 |
| XL.N1-AAV9 | 123 |
| XL.D1b-AAV-DJ | 124 |
| XL.D1c-AAV-DJ | 125 |
| wild-type AAV9 VP1 | 126 |

The variant AAV capsid can comprise VP1, VP2, and/or VP3. The variant AAV capsid can comprise an about 1:1:10 ratio of VP1:VP2:VP3. The structure of the variant AAV capsid can retain at least one surface epitope present on the corresponding parental AAV capsid. The at least one surface epitope can be responsible for targeting the variant AAV capsid to one or more cell types. The variant AAV capsid can be capable of being purified with an antigen-binding fragment versus the corresponding parental AAV capsid. The variant AAV capsid and/or the corresponding parental AAV capsid can comprise an icosahedral geometry. The VP1 of AAV9 can comprise an amino acid sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 126. In some embodiments, amino acid residue positions provided herein are in reference to the sequence of VP1.

In some embodiments, one or more of the variant AAV capsid, the corresponding parental AAV capsid, the parental AAV capsid protein, the tandem multimer, the HI loop variant capsid protein, the guided variant capsid protein, the rAAV, VP1, VP2, and/or VP3 disclosed herein have an AAV serotype selected from the group comprising AAV9, AAV9 K449R (or K449R AAV9), AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B 3 (PHP.B 3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2 Al 5/G2A3 (G2A3), AAVG2B4 (G2B4), AAVG2B5 (G2B5), PEEPS, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/rl 1.64, AAV4-9/rh.54, AAV4-19/rh.55, AAV5-3/rh.57, AAV5-22/rh.58, AAV7.3/hu.7, AAV16.8/hu.lO, AAV16.12/hu.1 1, AAV29.3/bb.1, AAV29.5/bb.2, AAV106. 1/hu.37, AAV1 14.3/hu.40, AAV127.2/hu.41, AAV127.5/hu.42, AAV128.3/hu.44, AAV130.4/hu.48, AAV145. 1/hu.53, AAV145.5/hu.54, AAV145.6/hu.55, AAV161. 10/hu.60, AAV161.6/hu.61, AAV33. 12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu. 12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu. 1, AAVhu.2, AAVhu.3, AAVhu.4, AAVhu.5, AAVhu.6, AAVhu.7, AAVhu.9, AAVhu.lO, AAVhu. 1 1, AAVhu. 13, AAVhu.15, AAVhu.16, AAVhu. 1 7, AAVhu.1 8, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu. 1 4/9, AAVhu.t 19, AAVrh.2, AAVrh.2R, AAVrh.8, AAVrh.8R, AAVrh. 10, AAVrh.12, AAVrh. 13, AAVrh.13R, AAVrh. 14, AAVrh.17, AAVrh. 18, AAVrh.19, AAVrh.20, AAVrh.21, AAVrh.22, AAVrh.23, AAVrh.24, AAVrh.25, AAVrh.3 1, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533 A mutant, AAAV, BAAV, caprine AAV, bovine AAV, AAVhE1.1, AAVhEr1.5, AAVhER1. 14, AAVhEr1.8, AAVhEr1. 16, AAVhEr1 0.18, AAVhEr1.35, AAVhEr1.7, AAVhEr1.36, AAVhEr2.29, AAVhEr2.4, AAVhEr2.16, AAVhEr2.30, AAVhEr2.3 1, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-lOl, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu. 11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128. 1/hu.43, true type AAV (ttAAV), EGRENN AAV 10, Japanese AAV 10 serotypes, AAV CBr-7.1, AAV CBr-7. 10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6. 1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-1 3, AAV CLvl-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-1 1, AAV CSp-2, AAV CSp-3, AAV CSp-4, AAV CSp-6, AAV CSp-7, AAV CSp-8, AAV CSp-8. 10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, variants thereof, a hybrid or chimera of any of the foregoing AAV serotypes, or any combination thereof.

The engineered AAV capsid proteins described herein have, in some cases, an insertion or substitution of an amino acid that is heterologous to the parental AAV capsid protein at the amino acid position of the insertion or substitution. In some embodiments, the amino acid is not endogenous to the parental AAV capsid protein at the amino acid position of the insertion or substitution. The amino acid may be a naturally occurring amino acid in the same or equivalent amino acid position as the insertion of the substitution in a different AAV capsid protein.

The rAAV may comprise a chimeric AAV capsid. A “chimeric” AAV capsid refers to a capsid that has an exogenous amino acid or amino acid sequence. The rAAV may comprise a mosaic AAV capsid. A “mosaic” AAV capsid refers to a capsid that made up of two or more capsid proteins or polypeptides, each derived from a different AAV serotype. The rAAV may be a result of transcapsidation, which, in some cases, refers to the packaging of an inverted terminal repeat (ITR) from a first serotype into a capsid of a second serotype, wherein the first and second serotypes are not the same. In some cases, the capsid genes of the parental AAV serotype is pseudotyped, which means that the ITRs from a first AAV serotype (e.g., AAV2) are used in a capsid from a second AAV serotype (e.g., AAV9), wherein the first and second AAV serotypes are not the same. As a non-limiting example, a pseudotyped AAV serotype comprising the AAV2 ITRs and AAV9 capsid protein may be indicated AAV2/9. The rAAV may additionally, or alternatively, comprise a capsid that has been engineered to express an exogenous ligand binding moiety (e.g., receptor), or a native receptor that is modified.

The reference AAV disclosed herein, in some cases, is AAV9. However, the reference AAV may be any serotype, e.g. a serotype selected from the group consisting of AAV1, AAV2, AAV3, AAV4, AAV5, AAV6, AAV7, AAV8, AAV9, AAV10, AAV11, AAV12, AAV-DJ, or variant thereof. In many instances, the reference AAV is the parental AAV, e.g., the corresponding unmodified AAV from which the variant AAV was engineered.

In some embodiments, the rAAV capsid proteins of the present disclosure comprise a substitution or insertion of one or more amino acids in an amino acid sequence of an AAV capsid protein. The AAV capsid protein from which the engineered AAV capsid protein of the present disclosure is produced is referred to as a “parental” AAV capsid protein, or a “corresponding unmodified capsid protein”. In some cases, the parental AAV capsid protein has a serotype selected from the group consisting of AAV1, AAV2, rAAV3, AAV4, AAV5, AAV6, AAV7, AAV8, AAV9, AAV10, AAV11, and AAV12. The complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. At least portions of the AAV-DJ genome are provided in Grimm, D. et al. J. Virol. 82, 5887-5911 (2008).

Recombinant Vectors and Methods of Producing rAAVs

Disclosed herein include recombinant vectors. In some embodiments, the recombinant vector comprises a nucleic acid encoding a variant AAV capsid provided herein. Disclosed herein include methods of manufacturing a recombinant AAV from the variant AAV capsids provided herein. In some embodiments, the method comprises: a) introducing into a cell: (i) one or more nucleic acids (e.g., a first nucleic acid) comprising a heterologous nucleic acid comprising a polynucleotide encoding a payload; (ii) one or more nucleic acids (e.g., a second nucleic acid) encoding a variant AAV capsid provided herein; and (iii) one or more nucleic acids (e.g., a third nucleic acid) encoding an AAV helper virus genome; and/or b) assembling the recombinant AAV, the recombinant AAV comprising the variant AAV capsid encapsulating a heterologous nucleic acid.

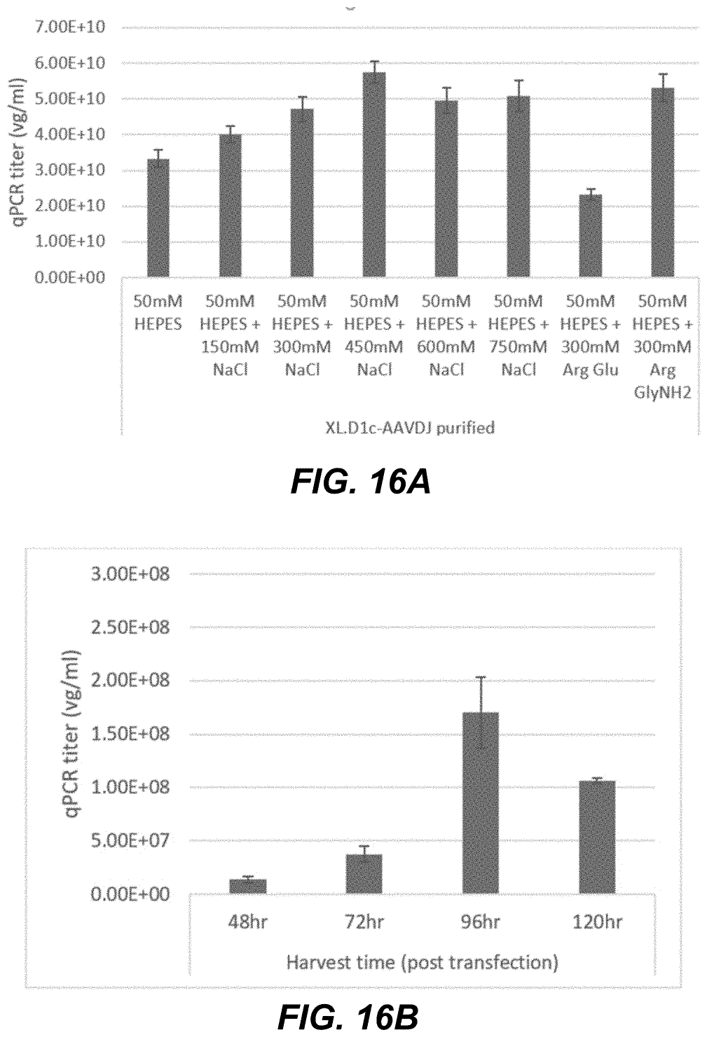

Disclosed herein include methods of purifying recombinant AAV (rAAV). There are provided, in some embodiments, improved methods of viral particle (e.g., rAAV) purification. An example of an improved purification method provided herein is described in Example 7. In some embodiments, said these improved methods of rAAV purification result in improved yield and/or stability of rAAV (See, e.g., FIGS. 16A-16B) as compared to currently available methods, such as those that have been previously disclosed in, for example, in U.S. Pat. Pub. No. 2018/0273907, the content of which is hereby expressly incorporated by reference in its entirety. In some embodiments, harvest time after transfection is increased from 2 day (as in currently available methods) to 3-4 days. In some embodiments, the final buffer used for buffer exchange and storage is changed from regular PBS (as in currently available methods) to high-salt buffer. In some such embodiments, without being bound by any particular theory, the improved yield and/or stability of purified viral particles prepared using the methods provided herein is due to the use of a high-salt buffer as the final buffer used for buffer exchange and storage and/or the change in the harvest time after transfection. In some embodiments, the method comprises: (a) generating a viral particle extract comprising a plurality of rAAV provided herein, wherein the viral particle extract comprises the supernatant of lysed producer cells, or a derivative thereof; (b) contacting the viral particle extract with a detergent (e.g., an ionic detergent) to generate a first mixture; (c) contacting the first mixture with an acid to generate a second mixture; (d) centrifuging the second mixture to generate a supernatant; (e) filtering the supernatant with one or more filters to generate a filtrate; (f) performing one or more cycles of buffer exchange of the filtrate to a final storage buffer.

The method can comprise: prior to step (b), contacting the viral particle extract with DNase I and/or MspI. The acid can comprise citric acid (e.g., 1 M citric acid). The quantity of the acid contacted with first mixture can be about 4% of the volume of the first mixture. The one or more filters can comprise an about 0.45 μm filter. Step (d) can comprise centrifugation at about 5000 g for 5 about minutes. Step (f) can comprise at least about 1, 2, 3, 4, 5, 6, 7, 8, 9, or 10, cycles of buffer exchange. Each cycle of buffer exchange can comprise performing centrifugation of concentrator tubes. The centrifugation can comprise about 1000 g for about 20 minutes. Step (f) can comprise buffer exchange with a 100 kD MWCO centrifugal concentrator. Step (f) can comprise an at least about at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) reduction in solution volume. The final storage buffer can comprise PBS, additional NaCl, and/or Pluronic F-68. The Pluronic F-68 can be 0.001% Pluronic F-68. The additional NaCl can be 300 mM additional NaCl. The detergent (e.g., an ionic detergent) can comprise sodium deoxycholate (e.g., 5% (m/v) sodium deoxycholate). The quantity of ionic detergent contacted with the viral particle extract can be about 10% of the volume of the viral particle extract. The detergent may be non-ionic, cationic, anionic or zwitterionic. Mixtures of detergents may also be used. Exemplary classes of detergents include alcohol ether sulfates, alcohol sulfates, alkanolamides, alkyl sulfonates, amine oxides, amphoteric detergents, anionic detergents, betaine derivatives, cationic detergents, disulfonates, dodecylbenzene sulfonic acid, ethoxylated alcohols, ethoxylated alkyl phenols, ethoxylated fatty acids, glycerol esters hydrotropes, lauryl sulfates, mono and diglycerides, non-ionic detergents, phosphate esters, quaternary detergents, and sorbitan derivatives.

Disclosed herein are methods of producing a recombinant AAV (rAAV). In some instances all elements that are required for AAV production in target cell (e.g., HEK293 cells) are transiently transfected into the target cell using suitable methods known in the art. For example, the rAAV of the present disclosure can be product by co-transfecting three plasmid vectors, a first vector with ITR-containing plasmid carrying the transgene (e.g., payload), a second vector that carries the AAV Rep and Cap genes (e.g., one or more variant capsid proteins provided herein, such as, for example, tandem multimers, HI loop variant capsid proteins, and/or guided variant capsid proteins); and (3), a third vector that provides the helper genes isolated from adenovirus. In some cases, rAAVs of the present disclosure are generated using the methods described in Challis, R. C. et al. Systemic AAV vectors for widespread and targeted gene delivery in rodents. Nat. Protoc. 14, 379 (2019), which is hereby incorporated by reference in its entirety. Briefly, triple transfection of HEK293T cells (ATCC) using polyethylenimine (PEI) is performed, viruses are collected after 120 hours from both cell lysates and media and purified over iodixanol.

Disclosed herein, are methods of manufacturing comprising: (a) introducing into a cell a nucleic acid comprising: (i) a first nucleic acid sequence (heterologous nucleic acid) encoding a payload product enclosed by a 5′ and a 3′ inverted terminal repeat (ITR) sequence; (ii) a second nucleic acid sequence encoding a viral genome comprising a 5′ ITR sequence, a Replication (Rep) gene, one or more (Cap) genes, and a 3′ ITR, wherein the one or more Cap genes encodes a variant AAV capsid protein described herein (e.g., tandem multimers, HI loop variant capsid proteins, and/or guided variant capsid proteins); and (iii) a third nucleic acid sequence encoding a first helper virus protein selected from the group consisting of E4orf6, E2a, and VA RNA, and optionally, a second helper virus protein comprising E1a or E1b55k; (b) expressing in the cell the AAV capsid protein described herein; (c) assembling an AAV particle comprising the AAV capsid proteins disclosed herein; and (d) packaging the first nucleic acid sequence in the AAV particle. In some instances, the methods further comprise packing the first nucleic acid sequence encoding the therapeutic gene expression product such that it becomes encapsidated by the rAAV capsid protein. In some embodiments, the rAAV particles are isolated, concentrated, and purified using suitable viral purification methods, such as those described herein.

In a non-limiting example, the rAAVs are generated by triple transfection of precursor cells (e.g., HEK293T) cells using a standard transfection protocol (e.g., with PEI). Viral particles are harvested from the media after a period of time (e.g., 72 h post transfection) and from the cells and media at a later point in time (e.g., 120 h post transfection). Virus present in the media is concentrated by precipitation with 8% poly(ethylene glycol) and 500 mM sodium chloride and the precipitated virus is added to the lysates prepared from the collected cells. The viruses are purified over iodixanol (Optiprep, Sigma) step gradients (15%, 25%, 40% and 60%). Viruses are concentrated and formulated in PBS. Virus titers are determined by measuring the number of DNasel-resistant vector genome copies (VGs) using qPCR and the linearized genome plasmid as a control.

The Rep protein can be selected from the group consisting of Rep78, Rep68, Rep52, and Rep40. The genome of the AAV helper virus comprises an AAV helper gene selected from the group consisting of E2, E4, and VA. In some instances, the first nucleic acid sequence and the second nucleic acid sequence are in trans. In some instances, the first nucleic acid sequence and the second nucleic acid sequence are in cis. In some instances, the first nucleic acid sequence, the second nucleic acid sequence and the third nucleic acid sequence, are in trans.

The cell can be selected from a group consisting of a human, a primate, a murine, a feline, a canine, a porcine, an ovine, a bovine, an equine, a caprine and a lupine host cell. In some instances, the cell is a progenitor or precursor cell, such as a stem cell. In some instances, the stem cell is a mesenchymal cell, embryonic stem cell, induced pluripotent stem cell (iPSC), fibroblast or other tissue specific stem cell. The cell can be immortalized. In some instances, the embryonic stem cell is a human embryonic stem cell. In some instances, the human embryonic stem cell is a human embryonic kidney 293 (HEK-293) cell. In some instances, the cell is a differentiated cell. Base on the disclosure provided, it is expected that this system can be used in conjunction with any transgenic line expressing a recombinase in the target cell type of interest to develop AAV capsids that more efficiently transduce that target cell population.

There are provided, in some embodiments, nucleic acids encoding one or more of the variant capsid proteins provided herein (e.g., tandem multimers, HI loop variant capsid proteins, guided variant capsid proteins). Provided here are plasmid vectors encoding the variant capsid proteins of the present disclosure (e.g., tandem multimers, HI loop variant capsid proteins, guided variant capsid proteins). Also disclosed are nucleic acids encoding the rAAV capsids comprising variant AAV capsid proteins (e.g., tandem multimers, HI loop variant capsid proteins, guided variant capsid proteins) of the present disclosure. Heterologous nucleic acids and transgenes of the present embodiment may also include plasmid vectors. Plasmid vectors are useful for the generation of the recombinant AAV (rAAV) particles described herein. An AAV vector can comprise a genome of a helper virus. Helper virus proteins are required for the assembly of a recombinant rAAV, and packaging of a transgene containing a heterologous nucleic acid into the rAAV. The helper virus genes are adenovirus genes E4, E2a and VA, that when expressed in the cell, assist with AAV replication. In some embodiments, an AAV vector comprises E2. In some embodiments, an AAV vector comprises E4. In some embodiments, an AAV vector comprises VA. In some instances, the AAV vector comprises one of helper virus proteins, or any combination thereof. In some instances, the plasmid vector is bacterial. In some instances, the plasmid vector is derived from Escherichia coli. In some instances, the nucleic acid sequence comprises, in a 5′ to 3′ direction: (1) a 5′ inverted terminal repeat (ITR) sequence, (2) a Replication (Rep) gene, (3) a Capsid (Cap) gene, and (4) a 3′ ITR, wherein the Cap gene encodes the variant AAV capsid protein described herein. In some instances, the plasmid vector encodes a pseudotyped AAV capsid protein.

Disclosed herein are modified viral genomes comprising genetic information (e.g., heterologous nucleic acid) that are assembled into a rAAV via viral packaging. In some instances, the viral genome is from an AAV serotypes selected from the group consisting of AAV1, AAV2, AAV3, AAV4, AAV5, AAV6, AAV7, AAV8, AAV9, AAV10, AAV11, and AAV12.

A viral genome, such as those described herein, can comprise a transgene, which in some cases encodes a heterologous gene expression product (e.g., therapeutic gene expression product, recombinant capsid protein, and the like). The transgene is in cis with two inverted terminal repeats (ITRs) flanking the transgene. The transgene may comprise a therapeutic nucleic acid encoding a therapeutic gene expression product

The viral genome, in some cases, is a single stranded viral DNA comprising the transgene. The AAV vector can be episomal. In some instances, the viral genome is a concatemer. An episomal viral genome can develop chromatin-like organization in the target cell that does not integrate into the genome of the target cell. When infected into non-dividing cells, the stability of the episomal viral genome in the target cell enable the long-term transgene expression. Alternatively, the AAV vector integrates the transgene into the genome of the target cell predominantly at a specific site (e.g., AAV5 1 on human chromosome 19).

rAAV genomes are provided herein. The genome can, for example, comprise at least one inverted terminal repeat configured to allow packaging into a vector and a cap gene. In some embodiments, it can further include a sequence within a rep gene required for expression and splicing of the cap gene. In some embodiments, the genome can further include a sequence capable of expressing a capsid protein provided herein.

Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)). The viral vector can incorporate sequences from the genome of any known organism. The sequences can be incorporated in their native form or can be modified in any way to obtain a desired activity. For example, the sequences can comprise insertions, deletions or substitutions.

In some embodiments, the viral vectors can include additional sequences that make the vectors suitable for replication and integration in eukaryotes. In other embodiments, the viral vectors disclosed herein can include a shuttle element that makes the vectors suitable for replication and integration in both prokaryotes and eukaryotes. In some embodiments, the viral vectors can include additional transcription and translation initiation sequences, such as promoters and enhancers; and additional transcription and translation terminators, such as polyadenylation signals. Various regulatory elements that can be included in an AAV vector have been described in details in US Patent Publication 2012/0232133 which is hereby incorporated by reference in its entirety.

Vectors comprising a nucleic acid sequence encoding the modified AAV capsid proteins of the present disclosure are also provided herein. For example, the vectors of the present disclosure can comprise a nucleic acid sequence encoding the two AAV viral genes, Rep (Replication), and a Cap (Capsid) gene, wherein the Cap gene, encoding viral capsid proteins VP1, VP2, and VP3 is modified to produce the modified AAV capsid proteins of the present disclosure.

The rAAV capsid proteins described herein may be isolated and purified. The AAV may be isolated and purified by methods standard in the art such as by column chromatography or cesium chloride gradients. Methods for purifying AAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

The rAAV capsid and/or rAAV capsid protein can be conjugated to a nanoparticle, a second molecule, or a viral capsid protein. In some cases, the nanoparticle or viral capsid protein would encapsidate the therapeutic nucleic acid described herein. In some instances, the second molecule is a therapeutic agent, e.g., a small molecule, antibody, antigen-binding fragment, peptide, or protein, such as those described herein. In some instances, the second molecule is a detectable moiety. For example, the modified AAV capsid and/or rAAV capsid protein conjugated to a detectable moiety may be used for in vitro, ex vivo, or in vivo biomedical research applications, the detectable moiety used to visualize the modified capsid protein. The modified AAV capsid and/or rAAV capsid protein conjugated to a detectable moiety may also be used for diagnostic purposes.

rAAV, Heterologous Nucleic Acids, and Payloads

Disclosed herein include recombinant AAV (rAAV). In some embodiments, the recombinant AAV (rAAV) comprises: a) a variant AAV capsid provided herein; and b) a heterologous nucleic acid provided herein. The heterologous nucleic acid can comprise a polynucleotide encoding a payload. The heterologous nucleic acid can comprise a single-stranded DNA molecule, a double-stranded DNA molecule, a single-stranded RNA molecule, a double-stranded RNA molecule, or any combination thereof. The single-stranded DNA molecule can be capable of self-hybridizing to form a double-stranded region. The single-stranded DNA molecule can comprise a self-complementary AAV (scAAV) vector.

Disclosed herein include populations of recombinant AAV (rAAV). Disclosed herein include populations of variant capsids. The average diameter of the viral capsids of the population of rAAV can be, or can be about, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, or a number or a range between any two of these values. The average diameter of the viral capsids of the population of rAAV can be at least, or can be at most, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, or 100 nm. The average can be the mean, median or mode. In some embodiments, the mean can be the arithmetic mean, geometric mean, and/or harmonic mean. The diameter can be calculated as the mean of the major axis length and the minor axis length. The diameter can be measured by transmission electron microscopy (TEM). The diameter can be hydrodynamic diameter. The hydrodynamic diameter can be measured by dynamic light scattering (DLS).

The diameter of the viral capsids of the population of rAAV can vary (e.g., can range from about 20 nm to about 100 nm). For example, the diameter and/or average diameter of the viral capsids of the population of rAAV can range from about 20 nm to about 100 nm, from about 20 nm to about 80 nm, from about 20 nm to about 60 nm, from about 20 nm to about 40 nm, from about 30 nm to about 100 nm, from about 30 nm to about 80 nm, from about 30 nm to about 60 nm, from about 20 nm to about 40 nm, from about 40 nm to about 100 nm, from about 40 nm to about 80 nm, from about 40 nm to about 60 nm, from about 50 nm to about 100 nm, from about 50 nm to about 80 nm, from about 50 nm to about 60 nm, from about 60 nm to about 100 nm, or from about 60 nm to about 80 nm or a number or a range between any two of these values. The minimum diameter of the viral capsids of the population of rAAV can be, or can be about, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, or a number or a range between any two of these values. The minimum diameter of the viral capsids of the population of rAAV can be at least, or can be at most, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, or 100 nm.

The maximum diameter of the viral capsids of the population of rAAV can vary. The maximum diameter of the viral capsids of the population of rAAV can be, or can be about, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, or a number or a range between any two of these values. The maximum diameter of the viral capsids of the population of rAAV can be at least, or can be at most, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, or 100 nm.

The length of the heterologous nucleic acid can be at least about 25% (e.g., 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of the genetic cargo capacity of the variant AAV capsid. The length of the heterologous nucleic acid can vary. The length of the heterologous nucleic acid can be, or can be about, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10.0 kb, or a number or a range between any two of these values. The length of the heterologous nucleic acid can be at least, or can be at most, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, or 10.0 kb.

The heterologous nucleic acid can comprise a 5′ inverted terminal repeat (ITR) and a 3′ ITR. The payload can comprise a payload protein. The heterologous nucleic acid can comprise a promoter operably linked to the polynucleotide encoding a payload. The promoter can be capable of inducing the transcription of the polynucleotide. Transcription of the polynucleotide can generate a payload transcript. The heterologous nucleic acid can comprise one or more of a 5′ UTR, 3′ UTR, a minipromoter, an enhancer, a splicing signal, a polyadenylation signal, a terminator, one or more silencer effector binding sequences, a protein degradation signal, and an internal ribosome-entry element (IRES). The silencer effector can comprise a microRNA (miRNA), a precursor microRNA (pre-miRNA), a small interfering RNA (siRNA), a short-hairpin RNA (shRNA), precursors thereof, derivatives thereof, or a combination thereof. The silencer effector can be capable of binding the one or more silencer effector binding sequences, thereby reducing the stability of the payload transcript and/or reducing the translation of the payload transcript. The polynucleotide further can comprise a transcript stabilization element. The transcript stabilization element can comprise woodchuck hepatitis post-translational regulatory element (WPRE), bovine growth hormone polyadenylation (bGH-polyA) signal sequence, human growth hormone polyadenylation (hGH-polyA) signal sequence, or any combination thereof. The payload can comprise a payload RNA agent. The payload RNA agent can comprise one or more of dsRNA, siRNA, shRNA, pre-miRNA, pri-miRNA, miRNA, stRNA, lncRNA, piRNA, and snoRNA. The payload RNA agent inhibits or suppresses the expression of a gene of interest in a cell. In some embodiments, the gene of interest can be selected from the group comprising SOD1, MAPT, APOE, HTT, C90RF72, TDP-43, APP, BACE, SNCA, ATXN1, ATXN2, ATXN3, ATXN7, SCN1A-SCN5A, and SCN8A-SCN11A. The heterologous nucleic acid further can comprise a polynucleotide encoding one or more secondary proteins, and the payload protein and the one or more secondary proteins can comprise a synthetic protein circuit. The heterologous nucleic acid can comprise a single-stranded AAV (ssAAV) vector or a self-complementary AAV (scAAV) vector.

The promoter can comprise a ubiquitous promoter. The ubiquitous promoter can be selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

The promoter can be an inducible promoter. In some embodiments, the inducible promoter is a tetracycline responsive promoter, a TRE promoter, a Tre3G promoter, an ecdysone responsive promoter, a cumate responsive promoter, a glucocorticoid responsive promoter, and estrogen responsive promoter, a PPAR-y promoter, or an RU-486 responsive promoter.

The promoter can comprise a tissue-specific promoter and/or a lineage-specific promoter. The tissue specific promoter can be a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. The tissue specific promoter can be a neuron-specific promoter. The neuron-specific promoter can comprise a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. The tissue specific promoter can be a muscle-specific promoter. In some embodiments, the muscle-specific promoter can comprise a creatine kinase (MCK) promoter.

The promoter can comprise an intronic sequence. The promoter can comprise a bidirectional promoter and/or an enhancer. In some embodiments, the enhancer can be a CMV enhancer. One or more cells of a subject can comprise an endogenous version of the payload, and the promoter can comprise or can be derived from the promoter of the endogenous version. In some embodiments, one or more cells of a subject comprise an endogenous version of the payload, and the payload is not truncated relative to the endogenous version.

In some embodiments, the promoter is less than 1 kb. In other embodiments, the promoter is greater than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. The promoter may provide expression of the therapeutic gene expression product for a period of time in targeted tissues such as, but not limited to, the central nervous system and peripheral organs (e.g., lung). Expression of the therapeutic gene expression product may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years. Expression of the payload may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years or 10-15 years, or 15-20 years, or 20-25 years, or 25-30 years, or 30-35 years, or 35-40 years, or 40-45 years, or 45-50 years, or 50-55 years, or 55-60 years, or 60-65 years.

The payload protein can comprise aromatic L-amino acid decarboxylase (AADC), survival motor neuron 1 (SMN1), frataxin (FXN), Cystic Fibrosis Transmembrane Conductance Regulator (CFTR), Factor X (FIX), RPE65, Retinoid Isomerohydrolase (RPE65), Sarcoglycan Alpha (SGCA), and sarco/endoplasmic reticulum Ca2+-ATPase (SERCA2a), ApoE2, GBA1, GRN, ASP A, CLN2, GLB1, SGSH, NAGLU, IDS, NPC1, GAN, CFTR, GDE, OTOF, DYSF, MYO7A, ABCA4, F8, CEP290, CDH23, DMD, ALMS1, or any combination thereof.

The payload protein can comprise a disease-associated protein. In some embodiments, the level of expression of the disease-associated protein correlates with the occurrence and/or progression of the disease. The payload protein can comprise methyl CpG binding protein 2 (MeCP2), DRK1A, KAT6A, NIPBL, HDAC4, UBE3A, EHMT1, one or more genes encoded on chromosome 9q34.3, NPHP1, LIMK1 one or more genes encoded on chromosome 7q11.23, P53, TPI1, FGFR1 and related genes, RA1, SHANK3, CLN3, NF-1, TP53, PFK, CD40L, CYP19A1, PGRN, CHRNA7, PMP22, CD40LG, derivatives thereof, or any combination thereof.

The payload protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The payload protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The payload protein can comprise a nuclear localization signal (NLS) or a nuclear export signal (NES).

The payload protein can comprise a CRE recombinase, GCaMP, a cell therapy component, a knock-down gene therapy component, a cell-surface exposed epitope, or any combination thereof. The payload protein can comprise a chimeric antigen receptor. The payload protein can comprise a diagnostic agent (e.g., green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof).

The payload protein can comprise a programmable nuclease. In some embodiments, the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cash, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. The heterologous nucleic acid and/or rAAV can comprise a polynucleotide encoding (i) a targeting molecule and/or (ii) a donor nucleic acid. The targeting molecule can be capable of associating with the programmable nuclease. The targeting molecule can comprise single strand DNA or single strand RNA. The targeting molecule can comprise a single guide RNA (sgRNA).

Disclosed herein are heterologous nucleic acids comprising a polynucleotide encoding one or more payload genes. The rAAV provided herein can comprise one or more of the heterologous nucleic acids disclosed herein. The heterologous nucleic acid can comprise a polynucleotide encoding a payload (e.g., a payload gene). The payload gene can encode a payload RNA agent and/or payload protein. The heterologous nucleic acid can comprise a promoter operably linked to the polynucleotide encoding a payload. As disclosed herein, the payload gene is operatively linked with appropriate regulatory elements in some embodiments. The one or more payload genes of the heterologous nucleic acid can comprise a siRNA, a shRNA, an antisense RNA oligonucleotide, an antisense miRNA, a trans-splicing RNA, a guide RNA, single-guide RNA, crRNA, a tracrRNA, a trans-splicing RNA, a pre-mRNA, a mRNA, or any combination thereof. The one or more payload genes of the heterologous nucleic acid can comprise one or more synthetic protein circuit components. The one or more payload genes of the heterologous nucleic acid can comprise can entire synthetic protein circuit comprising one or more synthetic protein circuit components. The one or more payload genes of the heterologous nucleic acid can comprise two or more synthetic protein circuits.

The payload protein can be any protein, including naturally-occurring and non-naturally occurring proteins. Examples of payload protein include, but are not limited to, luciferases; fluorescent proteins (e.g., GFP); growth hormones (GHs) and variants thereof; insulin-like growth factors (IGFs) and variants thereof; granulocyte colony-stimulating factors (G-CSFs) and variants thereof; erythropoietin (EPO) and variants thereof; insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof; clotting factors and variants thereof; cystic fibrosis transmembrane conductance regulator (CFTR) and variants thereof; and interferons and variants thereof.

In some embodiments, the payload protein is a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and the like; enzymes, such as -glucosidase, imiglucarase, β-glucocerebrosidase, and alglucerase; enzyme activators, such as tissue plasminogen activator; chemokines, such as IP-10, monokine induced by interferon-gamma (Mig), Gro/IL-8, RANTES, MIP-1, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; Th-1 receptor antagonists; and the like. Some other non-limiting examples of payload protein include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

In some embodiments, the payload protein is an active fragment of a protein, such as any of the aforementioned proteins. In some embodiments, the payload protein is a fusion protein comprising some or all of two or more proteins. In some embodiments a fusion protein can comprise all or a portion of any of the aforementioned proteins.

In some embodiments, the payload protein is a multi-subunit protein. For examples, the payload protein can comprise two or more subunits, or two or more independent polypeptide chains. In some embodiments, the payload protein can be an antibody. Examples of antibodies include, but are not limited to, antibodies of various isotypes (for example, IgG1, IgG2, IgG3, IgG4, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. In some embodiments, the antibody is a full-length antibody.

In some embodiments, the payload gene encodes a pro-survival protein (e.g., Bcl-2, Bcl-XL, Mcl-1 and A1). In some embodiments, the payload gene encodes a apoptotic factor or apoptosis-related protein such as, for example, AIF, Apaf (e.g., Apaf-1, Apaf-2, and Apaf-3), oder APO-2 (L), APO-3 (L), Apopain, Bad, Bak, Bax, Bcl-2, Bcl-x_{L}, Bcl-x_{S}, bik, CAD, Calpain, Caspase (e.g., Caspase-1, Caspase-2, Caspase-3, Caspase-4, Caspase-5, Caspase-6, Caspase-7, Caspase-8, Caspase-9, Caspase-10, and Caspase-11), ced-3, ced-9, c-Jun, c-Myc, crm A, cytochrom C, CdR1, DcR1, DD, DED, DISC, DNA-PKcs, DR3, DR4, DRS, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, JNK, Lamin A/B, MAP, MCL-1, Mdm-2, MEKK-1, MORT-1, NEDD, NF-ₖₐₚₚₐB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, and/or transglutaminase.

In some embodiments, the payload gene encodes a cellular reprogramming factor capable of converting an at least partially differentiated cell to a less differentiated cell, such as, for example, Oct-3, Oct-4, Sox2, c-Myc, Klf4, Nanog, Lin28, ASCL1, MYT1 L, TBX3b, SV40 large T, hTERT, miR-291, miR-294, miR-295, or any combinations thereof. In some embodiments, the payload gene encodes a programming factor that is capable of differentiating a given cell into a desired differentiated state, such as, for example, nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof.

In some embodiments, the payload gene encodes a human adjuvant protein capable of eliciting an innate immune response, such as, for example, cytokines which induce or enhance an innate immune response, including IL-2, IL-12, IL-15, IL-18, IL-21CCL21, GM-CSF and TNF-alpha; cytokines which are released from macrophages, including IL-1, IL-6, IL-8, IL-12 and TNF-alpha; from components of the complement system including C1q, MBL, C1r, C1s, C2b, Bb, D, MASP-1, MASP-2, C4b, C3b, C5a, C3a, C4a, C5b, C6, C7, C8, C9, CR1, CR2, CR3, CR4, C1qR, C11NH, C4 bp, MCP, DAF, H, I, P and CD59; from proteins which are components of the signaling networks of the pattern recognition receptors including TLR and IL-1 R1, whereas the components are ligands of the pattern recognition receptors including IL-1 alpha, IL-1 beta, Beta-defensin, heat shock proteins, such as HSP10, HSP60, HSP65, HSP70, HSP75 and HSP90, gp96, Fibrinogen, Typ111 repeat extra domain A of fibronectin; the receptors, including IL-1 RI, TLR1, TLR2, TLR3, TLR4, TLR5, TLR6, TLR7, TLR8, TLR9, TLR10, TLR11; the signal transducers including components of the Small-GTPases signaling (RhoA, Ras, Rac1, Cdc42 etc.), components of the PIP signaling (PI3K, Src-Kinases, etc.), components of the MyD88-dependent signaling (MyD88, IRAK1, IRAK2, etc.), components of the MyD88-independent signaling (TICAM1, TICAM2 etc.); activated transcription factors including e.g. INF-κB, c-Fos, c-Jun, c-Myc; and induced target genes including e.g. IL-1 alpha, IL-1 beta, Beta-Defensin, IL-6, IFN gamma, IFN alpha and IFN beta; from costimulatory molecules, including CD28 or CD40-ligand or PD1; protein domains, including LAMP; cell surface proteins; or human adjuvant proteins including CD80, CD81, CD86, trif, flt-3 ligand, thymopentin, Gp96 or fibronectin, etc., or any species homolog of any of the above human adjuvant proteins.

In some embodiments, the payload gene encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments the heterologous nucleic acids provided herein can be employed as vaccines for the prophylaxis or treatment of infectious diseases (e.g., as vaccines).

As described herein, the nucleotide sequence encoding the payload protein can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of payload gene expression in the target cell can vary. For example, in some embodiments, the payload gene encodes a payload protein. The amount of the payload protein expressed in the subject (e.g., the serum of the subject) can vary. For example, in some embodiments the protein can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the payload protein is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a payload protein is needed for the method to be effective can vary depending on non-limiting factors such as the particular payload protein and the subject receiving the treatment, and an effective amount of the protein can be readily determined by a skilled artisan using conventional methods known in the art without undue experimentation.

A payload protein encoded by a payload gene can be of various lengths. For example, the payload protein can be at least about 200 amino acids, at least about 250 amino acids, at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, or longer in length. In some embodiments, the payload protein is at least about 480 amino acids in length. In some embodiments, the payload protein is at least about 500 amino acids in length. In some embodiments, the payload protein is about 750 amino acids in length.

The payload genes can have different lengths in different implementations. The number of payload genes can be different in different embodiments. In some embodiments, the number of payload genes in a heterologous nucleic acid can be, or can be about, 1, 2, 3, 4, 5, 6, 7, 8, 9, 10, 11, 12, 13, 14, 15, 16, 17, 18, 19, 20, 21, 22, 23, 24, 25, or a number or a range between any two of these values. In some embodiments, the number of payload genes in a heterologous nucleic acid can be at least, or can be at most, 1, 2, 3, 4, 5, 6, 7, 8, 9, 10, 11, 12, 13, 14, 15, 16, 17, 18, 19, 20, 21, 22, 23, 24, 25. In some embodiments, a payload genes is, or is about, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, or a number or a range between any two of these values, nucleotides in length. In some embodiments, a payload gene is at least, or is at most, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, or 10000 nucleotides in length.

The payload can be an inducer of cell death. The payload can be induce cell death by a non-endogenous cell death pathway (e.g., a bacterial pore-forming toxin). In some embodiments, the payload can be a pro-survival protein. In some embodiments, the payload is a modulator of the immune system. The payload can activate an adaptive immune response, and innate immune response, or both. In some embodiments, the payload gene encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments the compositions provided herein can be employed as vaccines for the prophylaxis or treatment of infectious diseases (e.g., as vaccines). The payload protein can comprise a CRE recombinase, GCaMP, a cell therapy component, a knock-down gene therapy component, a cell-surface exposed epitope, or any combination thereof. In some embodiments, the payload comprises CFTR, GDE, OTOF, DYSF, MYO7A, ABCA4, F8, CEP290, CDH23, DMD, and ALMS1.

A payload can comprise a non-protein coding gene, such as a payload RNA agent, e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components described herein. The a non-protein coding gene may also encode a tRNA, rRNA, tmRNA, piRNA, double stranded RNA, snRNA, snoRNA, and/or long non-coding RNA (IncRNA). In some cases, the non-protein coding gene can modulate the expression or the activity of a target gene or gene expression product. a non-protein coding gene. For example, the RNAs described herein may be used to inhibit gene expression in a target cell, for example, a cell in the central nervous system (CNS) or peripheral organ (e.g., lung). In some cases, inhibition of gene expression refers to an inhibition by at least about 20%, 30%, 40%, 50%, 60%, 70%, 80%, 85%, 90%, 95% and 100%. In some cases, the protein product of the targeted gene may be inhibited by at least about 20%, 30%, 40%, 50%, 60%, 70%, 80%, 85%, 90%, 95% and 100%. The gene can be either a wild type gene or a gene with at least one mutation. The targeted protein may be either a wild type protein or a protein with at least one mutation.

Examples of payload genes include a sequence associated with a signaling biochemical pathway, e.g., a signaling biochemical pathway-associated gene or polynucleotide (e.g., a signal transducer). In some embodiments, the methods and compositions disclosed herein comprise knockdown of an endogenous signal transducer accompanied by tuned expression of a payload protein comprising an appropriate version of signal transducer. Examples of payloads contemplated herein include a disease-associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level. Signal transducers can be can be associated with one or more diseases or disorders. In some embodiments, a disease or disorder is characterized by an aberrant signaling of one or more signal transducers disclosed herein. In some embodiments, the activation level of the signal transducer correlates with the occurrence and/or progression of a disease or disorder. The activation level of the signal transducer can be directly responsible or indirectly responsible for the etiology of the disease or disorder. Non-limiting examples of signal transducers, signal transduction pathways, and diseases and disorders characterized by aberrant signaling of said signal transducers are listed in Tables 2-4. In some embodiments, the methods and compositions disclosed herein prevent or treat one or more of the diseases and disorders listed in Tables 2-4. In some embodiments, the payload comprises a replacement version of the signal transducer. In some embodiments, the methods and compositions further comprise knockdown of the corresponding endogenous signal transducer. The payload can comprise the product of a gene listed in listed in Tables 2-4. In some embodiments, the payload ameliorates a disease or disorder characterized by an aberrant signaling of one or more signaling transducers. In some embodiments, the payload diminishes the activation level of one or more signal transducers (e.g., signal transducers with aberrant overactive signaling, signal transducers listed in Tables 2-4). In some embodiments, the payload increases the activation level of one or more signal transducers (e.g., signal transducers with aberrant underactive signaling). In some such embodiments, the payload can modulate the abundance, location, stability, and/or activity of activators or repressors of said signal transducers.

TABLE 2
DISEASES AND DISORDERS OF INTEREST

| Diseases/Disorders | Genes |
|---|---|
| Neoplasia | PTEN; ATM; ATR; EGFR; ERBB2; ERBB3; ERBB4; Notch1; Notch2; Notch3; |
| | Notch4; AKT; AKT2; AKT3; HIF; HIF1a; HIF3a; Met; HRG; Bcl2; PPAR |
| | alpha; PPAR gamma; WT1 (Wilms Tumor); FGF Receptor Family members (5 |
| | members: 1, 2, 3, 4, 5); CDKN2a; APC; RB (retinoblastoma); MEN1; VHL; |
| | BRCA1; BRCA2; AR (Androgen Receptor); TSG101; IGF; IGF Receptor; Igf1 |
| | (4 variants); Igf2 (3 variants); Igf 1 Receptor; Igf 2 Receptor; Bax; Bcl2; |
| | caspases family (9 members: 1, 2, 3, 4, 6, 7, 8, 9, 12); Kras; Apc |
| Age-related Macular | Abcr; Ccl2; Cc2; cp (ceruloplasmin); Timp3; cathepsinD; Vldlr; Ccr2 |
| Degeneration | |
| Schizophrenia | Neuregulin1 (Nrg1); Erb4 (receptor for Neuregulin); Complexin1 (Cplx1); Tph1 |
| | Tryptophan hydroxylase; Tph2 Tryptophan hydroxylase 2; Neurexin 1; GSK3; |
| | GSK3a; GSK3b |
| Disorders | 5-HTT (Slc6a4); COMT; DRD (Drd1a); SLC6A3; DAOA; DTNBP1; Dao |
| | (Dao1) |
| Trinucleotide Repeat | HTT (Huntington's Dx); SBMA/SMAX1/AR (Kennedy's Dx); FXN/X25 |
| Disorders | (Friedrich's Ataxia); ATX3 (Machado- Joseph's Dx); ATXN1 and ATXN2 |
| | (spinocerebellar ataxias); DMPK (myotonic dystrophy); Atrophin-1 and Atn1 |
| | (DRPLA Dx); CBP (Creb-BP - global instability); VLDLR (Alzheimer's); |
| | Atxn7; Atxn10 |
| Fragile X Syndrome | FMR2; FXR1; FXR2; mGLUR5 |
| Secretase Related | APH-1 (alpha and beta); Presenilin (Psen1); nicastrin (Ncstn); PEN-2 |
| Disorders | |
| Others | Nos1; Parp1; Nat1; Nat2 |
| Prion-related disorders | Prp |
| ALS | SOD1; ALS2; STEX; FUS; TARDBP; VEGF (VEGF-a; VEGF-b; VEGF-c) |
| Drug addiction | Prkce (alcohol); Drd2; Drd4; ABAT (alcohol); GRIA2; Grm5; Grin1; Htr1b; |
| | Grin2a; Drd3; Pdyn; Gria1 (alcohol) |
| Autism | Mecp2; BZRAP1; MDGA2; Sema5A; Neurexin 1; Fragile X (FMR2 (AFF2); |
| | FXR1; FXR2; Mglur5) |
| Alzheimer's Disease | E1; CHIP; UCH; UBB; Tau; LRP; PICALM; Clusterin; PS1; SORL1; CR1; |
| | Vldlr; Uba1; Uba3; CHIP28 (Aqp1, Aquaporin 1); Uchl1; Uchl3; APP |
| Inflammation | IL-10; IL-1 (IL-1a; IL-1b); IL-13; IL-17 (IL-17a (CTLA8); IL- 17b; IL-17c; |
| | IL-17d; IL-17f); II-23; Cx3cr1; ptpn22; TNFa; NOD2/CARD15 for IBD; IL-6; |
| | IL-12 (IL-12a; IL-12b); CTLA4; Cx3cl1 |
| Parkinson's Disease | x-Synuclein; DJ-1; LRRK2; Parkin; PINK1 |

TABLE 3
SIGNAL TRANSDUCERS

| | |
|---|---|
| Blood and | Anemia (CDAN1, CDA1, RPS19, DBA, PKLR PK1, NT5C3, UMPH1, PSN1, RHAG, |
| coagulation | RH50A, NRAMP2, SPTB, ALAS2, ANH1, ASB, ABCB7, ABC7, ASAT); Bare |
| diseases and | lymphocyte syndrome (TAPBP, TPSN, TAP2, ABCB3, PSF2, RING11, MHC2TA, |
| disorders | C2TA, RFX5, RFXAP, RFX5); Bleeding disorders (TBXA2R, P2RX1, P2X1); Factor |
| | H and factor H-like 1 (HF1, CFH, HUS); Factor V and factor VIII (MCFD2); Factor |
| | VII deficiency (F7); Factor X deficiency (F10); Factor XI deficiency (F11); Factor |
| | XII deficiency (F12, HAF); Factor XIIIA deficiency (F13A1, F13A); Factor XIIIB |
| | deficiency (F13B); Fanconi anemia (FANCA, FACA, FA1, FA, FAA, FAAP95, |
| | FAAP90, FLJ34064, FANCB, FANCC, FACC, BRCA2, FANCD1, FANCD2, FANCD, |
| | FACD, FAD, FANCE, FACE, FANCF, XRCC9, FANCG, BRIP1, BACH1, FANCJ, |
| | PHF9, FANCL, FANCM, KIAA1596); Hemophagocytic lymphohistiocytosis |
| | disorders (PRF1, HPLH2, UNC13D, MUNC13-4, HPLH3, HLH3, FHL3); Hemophilia |
| | A (F8, F8C, HEMA); Hemophilia B (F9, HEMB), Hemorrhagic disorders (PI, ATT, |
| | F5); Leukocyde deficiencies and disorders (ITGB2, CD18, LCAMB, LAD, EIF2B1, |
| | EIF2BA, EIF2B2, EIF2B3, EIF2B5, LVWM, CACH, CLE, EIF2B4); Sickle cell anemia |
| | (HBB); Thalassemia (HBA2, HBB, HBD, LCRB, HBA1). |
| Cell | B-cell non-Hodgkin lymphoma (BCL7A, BCL7); Leukemia (TAL1 TCL5, SCL, |
| dysregulation | TAL2, FLT3, NBS1, NBS, ZNFN1A1, IK1, LYF1, HOXD4, HOX4B, BCR, CML, |
| and oncology | PHL, ALL, ARNT, KRAS2, RASK2, GMPS, AF10, ARHGEF12, LARG, KIAA0382, |
| diseases and | CALM, CLTH, CEBPA, CEBP, CHIC2, BTL, FLT3, KIT, PBT, LPP, NPM1, NUP214, |
| disorders | D9S46E, CAN, CAIN, RUNX1, CBFA2, AML1, WHSC1L1, NSD3, FLT3, AF1Q, |
| | NPM1, NUMA1, ZNF145, PLZF, PML, MYL, STAT5B, AF10, CALM, CLTH, |
| | ARL11, ARLTS1, P2RX7, P2X7, BCR, CML, PHL, ALL, GRAF, NF1, VRNF, WSS, |
| | NFNS, PTPN11, PTP2C, SHP2, NS1, BCL2, CCND1, PRAD1, BCL1, TCRA, GATA1, |
| | GF1, ERYF1, NFE1, ABL1, NQO1, DIA4, NMOR1, NUP214, D9S46E, CAN, CAIN). |
| Inflammation | AIDS (KIR3DL1, NKAT3, NKB1, AMB11, KIR3DS1, IFNG, CXCL12, SDF1); |
| and immune | Autoimmune lymphoproliferative syndrome (TNFRSF6, APTI, FAS, CD95, |
| related | ALPS1A); Combined immunodeficiency, (IL2RG, SCIDX1, SCIDX, IMD4); HIV-1 |
| diseases and | (CCL5, SCYA5, D17S136E, TCP228), HIV susceptibility or infection (IL10, CSIF, |
| disorders | CMKBR2, CCR2, CMKBR5, CCCKR5 (CCR5)); Immunodeficiencies (CD3E, CD3G, |
| | AICDA, AID, HIGM2, TNFRSF5, CD40, UNG, DGU, HIGM4, TNFSF5, CD40LG, |
| | HIGM1, IGM, FOXP3, IPEX, AIID, XPID, PIDX, TNFRSF14B, TACI); Inflammation |
| | (IL-10, IL-1 (IL-1a, IL-1b), IL-13, IL-17 (IL-17a (CTLA8), IL-17b, IL-17c, IL-17d, |
| | IL-17f), II-23, Cx3cr1, ptpn22, TNFa, NOD2/CARD15 for IBD, IL-6, IL-12 (IL-12a, |
| | IL-12b), CTLA4, Cx3cl1); Severe combined immunodeficiencies (SCIDs)(JAK3, JAKL, |
| | DCLRE1C, ARTEMIS, SCIDA, RAG1, RAG2, ADA, PTPRC, CD45, LCA, IL7R, |
| | CD3D, T3D, IL2RG, SCIDX1, SCIDX, IMD4). |
| Metabolic, | Amyloid neuropathy (TTR, PALB); Amyloidosis (APOA1, APP, AAA, CVAP, AD1, |
| liver, kidney | GSN, FGA, LYZ, TTR, PALB); Cirrhosis (KRT18, KRT8, CIRH1A, NAIC, TEX292, |
| and protein | KIAA1988); Cystic fibrosis (CFTR, ABCC7, CF, MRP7); Glycogen storage diseases |
| diseases and | (SLC2A2, GLUT2, G6PC, G6PT, G6PT1, GAA, LAMP2, LAMPB, AGL, GDE, GBE1, |
| disorders | GYS2, PYGL, PFKM); Hepatic adenoma (TCF1, HNF1A, MODY3), Hepatic failure, |
| | early onset, and neurologic disorder (SCOD1, SCO1), Hepatic lipase deficiency |
| | (LIPC), Hepatoblastoma, cancer and carcinomas (CTNNB1, PDGFRL, PDGRL, |
| | PRLTS, AXIN1, AXIN, CTNNB1, TP53, P53, LFS1, IGF2R, MPRI, MET, CASP8, |
| | MCH5); Medullary cystic kidney disease (UMOD, HNFJ, FJHN, MCKD2, |
| | ADMCKD2); Phenylketonuria (PAH, PKU1, QDPR, DHPR, PTS); Polycystic kidney |
| | and hepatic disease (FCYT, PKHD1, ARPKD, PKD1, PKD2, PKD4, PKDTS, |
| | PRKCSH, G19P1, PCLD, SEC63). |
| Muscular/ | Becker muscular dystrophy (DMD, BMD, MYF6), Duchenne Muscular Dystrophy |
| Skeletal | (DMD, BMD); Emery-Dreifuss muscular dystrophy (LMNA, LMN1, EMD2, FPLD, |
| diseases and | CMD1A, HGPS, LGMD1B, LMNA, LMN1, EMD2, FPLD, CMD1A); |
| disorders | Facioscapulohumeral muscular dystrophy (FSHMD1A, FSHD1A); Muscular |
| | dystrophy (FKRP, MDC1C, LGMD2I, LAMA2, LAMM, LARGE, KIAA0609, |
| | MDC1D, FCMD, TTID, MYOT, CAPN3, CANP3, DYSF, LGMD2B, SGCG, |
| | LGMD2C, DMDA1, SCG3, SGCA, ADL, DAG2, LGMD2D, DMDA2, SGCB, |
| | LGMD2E, SGCD, SGD, LGMD2F, CMD1L, TCAP, LGMD2G, CMD1N, TRIM32, |
| | HT2A, LGMD2H, FKRP, MDC1C, LGMD2I, TTN, CMD1G, TMD, LGMD2J, |
| | POMT1, CAV3, LGMD1C, SEPN1, SELN, RSMD1, PLEC1, PLTN, EBS1); |
| | Osteopetrosis (LRP5, BMND1, LRP7, LR3, OPPG, VBCH2, CLCN7, CLC7, OPTA2, |
| | OSTM1, GL, TCIRG1, TIRC7, OC116, OPTB1); Muscular atrophy (VAPB, VAPC, |
| | ALS8, SMN1, SMA1, SMA2, SMA3, SMA4, BSCL2, SPG17, GARS, SMAD1, |
| | CMT2D, HEXB, IGHMBP2, SMUBP2, CATF1, SMARD1). |
| Neurological | ALS (SOD1, ALS2, STEX, FUS, TARDBP, VEGF (VEGF-a, VEGF-b, VEGF-c); |
| and neuronal | Alzheimer disease (APP, AAA, CVAP, AD1, APOE, AD2, PSEN2, AD4, STM2, |
| diseases and | APBB2, FE65L1, NOS3, PLAU, URK, ACE, DCP1, ACE1, MPO, PACIP1, PAXIP1L, |
| disorders | PTIP, A2M, BLMH, BMH, PSEN1, AD3); Autism (Mecp2, BZRAP1, MDGA2, |
| | Sema5A, Neurexin 1, GLO1, MECP2, RTT, PPMX, MRX16, MRX79, NLGN3, |
| | NLGN4, KIAA1260, AUTSX2); Fragile X Syndrome (FMR2, FXR1, FXR2, |
| | mGLUR5); Huntington's disease and disease like disorders (HD, IT15, PRNP, PRIP, |
| | JPH3, JP3, HDL2, TBP, SCA17); Parkinson disease (NR4A2, NURR1, NOT, TINUR, |
| | SNCAIP, TBP, SCA17, SNCA, NACP, PARK1, PARK4, DJ1, PARK7, LRRK2, |
| | PARK8, PINK1, PARK6, UCHL1, PARK5, SNCA, NACP, PARK1, PARK4, PRKN, |
| | PARK2, PDJ, DBH, NDUFV2); Rett syndrome (MECP2, RTT, PPMX, MRX16, |
| | MRX79, CDKL5, STK9, MECP2, RTT, PPMX, MRX16, MRX79, x-Synuclein, DJ-1); |
| | Schizophrenia (Neuregulin1 (Nrg1), Erb4 (receptor for Neuregulin), Complexin1 |
| | (Cplx1), Tph1 Tryptophan hydroxylase, Tph2, Tryptophan hydroxylase 2, Neurexin 1, |
| | GSK3, GSK3a, GSK3b, 5-HTT (Slc6a4), COMT, DRD (Drd1a), SLC6A3, DAOA, |
| | DTNBP1, Dao (Dao1)); Secretase Related Disorders (APH-1 (alpha and beta), |
| | Presenilin (Psen1), nicastrin, (Ncstn), PEN-2, Nos1, Parp1, Nat1, Nat2); Trinucleotide |
| | Repeat Disorders (HTT (Huntington's Dx), SBMA/SMAX1/AR (Kennedy's Dx), |
| | FXN/X25 (Friedrich's Ataxia), ATX3 (Machado- Joseph's Dx), ATXN1 and ATXN2 |
| | (spinocerebellar ataxias), DMPK (myotonic dystrophy), Atrophin-1 and Atn1 (DRPLA |
| | Dx), CBP (Creb-BP - global instability), VLDLR (Alzheimer's), Atxn7, Atxn10). |
| Ocular | Age-related macular degeneration (Abcr, Ccl2, Cc2, cp (ceruloplasmin), Timp3, |
| diseases and | cathepsinD, Vldlr, Ccr2); Cataract (CRYAA, CRYA1, CRYBB2, CRYB2, PITX3, |
| disorders | BFSP2, CP49, CP47, CRYAA, CRYA1, PAX6, AN2, MGDA, CRYBA1, CRYB1, |
| | CRYGC, CRYG3, CCL, LIM2, MP19, CRYGD, CRYG4, BFSP2, CP49, CP47, HSF4, |
| | CTM, HSF4, CTM, MIP, AQP0, CRYAB, CRYA2, CTPP2, CRYBB1, CRYGD, |
| | CRYG4, CRYBB2, CRYB2, CRYGC, CRYG3, CCL, CRYAA, CRYA1, GJA8, CX50, |
| | CAE1, GJA3, CX46, CZP3, CAE3, CCM1, CAM, KRIT1); Corneal clouding and |
| | dystrophy (APOA1, TGFBI, CSD2, CDGG1, CSD, BIGH3, CDG2, TACSTD2, |
| | TROP2, M1S1, VSX1, RINX, PPCD, PPD, KTCN, COL8A2, FECD, PPCD2, PIP5K3, |
| | CFD); Cornea plana congenital (KERA, CNA2); Glaucoma (MYOC, TIGR, GLC1A, |
| | JOAG, GPOA, OPTN, GLC1E, FIP2, HYPL, NRP, CYP1B1, GLC3A, OPA1, NTG, |
| | NPG, CYP1B1, GLC3A); Leber congenital amaurosis (CRB1, RP12, CRX, CORD2, |
| | CRD, RPGRIP1, LCA6, CORD9, RPE65, RP20, AIPL1, LCA4, GUCY2D, GUC2D, |
| | LCA1, CORD6, RDH12, LCA3); Macular dystrophy (ELOVL4, ADMD, STGD2, |
| | STGD3, RDS, RP7, PRPH2, PRPH, AVMD, AOFMD, VMD2). |

TABLE 4
SIGNAL TRANSDUCTION PATHWAYS

| Pathway | Genes |
|---|---|
| PI3K/AKT Signaling | PRKCE; ITGAM; ITGA5; IRAK1; PRKAA2; EIF2AK2; PTEN; EIF4E; |
| | PRKCZ; GRK6; MAPK1; TSC1; PLK1; AKT2; IKBKB; PIK3CA; CDK8; |
| | CDKN1B; NFKB2; BCL2; PIK3CB; PPP2R1A; MAPK8; BCL2L1; MAPK3; |
| | TSC2; ITGA1; KRAS; EIF4EBP1; RELA; PRKCD; NOS3; PRKAA1; |
| | MAPK9; CDK2; PPP2CA; PIM1; ITGB7; YWHAZ; ILK; TP53; RAF1; |
| | IKBKG; RELB; DYRK1A; CDKN1A; ITGB1; MAP2K2; JAK1; AKT1; |
| | JAK2; PIK3R1; CHUK; PDPK1; PPP2R5C; CTNNB1; MAP2K1; NFKB1; |
| | PAK3; ITGB3; CCND1; GSK3A; FRAP1; SFN; ITGA2; TTK; CSNK1A1; |
| | BRAF; GSK3B; AKT3; FOXO1; SGK; HSP90AA1; RPS6KB1 |
| ERK/MAPK Signaling | PRKCE; ITGAM; ITGA5; HSPB1; IRAK1; PRKAA2; EIF2AK2; RAC1; |
| | RAP1A; TLN1; EIF4E; ELK1; GRK6; MAPK1; RAC2; PLK1; AKT2; |
| | PIK3CA; CDK8; CREB1; PRKCI; PTK2; FOS; RPS6KA4; PIK3CB; |
| | PPP2R1A; PIK3C3; MAPK8; MAPK3; ITGA1; ETS1; KRAS; MYCN; |
| | EIF4EBP1; PPARG; PRKCD; PRKAA1; MAPK9; SRC; CDK2; PPP2CA; |
| | PIM1; PIK3C2A; ITGB7; YWHAZ; PPP1CC; KSR1; PXN; RAF1; FYN; |
| | DYRK1A; ITGB1; MAP2K2; PAK4; PIK3R1; STAT3; PPP2R5C; MAP2K1; |
| | PAK3; ITGB3; ESR1; ITGA2; MYC; TTK; CSNK1A1; CRKL; BRAF; ATF4; |
| | PRKCA; SRF; STAT1; SGK |
| Glucocorticoid | RAC1; TAF4B; EP300; SMAD2; TRAF6; PCAF; ELK1; MAPK1; SMAD3; |
| Receptor Signaling | AKT2; IKBKB; NCOR2; UBE2I; PIK3CA; CREB1; FOS; HSPA5; NFKB2; |
| | BCL2; MAP3K14; STAT5B; PIK3CB; PIK3C3; MAPK8; BCL2L1; MAPK3; |
| | TSC22D3; MAPK10; NRIP1; KRAS; MAPK13; RELA; STAT5A; MAPK9; |
| | NOS2A; PBX1; NR3C1; PIK3C2A; CDKN1C; TRAF2; SERPINE1; NCOA3; |
| | MAPK14; TNF; RAF1; IKBKG; MAP3K7; CREBBP; CDKN1A; MAP2K2; |
| | JAK1; IL8; NCOA2; AKT1; JAK2; PIK3R1; CHUK; STAT3; MAP2K1; |
| | NFKB1; TGFBR1; ESR1; SMAD4; CEBPB; JUN; AR; AKT3; CCL2; MMP1; |
| | STAT1; IL6; HSP90AA1 |
| Axonal Guidance | PRKCE; ITGAM; ROCK1; ITGA5; CXCR4; ADAM12; IGF1; RAC1; |
| Signaling | RAP1A; E1F4E; PRKCZ; NRP1; NTRK2; ARHGEF7; SMO; ROCK2; |
| | MAPK1; PGF; RAC2; PTPN11; GNAS; AKT2; PIK3CA; ERBB2; PRKCI; |
| | PTK2; CFL1; GNAQ; PIK3CB; CXCL12; PIK3C3; WNT11; PRKD1; |
| | GNB2L1; ABL1; MAPK3; ITGA1; KRAS; RHOA; PRKCD; PIK3C2A; |
| | ITGB7; GLI2; PXN; VASP; RAF1; FYN; ITGB1; MAP2K2; PAK4; |
| | ADAM17; AKT1; PIK3R1; GLI1; WNT5A; ADAM10; MAP2K1; PAK3; |
| | ITGB3; CDC42; VEGFA; ITGA2; EPHA8; CRKL; RND1; GSK3B; AKT3; |
| | PRKCA |
| Ephrin Receptor | PRKCE; ITGAM; ROCK1; ITGA5; CXCR4; IRAK1; PRKAA2; EIF2AK2; |
| Signaling | RAC1; RAP1A; GRK6; ROCK2; MAPK1; PGF; RAC2; PTPN11; GNAS; |
| | PLK1; AKT2; DOK1; CDK8; CREB1; PTK2; CFL1; GNAQ; MAP3K14; |
| | CXCL12; MAPK8; GNB2L1; ABL1; MAPK3; ITGA1; KRAS; RHOA; |
| | PRKCD; PRKAA1; MAPK9; SRC; CDK2; PIM1; ITGB7; PXN; RAF1; FYN; |
| | DYRK1A; ITGB1; MAP2K2; PAK4, AKT1; JAK2; STAT3; ADAM10; |
| | MAP2K1; PAK3; ITGB3; CDC42; VEGFA; ITGA2; EPHA8; TTK; |
| | CSNK1A1; CRKL; BRAF; PTPN13; ATF4; AKT3; SGK |
| Actin Cytoskeleton | ACTN4; PRKCE; ITGAM; ROCK1; ITGA5; IRAK1; PRKAA2; EIF2AK2; |
| Signaling | RAC1; INS; ARHGEF7; GRK6; ROCK2; MAPK1; RAC2; PLK1; AKT2; |
| | PIK3CA; CDK8; PTK2; CFL1; PIK3CB; MYH9; DIAPH1; PIK3C3; MAPK8; |
| | F2R; MAPK3; SLC9A1; ITGA1; KRAS; RHOA; PRKCD; PRKAA1; |
| | MAPK9; CDK2; PIM1; PIK3C2A; ITGB7; PPP1CC; PXN; VIL2; RAF1; |
| | GSN; DYRK1A; ITGB1; MAP2K2; PAK4; PIP5K1A; PIK3R1; MAP2K1; |
| | PAK3; ITGB3; CDC42; APC; ITGA2; TTK; CSNK1A1; CRKL; BRAF; |
| | VAV3; SGK |
| Huntington's Disease | PRKCE; IGF1; EP300; RCOR1; PRKCZ; HDAC4; TGM2; MAPK1; CAPNS1; |
| Signaling | AKT2; EGFR; NCOR2; SP1; CAPN2; PIK3CA; HDAC5; CREB1; PRKC1; |
| | HSPA5; REST; GNAQ; PIK3CB; PIK3C3; MAPK8; IGF1R; PRKD1; |
| | GNB2L1; BCL2L1; CAPN1; MAPK3; CASP8; HDAC2; HDAC7A; PRKCD; |
| | HDAC11; MAPK9; HDAC9; PIK3C2A; HDAC3; TP53; CASP9; CREBBP; |
| | AKT1; PIK3R1; PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; BIRC4; GRK6; |
| | MAPK1; CAPNS1; PLK1; AKT2; IKBKB; CAPN2; CDK8; FAS; NFKB2; |
| | BCL2; MAP3K14; MAPK8; BCL2L1; CAPN1; MAPK3; CASP8; KRAS; |
| | RELA; PRKCD; PRKAA1; MAPK9; CDK2; PIM1; TP53; TNF; RAF1; |
| | IKBKG; RELB; CASP9; DYRK1A; MAP2K2; CHUK; APAF1; MAP2K1; |
| | NFKB1; PAK3; LMNA; CASP2; BIRC2; TTK; CSNK1A1; BRAF; BAX; |
| | PRKCA; SGK; CASP3; BIRC3; PARP1 |
| B Cell Receptor | RAC1; PTEN; LYN; ELK1; MAPK1; RAC2; PTPN11; AKT2; IKBKB; |
| Signaling | PIK3CA; CREB1; SYK; NFKB2; CAMK2A; MAP3K14; PIK3CB; PIK3C3; |
| | MAPK8; BCL2L1; ABL1; MAPK3; ETS1; KRAS; MAPK13; RELA; PTPN6; |
| | MAPK9; EGR1; PIK3C2A; BTK; MAPK14; RAF1; IKBKG; RELB; |
| | MAP3K7; MAP2K2; AKT1; PIK3R1; CHUK; MAP2K1; NFKB1; CDC42; |
| | GSK3A; FRAP1; BCL6; BCL10; JUN; GSK3B; ATF4; AKT3; VAV3; |
| | RPS6KB1 |
| Leukocyte | ACTN4; CD44; PRKCE; ITGAM; ROCK1; CXCR4; CYBA; RAC1; RAP1A; |
| Extravasation Signaling | PRKCZ; ROCK2; RAC2; PTPN11; MMP14; PIK3CA; PRKCI; PTK2; |
| | PIK3CB; CXCL12; PIK3C3; MAPK8; PRKD1; ABL1; MAPK10; CYBB; |
| | MAPK13; RHOA; PRKCD; MAPK9; SRC; PIK3C2A; BTK; MAPK14; |
| | NOX1; PXN; VIL2; VASP; ITGB1; MAP2K2; CTNND1; PIK3R1; CTNNB1; |
| | CLDN1; CDC42; F11R; ITK; CRKL; VAV3; CTTN; PRKCA; MMP1; MMP9 |
| Integrin Signaling | ACTN4; ITGAM; ROCK1; ITGA5; RAC1; PTEN; RAP1A; TLN1; |
| | ARHGEF7; MAPK1; RAC2; CAPNS1; AKT2; CAPN2; PIK3CA; PTK2; |
| | PIK3CB; PIK3C3; MAPK8; CAV1; CAPN1; ABL1; MAPK3; ITGA1; KRAS; |
| | RHOA; SRC; PIK3C2A; ITGB7; PPP1CC; ILK; PXN; VASP; RAF1; FYN; |
| | ITGB1; MAP2K2; PAK4; AKT1; PIK3R1; TNK2; MAP2K1; PAK3; ITGB3; |
| | CDC42; RND3; ITGA2; CRKL; BRAF; GSK3B; AKT3 |
| Acute Phase Response | IRAK1; SOD2; MYD88; TRAF6; ELK1; MAPK1; PTPN11; AKT2; IKBKB; |
| Signaling | PIK3CA; FOS; NFKB2; MAP3K14; PIK3CB; MAPK8; RIPK1; MAPK3; |
| | IL6ST; KRAS; MAPK13; IL6R; RELA; SOCS1; MAPK9; FTL; NR3C1; |
| | TRAF2; SERPINE1; MAPK14; TNF; RAF1; PDK1; IKBKG; RELB; |
| | MAP3K7; MAP2K2; AKT1; JAK2; PIK3R1; CHUK; STAT3; MAP2K1; |
| | NFKB1; FRAP1; CEBPB; JUN; AKT3; IL1R1; IL6 |
| PTEN Signaling | ITGAM; ITGA5; RAC1; PTEN; PRKCZ; BCL2L11; MAPK1; RAC2; AKT2; |
| | EGFR; IKBKB; CBL; PIK3CA; CDKN1B; PTK2; NFKB2; BCL2; PIK3CB; |
| | BCL2L1; MAPK3; ITGA1; KRAS; ITGB7; ILK; PDGFRB; INSR; RAF1; |
| | IKBKG; CASP9; CDKN1A; ITGB1; MAP2K2; AKT1; PIK3R1; CHUK; |
| | PDGFRA; PDPK1; MAP2K1; NFKB1; ITGB3; CDC42; CCND1; GSK3A; |
| | ITGA2; GSK3B; AKT3; FOXO1; CASP3; RPS6KB1 |
| p53 Signaling | PTEN; EP300; BBC3; PCAF; FASN; BRCA1; GADD45A; BIRC5; AKT2; |
| | PIK3CA; CHEK1; TP53INP1; BCL2; PIK3CB; PIK3C3; MAPK8; THBS1; |
| | ATR; BCL2L1; E2F1; PMAIP1; CHEK2; TNFRSF10B; TP73; RB1; HDAC9; |
| | CDK2; PIK3C2A; MAPK14; TP53; LRDD; CDKN1A; HIPK2; AKT1; |
| | PIK3R1; RRM2B; APAF1; CTNNB1; SIRT1; CCND1; PRKDC; ATM; SFN; |
| | CDKN2A; JUN; SNAI2; GSK3B; BAX; AKT3 |
| Aryl Hydrocarbon | HSPB1; EP300; FASN; TGM2; RXRA; MAPK1; NQO1; NCOR2; SP1; |
| Receptor Signaling | ARNT; CDKN1B; FOS; CHEK1; SMARCA4; NFKB2; MAPK8; ALDH1A1; |
| | ATR; E2F1; MAPK3; NRIP1; CHEK2; RELA; TP73; GSTP1; RB1; SRC; |
| | CDK2; AHR; NFE2L2; NCOA3; TP53; TNF; CDKN1A; NCOA2; APAF1; |
| | NFKB1; CCND1; ATM; ESR1; CDKN2A; MYC; JUN; ESR2; BAX; IL6; |
| | CYP1B1; HSP90AA1 |
| Xenobiotic Metabolism | PRKCE; EP300; PRKCZ; RXRA; MAPK1; NQO1; NCOR2; PIK3CA; ARNT; |
| Signaling | PRKC1; NFKB2; CAMK2A; PIK3CB; PPP2R1A; PIK3C3; MAPK8; PRKD1; |
| | ALDH1A1; MAPK3; NRIP1; KRAS; MAPK13; PRKCD; GSTP1; MAPK9; |
| | NOS2A; ABCB1; AHR; PPP2CA; FTL; NFE2L2; PIK3C2A; PPARGC1A; |
| | MAPK14; TNF; RAF1; CREBBP; MAP2K2; PIK3R1; PPP2R5C; MAP2K1; |
| | NFKB1; KEAP1; PRKCA; EIF2AK3; IL6; CYP1B1; HSP90AA1 |
| SAPK/JNK Signaling | PRKCE; IRAK1; PRKAA2; EIF2AK2; RAC1; ELK1; GRK6; MAPK1; |
| | GADD45A; RAC2; PLK1; AKT2; PIK3CA; FADD; CDK8; PIK3CB; |
| | PIK3C3; MAPK8; RIPK1; GNB2L1; IRS1; MAPK3; MAPK10; DAXX; |
| | KRAS; PRKCD; PRKAA1; MAPK9; CDK2; PIM1; PIK3C2A; TRAF2; TP53; |
| | LCK; MAP3K7; DYRK1A; MAP2K2; PIK3R1; MAP2K1; PAK3; CDC42; |
| | JUN; TTK; CSNK1A1; CRKL; BRAF; SGK |
| PPAr/RXR Signaling | PRKAA2; EP300; INS; SMAD2; TRAF6; PPARA; FASN; RXRA; MAPK1; |
| | SMAD3; GNAS; IKBKB; NCOR2; ABCA1; GNAQ; NFKB2; MAP3K14; |
| | STAT5B; MAPK8; IRS1; MAPK3; KRAS; RELA; PRKAA1; PPARGC1A; |
| | NCOA3; MAPK14; INSR; RAF1; IKBKG; RELB; MAP3K7; CREBBP; |
| | MAP2K2; JAK2; CHUK; MAP2K1; NFKB1; TGFBR1; SMAD4; JUN; |
| | IL1R1; PRKCA; IL6; HSP90AA1; ADIPOQ |
| NF-KB Signaling | IRAK1; EIF2AK2; EP300; INS; MYD88; PRKCZ: TRAF6; TBK1; AKT2; |
| | EGFR; IKBKB; PIK3CA; BTRC; NFKB2; MAP3K14; PIK3CB; PIK3C3; |
| | MAPK8; RIPK1; HDAC2; KRAS; RELA; PIK3C2A; TRAF2; TLR4: |
| | PDGFRB; TNF; INSR; LCK; IKBKG; RELB; MAP3K7; CREBBP; AKT1; |
| | PIK3R1; CHUK; PDGFRA; NFKB1; TLR2; BCL10; GSK3B; AKT3; |
| | TNFAIP3; IL1R1 |
| Neuregulin Signaling | ERBB4; PRKCE; ITGAM; ITGA5: PTEN; PRKCZ; ELK1; MAPK1; PTPN11; |
| | AKT2; EGFR; ERBB2; PRKCI; CDKN1B; STAT5B; PRKD1; MAPK3; |
| | ITGA1; KRAS; PRKCD; STAT5A; SRC; ITGB7; RAF1; ITGB1; MAP2K2; |
| | ADAM17; AKT1; PIK3R1; PDPK1; MAP2K1; ITGB3; EREG; FRAP1; |
| | PSEN1; ITGA2; MYC; NRG1; CRKL; AKT3; PRKCA; HSP90AA1; |
| | RPS6KB1 |
| Wnt & Beta catenin | CD44; EP300; LRP6; DVL3; CSNK1E; GJA1; SMO; AKT2; PIN1; CDH1; |
| Signaling | BTRC; GNAQ; MARK2; PPP2R1A; WNT11; SRC; DKK1; PPP2CA; SOX6; |
| | SFRP2: ILK; LEF1; SOX9; TP53; MAP3K7; CREBBP; TCF7L2; AKT1; |
| | PPP2R5C; WNT5A; LRP5; CTNNB1; TGFBR1; CCND1; GSK3A; DVL1; |
| | APC; CDKN2A; MYC; CSNK1A1; GSK3B; AKT3; SOX2 |
| Insulin Receptor | PTEN; INS; EIF4E; PTPN1; PRKCZ; MAPK1; TSC1; PTPN11; AKT2; CBL; |
| Signaling | PIK3CA; PRKCI; PIK3CB; PIK3C3; MAPK8; IRS1; MAPK3; TSC2; KRAS; |
| | EIF4EBP1; SLC2A4; PIK3C2A; PPP1CC; INSR; RAF1; FYN; MAP2K2; |
| | JAK1; AKT1; JAK2; PIK3R1; PDPK1; MAP2K1; GSK3A; FRAP1; CRKL; |
| | GSK3B; AKT3; FOXO1; SGK; RPS6KB1 |
| IL-6 Signaling | HSPB1; TRAF6; MAPKAPK2; ELK1; MAPK1; PTPN11; IKBKB; FOS; |
| | NFKB2: MAP3K14; MAPK8; MAPK3; MAPK10; IL6ST; KRAS; MAPK13; |
| | IL6R; RELA; SOCS1; MAPK9; ABCB1; TRAF2; MAPK14; TNF; RAF1; |
| | IKBKG; RELB; MAP3K7; MAP2K2; IL8; JAK2; CHUK; STAT3; MAP2K1; |
| | NFKB1; CEBPB; JUN; IL1R1; SRF; IL6 |
| Hepatic Cholestasis | PRKCE; IRAK1; INS; MYD88; PRKCZ; TRAF6; PPARA; RXRA; IKBKB; |
| | PRKCI; NFKB2; MAP3K14; MAPK8; PRKD1; MAPK10; RELA; PRKCD; |
| | MAPK9; ABCB1; TRAF2; TLR4; TNF; INSR; IKBKG; RELB; MAP3K7; |
| | IL8; CHUK; NR1H2; TJP2; NFKB1; ESR1; SREBF1; FGFR4; JUN; IL1R1; |
| | PRKCA; IL6 |
| IGF-1 Signaling | IGF1; PRKCZ; ELK1; MAPK1; PTPN11; NEDD4; AKT2; PIK3CA; PRKCI; |
| | PTK2; FOS; PIK3CB; PIK3C3; MAPK8; IGF1R; IRS1; MAPK3; IGFBP7; |
| | KRAS; PIK3C2A; YWHAZ; PXN; RAF1; CASP9; MAP2K2; AKT1; PIK3R1; |
| | PDPK1; MAP2K1; IGFBP2; SFN; JUN; CYR61; AKT3; FOXO1; SRF; |
| | CTGF; RPS6KB1 |
| NRF2-mediated | PRKCE; EP300; SOD2; PRKCZ; MAPK1; SQSTM1; NQO1; PIK3CA; |
| Oxidative Stress | PRKCI; FOS; PIK3CB; PIK3C3; MAPK8; PRKD1; MAPK3; KRAS; PRKCD; |
| Response | GSTP1; MAPK9; FTL; NFE2L2; PIK3C2A; MAPK14; RAF1; MAP3K7; |
| | CREBBP; MAP2K2; AKT1; PIK3R1; MAP2K1; PPIB; JUN; KEAP1; |
| | GSK3B; ATF4; PRKCA; EIF2AK3; HSP90AA1 |
| Hepatic | EDN1; IGF1; KDR; FLT1; SMAD2; FGFR1; MET; PGF; SMAD3; EGFR; |
| Fibrosis/Hepatic | FAS; CSF1; NFKB2; BCL2; MYH9; IGF1R; IL6R; RELA; TLR4; PDGFRB; |
| Stellate Cell Activation | TNF; RELB; IL8; PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; NCOR2; FOS; |
| | NFKB2; MAP3K14; STAT5B; MAPK3; NRIP1; KRAS; PPARG; RELA; |
| | STAT5A; TRAF2; PPARGC1A; PDGFRB; TNF; INSR; RAF1; IKBKG; |
| | RELB; MAP3K7; CREBBP; MAP2K2; CHUK; PDGFRA; MAP2K1; NFKB1; |
| | JUN; IL1R1; HSP90AA1 |
| Fc Epsilon RI Signaling | PRKCE; RAC1; PRKCZ; LYN; MAPK1; RAC2; PTPN11; AKT2; PIK3CA; |
| | SYK; PRKCI; PIK3CB; PIK3C3; MAPK8; PRKD1; MAPK3; MAPK10; |
| | KRAS; MAPK13; PRKCD; MAPK9; PIK3C2A; BTK; MAPK14; TNF; RAF1; |
| | FYN; MAP2K2; AKT1; PIK3R1; PDPK1; MAP2K1; AKT3; VAV3; PRKCA |
| G-Protein Coupled | PRKCE; RAP1A; RGS16; MAPK1; GNAS; AKT2; IKBKB; PIK3CA; |
| Receptor Signaling | CREB1; GNAQ; NFKB2; CAMK2A; PIK3CB; PIK3C3; MAPK3; KRAS; |
| | RELA; SRC; PIK3C2A; RAF1; IKBKG; RELB; FYN; MAP2K2; AKT1; |
| | PIK3R1; CHUK; PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; MAPK1; PLK1; AKT2; |
| Metabolism | PIK3CA; CDK8; PIK3CB; PIK3C3; MAPK8; MAPK3; PRKCD; PRKAA1; |
| | MAPK9; CDK2; PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; PIK3C3; |
| | MAPK8; CAV1; ABL1; MAPK3; KRAS; SRC; PIK3C2A; PDGFRB; RAF1; |
| | MAP2K2; JAK1; JAK2; PIK3R1; PDGFRA; STAT3; SPHK1; MAP2K1; |
| | MYC; JUN; CRKL; PRKCA; SRF; STAT1; SPHK2 |
| VEGF Signaling | ACTN4; ROCK1; KDR; FLT1; ROCK2; MAPK1; PGF; AKT2; PIK3CA; |
| | ARNT; PTK2; BCL2; PIK3CB; PIK3C3; BCL2L1; MAPK3; KRAS; HIF1A; |
| | NOS3; PIK3C2A; PXN; RAF1; MAP2K2; ELAVL1; AKT1; PIK3R1; |
| | MAP2K1; SFN; VEGFA; AKT3; FOXO1; PRKCA |
| Natural Killer Cell | PRKCE; RAC1; PRKCZ; MAPK1; RAC2; PTPN11; KIR2DL3; AKT2; |
| Signaling | PIK3CA; SYK; PRKCI; PIK3CB; PIK3C3; PRKD1; MAPK3; KRAS; |
| | PRKCD; PTPN6; PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; ATR; ABL1; E2F1; |
| Checkpoint Regulation | HDAC2; HDAC7A; RB1; HDAC11; HDAC9; CDK2; E2F2; HDAC3; TP53; |
| | CDKN1A; CCND1; E2F4; ATM; RBL2; SMAD4; CDKN2A; MYC; NRG1; |
| | GSK3B; RBL1;HDAC6 |
| T Cell Receptor | RAC1; ELK1; MAPK1; IKBKB; CBL; PIK3CA; FOS; NFKB2; PIK3CB; |
| Signaling | PIK3C3; MAPK8; MAPK3; KRAS; RELA, PIK3C2A; BTK; LCK; RAF1; |
| | IKBKG; RELB, FYN; MAP2K2; PIK3R1; CHUK; MAP2K1; NFKB1; ITK; |
| | BCL10; JUN; VAV3 |
| Death Receptor | CRADD; HSPB1; BID; BIRC4; TBK1; IKBKB; FADD; FAS; NFKB2; BCL2; |
| Signaling | MAP3K14; MAPK8; RIPK1; CASP8; DAXX; TNFRSF10B; RELA; TRAF2; |
| | TNF; IKBKG; RELB; CASP9; CHUK; APAF1; NFKB1; CASP2; BIRC2; |
| | CASP3; BIRC3 |
| FGF Signaling | RAC1; FGFR1; MET; MAPKAPK2; MAPK1; PTPN11; AKT2; PIK3CA; |
| | CREB1; PIK3CB; PIK3C3; MAPK8; MAPK3; MAPK13; PTPN6; PIK3C2A; |
| | MAPK14; RAF1; AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; STAT5B; |
| | PIK3CB; PIK3C3; GNB2L1; BCL2L1; MAPK3; ETS1; KRAS; RUNX1; |
| | PIM1; PIK3C2A; RAF1; MAP2K2; AKT1; JAK2; PIK3R1; STAT3; |
| | MAP2K1; CCND1; AKT3; STAT1 |
| Amyotrophic Lateral | BID; IGF1; RAC1; BIRC4; PGF; CAPNS1; CAPN2; PIK3CA; BCL2; |
| Sclerosis Signaling | PIK3CB; PIK3C3; BCL2L1; CAPN1; PIK3C2A; TP53; CASP9; PIK3R1; |
| | RAB5A; CASP1; APAF1; VEGFA; BIRC2; BAX; AKT3; CASP3; BIRC3 |
| JAK/Stat Signaling | PTPN1; MAPK1; PTPN11; AKT2; PIK3CA; STAT5B; PIK3CB; PIK3C3; |
| | MAPK3; KRAS; SOCS1; STAT5A; PTPN6; PIK3C2A; RAF1; CDKN1A; |
| | MAP2K2; JAK1; AKT1; JAK2; PIK3R1; STAT3; MAP2K1; FRAP1; AKT3; |
| | STAT1 |
| Nicotinate and | PRKCE; IRAK1; PRKAA2; EIF2AK2; GRK6; MAPK1; PLK1; AKT2; CDK8; |
| Nicotinamide | MAPK8; MAPK3; PRKCD; PRKAA1; PBEF1; MAPK9; CDK2; PIM1; |
| Metabolism | DYRK1A; MAP2K2; MAP2K1; PAK3; NT5E; TTK; CSNK1A1; BRAF; SGK |
| Chemokine Signaling | CXCR4; ROCK2; MAPK1; PTK2; FOS; CFL1; GNAQ; CAMK2A; CXCL12; |
| | MAPK8; MAPK3; KRAS; MAPK13; RHOA; CCR3; SRC; PPP1CC; |
| | MAPK14; NOX1; RAF1; MAP2K2; MAP2K1; JUN; CCL2; PRKCA |
| IL-2 Signaling | ELK1; MAPK1; PTPN11; AKT2; PIK3CA; SYK; FOS; STAT5B; PIK3CB; |
| | PIK3C3; MAPK8; MAPK3; KRAS; SOCS1; STAT5A; PIK3C2A; LCK; |
| | RAF1; MAP2K2; JAK1; AKT1; PIK3R1; MAP2K1; JUN; AKT3 |
| Synaptic Long Term | PRKCE; IGF1; PRKCZ; PRDX6; LYN; MAPK1; GNAS; PRKCI; GNAQ; |
| Depression | PPP2R1A; IGF1R; PRKD1; MAPK3; KRAS; GRN; PRKCD; NOS3; NOS2A; |
| | PPP2CA; YWHAZ; RAF1; MAP2K2; PPP2R5C; MAP2K1; PRKCA |
| Estrogen Receptor | TAF4B; EP300; CARM1; PCAF; MAPK1; NCOR2; SMARCA4; MAPK3; |
| Signaling | NRIP1; KRAS; SRC; NR3C1; HDAC3; PPARGC1A; RBM9; NCOA3; RAF1; |
| | CREBBP; MAP2K2; NCOA2; MAP2K1; PRKDC; ESR1; ESR2 |
| Protein Ubiquitination | TRAF6; SMURF1; BIRC4; BRCA1; UCHL1; NEDD4; CBL; UBE2I; BTRC; |
| Pathway | HSPA5; USP7; USP10; FBXW7; USP9X; STUB1; USP22; B2M; BIRC2; |
| | PARK2; USP8; USP1; VHL; HSP90AA1; BIRC3 |
| IL-10 Signaling | TRAF6; CCR1; ELK1; IKBKB; SP1; FOS; NFKB2; MAP3K14; MAPK8; |
| | MAPK13; RELA; MAPK14; TNF; IKBKG; RELB; MAP3K7; JAK1; CHUK; |
| | STAT3; NFKB1; JUN; IL1R1; IL6 |
| VDR/RXR Activation | PRKCE; EP300; PRKCZ; RXRA; GADD45A; HES1; NCOR2; SP1; PRKCI; |
| | CDKN1B; PRKD1; PRKCD; RUNX2; KLF4; YY1; NCOA3; CDKN1A; |
| | NCOA2; SPP1; LRP5; CEBPB; FOXO1; PRKCA |
| TGF-beta Signaling | EP300; SMAD2; SMURF1; MAPK1; SMAD3; SMAD1; FOS; MAPK8; |
| | MAPK3; KRAS; MAPK9; RUNX2; SERPINE1; RAF1; MAP3K7; CREBBP; |
| | MAP2K2; MAP2K1; TGFBR1; SMAD4; JUN; SMAD5 |
| Toll-like Receptor | IRAK1; EIF2AK2; MYD88; TRAF6; PPARA; ELK1; IKBKB; FOS; NFKB2; |
| Signaling | MAP3K14; MAPK8; MAPK13; RELA; TLR4; MAPK14; IKBKG; RELB; |
| | MAP3K7; CHUK; NFKB1; TLR2; JUN |
| p38 MAPK Signaling | HSPB1; IRAK1; TRAF6; MAPKAPK2; ELK1; FADD; FAS; CREB1; DDIT3; |
| | RPS6KA4; DAXX; MAPK13; TRAF2; MAPK14; TNF; MAP3K7; TGFBR1; |
| | MYC; ATF4; IL1R1; SRF; STAT1 |
| Neurotrophin/TRK | NTRK2; MAPK1; PTPN11; PIK3CA; CREB1; FOS; PIK3CB; PIK3C3; |
| Signaling | MAPK8; MAPK3; KRAS; PIK3C2A; RAF1; MAP2K2; AKT1; PIK3R1; |
| | PDPK1; MAP2K1; CDC42; JUN; ATF4 |
| FXR/RXR Activation | INS; PPARA; FASN; RXRA; AKT2; SDC1; MAPK8; APOB; MAPK10; |
| | PPARG; MTTP; MAPK9; PPARGC1A; TNF; CREBBP; AKT1; SREBF1; |
| | FGFR4; AKT3; FOXO1 |
| Synaptic Long Term | PRKCE; RAP1A; EP300; PRKCZ; MAPK1; CREB1; PRKCI; GNAQ; |
| Potentiation | CAMK2A; PRKD1; MAPK3; KRAS; PRKCD; PPP1CC; RAF1; CREBBP; |
| | MAP2K2; MAP2K1; ATF4; PRKCA |
| Calcium Signaling | RAP1A; EP300; HDAC4; MAPK1; HDAC5; CREB1; CAMK2A; MYH9; |
| | MAPK3; HDAC2; HDAC7A; HDAC11; HDAC9; HDAC3; CREBBP; CALR; |
| | CAMKK2; ATF4; HDAC6 |
| EGF Signaling | ELK1; MAPK1; EGFR; PIK3CA; FOS; PIK3CB; PIK3C3; MAPK8; MAPK3; |
| | PIK3C2A; RAF1; JAK1; PIK3R1; STAT3; MAP2K1; JUN; PRKCA; SRF; |
| | STAT1 |
| Hypoxia Signaling in | EDN1; PTEN; EP300; NQO1; UBE2I; CREB1; ARNT; HIF1A; SLC2A4; |
| the Cardiovascular | NOS3; TP53; LDHA; AKT1; ATM; VEGFA; JUN; ATF4; VHL; HSP90AA1 |
| System | |
| LPS/IL-1 Mediated | IRAK1; MYD88; TRAF6; PPARA; RXRA; ABCA1, MAPK8; ALDH1A1; |
| Inhibition of RXR | GSTP1; MAPK9; ABCB1; TRAF2; TLR4; TNF; MAP3K7; NR1H2; SREBF1; |
| Function | JUN; IL1R1 |
| LXR/RXR Activation | FASN; RXRA; NCOR2; ABCA1; NFKB2; IRF3; RELA; NOS2A; TLR4; |
| | TNF; RELB; LDLR; NR1H2; NFKB1; SREBF1; IL1R1; CCL2; IL6; MMP9 |
| Amyloid Processing | PRKCE; CSNK1E; MAPK1; CAPNS1; AKT2; CAPN2; CAPN1; MAPK3; |
| | MAPK13; MAPT; MAPK14; AKT1; PSEN1; CSNK1A1; GSK3B; AKT3; |
| | APP |
| IL-4 Signaling | AKT2; PIK3CA; PIK3CB; PIK3C3; IRS1; KRAS; SOCS1; PTPN6; NR3C1; |
| | PIK3C2A; JAK1; AKT1; JAK2; PIK3R1; FRAP1; AKT3; RPS6KB1 |
| Cell Cycle: G2/M DNA | EP300; PCAF; BRCA1; GADD45A; PLK1; BTRC; CHEK1; ATR; CHEK2; |
| Damage Checkpoint | YWHAZ; TP53; CDKN1A; PRKDC; ATM; SFN; CDKN2A |
| Regulation | |
| Nitric Oxide Signaling | KDR; FLT1; PGF; AKT2; PIK3CA; PIK3CB; PIK3C3; CAV1; PRKCD; |
| in the Cardiovascular | NOS3; PIK3C2A; AKT1; PIK3R1; VEGFA; AKT3; HSP90AA1 |
| System | |
| Purine Metabolism | NME2; SMARCA4; MYH9; RRM2; ADAR; EIF2AK4; PKM2; ENTPD1; |
| | RAD51; RRM2B; TJP2; RAD51C; NT5E; POLD1; NME1 |
| cAMP-mediated | RAP1A; MAPK1; GNAS; CREB1; CAMK2A; MAPK3; SRC; RAF1; |
| Signaling | MAP2K2; STAT3; MAP2K1; BRAF; ATF4 |
| Mitochondrial | SOD2; MAPK8; CASP8; MAPK10; MAPK9; CASP9; PARK7; PSEN1; |
| Dysfunction | PARK2; APP; CASP3 |
| Notch Signaling | HES1; JAG1; NUMB; NOTCH4; ADAM17; NOTCH2; PSEN1; NOTCH3; |
| | NOTCH1; DLL4 |
| Endoplasmic Reticulum | HSPA5; MAPK8; XBP1; TRAF2; ATF6; CASP9; ATF4; EIF2AK3; CASP3 |
| Stress Pathway | |
| Pyrimidine Metabolism | NME2; AICDA; RRM2; EIF2AK4; ENTPD1; RRM2B; NT5E; POLD1; |
| | NME1 |
| Parkinson's Signaling | UCHL1; MAPK8; MAPK13; MAPK14; CASP9; PARK7; PARK2; CASP3 |
| Cardiac & Beta | GNAS; GNAQ; PPP2R1A; GNB2L1; PPP2CA; PPP1CC; PPP2R5C |
| Adrenergic Signaling | |
| Glycolysis/ | HK2; GCK; GPI; ALDH1A1; PKM2; LDHA; HK1 |
| Gluconeogenesis | |
| Interferon Signaling | IRF1; SOCS1; JAK1; JAK2; IFITM1; STAT1; IFIT3 |
| Sonic Hedgehog | ARRB2; SMO; GLI2; DYRK1A; GLI1; GSK3B; DYRKIB |
| Signaling | |
| Glycerophospholipid | PLD1; GRN; GPAM; YWHAZ; SPHK1; SPHK2 |
| Metabolism | |
| Phospholipid | PRDX6; PLD1; GRN; YWHAZ; SPHK1; SPHK2 |
| Degradation | |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision | ERCC5; ERCC4; XPA; XPC; ERCC1 |
| Repair Pathway | |
| Starch and Sucrose | UCHL1; HK2; GCK; GPI; HK1 |
| Metabolism | |
| Aminosugars | NQO1; HK2; GCK; HK1 |
| Metabolism | |
| Arachidonic Acid | PRDX6; GRN; YWHAZ; CYP1B1 |
| Metabolism | |
| Circadian Rhythm | CSNK1E; CREB1; ATF4; NR1D1 |
| Signaling | |
| Coagulation System | BDKRB1; F2R; SERPINE1; F3 |
| Dopamine Receptor | PPP2R1A; PPP2CA; PPP1CC; PPP2R5C |
| Signaling | |
| Glutathione | IDH2; GSTP1; ANPEP; IDH1 |
| Metabolism | |
| Glycerolipid | ALDH1A1; GPAM; SPHK1; SPHK2 |
| Metabolism | |
| Linoleic Acid | PRDX6; GRN; YWHAZ; CYP1B1 |
| Metabolism | |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine | PRDX6; PRDX1; TYR |
| and Lignin | |
| Biosynthesis | |
| Antigen Presentation | CALR; B2M |
| Pathway | |
| Biosynthesis of | NQO1; DHCR7 |
| Steroids | |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of | GSTP1; CYP1B1 |
| Xenobiotics by | |
| Cytochrome p450 | |
| Methane Metabolism | PRDX6; PRDX1 |
| Phenylalanine | PRDX6; PRDX1 |
| Metabolism | |
| Propanoate Metabolism | ALDH1A1; LDHA |
| Selenoamino Acid | PRMT5; AHCY |
| Metabolism | |
| Sphingolipid | SPHK1; SPHK2 |
| Metabolism | |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated | PRDX1 |
| Oxidative Stress | |
| Response | |
| Pentose Phosphate | GPI |
| Pathway | |
| Pentose and | UCHL1 |
| Glucuronate | |
| Interconversions | |
| Retinol Metabolism | ALDH1A1 |
| Riboflavin Metabolism | TYR |
| Tyrosine Metabolism | PRMT5, TYR |
| Ubiquinone | PRMT5 |
| Biosynthesis | |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; Trpa1; Pomc; Cgrp; Crf; |
| | Pka; Era; Nr2b; TRPM5; Prkaca; Prkacb; Prkar1a; Prkar2a |
| Mitochondrial Function | AIF; CytC; SMAC (Diablo); Aifm-1; Aifm-2 |
| Developmental | BMP-4; Chordin (Chrd); Noggin (Nog); WNT (Wnt2; Wnt2b; Wnt3a; Wnt4; |
| Neurology | Wnt5a; Wnt6; Wnt7b; Wnt8b; Wnt9a; Wnt9b; Wnt10a; Wnt10b; Wnt16); beta- |
| | catenin; Dkk-1; Frizzled related proteins; Otx-2; Gbx2; FGF-8; Reelin; Dab1; |
| | unc-86 (Pou4fl or Brn3a); Numb; Reln |

### Methods of Treatment, Formulations, Dosages, and Routes of Administration

Disclosed herein include methods of treating a disease or disorder in a subject. In some embodiments, the method comprises: administering to the subject a composition comprising a variant AAV capsid provided herein, or a therapeutically effective amount of an rAAV provided herein, or a pharmaceutical composition provided herein. The administering can comprise systemic administration. The systemic administration can be intravenous, intramuscular, intraperitoneal, or intraarticular. Administering can comprise intrathecal administration, intracranial injection, aerosol delivery, nasal delivery, vaginal delivery, direct injection to any tissue in the body, intraventricular delivery, intraocular delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof.

Administering can comprise an injection into a brain region. For example, the brain region can comprise the Lateral parabrachial nucleus, brainstem, Medulla oblongata, Medullary pyramids, Olivary body, Inferior olivary nucleus, Rostral ventrolateral medulla, Respiratory center, Dorsal respiratory group, Ventral respiratory group, Pre-Botzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Subparabrachial nucleus (Kölliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyms, Cornu ammonis, Cornu ammonis area 1, Cornu ammonis area 2, Cornu ammonis area 3, Cornu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyms, Middle frontal gyms, Inferior frontal gyms, Brodmann areas (4, 6, 8, 9, 10, 11, 12, 24, 25, 32, 33, 44, 45, 46, and/or 47), Parietal lobe, Parietal cortex, Primary somatosensory cortex (51), Secondary somatosensory cortex (S2), Posterior parietal cortex, postcentral gyms, precuneus, Brodmann areas (1, 2, 3 (Primary somesthetic area), 5, 7, 23, 26, 29, 31, 39, and/or 40), Occipital lobe, Primary visual cortex (V1), V2, V3, V4, V5/MT, Lateral occipital gyms, Cuneus, Brodmann areas (17 (V1, primary visual cortex), 18, and/or 19), temporal lobe, Primary auditory cortex (A1), secondary auditory cortex (A2), Inferior temporal cortex, Posterior inferior temporal cortex, Superior temporal gyms, Middle temporal gyms, Inferior temporal gyms, Entorhinal Cortex, Perirhinal Cortex, Parahippocampal gyms, Fusiform gyms, Brodmann areas (9, 20, 21, 22, 27, 34, 35, 36, 37, 38, 41, and/or 42), Medial superior temporal area (MST), insular cortex, cingulate cortex, Anterior cingulate, Posterior cingulate, dorsal cingulate, Retrosplenial cortex, Indusium griseum, Subgenual area 25, Brodmann areas (23, 24; 26, 29, 30 (retrosplenial areas), 31, and/or 32), cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), or any combination thereof.

The brain region can comprise neural pathways Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract, or any combination thereof. Administering can comprise direct administration to the brain parenchyma. Administering can comprise delivery to dorsal root ganglia, visceral organs, astrocytes, neurons, or a combination thereof of the subject.

The variant AAV capsid can comprise tropism for a target tissue or a target cell. The target tissue or the target cell can comprise a tissue or a cell of a central nervous system (CNS) or a peripheral nervous system (PNS), or a combination thereof. The target cell can be a neuronal cell, a neural stem cell, an astrocytes, a tumor cell, a hematopoetic stem cell, an insulin producing beta cell, a lung epithelium, a skeletal cell, or a cardiac muscle cell. The target cell can be located in a brain or spinal cord. The target cell can comprise an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof.

Administering can comprise contacting one or more cells from the subject with a composition comprising a variant AAV capsid provided herein, or a therapeutically effective amount of a rAAV provided herein, or a pharmaceutical composition provided herein. Administering can comprise: (i) isolating one or more cells from the subject; (ii) contacting said one or more cells with a composition comprising a variant AAV capsid provided herein, or a therapeutically effective amount of a rAAV provided herein, or a pharmaceutical composition provided herein; and (iii) administering the one or more cells into a subject after the contacting step. The contacting can be performed in vivo, in vitro, and/or ex vivo. The contacting can comprise calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof. The method can comprise: administering an inducer (e.g., doxycycline) of the inducible promoter to the subject and/or the one or more cells. In some embodiments, contacting one or more cells with the composition comprising a variant AAV capsid provided herein, or a therapeutically effective amount of a rAAV provided herein, or a pharmaceutical composition provided herein, is in a subject's body. In some embodiments, the one or more cells are contacted with the composition comprising a variant AAV capsid provided herein, or a therapeutically effective amount of a rAAV provided herein, or a pharmaceutical composition provided herein, in a cell culture.

The disease or disorder can be selected from the group consisting of pulmonary fibrosis, surfactant protein disorders, peroxisome biogenesis disorders, or chronic obstructive pulmonary disease (COPD). The disease or disorder can comprise a central nervous system (CNS) disorder or peripheral nervous system (PNS) disorder. The subject can be a subject suffering from or at a risk to develop one or more of chronic pain, cardiac failure, cardiac arrhythmias, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, and lysosomal storage disorders that involve cells within the CNS. The lysosomal storage disorder can be Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease.

The disease or disorder can be a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof.

The disease or disorder can comprise a neurological disease or disorder. For example, the neurological disease or disorder can comprise epilepsy, Dravet Syndrome, Lennox Gastaut Syndrome, mycolonic seizures, juvenile mycolonic epilepsy, refractory epilepsy, schizophrenia, juvenile spasms, West syndrome, infantile spasms, refractory infantile spasms, Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic clerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, or any combination thereof. The disease or disorder can be any one of the diseases or disorders shown in the tables provided herein. Diseases and disorders contemplated herein include cystic fibrosis, glycogen storage disease III, nonsyndromic deafness, dysferlinopathies, Usher 1b, Stargardt disease, Hemophilia A, Lebar congenital amaurosis, Usher 1d, Duchenne Muscular Dystrophy, and Alstrom Syndrome.

Also disclosed herein are pharmaceutical compositions comprising one or more of the compositions provided herein and one or more pharmaceutically acceptable carriers. The compositions can also comprise additional ingredients such as diluents, stabilizers, excipients, and adjuvants. As used herein, “pharmaceutically acceptable” carriers, excipients, diluents, adjuvants, or stabilizers are the ones nontoxic to the cell or subject being exposed thereto (preferably inert) at the dosages and concentrations employed or that have an acceptable level of toxicity as determined by the skilled practitioners.

The carriers, diluents and adjuvants can include buffers such as phosphate, citrate, or other organic acids: antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, di saccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

Disclosed herein include pharmaceutical compositions. In some embodiments, the pharmaceutical composition comprises: a rAAV provided herein, and a pharmaceutical excipient. The pharmaceutical composition can be for intraventricular, intraperitoneal, intraocular, intravenous, intraarterial, intranasal, intrathecal, intracistemae magna, or subcutaneous injection, and/or for direct injection to any tissue in the body. The pharmaceutical composition can comprise: a therapeutic agent. The pharmaceutical composition can comprise: (i) a targeting molecule or a nucleic acid encoding the targeting molecule and/or (ii) a donor nucleic acid or a nucleic acid encoding the donor nucleic acid. The targeting molecule can be capable of associating with the programmable nuclease. The targeting molecule can comprise single strand DNA or single strand RNA. The targeting molecule can comprise a single guide RNA (sgRNA).

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration may be changed, or an additional agent may be administered to the subject, along with the therapeutic rAAV composition. In some embodiments, as a patient is started on a regimen of a therapeutic rAAV composition, the patient is also weaned off (e.g., step-wise decrease in dose) a second treatment regimen.

In some embodiments, pharmaceutical compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect. It will be understood that the above dosing concentrations may be converted to vg or viral genomes per kg or into total viral genomes administered by one of skill in the art.

In some cases, a dose of the pharmaceutical composition may comprise a concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, 2×10¹⁷, or a range between any two of these values. In some cases the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, 3×10¹⁷, or a range between any two of these values. In some cases the concentration of the infectious particles is 4×10⁷, 4×10⁸, 4×10⁹, 4×10¹⁰, 4×10¹¹, 4×10¹², 4×10¹³, 4×10¹⁴, 4×10¹⁵, 4×10¹⁶, 4×10¹⁷, or a range between any two of these values. In some cases the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, 5×10¹⁷, or a range between any two of these values. In some cases the concentration of the infectious particles is 6×10⁷, 6×10⁸, 6×10⁹, 6×10¹¹, 6×10¹³, 6×10¹⁴, 6×10¹⁵, 6×10¹⁶, 6×10¹⁷, or a range between any two of these values. In some cases the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10¹⁶, 7×10¹⁷, or a range between any two of these values. In some cases the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, 8×10¹⁷, or a range between any two of these values. In some cases the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10¹⁶, 9×10¹⁷, or a range between any two of these values.

Disclosed herein, in some embodiments are formulations of pharmaceutically-acceptable excipients and carrier solutions suitable for delivery of the rAAV compositions described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. In some instances, the rAAV compositions are suitably formulated pharmaceutical compositions disclosed herein, to be delivered either intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

In some embodiments, the pharmaceutical forms of the AAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some cases, for administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the rAAV compositions disclosed herein, which are prepared by incorporating the rAAV compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions may be advantageous for systemic administration, for example by intravenous administration.

Also provided herein are formulations in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Pulmonary administration may be advantageously achieved via the buccal administration. In some embodiments, formulations may comprise dry particles comprising active ingredients. In such embodiments, dry particles may have a diameter in the range from about 0.5 nm to about 7 nm or from about 1 nm to about 6 nm. In some embodiments, formulations may be in the form of dry powders for administration using devices comprising dry powder reservoirs to which streams of propellant may be directed to disperse such powder. In some embodiments, self-propelling solvent/powder dispensing containers may be used. In such embodiments, active ingredients may be dissolved and/or suspended in low-boiling propellant in sealed containers. Such powders may comprise particles wherein at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form. Low boiling propellants generally include liquid propellants having a boiling point of below 65° F. at atmospheric pressure. Generally, propellants may constitute 50% to 99.9% (w/w) of the composition, and active ingredient may constitute 0.1% to 20% (w/w) of the composition. Propellants may further comprise additional ingredients such as liquid non-ionic and/or solid anionic surfactant and/or solid diluent (which may have particle sizes of the same order as particles comprising active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, comprising active ingredients, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm. Formulations described herein useful for pulmonary delivery may also be useful for intranasal delivery. In some embodiments, formulations for intranasal administration comprise a coarse powder comprising the active ingredient and having an average particle size from about 0.2 μm to 500 Such formulations are administered in the manner in which snuff is taken, e.g. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, comprise 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of AAV compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. This is made possible, at least in part, by the fact that certain target cells (e.g., neurons) do not divide, obviating the need for multiple or chronic dosing.

Alternatively, in some circumstances, it may be desirable to provide multiple, or successive administrations of the AAV vector compositions, either over a relatively short, or a relatively prolonged period of time, as may be determined by the medical practitioner overseeing the administration of such compositions. For example, the number of infectious particles administered to a mammal may be on the order of about 10⁷, 10⁸, 10⁹, 10¹⁰, 10¹¹, 10¹², 10¹³, or even higher, infectious particles/ml given either as a single dose, or divided into two or more administrations as may be required to achieve therapy of the particular disease or disorder being treated. In fact, in certain embodiments, it may be desirable to administer two or more different AAV vector compositions, either alone, or in combination with one or more other therapeutic drugs to achieve the desired effects of a particular therapy regimen. In various embodiments, the daily and unit dosages are altered depending on a number of variables including, but not limited to, the activity of the therapeutic rAAV composition used, the disease or condition to be treated, the mode of administration, the requirements of the individual subject, the severity of the disease or condition being treated, and the judgment of the practitioner.

In some embodiments, the administration of the therapeutic rAAV composition is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, 5 years, or 10 years. The effective dosage ranges may be adjusted based on subject's response to the treatment. Some routes of administration will require higher concentrations of effective amount of therapeutics than other routes.

Although not anticipated given the advantages of the present disclosure, in certain embodiments wherein the patient's condition does not improve, upon the doctor's discretion the administration of therapeutic rAAV composition is administered chronically, that is, for an extended period of time, including throughout the duration of the patient's life in order to ameliorate or otherwise control or limit the symptoms of the patient's disease or condition. In certain embodiments wherein a patient's status does improve, the dose of therapeutic rAAV composition being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the dosage amount of the therapeutic rAAV composition described herein lies within a range of circulating concentrations that include the ED50 with minimal toxicity. In certain embodiments, the daily dosage range and/or the unit dosage amount varies within this range depending upon the dosage form employed and the route of administration utilized.

### Kits

Disclosed herein include kits. In some embodiments, the kit comprises: a) one or more nucleic acids (e.g., a recombinant vector) comprising a nucleic acid encoding a variant AAV capsid provided herein; b) one or more nucleic acids (e.g., a helper vector) encoding a helper virus protein; and/or c) one or more nucleic acids (e.g., a payload vector) comprising a heterologous nucleic acid, wherein said heterologous nucleic acid comprises a polynucleotide encoding a payload.

Disclosed herein are kits comprising compositions disclosed herein. Also disclosed herein are kits for the treatment or prevention of a disease or conditions of the central nervous system (CNS), peripheral nervous system (PNS), or target organ or environment (e.g., lung, heart, liver). In some instances, the disease or condition is cancer, a pathogen infection, pulmonary disease or condition, neurological disease, muscular disease, or an immune disorder, such as those described herein. In one embodiment, a kit can include a therapeutic or prophylactic composition containing an effective amount of a composition of a rAAV particle encapsidating a heterologous nucleic acid provided herein and a recombinant AAV (rAAV) capsid protein of the present disclosure. In another embodiment, a kit can include a therapeutic or prophylactic composition containing an effective amount of cells modified by the rAAV described herein (“modified cell”), in unit dosage form that express therapeutic nucleic acid. In some embodiments, a kit comprises a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some cases, rAAV are provided together with instructions for administering the rAAV to a subject having or at risk of developing the disease or condition. Instructions can generally include information about the use of the composition for the treatment or prevention of the disease or condition.

In some cases, a kit can include allogenic cells. In some cases, a kit can include cells that can comprise a genomic modification. In some cases, a kit can comprise “off-the-shelf” cells. In some cases, a kit can include cells that can be expanded for clinical use. In some cases, a kit can contain contents for a research purpose.

In some cases, the instructions include at least one of the following: description of the therapeutic rAAV composition; dosage schedule and administration for treatment or prevention of the disease or condition disclosed herein; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. In some cases, instructions provide procedures for administering the rAAV to the subject alone. In some cases, instructions provide procedures for administering the rAAV to the subject at least about 1 hour (hr), 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 25 hrs, 26 hrs, 27 hrs, 28 hrs, 29 hrs, 30 hrs, or up to 2 days, 3 days, 4 days, 5 days, 6 days, or 7 days after or before administering an additional therapeutic agent disclosed herein. In some instances, the instructions provide that the rAAV is formulated for intravenous injection. In some instances, the instructions provide that the rAAV is formulated for intranasal administration.

### EXAMPLES

Some aspects of the embodiments discussed above are disclosed in further detail in the following examples, which are not in any way intended to limit the scope of the present disclosure.

### Materials and Methods

The following experimental materials and methods were used for Examples 1-7 described below.

Construction of Plasmids

The InFusion (Takara) assembly method was used for the construction of plasmids used in this study. The plasmid backbones were digested with commercial restriction enzymes (New England Biolabs), the inserts were synthesized (Integrated DNA Technology and Wuxi Qinglan Biotech) and/or amplified with Q5 high-fidelity DNA polymerase (New England Biolabs), and the primers were synthesized by Integrated DNA Technologies (Integrated DNA Technology). The digested backbone and inserts were purified with agarose gel purification. InFusion (Takara) assembly products were transformed into either Stb13 (Invitrogen) or NEB Stable (New England Biolabs) competent cells. Sanger sequencing was used to confirm the correct insert sequences (performed by Laragen). Plasmids, insert sequences, and construction methods are depicted in Tables 5-7.

Cell Culture and Production of Viral Particle Extracts

HEK293T cell culture and triple transfection with polyethylenimine (PEI) were conducted according to a published protocol. HEK293T cells were cultured in DMEM (ThermoFisher, cat. no. 10569044) with 5% FBS, 10 mM HEPES, and 1× non-essential amino acids. The cells were passaged to 15-cm dishes or 6-well plates at 40% confluence one day before transfection. In the case of 15-cm dishes, HEK293T producer cells (˜80% confluent) were transfected with PEI with 20 μg iCAP or REP-CAP plasmid, 10 μg pHelper plasmid (Agilent, cat. no. 240071), 5 μg CMV-AAP plasmid, and 5 μg rAAV genome plasmid. In the case of 6-well plates, the plasmid masses were linearly scaled down to 2 μg iCAP plasmid, 1 μg pHelper plasmid, 0.5 μg CMV-AAP plasmid, and 0.5 μg rAAV genome plasmid for each well. Sequences for the plasmids as well as rAAV genomes are included in Tables 5-7. Plasmids were transfected to cells with PEI at a 3.5:1 (μg PEI: μg DNA) ratio.

48-72 hr after transfection, 1/80 media volume of 0.5 M EDTA was added to each dish/well to detach the cells from culture dishes. After 15 min incubation at room temperature, the mixture of cells and media was collected and centrifuged at 2000 g for 10 min at 4° C., and the supernatant (media) is removed completely.

Producer cell pellets were immediately lysed following a generic protocol for nonenveloped viruses with some modifications. Lysis buffer (100 mM magnesium chloride, 38.1 mM citric acid, 74.8 mM sodium citrate, 75 mM sodium chloride, 0.001% Pluronic™ F-68 (Thermo Fisher, cat. 24040032); the pH of the resulting solution should be ˜5) was sterilized with a 0.22 μm PES, supplemented with 1 tablet protease inhibitor (ThermoFisher, cat. no. A32963) per 50 mL solution, and stored in 4 C. The lysis buffer was added to cell pellet samples at a ratio of 1 mL/1e7 cells. The mixture was vortexed for 20 s to fully resuspend the pellet. The resulting sample was then incubated for 5-15 min at room temperature and vortexed for 20 s again before being centrifuged at 5000-9000 g for 10 min. The supernatant was moved to a clean tube and neutralized with 1/10 volume of neutralization buffer (2 M Tris-HCl, pH 9.5). Depending on the purpose of the experiment, the samples were either further purified or used for experiments, as specified in figure legends. In case when the sample was not being used for further purification, 0.05% BSA was added to the sample for better stability.

Virus Purification (Precipitation-Based Method)

Unless otherwise specified, virus samples for TEM, DLS, and transduction assays were prepared via the following precipitation-based purification method, a modified version of a generic protocol for purifying nonenveloped viruses (Kawano et al, 2018). The viral particle extract was added with 1/100 volume of DNase I (Roche, cat. no. 4716728001) and 1/100 volume of MspI (NEB, cat. no. R0106L). The reaction was incubated at 37° C. for 1 hr. Afterward, the sample was mixed well with 1/10 volume of precipitation buffer A (5% (m/v) sodium deoxycholate and 500 mM NaCl), and the mixture was incubated at 37° C. for another 30 min. Finally, the sample was mixed with 1/20 volume of precipitation buffer B (1 M citric acid), vortexed for 10 s, and centrifuged at 5000-9000 g for 5 min at 4° C.

After centrifugation, the supernatant with capsids was filtered with a Millex-HP 0.45 μm PES filter (cat. no. SLHP033RS) and buffer exchanged for at least 5 cycles with 100 kD MWCO centrifugal concentrator (Thermo Scientific, cat. no. 88503) to the final storage buffer (PBS with 200 mM additional NaCl and 0.001% Pluronic F-68 non-ionic surfactant (Thermo Scientific, cat. no. 24040032)). In each cycle of buffer exchange, the concentrator tubes were centrifuged at 1000 g for 20 min at 4° C. The purified virus samples were stored at 4° C. for further characterization.

For TEM and DLS analyses, sample purity was further improved by 3-5 cycles of buffer exchange with a 300 kD MWCO centrifugal concentrator (Pall, Cat. OD300C33).

Virus Purification (Affinity-Based Method)

The viral particle extract was added with 1/100 volume of DNase I (Roche, cat. 4716728001) and 1/100 volume of MspI (NEB, cat. no. R0106L). The reaction was incubated at 37° C. for 1 hr and then incubated with POROS CaptureSelect AAV9 Affinity Resin (ThermoFisher Scientific, cat. A27356) at a ratio of 200 μL slurry per 1 mL extract at room temperature for 2 hr on a rotator. The resulting resins were collected by centrifugation at 500 g for 5 min. The supernatant was discarded, and the resin pellets were washed with 10× slurry volume of PBS and centrifuged again at 500 g for 5 mins. The resulting resin pellets were loaded into a micro-spin column (˜100 μL slurry in each column) and washed with 300 μL wash buffer 1 (PBS+additional 100 mM NaCl) twice and 300 μL wash buffer 2 (PBS+additional 250 mM NaCl+0.025% Tween 20) once. The bound virus particles were then incubated with 100 μL of elution buffer (2 M MgCl₂) at room temperature for 15 min before centrifuged at 500 g for 5 min. The eluted solution was collected and used for TEM imaging.

qPCR Titration of Viral Genome Copy Number Protected from DNase I Digestion

qPCR titration was performed in triplicate for each sample. As an overview, the samples were first treated with DNasel to remove unpackaged genome DNA, and then the capsid-protected genomes were released by proteinase K digestion and heat denaturation. DNasel solution was prepared fresh by diluting DNasel recombinant (RNase-free; 10 U/μL; Roche Diagnostics, cat. no. 4716728001) 200-fold in 1× DNasel buffer supplied by the manufacturer to a final concentration of 50 U/mL. Proteinase K solution was prepared fresh by dilution of Proteinase K (recombinant, PCR grade; 50 U/mL (2.5 U/mg); Roche Diagnostics, cat. no. 03115828001) 200-fold in Proteinase K buffer (1 M NaCl and 1% (wt/vol) N-lauroylsarcosine sodium salt) to a final concentration of 0.25 U/mL. 2 μL of each sample replicate was treated with 50 μL DNasel solution for 30 min at 37° C. before inactivation, which was done by adding 2.5 μL of 5 M EDTA to the sample and heating it at 70° C. for 10 min. Each reaction was then added to 60 μL of Proteinase K solution and incubated at 56° C. for 2 hr or overnight. The resulting reaction was inactivated at 95° C. for 10 min. Depending on the starting material, the treated sample was either diluted 300-fold in water or column purified (eluted in 10 μL TE buffer) before qPCR titration so that the resulting CT values can fit in the linear dynamic range of the qPCR assay. The qPCR reaction was composed of 12.5 μL of SYBR Green master mix (Roche Diagnostics, cat. no. 04913850001), 9.5 μL of water, 0.5 μL of each primer (from a 2.5-04 stock concentration), and 2 μL of the diluted or purified sample. A-100 bp amplicon in the rAAV genome was amplified for quantification. Exact amplicon locations and primer sequences used for amplification are included in Table 6B. The thermocycler parameters were: (1) 95° C. for 10 min; (2) 95° C. for 15 s; (3) 60° C. for 20 s; (4) 60° C. for 40 s; and (5) Repeat steps 2-4 for 40 cycles.

Rosetta Modeling of HI-Loop-Shortened Variants

12 AAV9 variants with shortened HI loops were designed by removing a step series of peptides from the center of the loop and refilling the gap with different numbers of flexible residues (glycine/serine). The new designs were then evaluated with RosettaRemodel with the cyclic coordinate descent (CCD) closure algorithm. In brief, a trimer model of AAV9 capsid protein (PDB ID: 3ux1) was used as the modeling template. In the modeling blueprint, 4 residues adjacent to the truncation site (2 residues on each side) and the flexible (glycine/serine) peptide were set to be movable, while the rest of the capsid protein trimer was fixed. The Rosetta design score reported for the 5 lowest-energy poses as well as the total number of accepted poses for each design calculated within 3 hours with the same computational hardware settings were recorded. The relative design scores were calculated as the absolute value of the difference between each variant and the variant with the highest score.

Qualitative Docking of Capsid Subunits into a Hypothetical Planar Hexamer of Trimers

Qualitative rigid-body docking was performed in PyMOL software (Schrodinger, Version 2.3.3) based on geometrical calculations. In brief, the entire biological assembly structure of the unmodified AAV9 capsid (PDB ID:3ux1) was loaded into PyMOL, and five trimers around a 5-fold axis were retained for further modeling. One of the trimers was re-positioned into a “plane” perpendicular to the original 5-fold axis by rotation with respect to the axis connecting the centers of its two neighboring trimers. The rotation angle to achieve this new position was determined by measuring the congruent angle between the 5-fold axes and the 3-fold axes within the crystal structure. This trimer was then replicated so that there were five additional copies at the same coordinates, and the copies were then rotated in 60° steps until they formed a planar hexamer of trimers around the original 5-fold axis (now the 6-fold axis for the new hexamer of trimers). Further structural analysis was then performed.

For the qualitative docking of the HI-loop-shortened variant, six copies of the Rosetta-modeled trimer structures of the variant were aligned to the above-mentioned hexamer of trimers formed by unmodified AAV9 subunits with the CE align algorithm within PyMOL 2.3.3.

Negative Staining Transmission Electron Microscopy (TEM) and Quantification

Ultrathin carbon EM grids (Ted Pella, cat. no. 01822-F) were glow discharged and positioned with a pair of reverse tweezers. A 3-μL droplet of capsid samples was placed onto the grids for 30 s-1 min and blotted dry with filter paper. A 3-μL droplet of 2%-3% uranyl acetate solution was then placed onto the grid to stain the sample for 30 s before being blotted dry with filter paper. The grids were imaged with an FEI Tecnai T12 120 kV electron microscope equipped with a Gatan 2k×2k CCD.

The size and shape of particles in the TEM images were segmented and quantified with a customized Python script. Briefly, the TEM images were normalized, and a mask that labels pixels corresponding to individual particles was generated using the Cellpose algorithm. Segmentation artifacts and outliers were removed by filtering out any particle with perimeter, roundness, or area that were two standard deviations away from the mean. Segmentation results were manually inspected to make sure that real particles were correctly identified. The size and shape parameters of the particles were then measured and calculated on the basis of the mask.

Dynamic Light Scattering (DLS)

Purified capsid samples were diluted to 120 μL in PBS and filtered with a 0.45 μm PES Spin-X centrifuge tube filter (Corning, cat. no. CLS8162) before being loaded into a cuvette. DLS was measured at 25° C. with a NanoStar DLS instrument (Wyatt Technology). The correlation function of scattered light intensity was measured 10 times for each sample. The hydrodynamic diameter distribution of particles was calculated by fitting the correlation function with the regularization method.

Size Exclusion Chromatography (SEC) Assay

Size exclusion chromatography was performed with a qEV2/35 nm size exclusion column (Izon Science, Product Code: SP4) according to the manufacturer's manual. The column was pre-equilibrated with 3 column volumes of PBS, and the sample (with a volume of less than 2 mL) was loaded to the frit. 14 mL of the void volume was collected before collecting 18*2-mL fractions. The column was then washed again with three column volumes of PBS and stored at 4° C. DNasel-protected genome copy number in each fraction was quantified with qPCR, and the absorption at 280 nm and 260 nm was measured with a spectrometer.

In Vitro Transduction Assay in Mammalian Cells with Cre-Dependent Reporter

For transduction assay with HEK293T cells, the cells were passaged to 6-well plates at 40% confluence in DMEM media with 5% FBS. 24 hr later, each well of cells was co-transfected with 0.8 μg EF1α-Flex-GFP plasmid and 0.2 μg CAG-H2B-tdTomato (transfection marker) using FuGENE6 transfection reagent (Promega, cat. no E2691). 18 hr later, the cells were seeded to 96-well plates at 10,000 cells/well in 200 μL DMEM media with 20% FBS, 4 mM GlutaMAX (ThermoFisher, cat. no. 35050061) and 10 mM HEPES. 4 hr after seeding, each well of cells was treated with 100 μL of viral samples carrying a Cre expression genome (purified with the precipitation-based method; stock concentration=1e9 vg/ml). 15 μg/mL ZnCl₂ was added at the same time to increase transduction efficiency. After 18 hr of incubation, the media was replaced by 200 μL of imaging media (Fluorobrite DMEM (ThermoFisher, cat. no. A1896701) with 2% FBS, 4 mM GlutaMAX, 10 mM HEPES). Media was replaced every two days. Images were taken 7 days after transduction with a LSM800 inverted confocal microscope.

For transduction assay with primary cortical neurons, the neurons were prepared from C57 mice or Ai14 at E16 and seeded to 96-well or 24-well optical plates at ˜40,000 cells/cm² in neurobasal media with B-27 supplement (ThermoFisher, cat. no. A3653401). 4 days after seeding, half of the media is replaced with fresh media. In the case of C57 neurons, each well of cell in the 96-well plate was co-transduced with 50 μL of viral samples carrying a Cre expression genome (purified with the precipitation-based method; stock concentration=6e9 vg/ml) and 1.5 μL of AAV9 carrying a CAG-Flex-GFP reporter genome (stock concentration=1e12 vg/ml) four days after seeding. 3 days after infection, cells were imaged with a LSM800 inverted confocal microscope with incubation system. 4 days after infection, half of the media was replaced with fresh media. Cells were fixed 5 days after infection and imaged again with the same LSM800 microscope. In the case of Ai14 neurons, each well of cell in the 24-well plate, with 1.5 ml media per well, was incubated with 500 μL additional DNasel-treated viral extracts (concentrated with a 100 kD concentrator to 1.5e11 vg/ml) for a 2 hr period 5 days after seeding. The media containing virus extract was removed and replaced by 500 μL fresh media. Cells were imaged 3 days after infection with a Keyence BZ-X700 microscope.

Western Blot

Viral particle extracts were separated with SDS-PAGE using 4-15% precast TGX protein gels (BioRad, cat. no. 4568086). The gel was run in Tris/Glycine/SDS buffer for 30 min at 80V, followed by 90 min at 120V. Protein bands were transferred to PVDF overnight (0.45 μm) in Tris/Glycine with 20% methanol at 30 mA for 48 hr in a cold room. The membrane with protein bands was blocked with 5% milk at room temperature for 1 hr and then incubated with 1:80 anti-VP3 (clone B1) primary antibody (ARP, cat. no. 03-61058) at 4 C overnight. The membrane was then washed with TBS-T for 5 times, incubated with 1:1000 goat anti-mouse IgG1 heavy chain (HRP) (Abcam, cat. no. ab97240) at room temperature for 1 hr and washed again with TBS-T for 5 times. Finally, the PVDF membrane was incubated with Clarity Max™ Western ECL Substrate (BioRad, cat. no. 1705062) and imaged for luminescence using a ChemiDoc gel imager.

Southern Blot

Viral particle extracts were treated with 500 U/mL DNase I (Roche, cat. no. 4716728001) and 200 U/mL DpnI (NEB, cat. no. R0176L) at 37° C. for 2 hr to remove unpackaged DNA, and the reaction was stopped by adding EDTA to a final concentration of 100 mM followed by heating at 70° C. for 10 min. The reaction was then added with 1/6 volume of 8× Laemmli buffer, 1/6 volume of 5M NaCl, and 1/20 volume of proteinase K (NEB, cat. no. P8107S) at 50° C. overnight to fully denature and digest proteins. DNA from the resulting reaction was then extracted with a round of phenol-chloroform extraction, a round of chloroform extraction. Aqueous phase after two rounds of extraction was precipitated with 1/10 volume of 3M sodium acetate, 1/500 volume of co-precipitant (Meridian, cat. no. BIO-37075), and 7/10 volume of isopropanol at 20 C for 1 hr. The resulting sample was spun down at 20,000×g, 4 C for 30 min and resuspended in TE buffer. The resulting sample is quantified with qPCR, and 3×10⁹ genome copies were used for alkaline agarose gel electrophoreses and southern blot analyses using the method described in Wu, Z., Yang, H. & Colosi, P. Effect of Genome Size on AAV Vector Packaging. Mol. Ther. 18, 80-86 (2010). A DIG-labeled probe against the GFP coding gene was used for hybridization. DIG-labeled DNA markers were used for size determination. The alkaline gel electrophoreses and southern blot analyses were performed by Celplor LLC.

TABLE 5A
CAPSID VARIANTS

| CLONING NAME | |
|---|---|
| (Variant Name) | DESCRIPTION |
| AAV9 | Wild-type variant; AAV9 CAP gene in the same backbone as |
| | AddGene ID #103002 |
| djrAAV9 [wt-(G/S)3-wt] | Tandem-homodimer variant |
| (XL.D0-AAV9) | |
| AAV9 CAP Asp657_Asn668delinsGlySer | HI-loop-shortened variant (hit 1 of 2) |
| (AAV9d3) | |
| AAV9 CAP Pro659_Lys666delinsGlySer | HI-loop-shortened variant (hit 2 of 2) |
| (AAV9d6) | |
| djrAAV9 [wt-(G/S)3-d6] | Tandem-heterodimer wt-d6 variant with (G/S)3 linker |
| (XL.D1-AAV9) | |
| djrAAV9 [d6-(G/S)3-wt] | Tandem-heterodimer d6-wt variant with (G/S)3 linker |
| djrAAV9 [d6-(G/S)3-d6] | Tandem-heterodimer d6-d6 variant with (G/S)3 linker |
| djrAAV9 [wt-(G/S)9-d6] | Tandem-heterodimer linker screening variant (hit 1 of 2), (G/S)9 |
| | linker |
| djrAAV9 [wt-(G/S)7-d6] | Tandem-heterodimer linker screening variant (hit 2 of 2), (G/S)7 |
| | linker |
| djrAAV9 [wt-(G/S)5-d6] | Tandem-heterodimer linker screening variant, (G/S)5 linker |
| djrAAV9 [wt-(G/S)3+13-d6] | Tandem-heterodimer linker screening variant, (G/S)3, with first |
| | 3 AAV9 N-terminal residues of the second subunit truncated |
| djrAAV9 [wt-(G/S)3 + 11-d6] | Tandem-heterodimer linker screening variant, (G/S)3, with first |
| | 5 AAV9 N-terminal residues of the second subunit truncated |
| djrAAV9 [wt-(G/S)3+9-d6] | Tandem-heterodimer linker screening variant, (G/S)3, with first |
| | 7 AAV9 N-terminal residues of the second subunit truncated |
| djrAAV9 [wt-(G/S)3+7-d6] | Tandem-heterodimer linker screening variant, (G/S)3, with first |
| | 9 AAV9 N-terminal residues of the second subunit truncated |
| djrAAV9 [wt-(G/S)3+5-d6] | Tandem-heterodimer linker screening variant, (G/S)3, with first |
| | 11 AAV9 N-terminal residues of the second subunit truncated |
| djrAAV9 [wt-(ENLYFQG)-d6] | Tandem-heterodimer variant (TEVp site linker) |
| (XL.D1b-AAV9) | |
| djrAAV2 [wt-(ENLYFQG)-d6] | Tandem-heterodimer variant (TEVp site linker, AAV2) |
| (XL.D1b-AAV2) | |
| djrAAV5 [wt-(ENLYFQG)-d6] | Tandem-heterodimer variant (TEVp site linker, AAV5) |
| (XL.D1b-AAV5) | |
| djrAAVDJ [wt-(ENLYFQG)-d6] | Tandem-heterodimer variant (TEVp site linker, AAVDJ) |
| (XL.D1b-AAVDJ) | |
| djrAAV9 [wt-(GGENLYFQG)-d6] | Tandem-heterodimer variant (GG + TEVp site linker) |
| (XL.D1c-AAV9) | |
| djrAAVDJ [wt-(GGENLYFQG)-d6] | Tandem-heterodimer variant (GG + TEVp site linker, AAVDJ) |
| (XL.D1c-AAVDJ) | |
| tjrAAV9 [wt-(G/S)5-(G/S)5-d6] | Tandem-heterotrimer variant ((G/S)5 linkers, AAV9) |
| (XL.T1-AAV9) | |
| qjrAAV9 [wt-(G/S)5-(G/S)5-(G/S)9-d6] | Tandem-heterotetramer variant ((G/S)5 and (G/S)9 linkers, |
| (XL.Q1-AAV9) | AAV9) |
| djrAAV9 [wt-(G)-cpVP3d6] | Tandem-heterodimer variant wt-cpVP3d6, where the second |
| | subunit is a circularly permuted VP3d6 with peptide termini at |
| | H624 and P623 (the native N- and C- termini are linked with a |
| | doublete of the flexible linker used in tdTomato) |
| djrAAV9 [wt-(T2A)-d6] | Tandem-teterodimer variant wt-(T2A)-d6, where the linker is a |
| | self-cleaving peptide |
| TBSV-69-82s-CAP | TBSV quasi-equivalent peptide graft screening variant (hit) |
| (XL.N1-AAV9) | |
| TBSV_74-82i-CAP | TBSV quasi-equivalent peptide graft screening variant |
| TBSV-74-82s-CAP | TBSV quasi-equivalent peptide graft screening variant |
| TBSV-77-82i-CAP | TBSV quasi-equivalent peptide graft screening variant |
| TBSV_69-82i-CAP | TBSV quasi-equivalent peptide graft screening variant |
| TBSV_69-83i-CAP | TBSV quasi-equivalent peptide graft screening variant |
| TBSV_50-82i-CAP | TBSV quasi-equivalent peptide graft screening variant |
| TBSV-50-82s-CAP | TBSV quasi-equivalent peptide graft screening variant |
| TBSV-69-82s-CAPd6 | TBSV quasi-equivalent peptide graft screening variant, on an |
| | AAV9d6 backbone |
| TBSV_74-82i-CAPd6 | TBSV quasi-equivalent peptide graft screening variant, on an |
| | AAV9d6 backbone |
| TBSV-74-82s-CAPd6 | TBSV quasi-equivalent peptide graft screening variant, on an |
| | AAV9d6 backbone |
| TBSV-77-82i-CAPd6 | TBSV quasi-equivalent peptide graft screening variant, on an |
| | AAV9d6 backbone |
| TBSV_69-82i-CAPd6 | TBSV quasi-equivalent peptide graft screening variant, on an |
| | AAV9d6 backbone |
| TBSV_69-83i-CAPd6 | TBSV quasi-equivalent peptide graft screening variant, on an |
| | AAV9d6 backbone |
| TBSV_50-82i-CAPd6 | TBSV quasi-equivalent peptide graft screening variant, on an |
| | AAV9d6 backbone |
| TBSV-50-82s-CAPd6 | TBSV quasi-equivalent peptide graft screening variant, on an |
| | AAV9d6 backbone |

TABLE 5B
CAPSID VARIANTS

| | | | | | INSERT |
|---|---|---|---|---|---|
| CLONING NAME | | PLASMID | COLONY | INSERT | GENERATION |
| (Variant Name) | BACKBONE | CODE | CODE | CODE | METHOD |
| AAV9 | pUCmini- | XD.AAV56 | N/A | N/A | N/A |
| | iCAP | | | | |
| djrAAV9 [wt-(G/S)3-wt] | pUCmini- | XD.AAV124 | XA.267-2 | XF.272 | IDT gblock |
| (XL.D0-AAV9) | iCAP | | | | |
| AAV9 CAP Asp657_Asn668delinsGlySer | pUCmini- | XD.AAV119 | XA.187-1 | XF.233 | IDT gblock |
| (AAV9d3) | iCAP | | | | |
| AAV9 CAP Pro659_Lys666delinsGlySer | pUCmini- | XD.AAV121 | XA.190-2 | XF.236 | IDT gblock |
| (AAV9d6) | iCAP | | | | |
| djrAAV9 [wt-(G/S)3-d6] | pUCmini- | XD.AAV125 | XA.268-1 | XF.273 | IDT gblock |
| (XL.D1-AAV9) | iCAP | | | | |
| djrAAV9 [d6-(G/S)3-wt] | pUCmini- | XD.AAV126 | XA.269-2 | XF.272 | IDT gblock |
| | iCAP | | | | |
| djrAAV9 [d6-(G/S)3-d6] | pUCmini- | XD.AAV127 | XA.270-1 | XF.273 | IDT gblock |
| | iCAP | | | | |
| djrAAV9 [wt-(G/S)9-d6] | pUCmini- | XD.AAV158 | XA.306-1 | XF.308 | IDT gblock |
| | iCAP | | | | |
| djrAAV9 [wt-(G/S)7-d6] | pUCmini- | XD.AAV159 | XA.307-3 | XF.309 | IDT gblock |
| | iCAP | | | | |
| djrAAV9 [wt-(G/S)5-d6] | pUCmini- | XD.AAV160 | XA.308-3 | XF.310 | IDT gblock |
| | iCAP | | | | |
| djrAAV9 [wt-(G/S)3 + 13-d6] | pUCmini- | XD.AAV161 | XA.309-3 | XF.311 | IDT gblock |
| | iCAP | | | | |
| djrAAV9 [wt-(G/S)3 + 11-d6] | pUCmini- | XD.AAV162 | XA.310-2 | XF.312 | IDT gblock |
| | iCAP | | | | |
| djrAAV9 [wt-(G/S)3 + 9-d6] | pUCmini- | XD.AAV163 | XA.311-3 | XF.313 | IDT gblock |
| | iCAP | | | | |
| djrAAV9 [wt-(G/S)3 + 7-d6] | pUCmini- | XD.AAV164 | XA.312-3 | XF.314 | IDT gblock |
| | iCAP | | | | |
| djrAAV9 [wt-(G/S)3 + 5-d6] | pUCmini- | XD.AAV165 | XA.313-3 | XF.315 | IDT gblock |
| | iCAP | | | | |
| djrAAV9 [wt-(ENLYFQG)-d6] | pUCmini- | XD.AAV195 | XA.343-3 | XF.349 | IDT gblock |
| (XL.D1b-AAV9) | iCAP | | | | |
| djrAAV2 [wt-(ENLYFQG)-d6] | pUC-RC2 | XD.AAV222 | XA.366-1 | XF.375 | IDT gblock |
| (XL.D1b-AAV2) | | | | | |
| djrAAV5 [wt-(ENLYFQG)-d6] | pUC-RC5 | XD.AAV223 | XA.367-2 | XF.376 | IDT gblock |
| (XL.D1b-AAV5) | | | | | |
| djrAAVDJ [wt-(ENLYFQG)-d6] | pUC-RCDJ | XD.AAV221 | XA.368-4 | XF.377 | IDT gblock |
| (XL.D1b-AAVDJ) | | | | | |
| djrAAV9 [wt-(GGENLYFQG)-d6] | pUCmini- | XD.AAV240 | N/A | N/A | IDT gblock |
| (XL.D1c-AAV9) | iCAP | | | | |
| djrAAVDJ [wt-(GGENLYFQG)-d6] | pUC-RCDJ | XD.AAV261 | N/A | N/A | IDT gblock |
| (XL.D1c-AAVDJ) | | | | | |
| tjrAAV9 [wt-(G/S)5-(G/S)5-d6] | pUCmini- | XD.AAV189 | XA.323-3 | XF.325 | Qinglan synthesis |
| (XL.T1-AAV9) | iCAP | | | | |
| qjrAAV9 [wt-(G/S)5-(G/S)5-(G/S)9-d6] | pUCmini- | XD.AAV199 | XA.348-2 | XF.352 | Qinglan synthesis |
| (XL.Q1-AAV9) | iCAP | | | | |
| djrAAV9 [wt-(G)-cpVP3d6] | pUCmini- | XD.AAV192 | XA.339-3 | XF.344 | IDT gblock |
| | iCAP | | | | |
| djrAAV9 [wt-(T2A)-d6] | pUCmini- | XD.AAV194 | XA.342-1 | XF.348 | IDT gblock |
| | iCAP | | | | |
| TBSV-69-82s-CAP | pUCmini- | XD.AAV205 | XA.361-1 | XF.367 | IDT gblock |
| (XL.N1-AAV9) | iCAP | | | | |
| TBSV_74-82i-CAP | pUCmini- | XD.AAV206 | XA.362-4 | XF.368 | IDT gblock |
| | iCAP | | | | |
| TBSV-74-82s-CAP | pUCmini- | XD.AAV207 | XA.363-1 | XF.369 | IDT gblock |
| | iCAP | | | | |
| TBSV-77-82i-CAP | pUCmini- | XD.AAV208 | XA.364-1 | XF.370 | IDT gblock |
| | iCAP | | | | |
| TBSV_69-82i-CAP | pUCmini- | XD.AAV209 | XA.369-2 | XF.378 | IDT gblock |
| | iCAP | | | | |
| TBSV_69-83i-CAP | pUCmini- | XD.AAV210 | XA.370-1 | XF.379 | IDT gblock |
| | iCAP | | | | |
| TBSV_50-82i-CAP | pUCmini- | XD.AAV211 | XA.371-3 | XF.380 | IDT gblock |
| | iCAP | | | | |
| TBSV-50-82s-CAP | pUCmini- | XD.AAV212 | XA.372-2 | XF.381 | IDT gblock |
| | iCAP | | | | |
| TBSV-69-82s-CAPd6 | pUCmini- | XD.AAV213 | XA.376-1 | XF.367 | IDT gblock |
| | iCAP | | | | |
| TBSV_74-82i-CAPd6 | pUCmini- | XD.AAV214 | XA.377-2 | XF.368 | IDT gblock |
| | iCAP | | | | |
| TBSV-74-82s-CAPd6 | pUCmini- | XD.AAV215 | XA.378-2 | XF.369 | IDT gblock |
| | iCAP | | | | |
| TBSV-77-82i-CAPd6 | pUCmini- | XD.AAV216 | XA.379-1 | XF.370 | IDT gblock |
| | iCAP | | | | |
| TBSV_69-82i-CAPd6 | pUCmini- | XD.AAV217 | XA.381-3 | XF.378 | IDT gblock |
| | iCAP | | | | |
| TBSV_69-83i-CAPd6 | pUCmini- | XD.AAV218 | XA.382-4 | XF.379 | IDT gblock |
| | iCAP | | | | |
| TBSV_50-82i-CAPd6 | pUCmini- | XD.AAV219 | XA.383-3 | XF.380 | IDT gblock |
| | iCAP | | | | |
| TBSV-50-82s-CAPd6 | pUCmini- | XD.AAV220 | XA.384-3 | XF.381 | IDT gblock |
| | iCAP | | | | |

TABLE 5C
CAPSID VARIANTS

| | | | INSERT |
|---|---|---|---|
| CLONING NAME | INFUSION CLONING | | SEQ ID |
| (Variant Name) | VECTOR BACKBONE | INSERT 1 CLONING NAME | NO: |
| djrAAV9 [wt-(G/S)3-wt] | XD.AAV56 (AfeI/PmeI, | AfeI_VP3um_wt_PmeI | 1 |
| (XL.D0-AAV9) | 9.1 kb fragment) | | |
| AAV9 CAP Asp657_Asn668delinsGlySer | XD.AAV56 (AgeI/PmeI, | d3_N656C669_GS | 55 |
| (AAV9d3) | 8.8 kb fragment) | | |
| AAV9 CAP Pro659_Lys666delinsGlySer | XD.AAV56 (AgeI/PmeI, | d6_N658C667_GS | 56 |
| (AAV9d6) | 8.8 kb fragment) | | |
| djrAAV9 [wt-(G/S)3-d6] | XD.AAV56 (AfeI/PmeI, | AfeI_VP3um_d6_PmeI | 2 |
| (XL.D1-AAV9) | 9.1 kb fragment) | | |
| djrAAV9 [d6-(G/S)3-wt] | XD.AAV121 (AfeI/PmeI, | AfeI_VP3um_wt_PmeI | 57 |
| | 9.1 kb fragment) | | |
| djrAAV9 [d6-(G/S)3-d6] | XD.AAV121 (AfeI/PmeI, | AfeI_VP3um_d6_PmeI | 58 |
| | 9.1 kb fragment) | | |
| djrAAV9 [wt-(G/S)9-d6] | XD.AAV56 (AfeI/PmeI, | AfeI_VP3um_d6_9plus15_PmeI | 59 |
| | 9.1 kb fragment) | | |
| djrAAV9 [wt-(G/S)7-d6] | XD.AAV56 (AfeI/PmeI, | AfeI_VP3um_d6_7plus15_PmeI | 60 |
| | 9.1 kb fragment) | | |
| djrAAV9 [wt-(G/S)5-d6] | XD.AAV56 (AfeI/PmeI, | AfeI_VP3um_d6_5plus15_PmeI | 61 |
| | 9.1 kb fragment) | | |
| djrAAV9 [wt-(G/S)3 + 13-d6] | XD.AAV56 (AfeI/PmeI, | AfeI_VP3um_d6_3plus13_PmeI | 62 |
| | 9.1 kb fragment) | | |
| djrAAV9 [wt-(G/S)3 + 11-d6] | XD.AAV56 (AfeI/PmeI, | AfeI_VP3um_d6_3plus11_PmeI | 63 |
| | 9.1 kb fragment) | | |
| djrAAV9 [wt-(G/S)3 + 9-d6] | XD.AAV56 (AfeI/PmeI, | AfeI_VP3um_d6_3plus9_PmeI | 64 |
| | 9.1 kb fragment) | | |
| djrAAV9 [wt-(G/S)3 + 7-d6] | XD.AAV56 (AfeI/PmeI, | AfeI_VP3um_d6_3plus7_PmeI | 65 |
| | 9.1 kb fragment) | | |
| djrAAV9 [wt-(G/S)3 + 5-d6] | XD.AAV56 (AfeI/PmeI, | AfeI_VP3um_d6_3plus5_PmeI | 66 |
| | 9.1 kb fragment) | | |
| djrAAV9 [wt-(ENLYFQG)-d6] | XD.AAV56 (AfeI/PmeI, | AfeI_VP3-TEV-d6_PmeI | 3 |
| (XL.D1b-AAV9) | 9.1 kb fragment) | | |
| djrAAV2 [wt-(ENLYFQG)-d6] | Rep-Cap AAV2 (Cell | XcmI_AAV2_TEV-d6_PmeI | 4 |
| (XL.D1b-AAV2) | Biolabs cat. VPK-422) | | |
| | (XcmI/PmeI, 6.7 kb | | |
| | fragment) | | |
| djrAAV5 [wt-(ENLYFQG)-d6] | Rep-Cap AAV5 (Cell | PflMI_AAV5_TEV-d6_AgeI | 5 |
| (XL.D1b-AAV5) | Biolabs cat. VPK-425) | | |
| | (PflMI/AgeI, 7.1 kb | | |
| | fragment) | | |
| djrAAVDJ [wt-(ENLYFQG)-d6] | Rep-Cap AAVDJ (Cell | AfeI_AAVDJ_TEV-d6_AgeI | 6 |
| (XL.D1b-AAVDJ) | Biolabs cat. VPK-420-DJ) | | |
| | (AfeI/AgeI, 7.1 kb | | |
| | fragment) | | |
| djrAAV9 [wt-(GGENLYFQG)-d6] | XD.AAV56 (AfeI/PmeI, | AfeI_VP3-GG-TEV-d6_PmeI | 7 |
| (XL.D1c-AAV9) | 9.1 kb fragment) | | |
| djrAAVDJ [wt-(GGENLYFQG)-d6] | Rep-Cap AAVDJ (Cell | AfeI_AAVDJ_GG-TEV-d6_AgeI | 8 |
| (XL.D1c-AAVDJ) | Biolabs cat. VPK-420-DJ) | | |
| | (AfeI/AgeI, 7.1 kb | | |
| | fragment) | | |
| tjrAAV9 [wt-(G/S)5-(G/S)5-d6] | XD.AAV56 (AfeI/PmeI, | AfeI_tjrAAV9-d6x2_PmeI | 9 |
| (XL.T1-AAV9) | 9.1 kb fragment) | | |
| qjrAAV9 [wt-(G/S)5-(G/S)5-(G/S)9-d6] | XD.AAV56 (AfeI/PmeI, | AfeI_qjrAAV9-d6x3_PmeI | 10 |
| (XL.Q1-AAV9) | 9.1 kb fragment) | | |
| djrAAV9 [wt-(G)-cpVP3d6] | XD.AAV56 (AfeI/PmeI, | AfeI_G-cpVP3d6(H624- | 67 |
| | 9.1 kb fragment) | P623)_PmeI | |
| djrAAV9 [wt-(T2A)-d6] | XD.AAV56 (AfeI/PmeI, | AfeI_wt-(T2A)-d6_PmeI | 68 |
| | 9.1 kb fragment) | | |
| TBSV-69-82s-CAP | XD.AAV123 (Bsu36I/BsiWI, | Bsu36I_TBSV-68-82s_BsiWI | 12 |
| (XL.N1-AAV9) | 8.8 kb fragment) | | |
| TBSV_74-82i-CAP | XD.AAV123 (Bsu36I/BsiWI, | Bsu36I_TBSV_74-82i_BsiWI | 71 |
| | 8.8 kb fragment) | | |
| TBSV-74-82s-CAP | XD.AAV123 (Bsu36I/BsiWI, | Bsu36I_TBSV-74-82s_BsiWI | 72 |
| | 8.8 kb fragment) | | |
| TBSV-77-82i-CAP | XD.AAV123 (Bsu36I/BsiWI, | Bsu36I_TBSV-77-82i_BsiWI | 73 |
| | 8.8 kb fragment) | | |
| TBSV_69-82i-CAP | XD.AAV123 (Bsu36I/BsiWI, | Bsu36I_TBSV_68-82i_BsiWI | 74 |
| | 8.8 kb fragment) | | |
| TBSV_69-83i-CAP | XD.AAV123 (Bsu36I/BsiWI, | Bsu36I_TBSV_68-83i_BsiWI | 75 |
| | 8.8 kb fragment) | | |
| TBSV_50-82i-CAP | XD.AAV123 (Bsu36I/BsiWI, | Bsu36I_TBSV_50-82i_BsiWI | 76 |
| | 8.8 kb fragment) | | |
| TBSV-50-82s-CAP | XD.AAV123 (Bsu36I/BsiWI, | Bsu36I_TBSV-50-82s_BsiWI | 77 |
| | 8.8 kb fragment) | | |
| TBSV-69-82s-CAPd6 | XD.AAV121 (Bsu36I/BsiWI, | Bsu36I_TBSV-68-82s_BsiWI | 78 |
| | 8.8 kb fragment) | | |
| TBSV_74-82i-CAPd6 | XD.AAV121 (Bsu36I/BsiWI, | Bsu36I_TBSV_74-82i_BsiWI | 79 |
| | 8.8 kb fragment) | | |
| TBSV-74-82s-CAPd6 | XD.AAV121 (Bsu36I/BsiWI, | Bsu36I_TBSV-74-82s_BsiWI | 80 |
| | 8.8 kb fragment) | | |
| TBSV-77-82i-CAPd6 | XD.AAV121 (Bsu36I/BsiWI, | Bsu36I_TBSV-77-82i_BsiWI | 81 |
| | 8.8 kb fragment) | | |
| TBSV_69-82i-CAPd6 | XD.AAV121 (Bsu36I/BsiWI, | Bsu36I_TBSV_68-82i_BsiWI | 82 |
| | 8.8 kb fragment) | | |
| TBSV_69-83i-CAPd6 | XD.AAV121 (Bsu36I/BsiWI, | Bsu36I_TBSV_68-83i_BsiWI | 83 |
| | 8.8 kb fragment) | | |
| TBSV_50-82i-CAPd6 | XD.AAV121 (Bsu36I/BsiWI, | Bsu36I_TBSV_50-82i_BsiWI | 84 |
| | 8.8 kb fragment) | | |
| TBSV-50-82s-CAPd6 | XD.AAV121 (Bsu36I/BsiWI, | Bsu36I_TBSV-50-82s_BsiWI | 85 |
| | 8.8 kb fragment) | | |

TABLE 5D
N-TERMINAL CAPSID VARIANTS

| CLONING NAME | |
|---|---|
| (Variant Name) | DESCRIPTION |
| TBSV_2-82i-VP3 | TBSV N-terminal domain graft |
| | screening variant (hit) |
| TBSV_2-76i-VP3 | TBSV N- terminal domain graft |
| (XL.N0-AAV9) | screening variant |
| TBSV_2-101i-VP3 | TBSV N- terminal domain graft |
| | screening variant |

TABLE 5E
N-TERMINAL CAPSID VARIANTS

| | | | | | INSERT |
|---|---|---|---|---|---|
| CLONING NAME | | PLASMID | COLONY | INSERT | GENERATION |
| (Variant Name) | BACKBONE | CODE | CODE | CODE | METHOD |
| TBSV_2-82i-VP3 | pMV | XD.AAV138 | XA.290-6 | XF.291 | IDT gblock |
| TBSV_2-76i-VP3 | pMV | XD.AAV141 | XA.293-2 | XF.292 | IDT gblock |
| (XL.N0-AAV9) | | | | | |
| TBSV_2-101i-VP3 | pMV | XD.AAV144 | XA.296-3 | XF.293 | IDT gblock |

TABLE 5F
N-TERMINAL CAPSID VARIANTS

| CLONING NAME | INFUSION CLONING VECTOR | | INSERT SEQ |
|---|---|---|---|
| (Variant Name) | BACKBONE | INSERT 1 CLONING NAME | ID NO: |
| TBSV_2-82i-VP3 | pMV backbone (NotI/PmeI, 3kb) | NotI_TBSV_cherry_M1G76 | 69 |
| TBSV_2-76i-VP3 | pMV backbone (NotI/PmeI, 3kb) | NotI_TBSV_cherry_M1P82 | 11 |
| (XL.N0-AAV9) | | | |
| TBSV_2-101i-VP3 | pMV backbone (NotI/PmeI, 3kb) | NotI_TBSV_cherry_M1S101 | 70 |

TABLE 5G
N-TERMINAL CAPSID VARIANTS

| | | | | INSERT 2 |
|---|---|---|---|---|
| | | | INSERT 2 | PCR PRIMER |
| CLONING NAME | INSERT 2 | INSERT 2 | SEOUENCE OR | SEQ ID |
| (Variant Name) | CODE | CLONING NAME | TEMPLATE | NO: |
| TBSV_2-82i-VP3 | XF.294 | V221-VP3(AAV9)- | XD.AAV56 | 110, 111 |
| | | PmeI | | |
| TBSV_2-76i-VP3 | XF.294 | V221-VP3(AAV9)- | XD.AAV56 | 110, 111 |
| (XL.N0-AAV9) | | PmeI | | |
| TBSV_2-101i-VP3 | XF.297 | G236-VP3(AAV9)- | XD.AAV56 | 112, 111 |
| | | PmeI | | |

TABLE 6A
rAAV GENOMES

| | | | | | VECTOR | INSERT |
|---|---|---|---|---|---|---|
| | | | | PLASMID | SEO ID | SEO ID |
| NAME | DESIGN | LENGTH | | CODE | NO: | NO: |
| genome 1 | pAAV2-CAG-GFP | 3.4 | kb | XD.AAV16 | 25 | 15 |
| genome 2 | pAAV2-CMV-mCh-CAG-GFP | 4.9 | kb | XD.AAV109 | 26 | 16 |
| genome 3 | pAAV2-CAG-GFP-stuffer1- | 7.0 | kb | XD.AAV113 | 27 | 17 |
| | CMV-mCh | | | | | |
| genome 4 | pAAV2-CMV-SpCas9-GFP | 6.9 | kb | XD.AAV154 | 28 | 18 |
| genome 5 | pAAV2-Ple261-GFP | 5.2 | kb | N/A | 29 | 19 |
| genome 6 | pAAV2-EF1a-Cre | 3.8 | kb | XD.AAV246 | 30 | 20 |
| genome 7 | pAAV2-EF1a-S-Cre | 8.2 | kb | XD.AAV249 | 31 | 21 |
| genome 8 | pAAV2-CAG-GFP-stuffer2- | 6 | kb | XD.AAV110 | 32 | 22 |
| | CMV-mCh | | | | | |
| genome 9 | pAAV2-CAG-GFP-stuffer3- | 7.5 | kb | XD.AAV169 | 33 | 23 |
| | CMV-mCh | | | | | |
| genome 10 | pAAV2-CAG-GFP-stuffer4- | 8.5 | kb | XD.AAV167 | 34 | 24 |
| | CMV-mCh | | | | | |

TABLE 6B
rAAV GENOMES AND QPCR Primers

| | | Primer Sea | |
|---|---|---|---|
| Name | Amplicon Length | Id Nos: | Source |
| genome 1 | 100 bp sequence within GFP | 35, 36 | Addgene #37825 |
| genome 2 | 100 bp sequence within GFP | 37, 38 | Subcloning of a synthesized insert into a |
| | | | pAAV2-CAG-GFP (Genome #1) backbone |
| genome 3 | 100 bp sequence within GFP | 39, 40 | Subcloning of a synthesized insert into a |
| | | | pAAV2-CAG-GFP (Genome #1) backbone |
| genome 4 | 100 bp sequence within GFP | 41, 42 | Cloning of PX458 (Addgene #48138) into a |
| | | | pAAV backbone |
| genome 5 | 100 bp sequence within GFP | 43, 44 | Subcloning a Ple261 promoter into a |
| | | | pAAV2-CAG-GFP (Genome #1) backbone |
| genome 6 | 120 bp sequence within WPRE | 45, 46 | Addgene #55636 |
| genome 7 | 120 bp sequence within WPRE | 47, 48 | Subcloning of S protein coding gene |
| | | | (Addgene #141347) into apAAV2-EFla- |
| | | | Cre (Genome #6) backbone |
| genome 8 | 100 bp sequence within GFP | 49, 50 | Subcloning of a synthesized insert into a |
| | | | pAAV2-CAG-GFP (Genome #1) backbone |
| genome 9 | 100 bp sequence within GFP | 51, 52 | Subcloning of a synthesized insert into a |
| | | | pAAV2-CAG-GFP (Genome #1) backbone |
| genome 10 | 100 bp sequence within GFP | 53, 54 | Subcloning of a synthesized insert into a |
| | | | pAAV2-CAG-GFP (Genome #1) backbone |

TABLE 7
ACCESSORY AAP PLASMIDS

| PLASMID | PLASMID | | SEQ ID | |
|---|---|---|---|---|
| CODE | NAME | DESCRIPTION | NO: | SOURCE |
| XD.AAV40 | CMV- | Mammalian expression of | 87 | Subcloning of a |
| | AAP9cm | a codon-modified AAP9 in | | synthesized insert |
| | | HEK cells, driven by a | | into a pMV backbone |
| | | CMV promoter | | |
| XD.AAV54 | REP- | Mammalian expression of | 88 | Subcloning of a synthesized |
| | AAP9cm- | a codon-modified AAP9 | | insert into a |
| | FLAG | and Rep proteins in HEK | | REP-CAP AAV9 |
| | | cells, driven by promoters | | backbone |
| | | within REP gene | | |

### Example 1

### Tandem Dimerization Design Concept

This example provides validation for the tandem dimer strategy provided herein for design of capsid nanoparticles of expanded size.

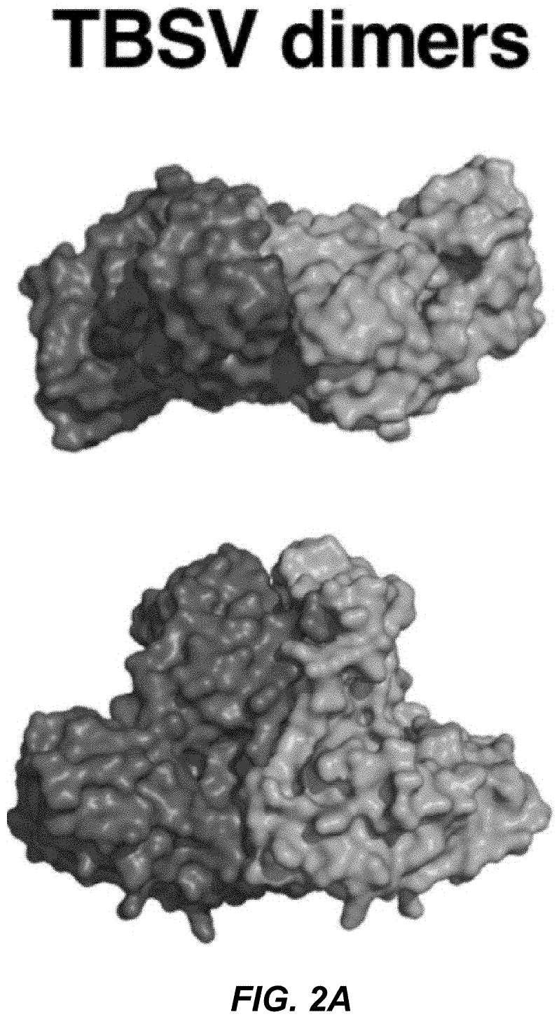

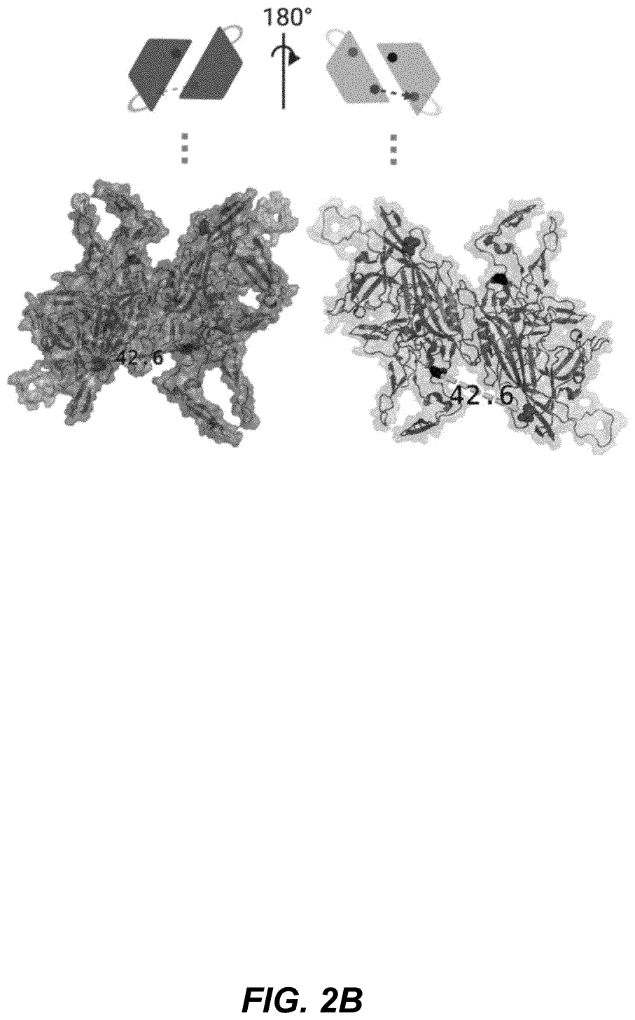

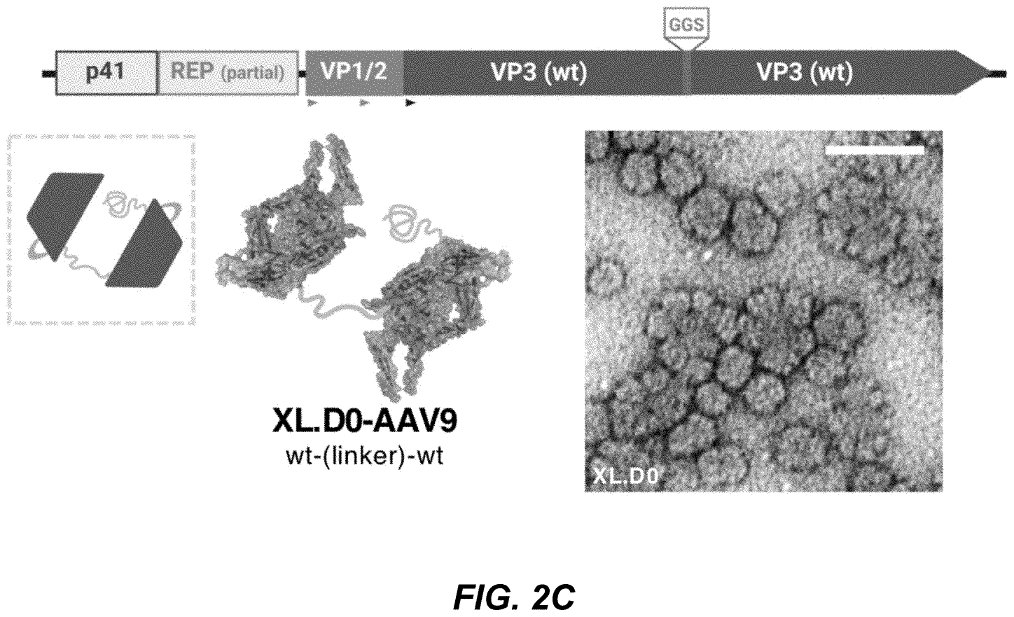

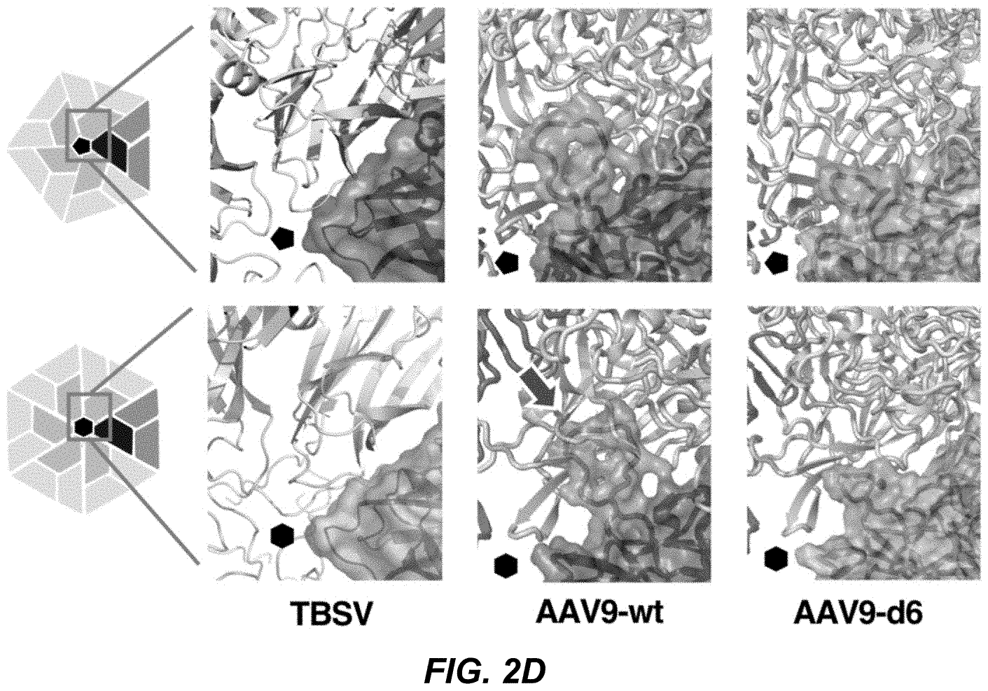

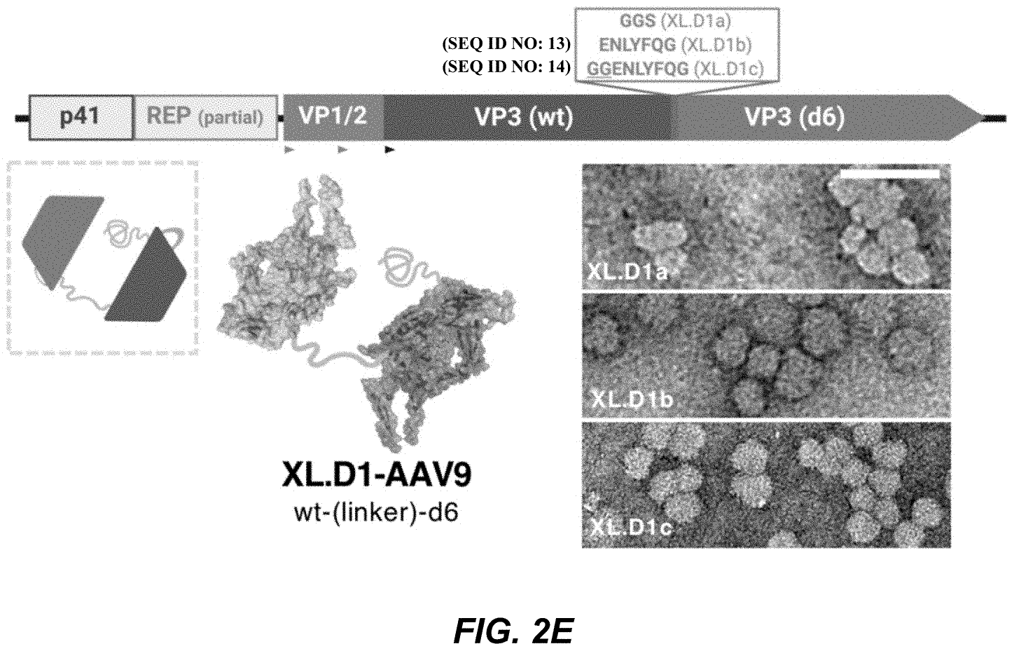

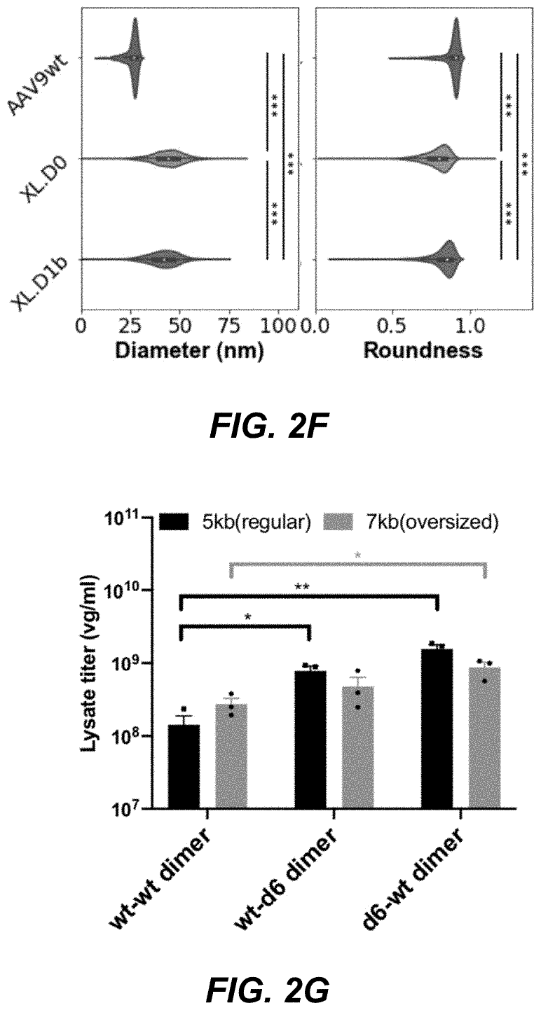

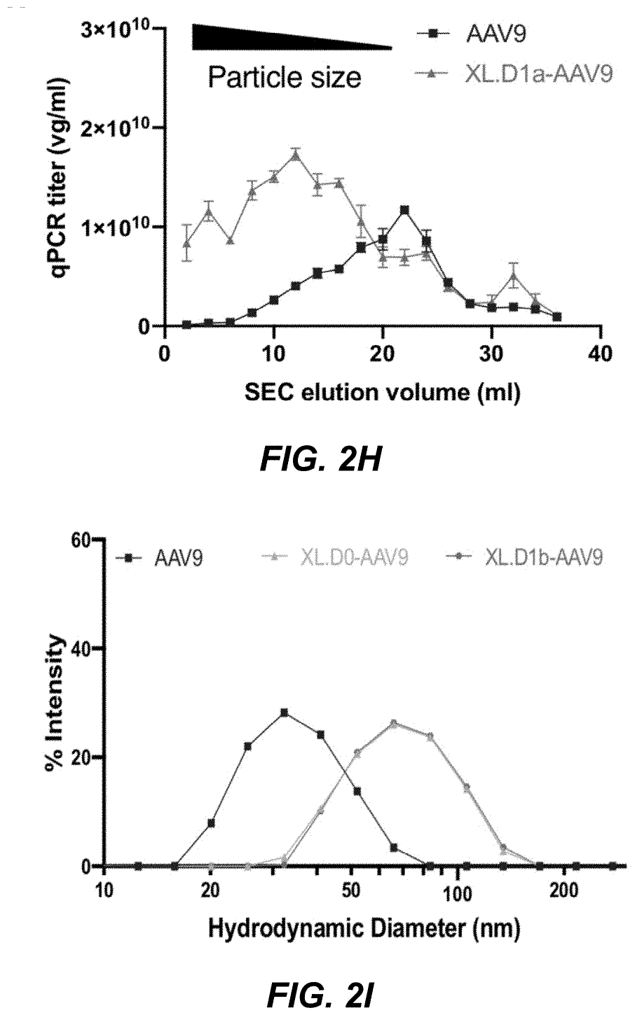

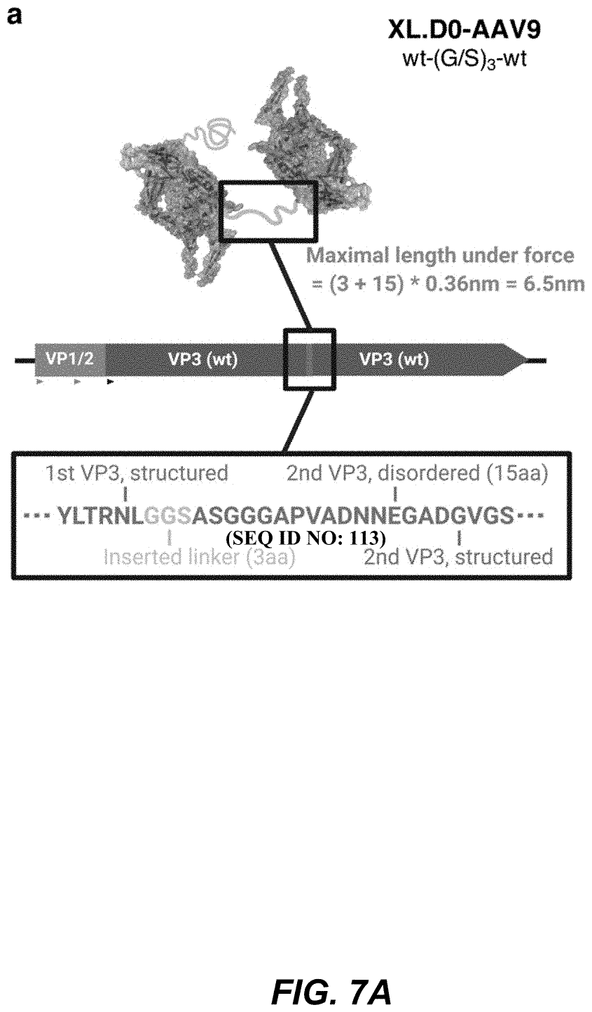

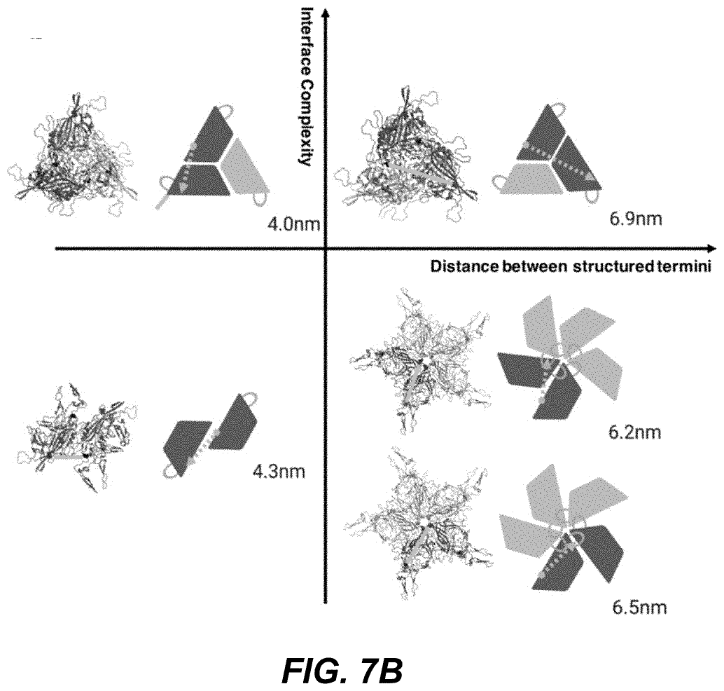

Tandem multimerization aims to strengthen pre-existing inter-subunit interactions at the rigid interfaces, particularly the dimeric interaction, to promote assembly pathways similar to those shown in FIG. 1C. Without being bound by any particular theory, covalently linking two AAV capsid subunits may restrict the subunits' mobility, which may enhance the apparent association rate and decrease the apparent dissociation rate of the dimeric interaction. Such intramolecular sub-assembly is permitted by the short distance between the termini of the two neighboring subunits (4.3 nm) that form dimers in a natural AAV capsid (FIG. 2B, FIGS. 7A-7B). Although the linker length also permits a counter-clockwise 3-fold interaction (C-terminus-to-N-terminus distance=4 nm), the structurally complicated 3-fold interaction is presumably kinetically less favored when competing with the much simpler 2-fold interaction (FIG. 7B). Other types of intramolecular symmetrical interaction are less probable due to long C-terminus-to-N-terminus distances (FIG. 7B).

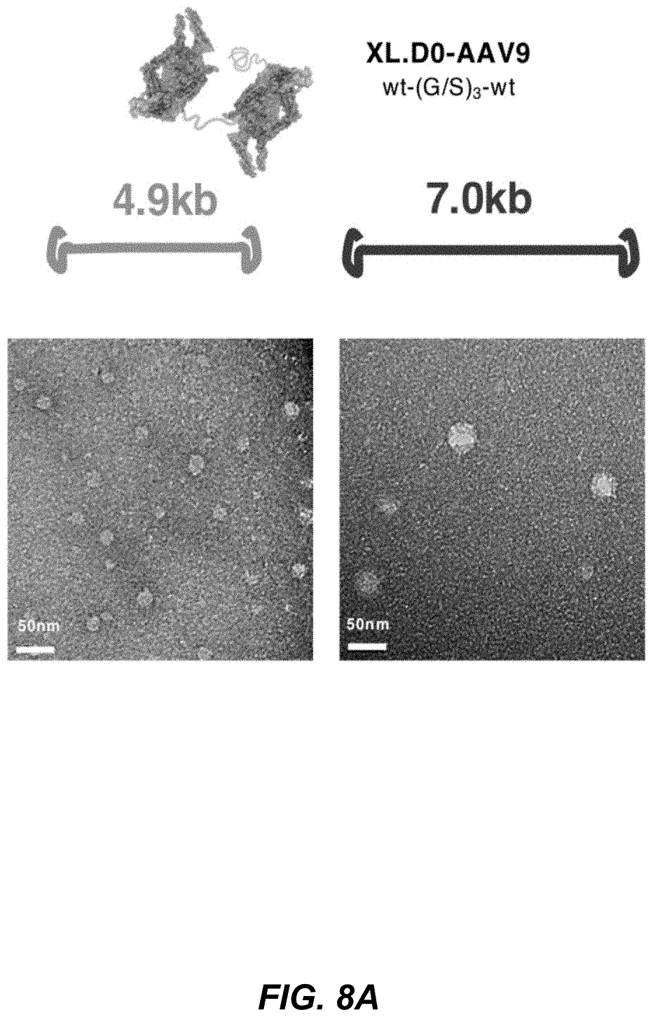

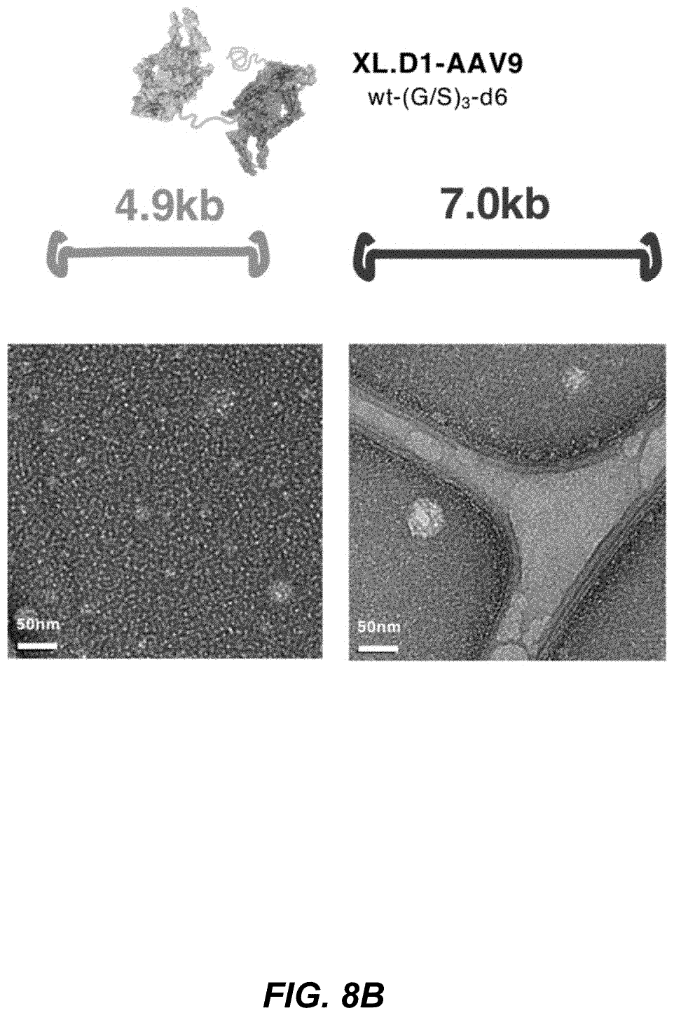

A genetically fused tandem dimer was created comprising two copies of the AAV9 capsid subunit with a three-residue flexible linker between the C-terminus and the unstructured N-terminus of the two subunits. The tandem-dimer variant formed not only 25-nm particles but also 35-nm to 60-nm particles, as found in the cell lysate samples purified with either an affinity-based method (FIG. 8A) or a precipitation-based method (FIG. 2C). This tandem-homodimer (wt-wt) design was named XL.D0-AAV9, where “XL” denotes the eXtraLarge size of the capsids and “D” denotes the dimeric design. As a side note, the viral nucleotide sequence used to encode the first monomeric subunit retains the same alternative splicing signals and alternative translation start sites as in wild-type AAV9 sequence (FIG. 2C, black triangular arrows denote the start codons), which are responsible for producing three structurally indifferentiable protein isoforms (VP1, VP2, and VP3).

### Example 2

### Computational Screening for Energetically Favorable HI Loops

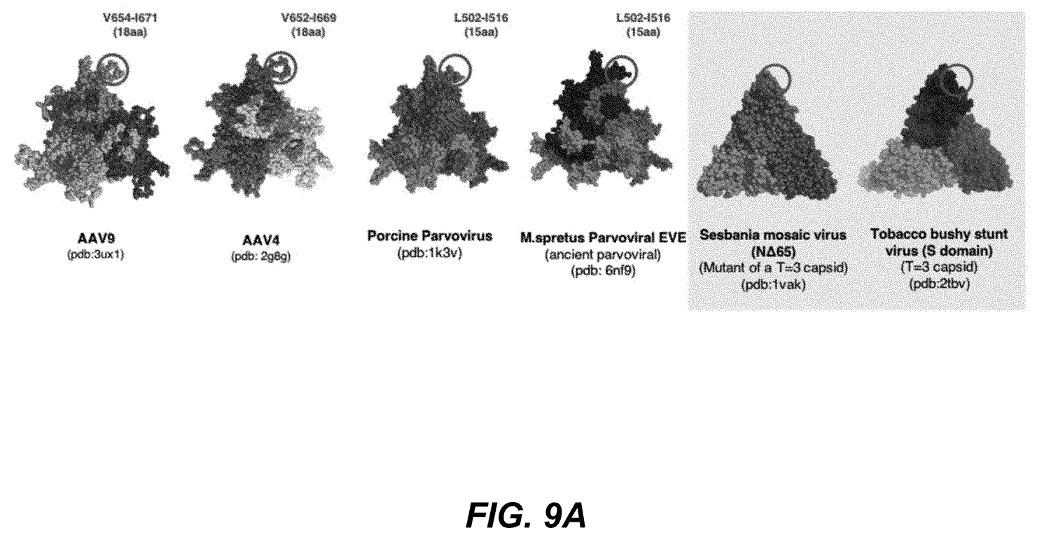

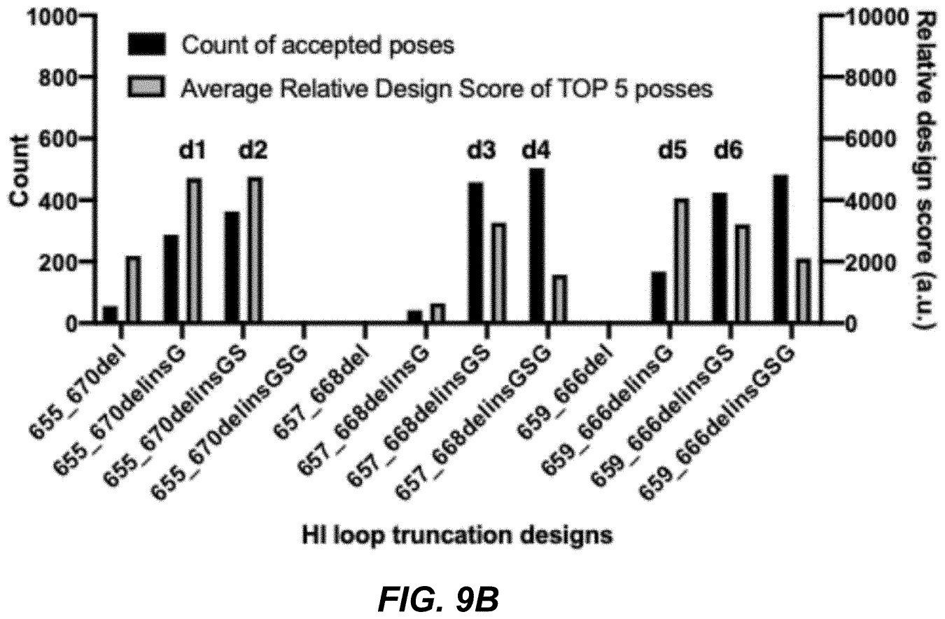

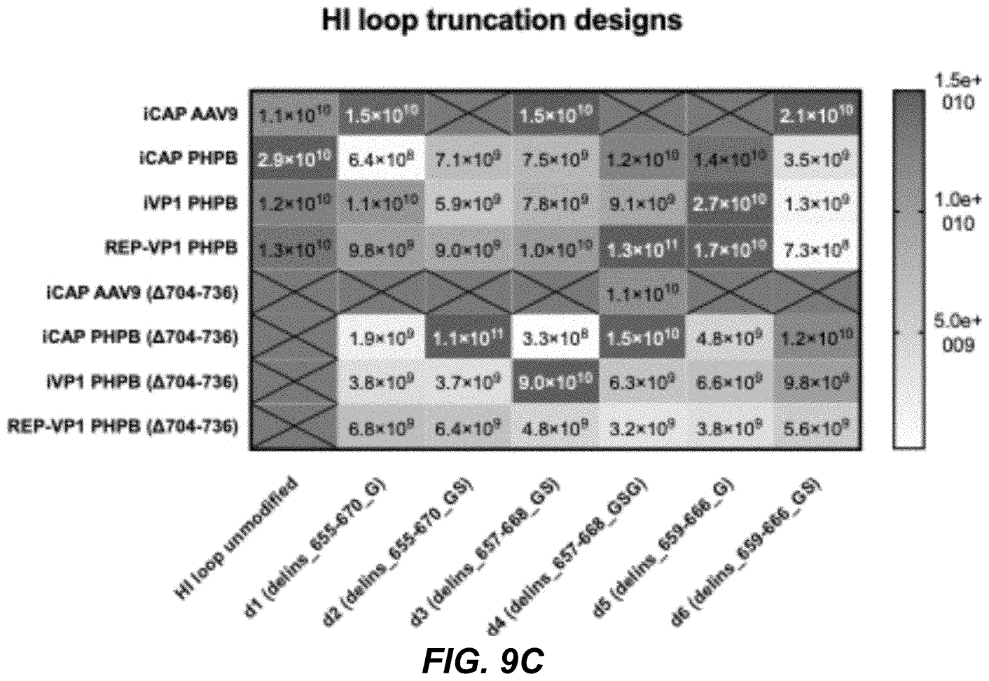

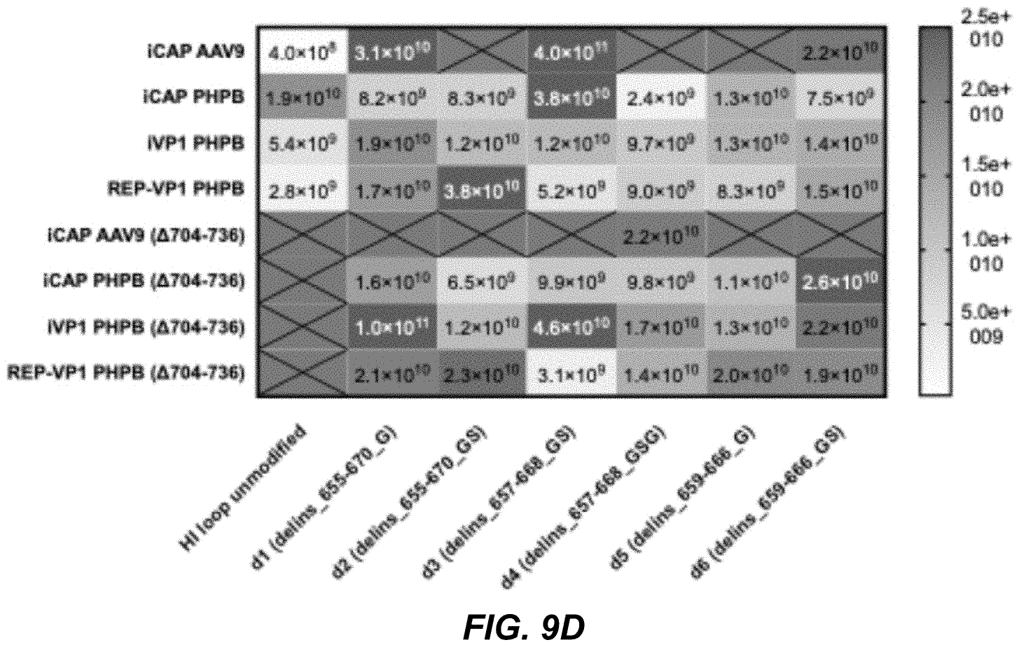

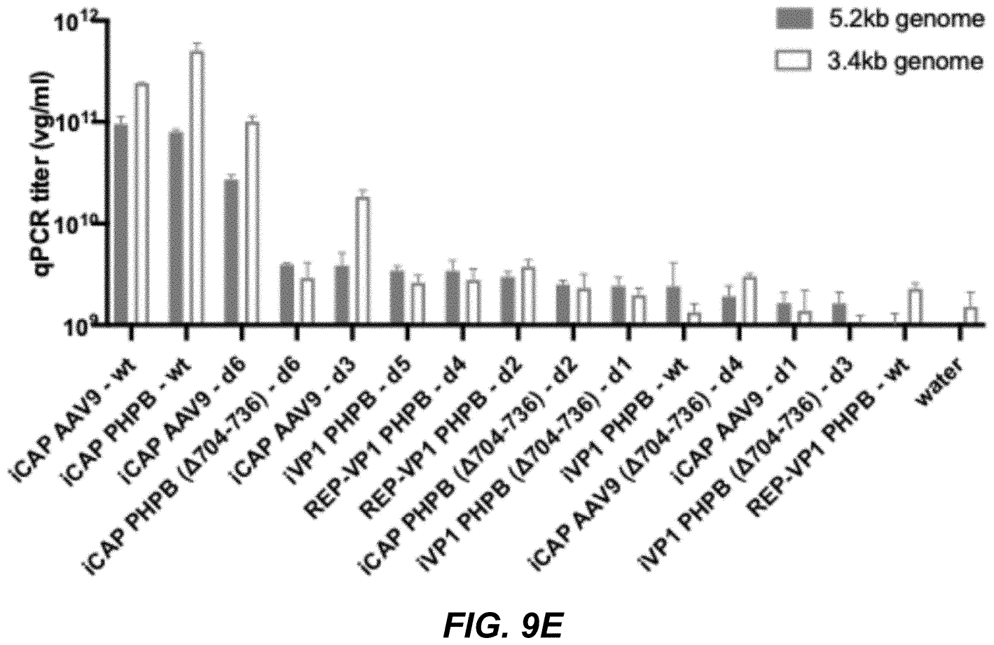

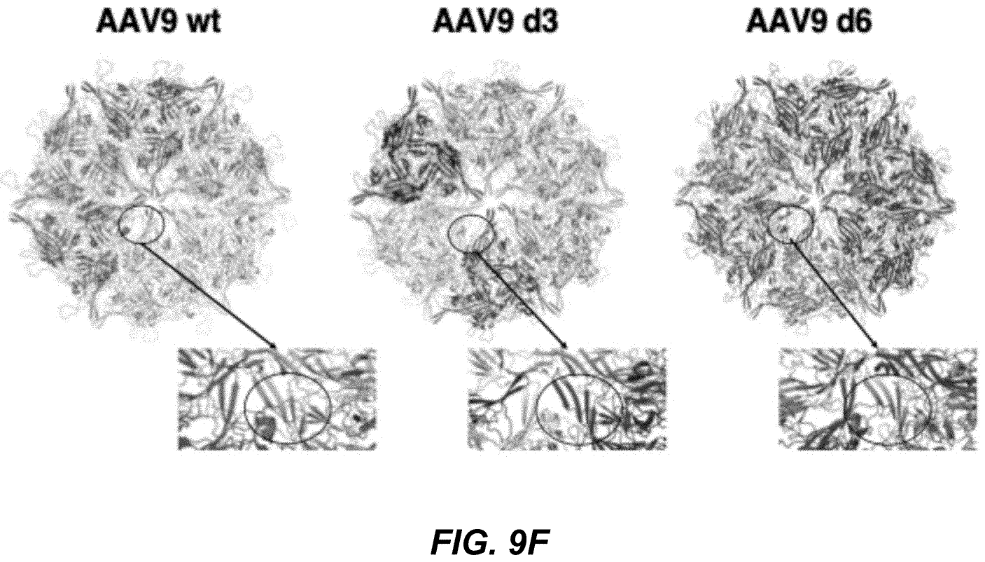

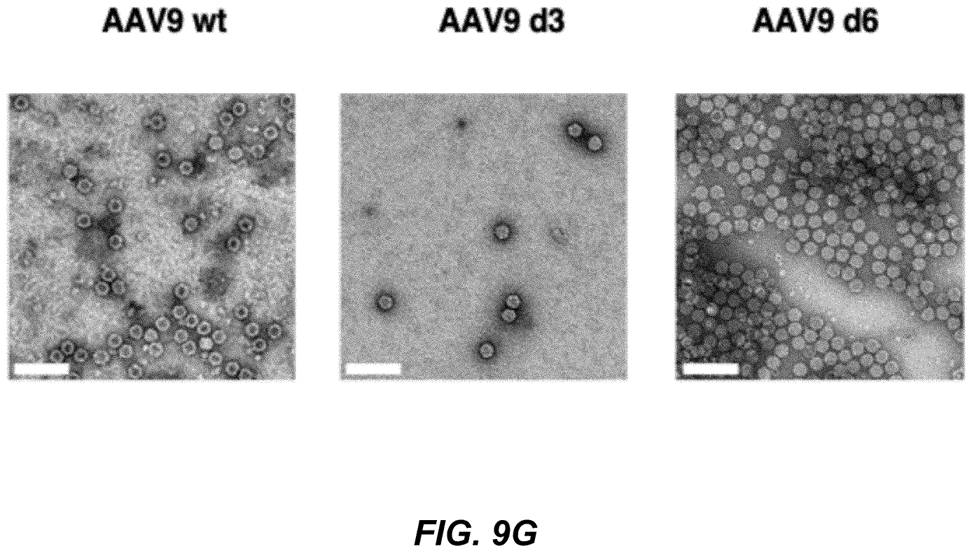

Wild-type AAV capsid structure suggests several features that might obstruct the formation of canonical T≥3 capsids, even after tandem dimerization. For example, the wild-type AAV capsid possesses interlaced inter-subunit interactions around the 5-fold axis, whereas most native T=3 capsids with similar protein folds have relatively simple interfaces (FIG. 9A). In particular, the HI loop of each AAV subunit is 20% longer than those of other parvoviral capsids and appears to be important for the stability and function of the 5-fold pore. However, this loop may sterically hinder the formation of a potential quasi-equivalent 6-fold interface—an additional type of symmetrical interaction that is present in completed T≥3 capsids but not T=1 capsids (FIG. 1A)—by clashing with the jelly-roll beta-strands of its counter-clockwise neighbor (FIG. 2D). Without being bound by any particular theory, this spatial conflict likely causes an energy penalty against the completion of T≥3 icosahedral-like assemblies in XL.D0-AAV9 in the final steps of assembly. In an attempt to reduce the energy penalty against 6-fold interactions, a computationally aided screen of monomeric subunit mutants with shortened HI loops was conducted (FIGS. 9B-9E). A screen for efficient capsid production and genome packaging by TEM and titering yielded truncated monomeric subunit variants that retain their ability to assemble into wild-type-like, 25-nm capsids (FIGS. 9F-9G). AAV9-d6, a variant that replaces HI loop residues P659-K666 by two residues (GS), produced the highest titer DNasel-protected titer (FIG. 9E). Qualitative docking indicated that the AAV9-d6 subunit can be adapted into a planar hexamer without apparent steric hindrance (FIG. 2D).

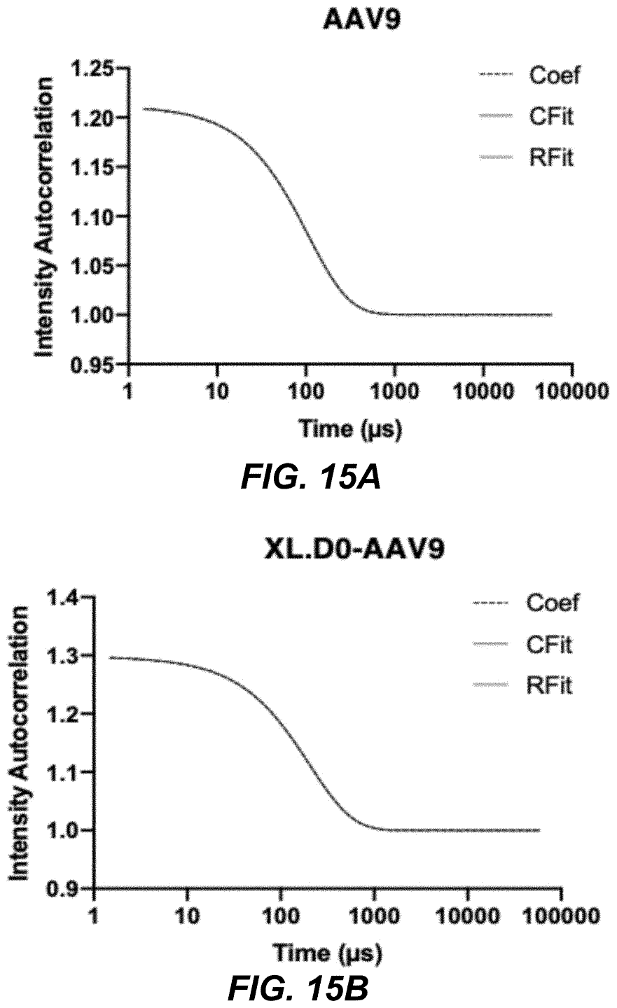

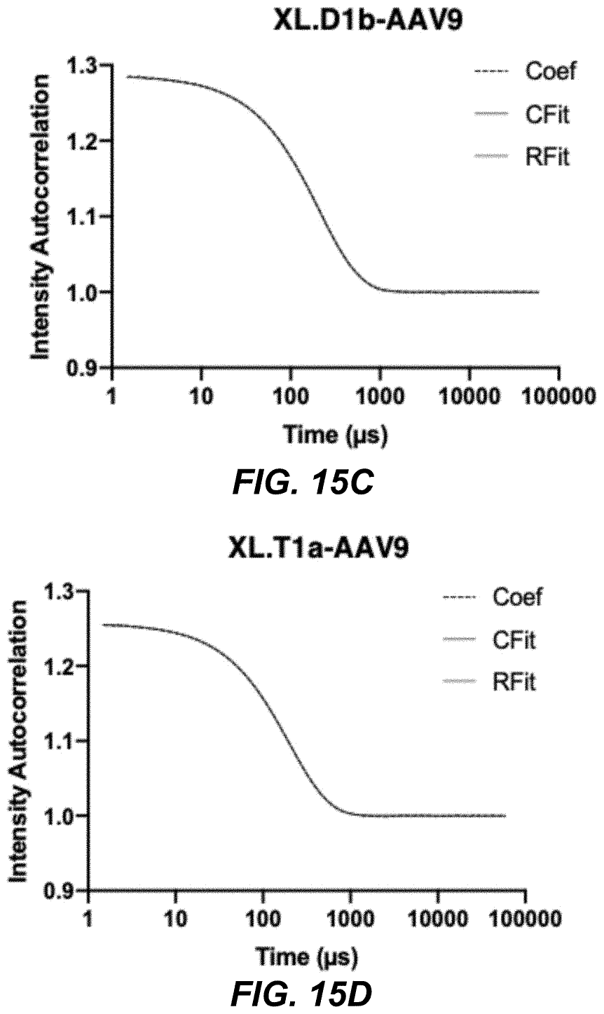

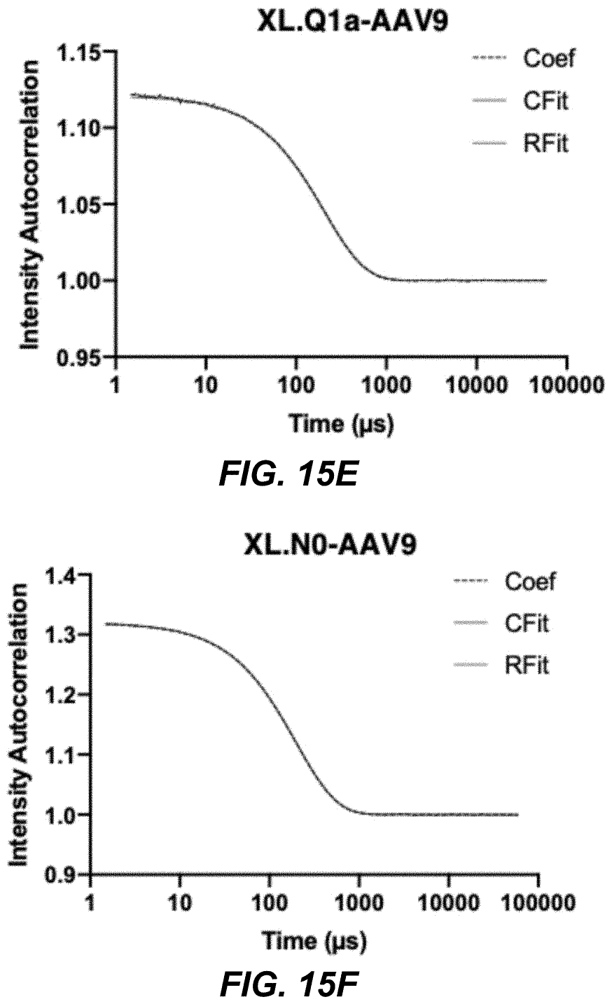

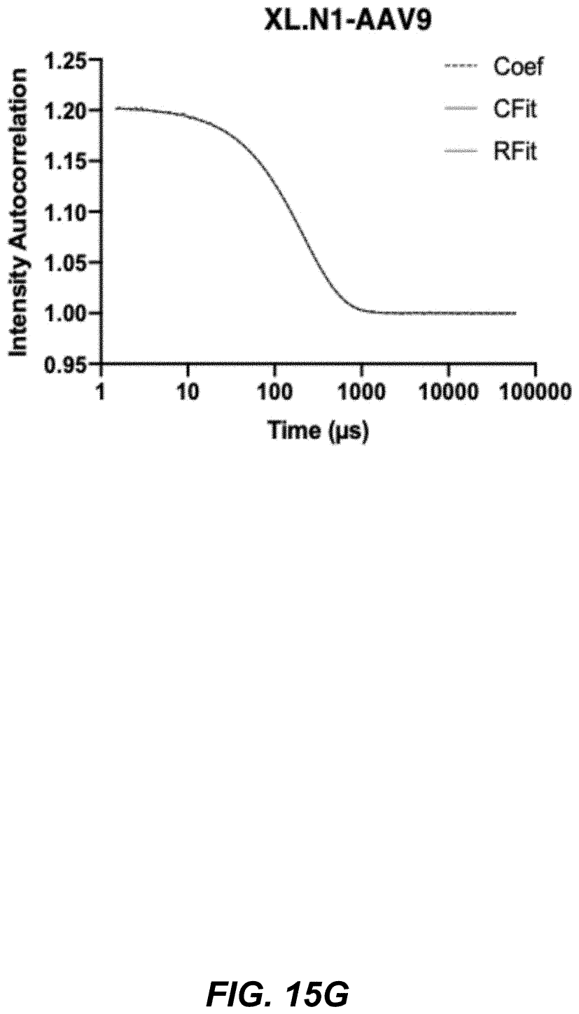

The AAV9-d6 subunit was next incorporated into the tandem-dimeric design employed in XL.D0-AAV9. Both heterodimeric designs (dimers of wt-d6 and d6-wt) formed genome-protecting capsid structures and produced higher titer of DNasel-protected genomes (FIG. 2G) compared to homodimers. The higher yield of d6-containing dimers is consistent with the expected stabilization effects of AAV9-d6 subunits. The wt-d6 tandem dimer was characterized by transmission electron microscopy (TEM) (FIGS. 2E-2F, FIG. 8B, FIG. 10D), size-exclusion chromatography (SEC) (FIG. 2H), and dynamic light scattering (DLS) (FIG. 2I, FIG. 15). These results confirm that the capsids formed by these heterodimeric structural units indeed had expanded sizes (ranging between 35 nm and 60 nm) and carried rAAV genomes (FIGS. 2G-21I). The wt-d6 tandem dimer was termed XL.D1a-AAV9. Notably, the expanded capsids formed by both XL.D0-AAV9 and XL.D1a-AAV9 can be purified with an anti-native-AAV9 antigen-binding fragment (FIG. 8), indicating that the structure of the expanded particles may retain at least one surface epitope present on wild-type AAV9 capsids.

### Example 3

### Screen for Linkers to Increase Capsid Homogeneity and Production Efficiency

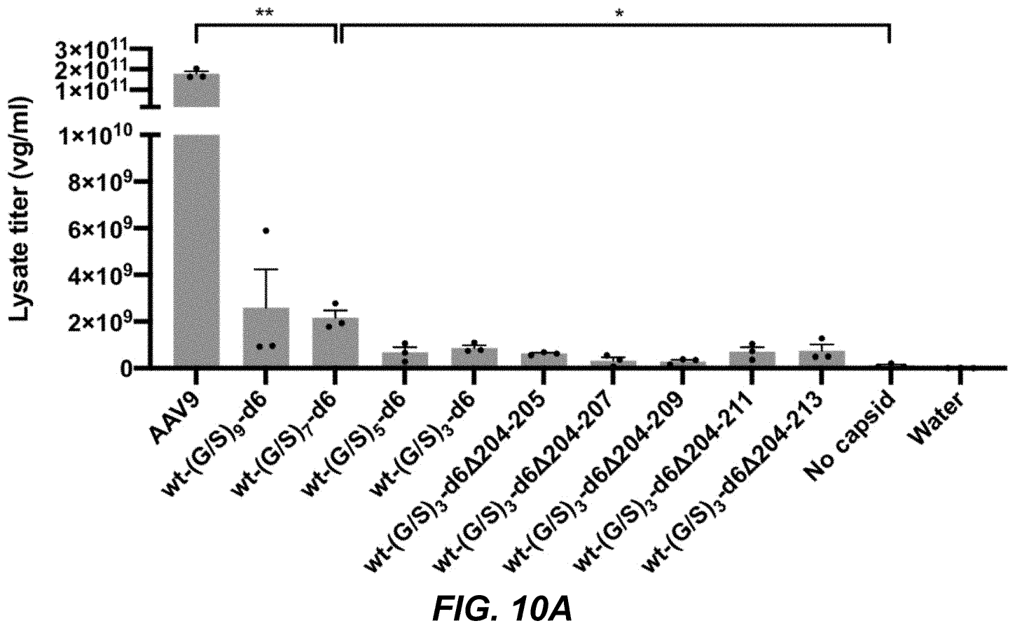

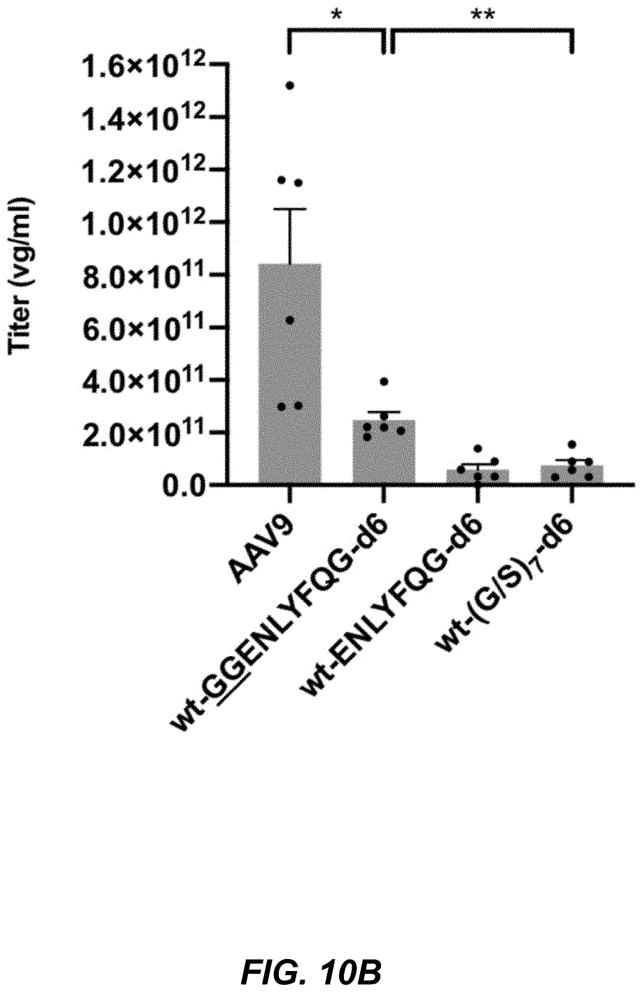

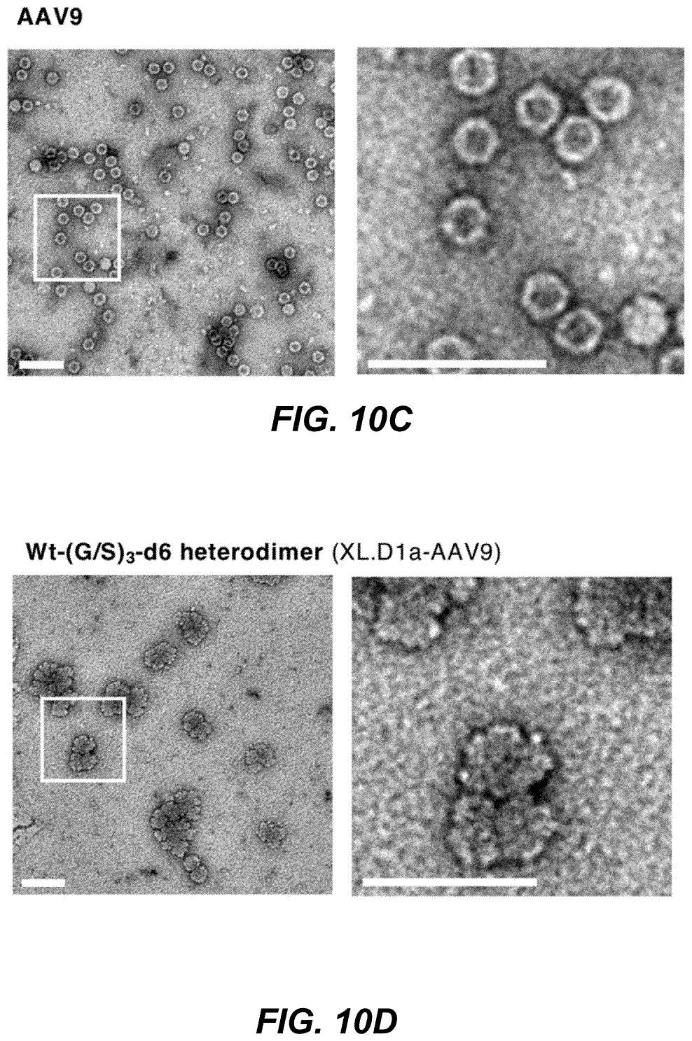

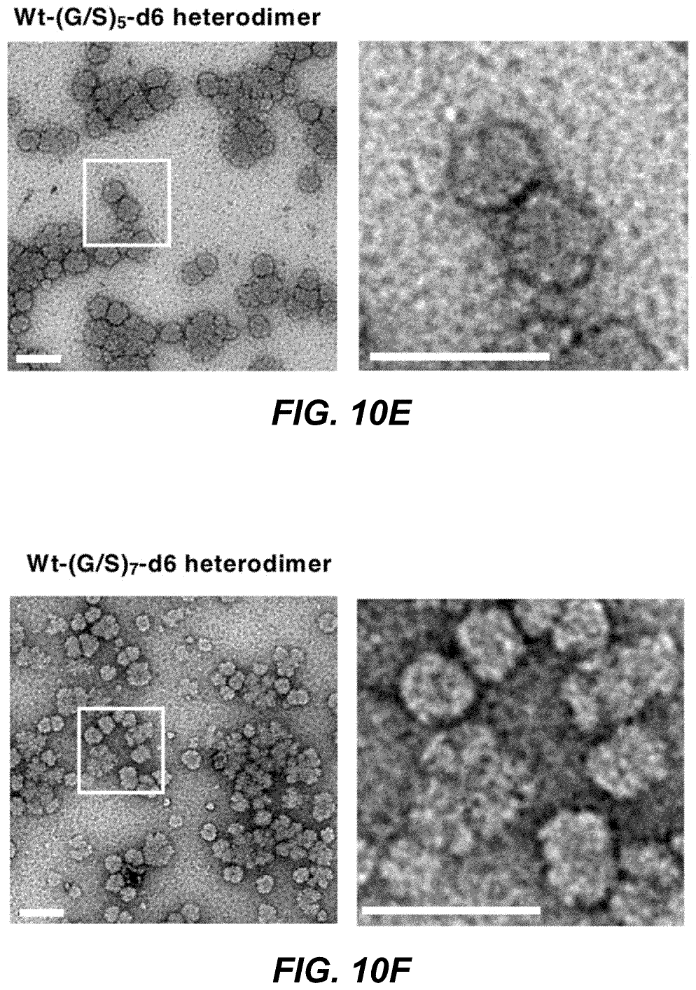

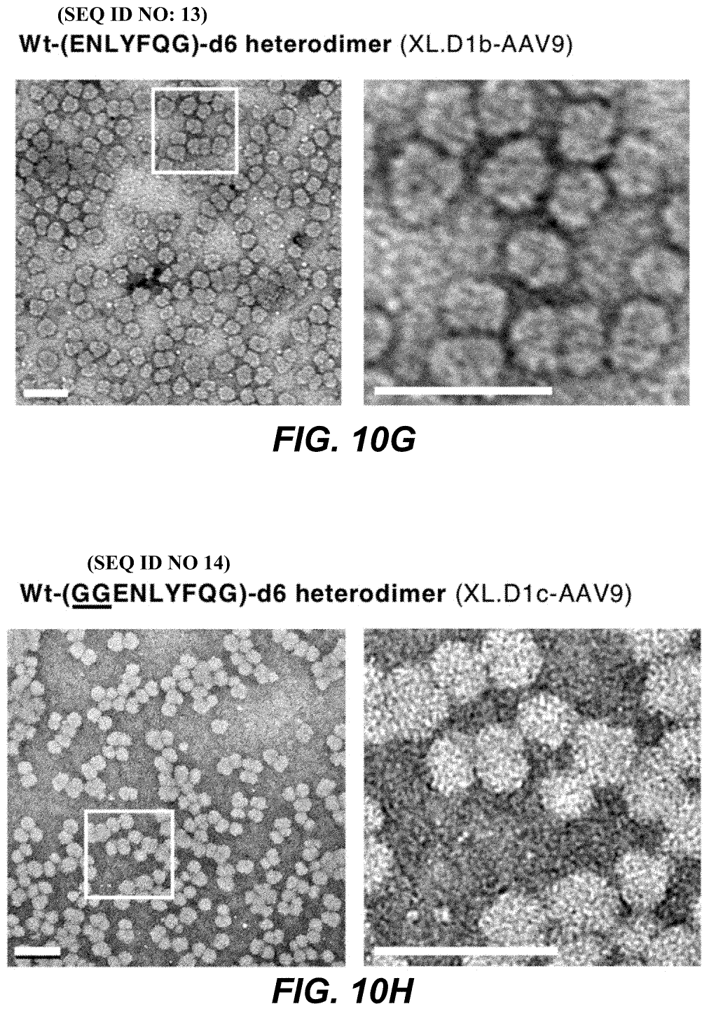

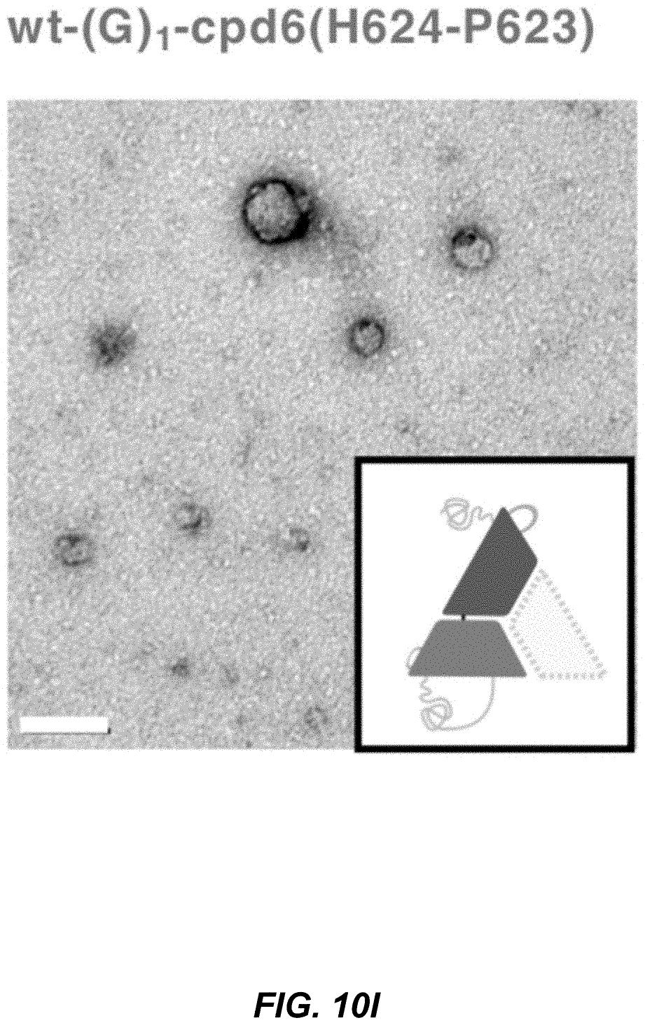

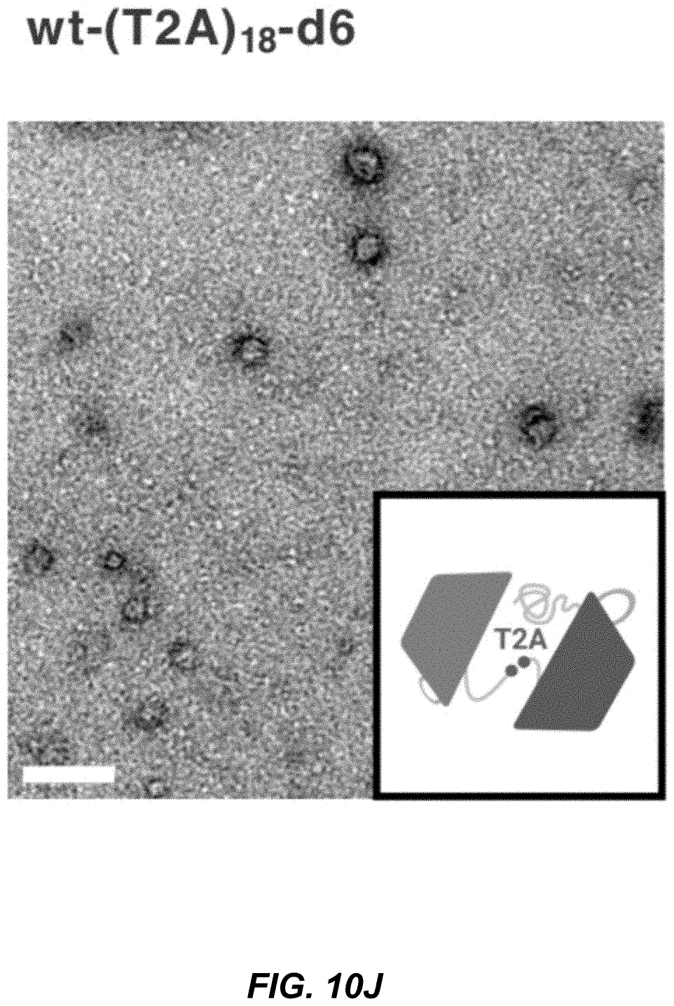

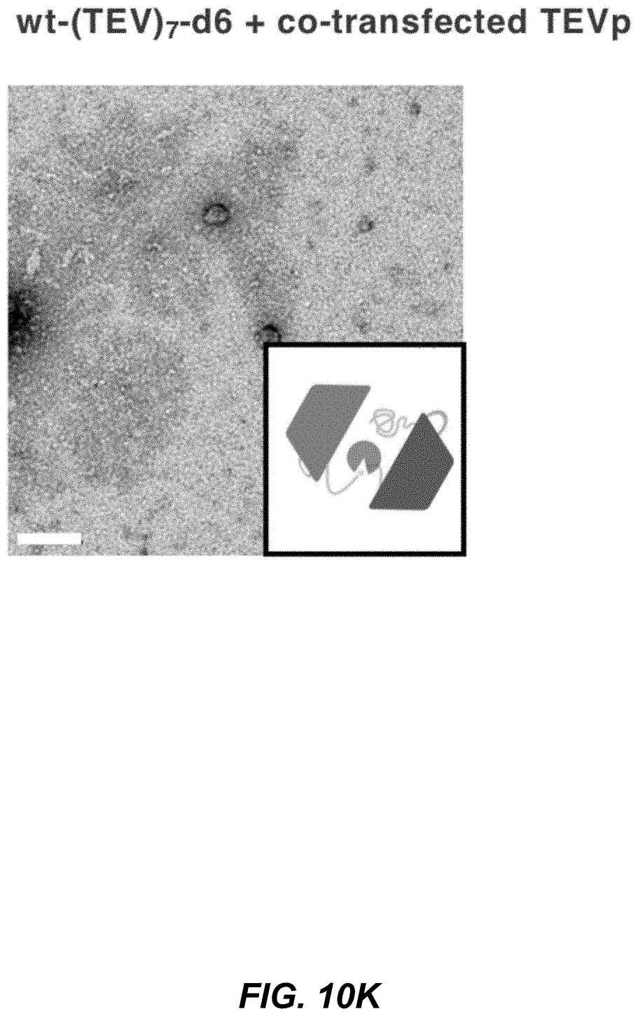

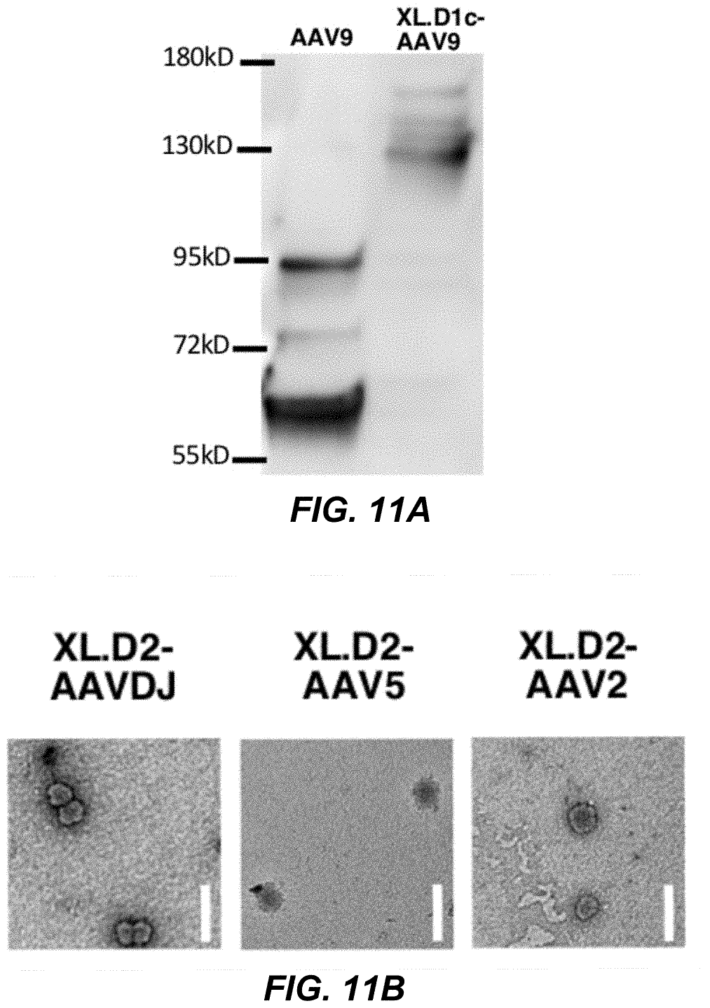

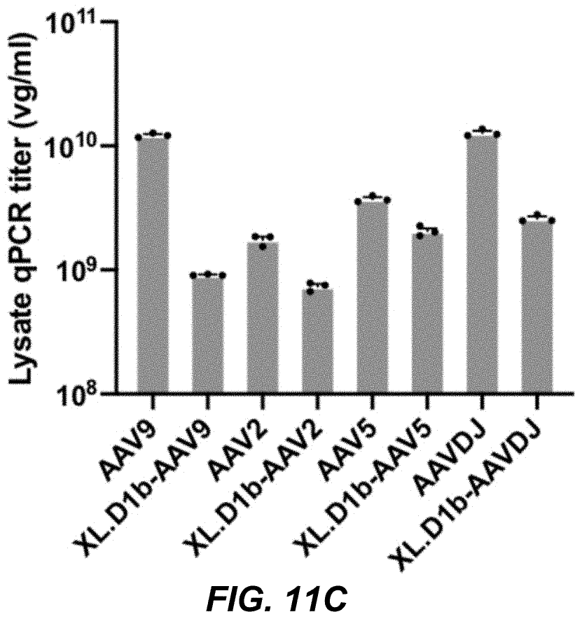

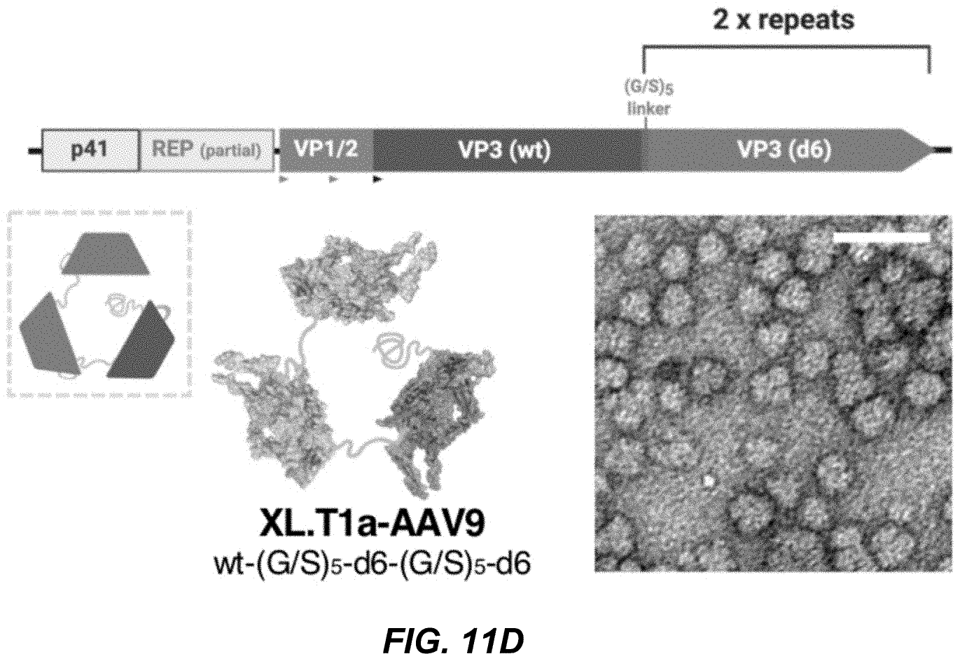

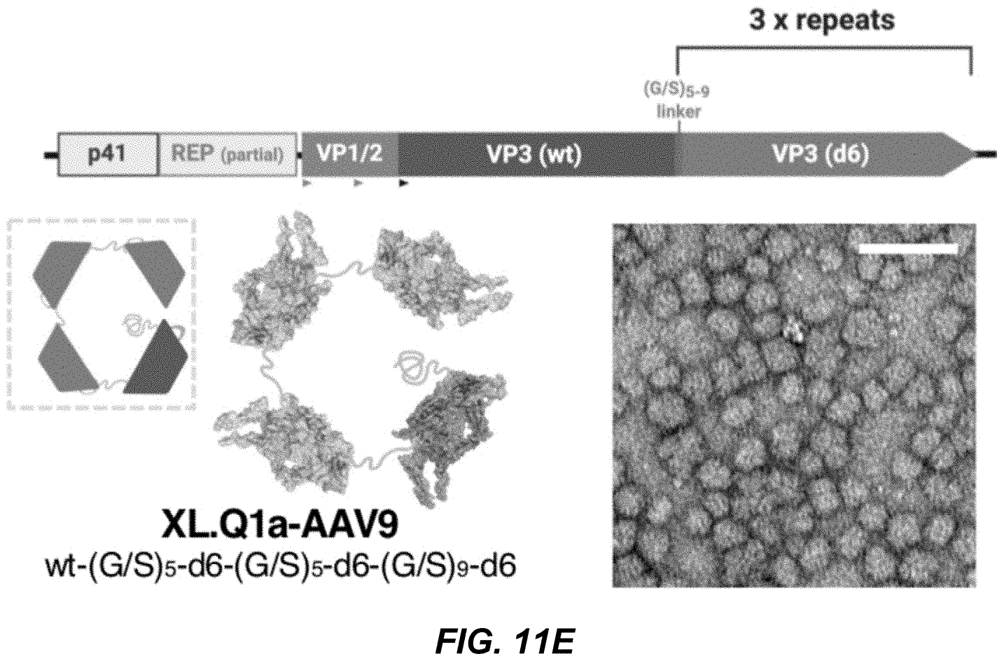

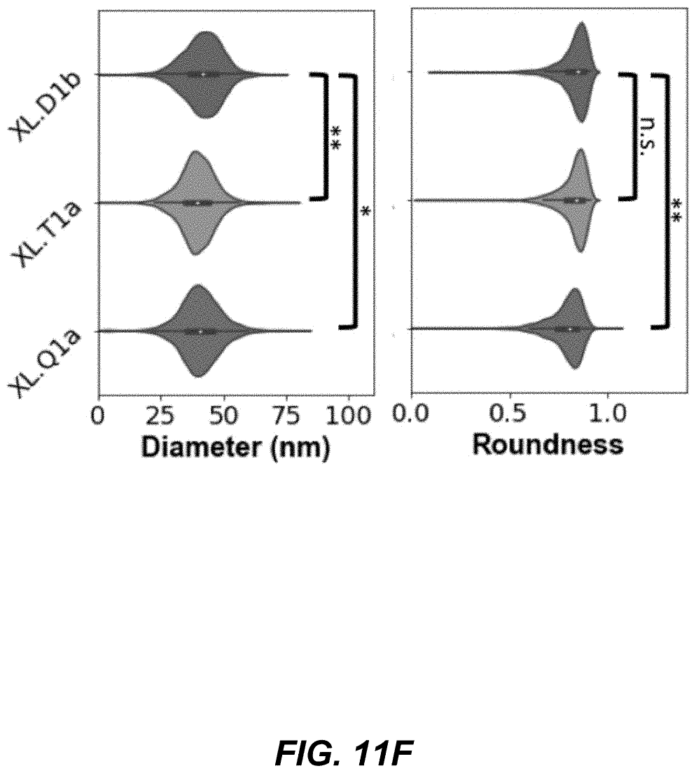

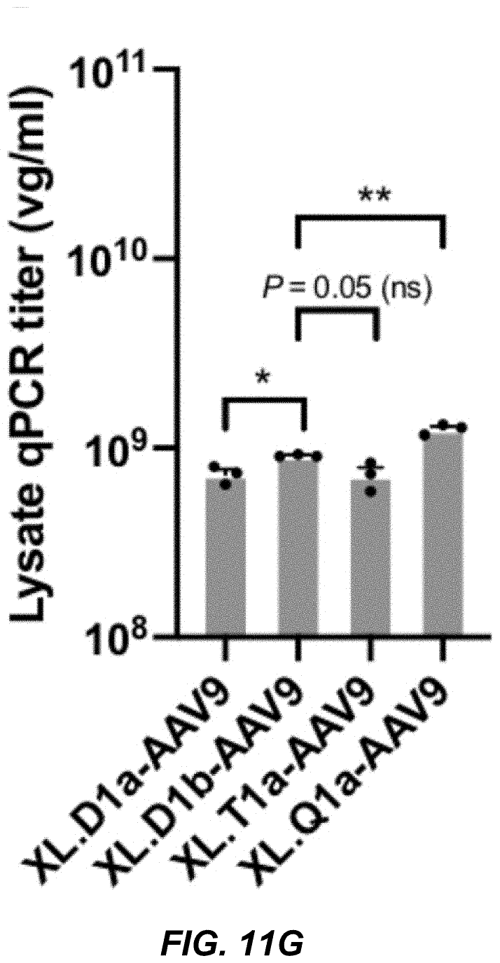

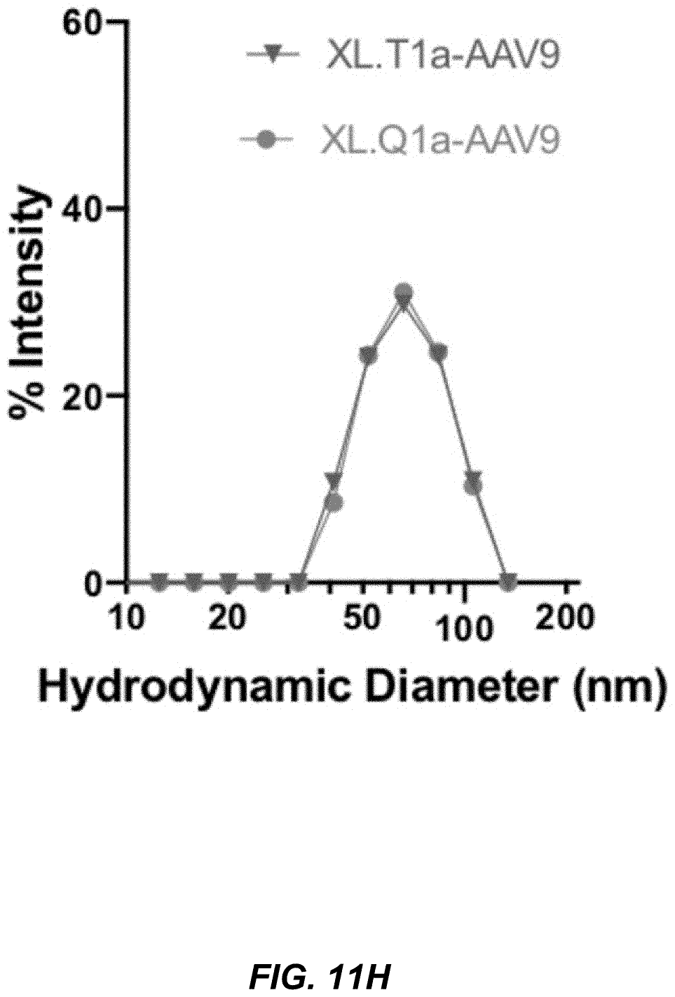

This example relates to a screen for linkers that increase the homogeneity and production efficiency of the expanded capsid formed by XL.D1a-AAV9. Different flexible linker lengths and different insertion sites at the second monomeric subunit were screened for initially (FIG. 10A, FIGS. 10D-10F). 7mer or 9mer linker variants showed a 2-3 fold increase in copy number of protected genomes in cell lysate compared with XL.D0-AAV9 when packaged with oversized (7 kb) genomes (FIG. 10A). Changing the flexible linkers to a 7mer TEV protease recognition site (ENLYFQG; SEQ ID NO: 13) or a 9mer derivative of the recognition site (GGENLYFQG; SEQ ID NO: 14) reduced aggregation of the capsid (FIG. 10B, FIGS. 10G-10H). Quantification of the size and shape of the particles in TEM images show that the optimized variant had a smaller variance in size (Bartlett's test for equal variance, P<0.001) and a rounder shape (Student's t-test, P<0.001) (FIG. 2F). These two new variants were named XL.D1b-AAV9 and XL.D1c-AAV9, respectively. The particles made of heterodimeric subunits were found to contain at least three isoforms corresponding to that observed in wild-type AAV9 (FIG. 11A). The same heterodimeric design can also be applied in AAV2, AAV5, and AAV-DJ, which yields size-expanded, genome-protecting particles in all tested serotypes (FIGS. 11B-E).

### Example 4

### Multimeric Design Concepts

This example demonstrates multimeric design strategies for expanded capsid sizes provided herein. In natural viral capsids, sub-assembly is not limited to the formation of dimers. Picornavirus capsids (T=3), for example, initiate assembly by sub-assembling into protomers comprising three structural subunits. To explore the generalizability of the tandem-multimerization strategy, a tandem trimer (wt-(d6)₂) and a tandem tetramer (wt-(d6)₃) were designed with re-optimized flexible linkers. Both the tandem trimer (FIG. 11D) and tandem tetramer (FIG. 11E) formed size-expanded capsids (FIGS. 11D-H). The tandem-trimer/tandem-tetramer variants were named XL.T1a-AAV9 and XL.Q1a-AAV9, respectively. To further investigate whether the increase in capsid size induced by tandem multimerization could merely be due to the increased local concentration of the capsid protein at the translation site tandem dimers with self-cleaving peptide linkers were designed. TEM imaging showed that these self-cleaving dimers no longer formed size-expanded capsids, indicating that the presence of the covalent linker is essential for moving forward in the post-translational assembly pathway (FIGS. 10I-10K). Without being bound by any particular theory, these results collectively support that the driving force for capsid size expansion in tandem-multimer XL-AAVs involves alterations in the assembly pathway.

### Example 5

### Guide Peptide Embodiments

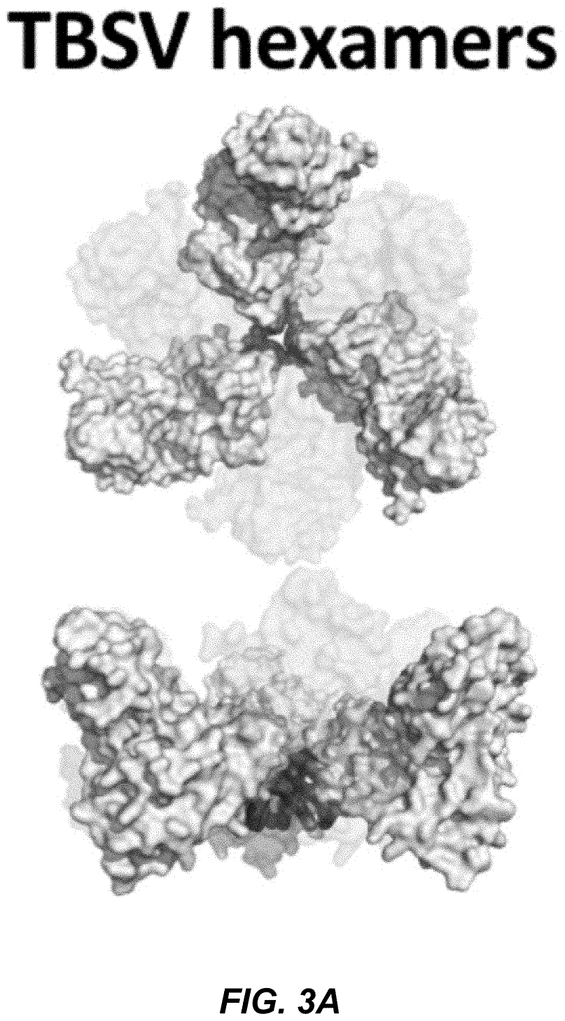

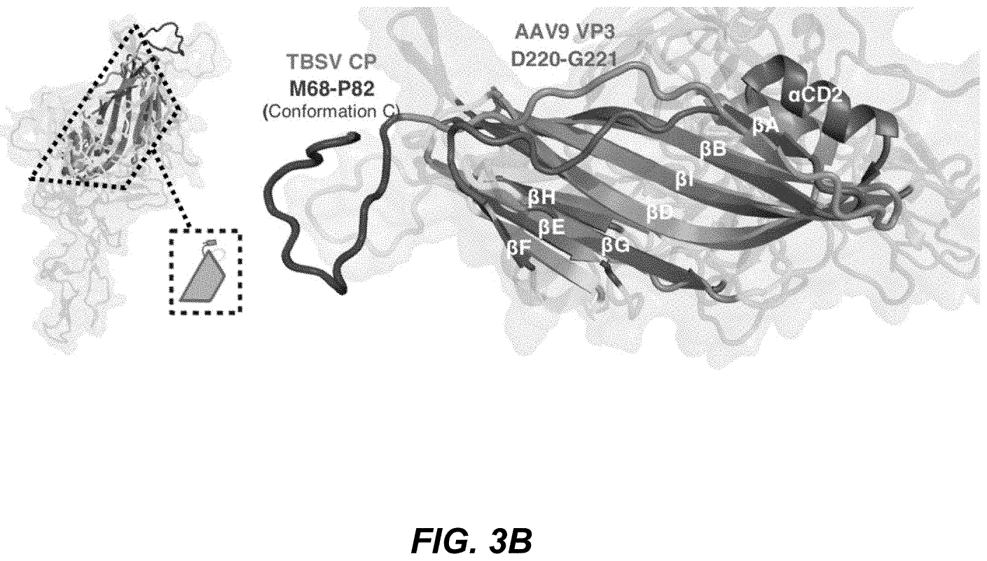

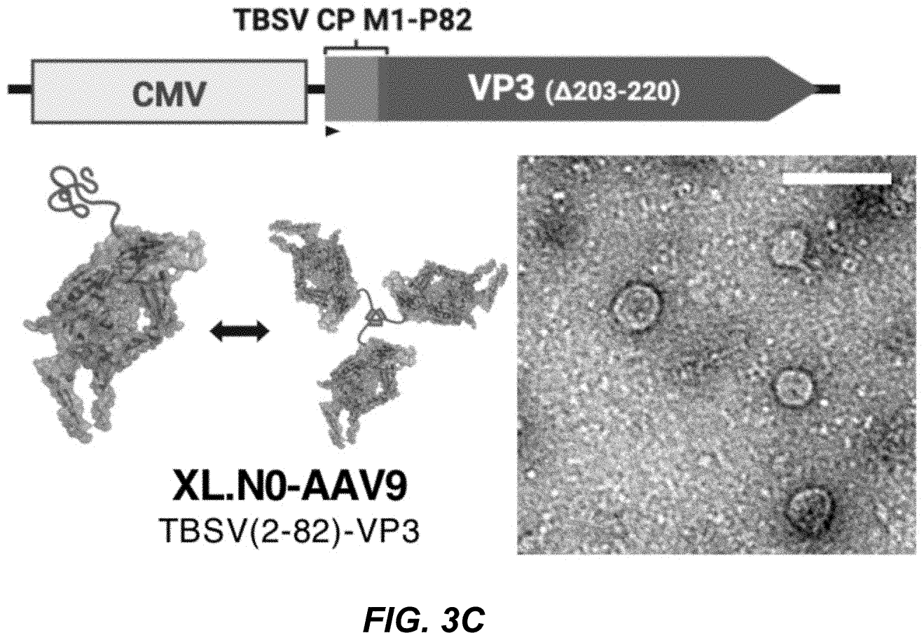

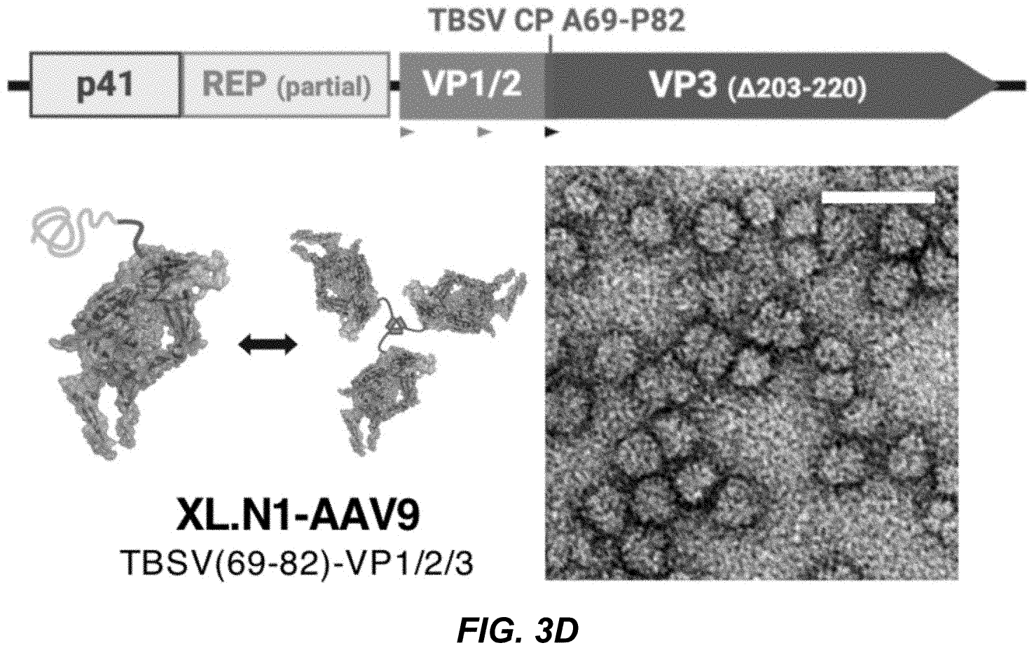

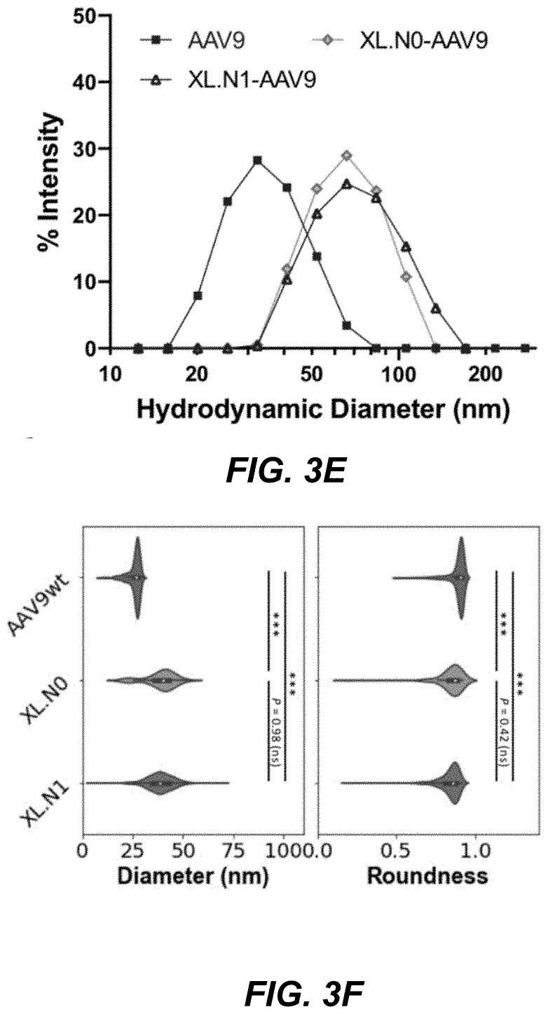

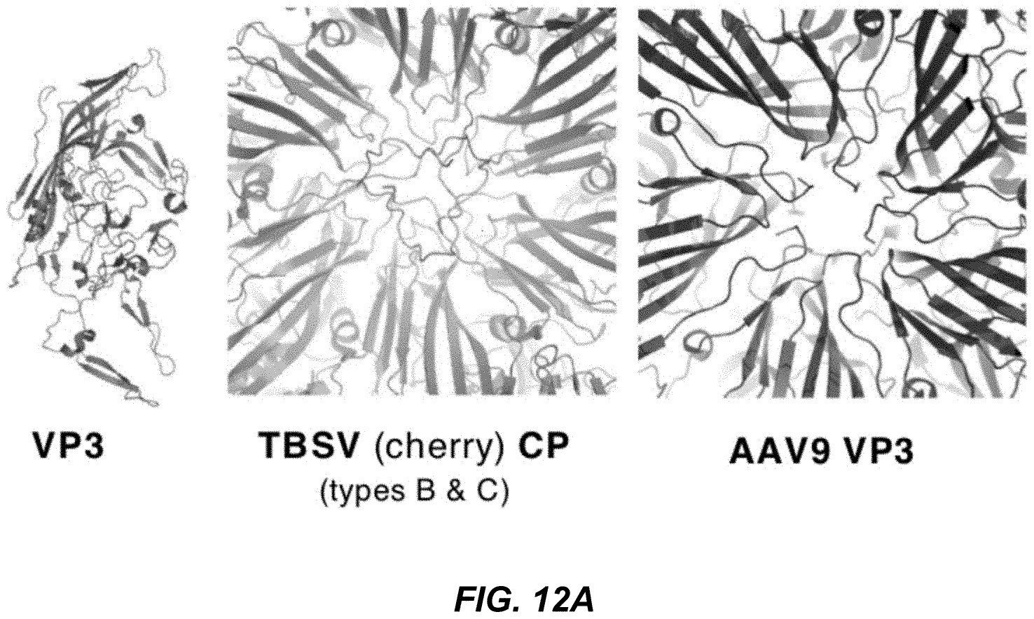

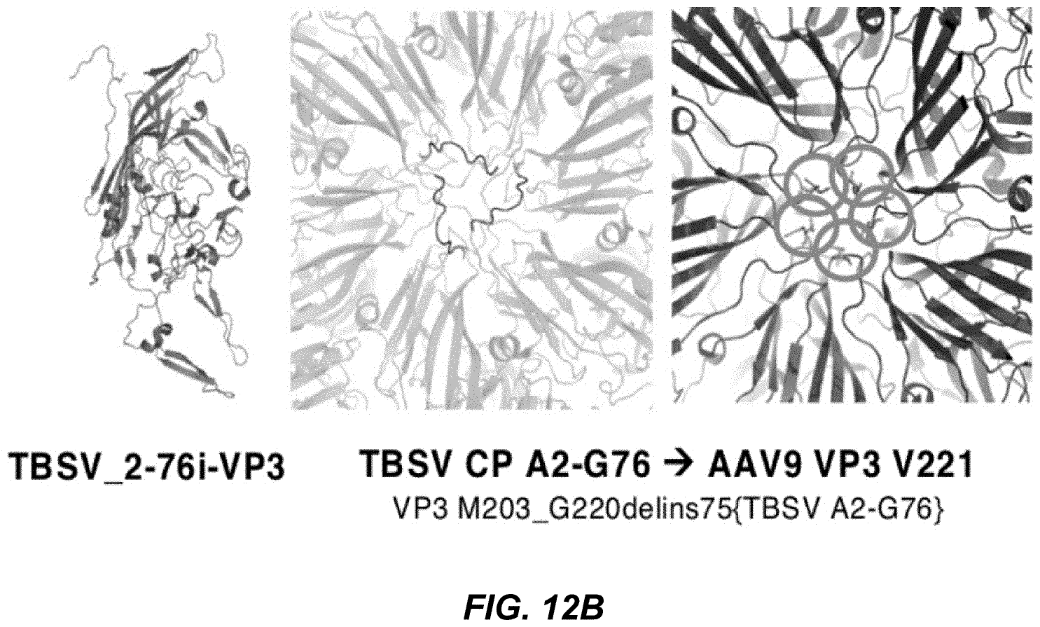

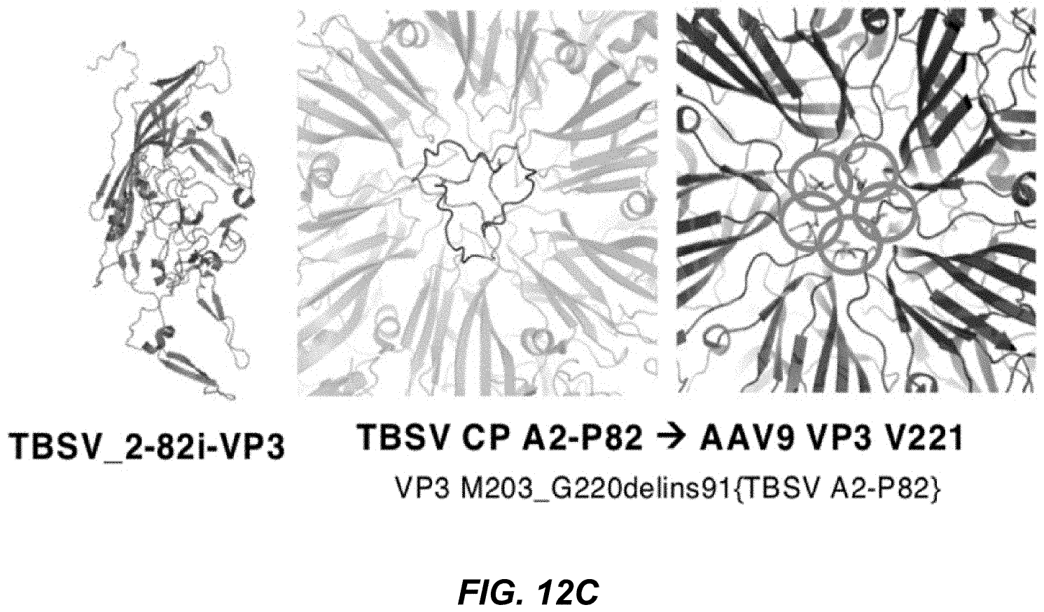

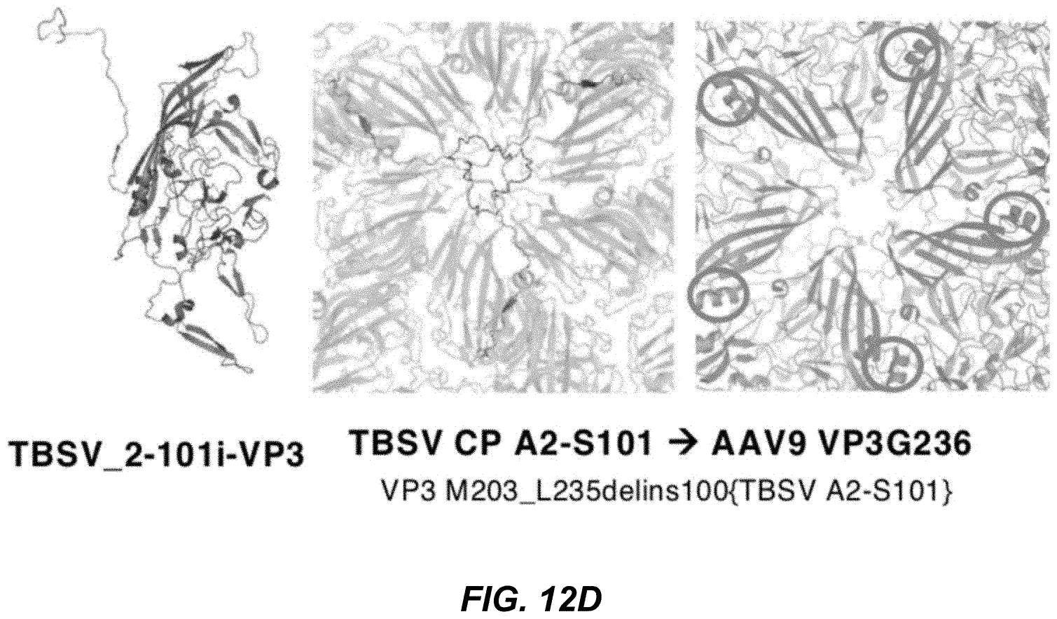

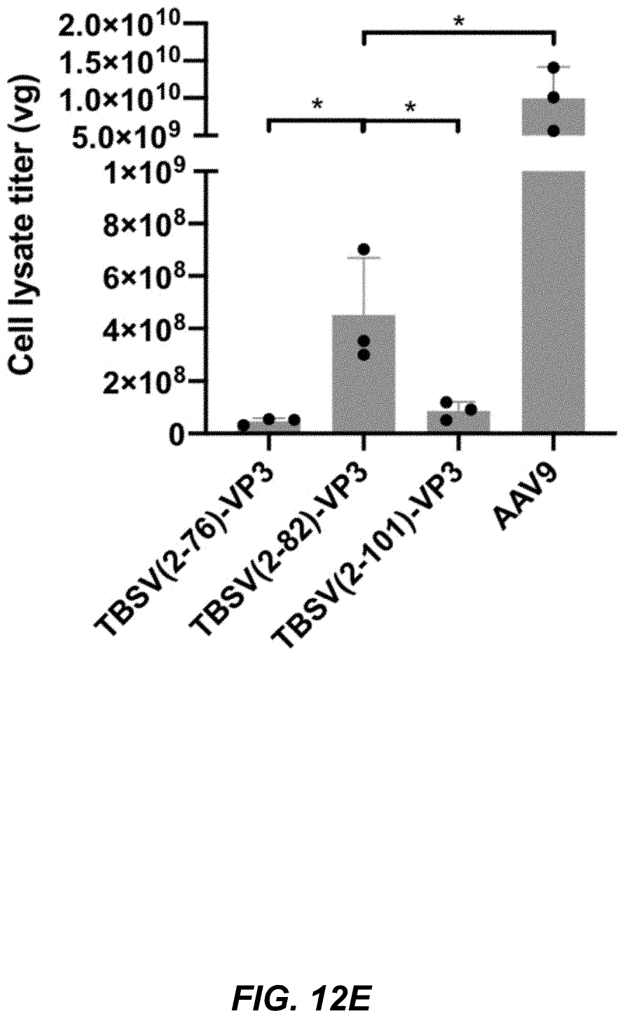

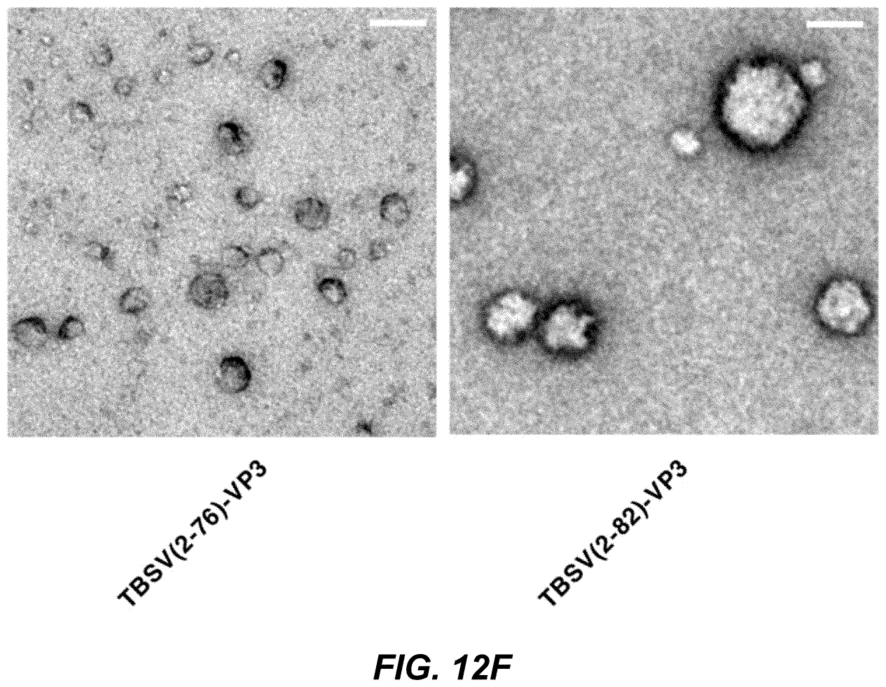

This example demonstrates a second strategy for altering the geometry of the AAV capsid by grafting a “guide peptide”. This strategy is based on the hypothesis that certain peptide sequences involved in the assembly of natively T≥3 viruses guide the formation of essential interactions needed for the more complicated geometry. Several ssRNA T=3 plant viral capsids, such as TBSV, have certain N-terminal peptides that are necessary and/or sufficient for the formation of larger-sized spherical assemblies (FIG. 6B, Tables 8-10). On the one hand, the removal of N-terminal residues M1-V72 from the TBSV coat protein (CP) results in shrinkage in capsid sizes and adoption of T=1 symmetry. On the other hand, a 24mer N-terminal peptide (I69-S92) self-assembled into 30-50-nm capsid-like particles, equivalent in size to the T≥3 capsids that can be formed by the CP. Together, these observations hinted at a role for this peptide in guiding the higher-order assembly of the T=3 capsid (FIG. 1C). In addition to its possible role in guiding assembly, the TBSV N-terminal peptide may also form interactions that stabilize the pseudo-6-fold interface. Part of the N-terminal peptide is conditionally structured and involved in an intertwined β-annulus structure at the pseudo-6-fold axis (FIGS. 3A-3B, FIG. 12A). Based on these reports, the N-terminal sequence of the TBSV CP was used as a source of guide peptides.

TABLE 8
EXAMPLE LARGER VIRUS SOURCE SPECIES AND THEIR LINEAGES

| REALM | | | | | | |
|---|---|---|---|---|---|---|
| AND | | | | | | |
| KINGDOM | | | | FAMILY AND | | SPECIES |
| (*GROUP #) | PHYLUM | CLASS | ORDER | SUBFAMILY | GENUS | NAME |
| Viruses> | Kitrinoviricota | Tolucaviricetes | Tolivirales | Tombusviridae> | Tombusvirus | Cucumber |
| Riboviria> | | | | Procedovirinae | | necrosis |
| Orthornavirae | | | | | | virus |
| (#1) | | | | | | |
| Viruses> | Kitrinoviricota | Tolucaviricetes | Tolivirales | Tombusviridae> | Tombusvirus | Tomato |
| Riboviria> | | | | Procedovirinae | | bushy stunt |
| Orthornavirae | | | | | | virus, cherry |
| (#1) | | | | | | strain |
| Viruses> | Kitrinoviricota | Tolucaviricetes | Tolivirales | Tombusviridae> | Tombusvirus | Tomato |
| Riboviria> | | | | Procedovirinae | | bushy stunt |
| Orthornavirae | | | | | | virus, BS-3 |
| (#1) | | | | | | strain |
| Viruses> | Kitrinoviricota | Tolucaviricetes | Tolivirales | Tombusviridae> | Betacarmovirus | Turnip |
| Riboviria> | | | | Procedovirinae | | crinkle virus |
| Orthornavirae | | | | | | |
| (#1) | | | | | | |
| Viruses> | Kitrinoviricota | Tolucaviricetes | Tolivirales | Tombusviridae> | Umbravirus | Carrot |
| Riboviria> | | | | Calvusvirinae | | mottle virus |
| Orthornavirae | | | | | | |
| (#1) | | | | | | |
| Viruses> | Kitrinoviricota | Tolucaviricetes | Tolivirales | Tombusviridae> | Dianthovirus | Carnation |
| Riboviria> | | | | Regressovirinae | | ringspot |
| Orthornavirae | | | | | | virus |
| (#1) | | | | | | |
| Viruses> | Pisuviricota | Pisoniviricetes | Sobelivirales | Solemoviridae> | Sobemovirus | Sesbania |
| Riboviria> | | | | N/A | | mosaic |
| Orthornavirae | | | | | | virus |
| (#2) | | | | | | |
| Viruses> | Pisuviricota | Pisoniviricetes | Sobelivirales | Solemoviridae> | Sobemovirus | Southern |
| Riboviria> | | | | N/A | | bean mosaic |
| Orthornavirae | | | | | | virus |
| (#2) | | | | | | |
| Viruses> | Pisuviricota | Pisoniviricetes | Sobelivirales | Solemoviridae> | Sobemovirus | Southern |
| Riboviria> | | | | N/A | | cowpea |
| Orthornavirae | | | | | | mosaic |
| (#2) | | | | | | virus |
| Viruses> | Kitrinoviricota | Alsuviricetes | Martellivirales | Martellivirales> | Bromovirus | Brome |
| Riboviria> | | | | Bromoviridae | | mosaic |
| Orthornavirae | | | | | | virus |
| (#3) | | | | | | |
| Viruses> | Pisuviricota | Pisoniviricetes | Picornavirales | Picornavirales> | Lagovirus | Rabbit |
| Riboviria> | | | | Caliciviridae | | hemorrhagic |
| Orthornavirae | | | | | | disease |
| (#4) | | | | | | virus |
| Viruses> | Pisuviricota | Pisoniviricetes | Picornavirales | Picornavirales> | Vesivirus | Feline |
| Riboviria> | | | | Caliciviridae | | Calicivirus |
| Orthornavirae | | | | | | |
| (#4) | | | | | | |
| Viruses> | Pisuviricota | Pisoniviricetes | Picornavirales | Picornavirales> | Norovirus | Nowalk |
| Riboviria> | | | | Caliciviridae | | virus |
| Orthornavirae | | | | | | |
| (#4) | | | | | | |

*Note:
“Group #” is an arbitrarily defined classification based on the similarity of capsid structures.

TABLE 9
EXAMPLE LARGER VIRUS PEPTIDE SEQUENCES

| | CAPSID | | SEO ID |
|---|---|---|---|
| SOURCE SPECIES | LENGTH | LOCATION | NO: |
| Cucumber necrosis virus | 380aa | residues 1-70 | 89 |
| Tomato bushy stunt virus, cherry | 388aa | residues 1-84 | 90 |
| strain | | | |
| Sesbania mosaic virus | 268aa | residues 1-77 | 91 |
| Southern bean mosaic virus | 266aa | residues 1-73 | 92 |
| Southern cowpea mosaic virus | 279aa | residues 1-86 | 93 |
| Brome mosaic virus | 189aa | residues 1-47 | 94 |
| Rabbit hemorrhagic disease virus | 579aa | residues 1-54 | 95 |
| Cucumber necrosis virus | 380aa | residues 55-70 | 96 |
| Tomato bushy stunt virus, cherry | 386aa | residues 68-83 | 97 |
| strain | | | |
| Sesbania mosaic virus | 268aa | residues 64-75 | 98 |
| Southern bean mosaic virus | 266aa | residues 59-71 | 99 |
| Southern cowpea mosaic virus | 279aa | residues 73-84 | 100 |
| Brome mosaic virus | 189aa | residues 33-45 | 101 |
| Rabbit hemorrhagic disease | 579aa | residues 38-52 | 102 |
| virus | | | |

TABLE 10
FULL LENGTH CAPSID SEQUENCES FOR
EXAMPLE LARGER VIRUS SPECIES

| | LENGTH OF | SEO ID |
|---|---|---|
| SPECIES NAME | CAPSID PROTEIN | NO: |
| Cucumber necrosis virus | 380aa | 103 |
| Tomato bushy stunt virus, cherry | 388aa | 104 |
| strain | | |
| Sesbania mosaic virus | 268aa | 105 |
| Southern bean mosaic virus | 266aa | 106 |
| Southern cowpea mosaic virus | 279aa | 107 |
| Brome mosaic virus | 189aa | 108 |
| Rabbit hemorrhagic disease | 579aa | 109 |
| virus | | |

Because the TBSV CP backbone has a similar jelly-roll fold as the AAV capsid protein backbone (FIG. 3B), three structurally analogous turns in both proteins as candidate graft sites were identified. Coat protein peptides A2-G76, A2-P82, and A2-S101 from TBSV (cherry strain) were grafted to the corresponding sites in the AAV9 VP3, yielding three variants (FIGS. 12B-12E). Among the three, the variant with the A2-P82 insertion produced the highest DNasel-protected genome titer (FIG. 12E). TEM and DLS examination of the variant showed that it formed heterogeneous particles that were significantly larger than regular AAV capsids (FIG. 3C, FIGS. 3E-3F, FIG. 12F). This variant was named XL.N0-AAV9 to indicate the critical modification in the N-terminal sequence. Owing to the large size of this TBSV graft (TBSV CP A2-P82), the entire unstructured N-terminal sequence of AAV9 (A2-G221) was removed in XL.N0-AAV9 (FIG. 3C) to avoid steric interference. Therefore, the functional sequences within the VP1/2-unique region (A2-T202) of the natural AAV9 capsid protein were not retained in this variant.

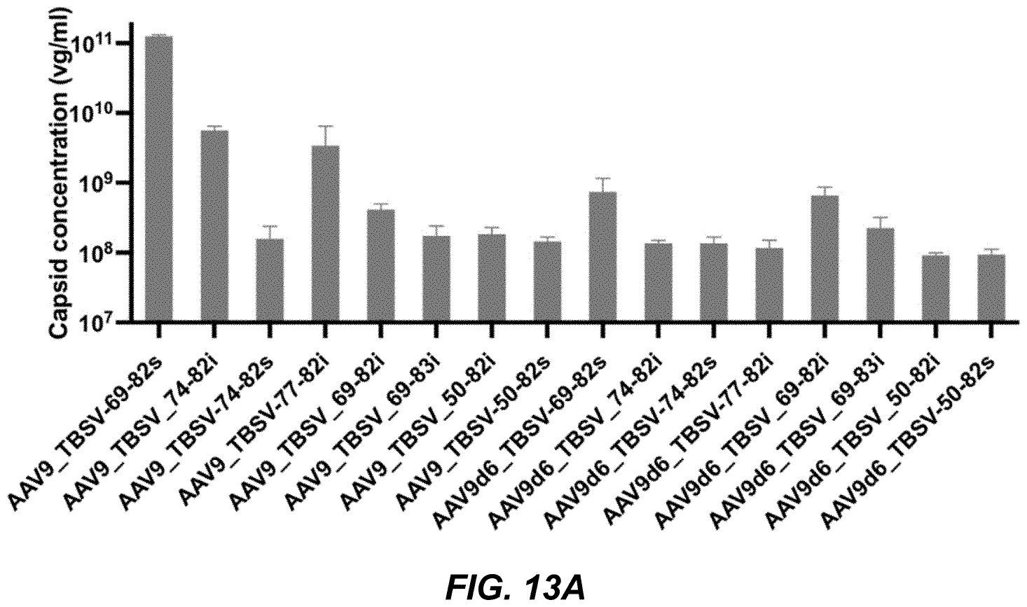

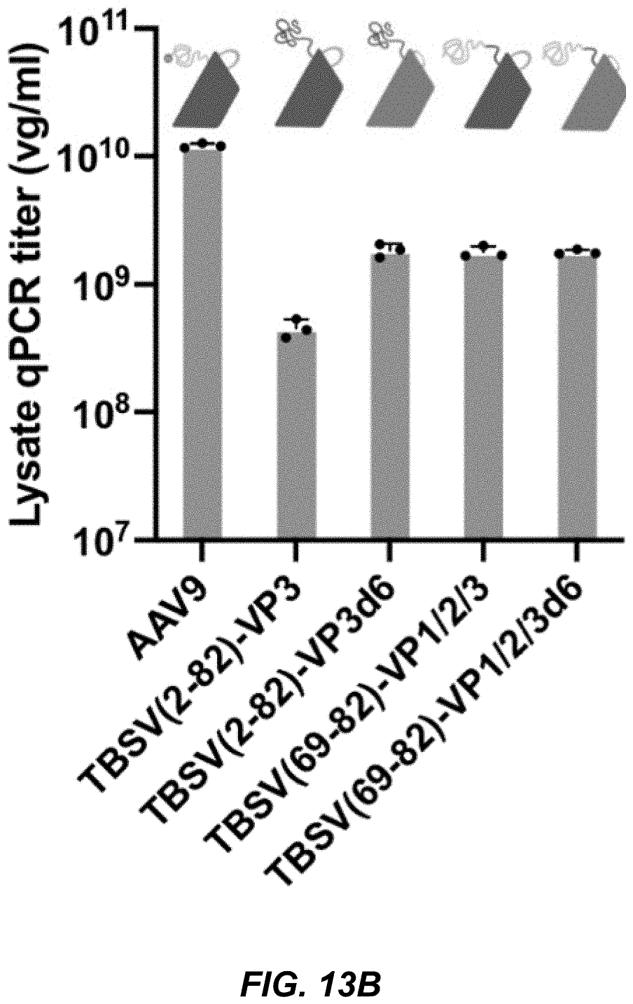

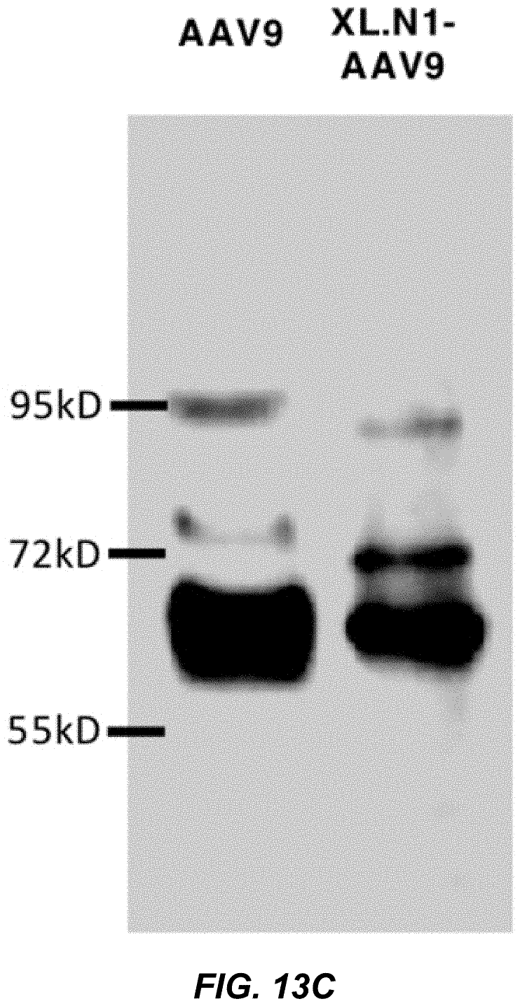

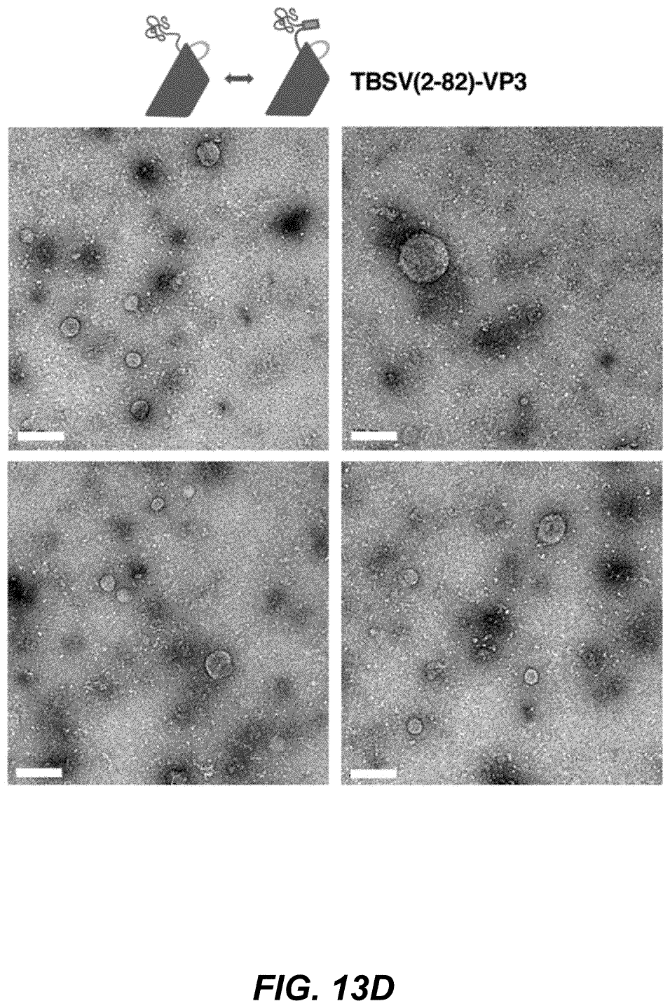

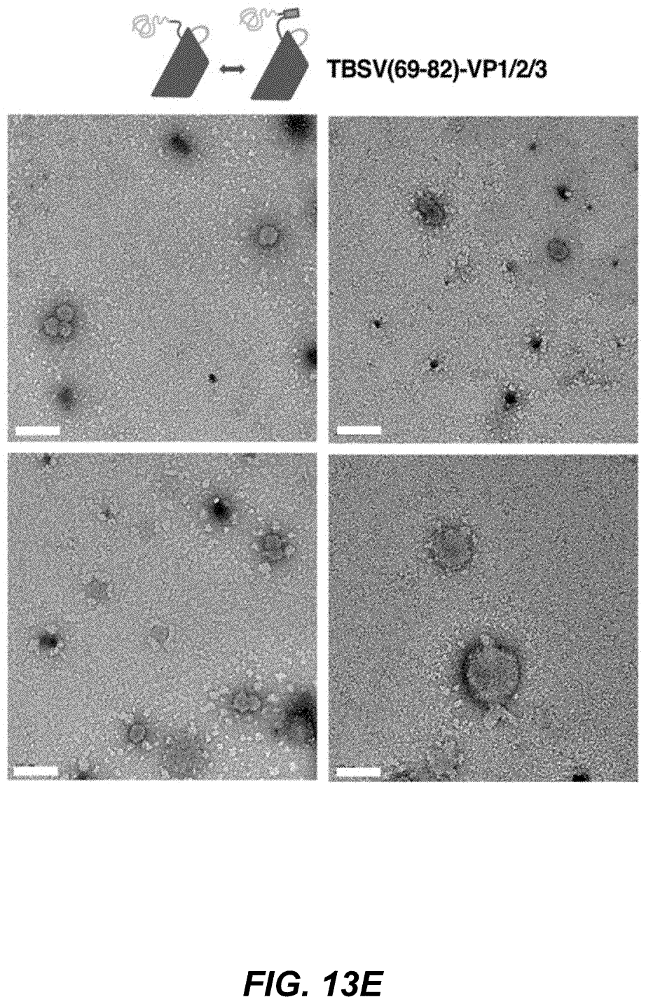

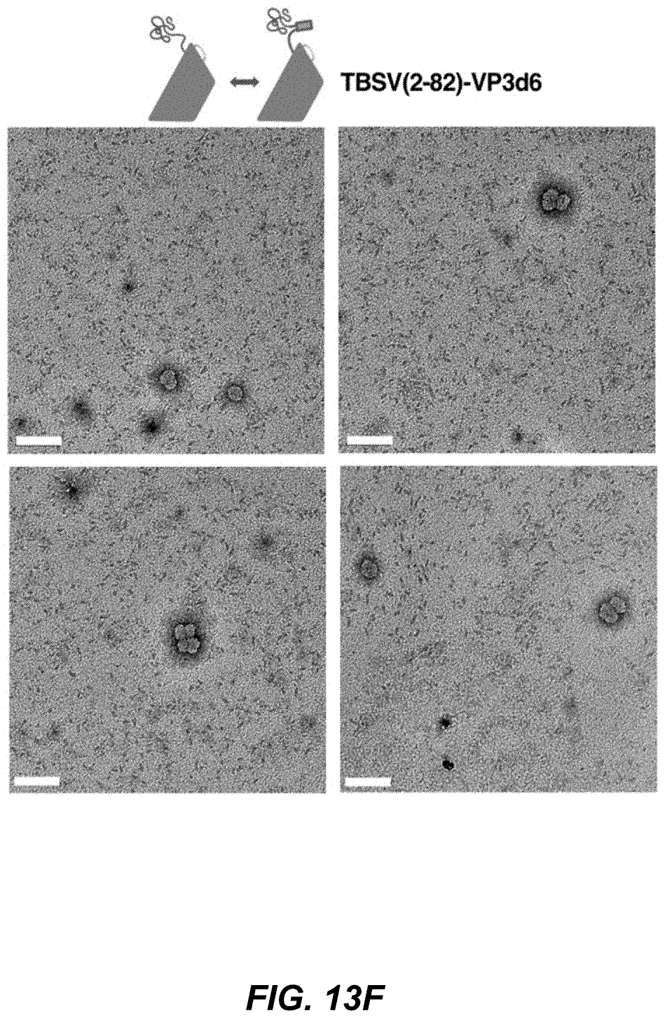

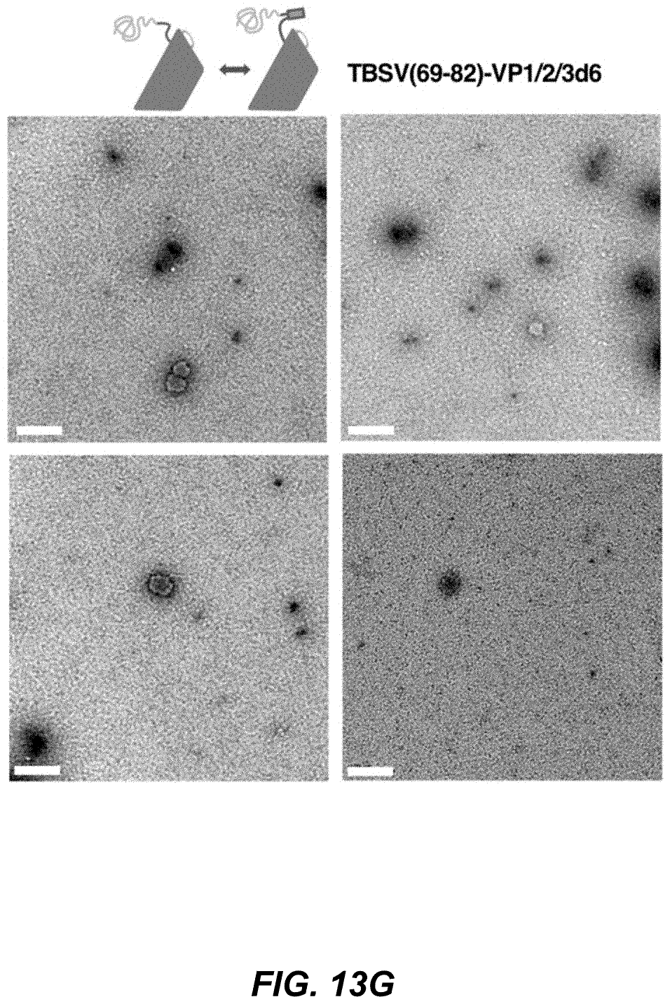

This example next demonstrates that grafting only a short stretch of the region modified in XL.N0-AAV9 can be sufficient to produce larger AAV capsids. A selection of peptides within TBSV CP A2-P82 were tested, prioritizing sequences that contribute to the β-annulus, by inserting them into the N-terminal region of AAV9 VP3. The exact insertion sites were chosen manually to align the sequence and structure of the TBSV graft and those of the native AAV capsid protein. Several variants produced DNasel-protected viral genomes (FIGS. 13A-13B). The variant with the highest genome titer had a 14aa TBSV CP peptide (I69-P82) insertion, replacing 17 residues following the VP3 start codon (A204-G220). Since the 14aa-replacement variant produced all three subunit isoforms (FIG. 13C) and showed morphology similar to XL.N0-AAV9 (FIGS. 3D-3F, FIGS. 13D-13G), it was termed XL.N1-AAV9.

### Example 6

### Delivery of Oversized Cargo by XL-AAV

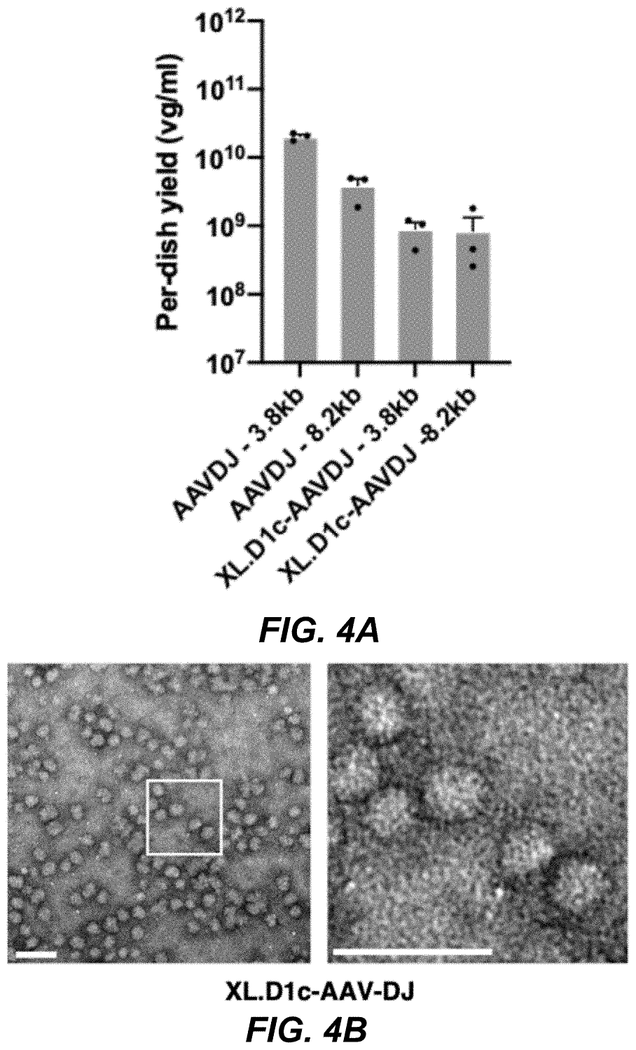

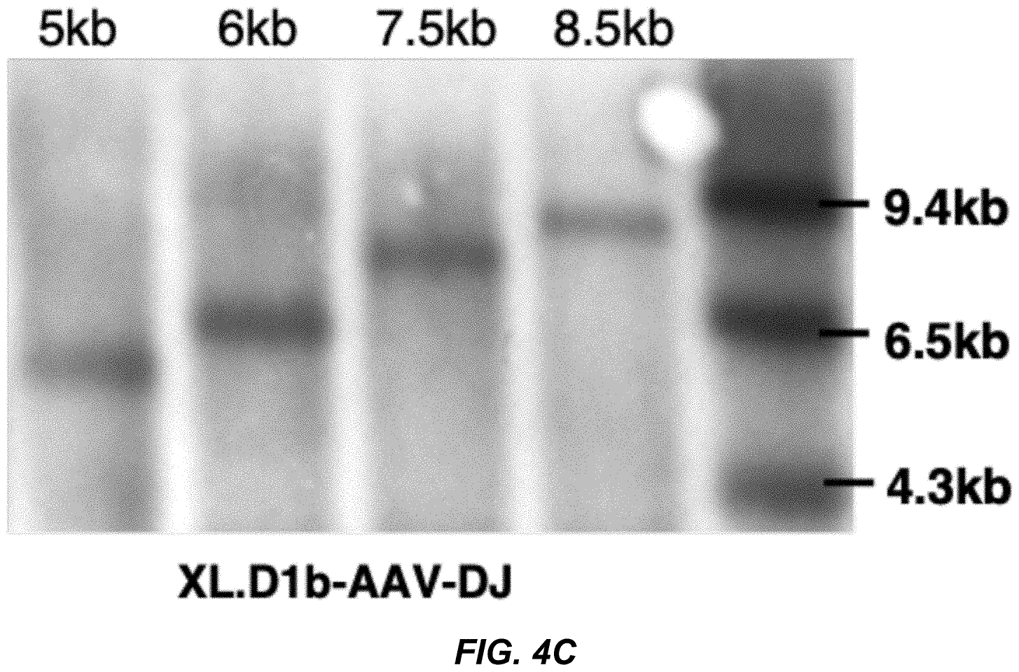

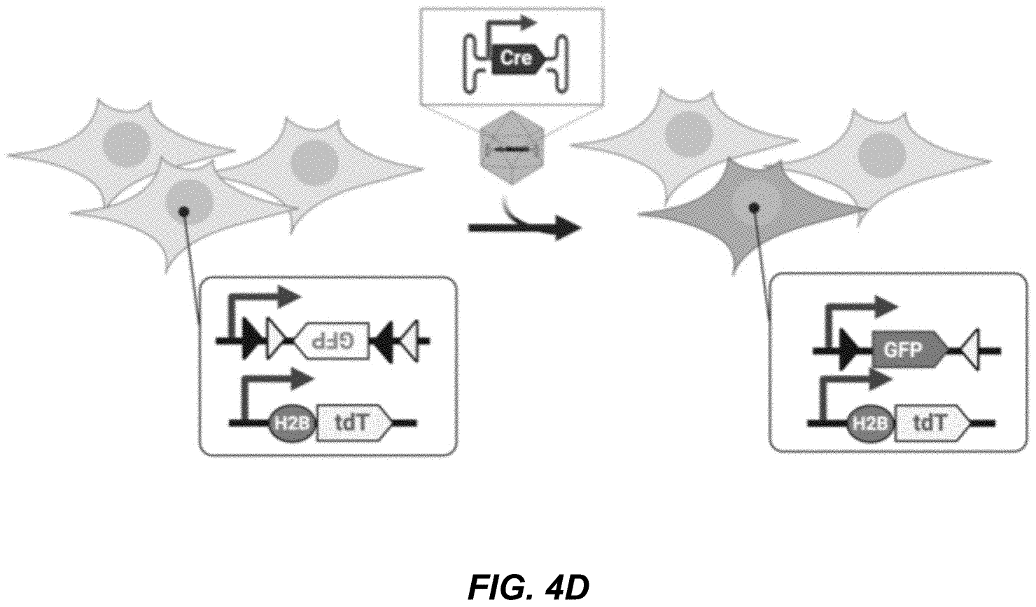

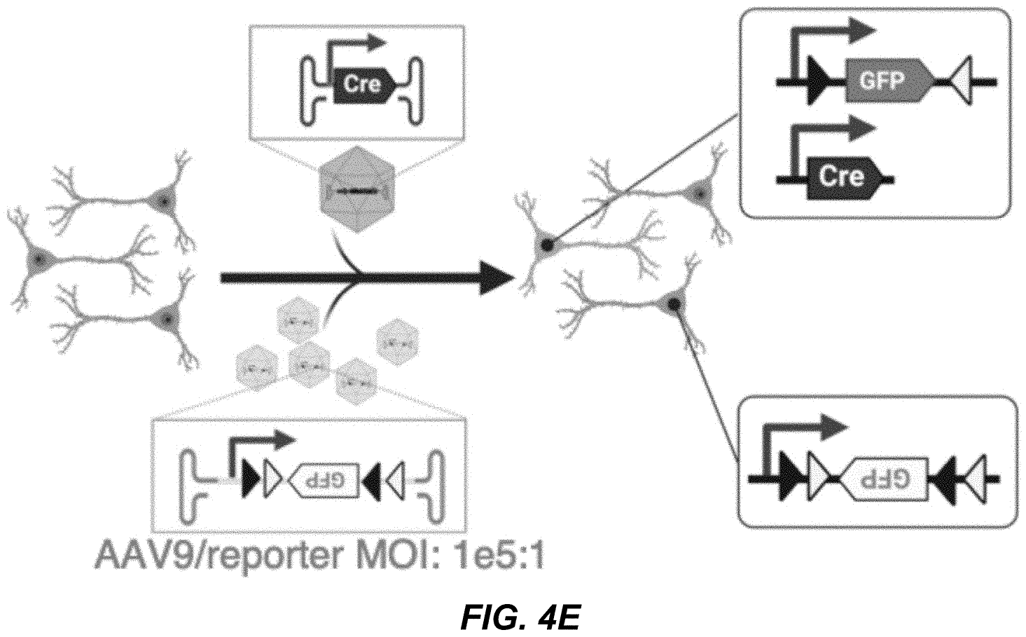

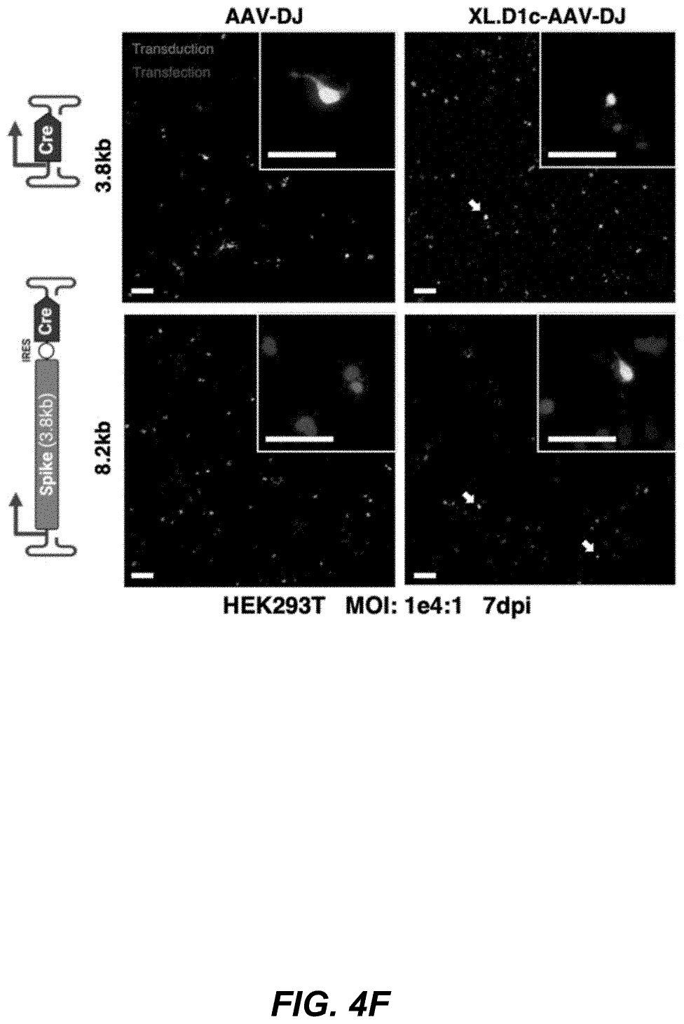

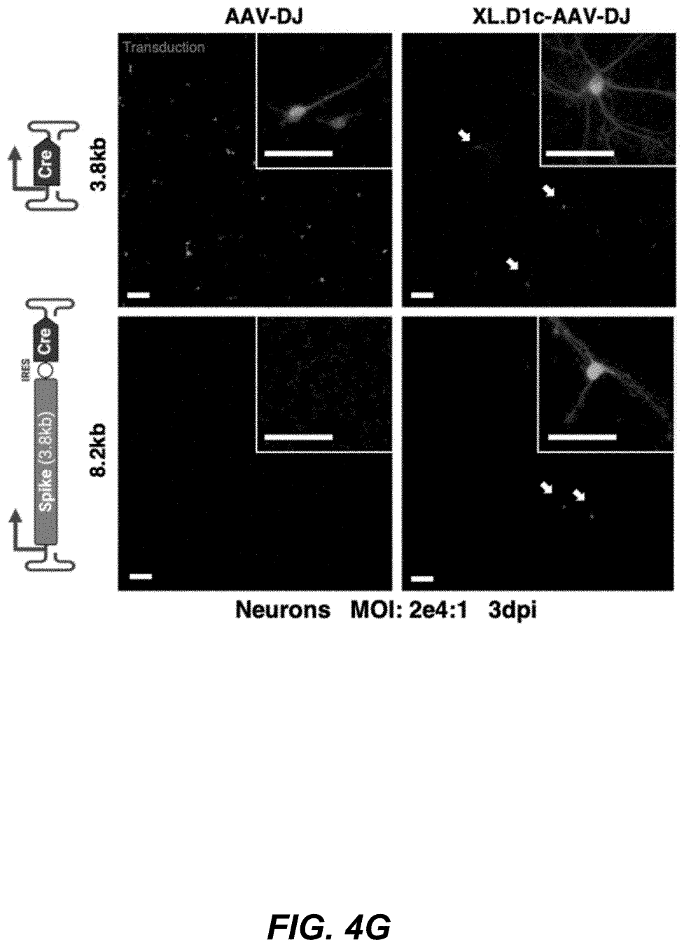

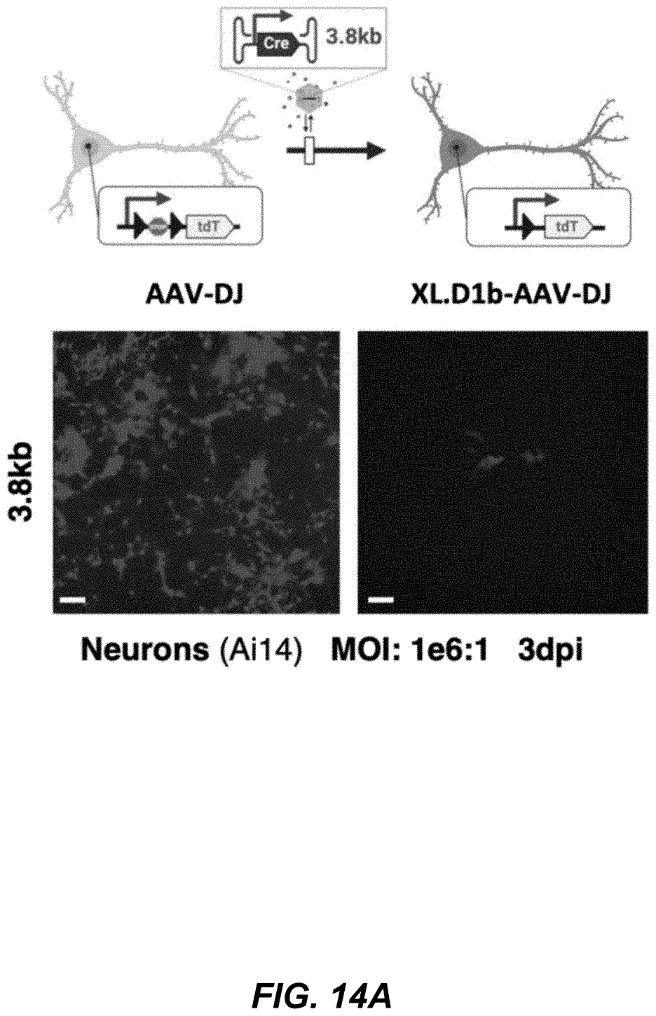

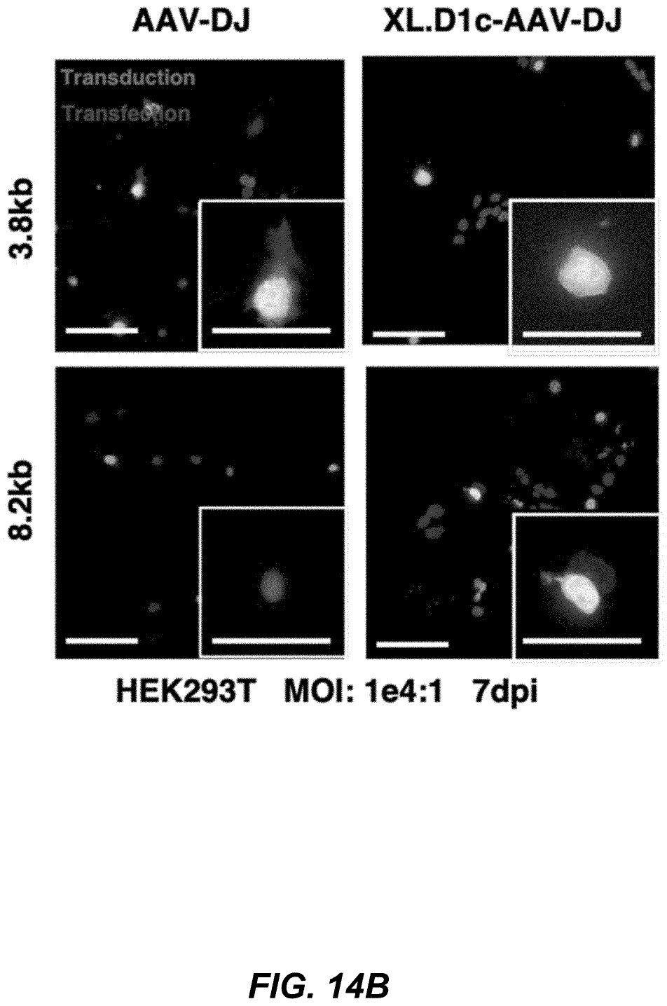

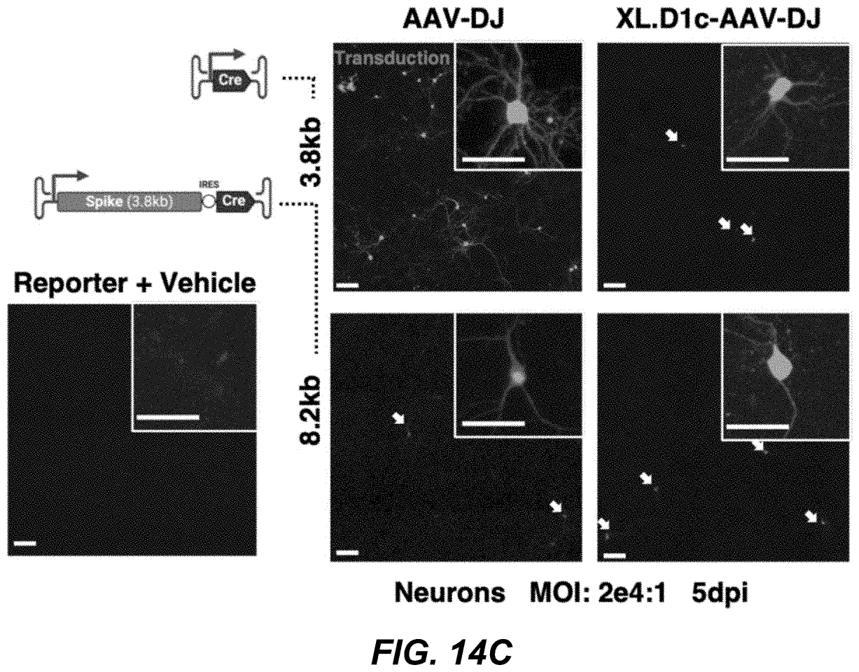

This example demonstrates the ability of XL-AAV to deliver oversized genetic cargo. For this purpose, the backbone was switched to another AAV serotype, AAV-DJ (FIGS. 4A-4B), which has superior production and transduction efficiency. Alkaline gel electrophoresis and Southern blot of cargo DNA extracted from DNase-I-treated XL.D1b-AAV-DJ viral particles indicate that the capsid can protect full-length rAAV genomes that are as long as 8.5 kb (FIG. 4C). To evaluate whether the viral particles have the potential of transducing cells in vitro, cell culture assays based on Cre recombinase-dependent reporter expression were used. A regular-sized, 3.8 kb genome with an EF1α-Cre expression cassette was tested first (rAAV genome #6, Table 6). DNasel-treated viral particle extracts carrying the genome were applied to brain cell cultures from a mouse strain with a Cre-dependent red fluorescent protein reporter in its genome (FIG. 14A). Fluorescent protein expression was observed in cells briefly incubated with the XL-D1b-AAV-DJ extract, demonstrating that the XL-AAV design retains basic cell entry and genome delivery ability. Following this encouraging result, next it was tested whether XL-AAV particles can also deliver an oversized genome to both HEK293T human immortal cells (FIG. 4D, FIG. 4F) and primary neuronal cultures (FIG. 4E, FIG. 4G). A 8.2 kb oversized genome (rAAV genome #7, Table 6) was constructed by inserting 4.4 kb spacer sequences between the promoter and the Cre coding gene of the 3.8 kb genome (FIG. 4D, left). This genome design made sure that truncations from either end of the genome would not result in a 5 kb fragment that contains both the genome and the promoter. Both the regular-sized, 3.8 kb rAAV genome containing an EF1α-Cre expression cassette and the oversized 8.2 kb rAAV genome were packaged into both AAV-DJ and XL.D1c-AAVDJ. As expected, wild-type AAV-DJ could efficiently deliver the 3.8 kb genome to HEK293T cells pre-transfected with a Cre-dependent reporter plasmid. However, no Cre-driven reporter expression was detectable in the same HEK293T cells transduced with AAV-DJ capsids packaging the 8.2 kb genome (FIG. 4F, FIG. 14B). In contrast, although the overall transduction efficiency is low, XL.D1c-AAV-DJ does deliver both 3.8 kb and 8.2 kb genomes (FIG. 4F, FIG. 14B) to HEK293T cells. In addition, XL.D1c-AAV-DJ can also transduce primary cultured neurons with both genomes at similar efficiencies (FIG. 4G), while Cre-driven expression by AAV-DJ drastically declined when packaging the 8.2 kb genome (FIG. 4G). It is worth noting that regular-sized AAVs can result in expression of an “oversized” expression cassette not because of delivery of the full-length oversized genome, but as a result of intracellular reassembly of co-transduced vectors with truncated genomes from both ends; these events are characterized by decreased transduction efficiency, lower expression level, and slower expression kinetics of transgene expression, which was also observed for AAV-DJ (FIG. 14C). To summarize, the XL-AAV capsids of the present disclosure can package and protect oversized rAAV genomes. XL.D1c-AAV capsids can also transduce cultured human and rodent cell types.

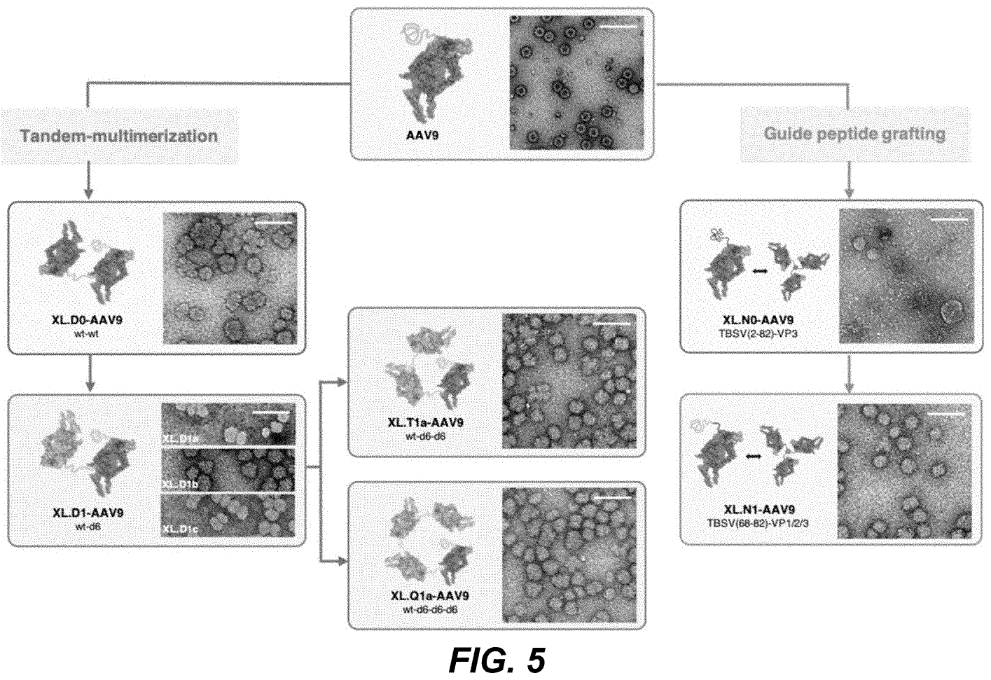

In conclusion, this work provides proof of concept that the size and geometry of protein nanocages can be reprogrammed by assembly-pathway engineering (FIG. 5). Prior work on the geometric design of protein nanocages has focused on rigid interfaces for identical interactions; by contrast, the compositions and methods provided herein are focused on mimicking the assembly pathways of natural viral capsids and permitting the possibility of quasi-equivalent interactions as disclosed herein.

### Example 7

### Optimization of Virus Purification

This example describes an exemplary XL-AAV purification workflow modifying the purification steps described above.

Cell Culture and Production of Viral Particle Extracts

HEK293T cell culture and triple transfection with polyethylenimine (PEI) were conducted according to a published protocol (Hirsch, M. L. et al., 2013). HEK293T cells were cultured in DMEM (ThermoFisher, cat. no. 10569044) with 5% FBS, 10 mM HEPES, and 1× non-essential amino acids. The cells were passaged to 15-cm dishes or 6-well plates at 40% confluence one day before transfection. In the case of 15-cm dishes, HEK293T producer cells (about 80% to about 90% confluent) were transfected with PEI with 20 μg iCAP or REP-CAP plasmid, 10 μg pHelper plasmid (Agilent, cat. no. 240071), 5 μg CMV-AAP plasmid, and 5 μg rAAV genome plasmid. In the case of 6-well plates, the plasmid masses were linearly scaled down to 2 μg iCAP plasmid, 1 μg pHelper plasmid, 0.5 μg CMV-AAP plasmid, and 0.5 μg rAAV genome plasmid for each well. Sequences for the plasmids as well as rAAV genomes are depicted in Tables 5-7. Plasmids were transfected to cells with PEI at a 3.5:1 (μg PEI: μg DNA) ratio.

72-96 hr after transfection, 1/80 media volume of 0.5 M EDTA was added to each dish/well to detach the cells from culture dishes. After 15 min incubation at room temperature, the mixture of cells and media was collected and centrifuged at 2000 g for 10 min at 4° C., and the supernatant (media) is removed completely.

Producer cell pellets were immediately lysed following a protocol for nonenveloped viruses (Kawano et al, 2018) with some modifications. Lysis buffer (100 mM magnesium chloride, 38.1 mM citric acid, 74.8 mM sodium citrate, 75 mM sodium chloride; the pH of the resulting solution should be about 4) was sterilized with a 0.22 μm PES, supplemented with 1 tablet protease inhibitor (ThermoFisher, cat. no. A32963) per 50 mL solution, and stored in 4° C.

The cell pellet samples were lysed with two rounds of lysis cycles. In each round, fresh lysis buffer was added to cell pellets at a ratio of 1 mL/1e7 cells. The mixture was vortexed for 20 s to fully resuspend the pellet. The resulting sample was then incubated for 5-15 min at room temperature and vortexed for 20 s again before being centrifuged at 5000-9000 g for 10 min. The supernatant was moved to a clean tube. Supernatant from two rounds of lysis were combined, and the total volume of the extract solution should be ˜2 mL/1e7 cells. The extract solution was neutralized with 1/10 volume of neutralization buffer (2 M Tris-HCl, pH 9.5) and then diluted with 1/3 volume of ddH₂O. Depending on the purpose of the experiment, the samples were either further purified or used for experiments, as specified in figure descriptions. In case when the sample was not being used for further purification, 0.05% BSA was added to the sample for better stability.

Virus Purification (Precipitation-Based Method)

Unless otherwise specified, virus samples for TEM, DLS, and transduction assays were prepared via the following precipitation-based purification method, a modified version of a generic protocol for purifying nonenveloped viruses (Kawano et al, 2018). The viral particle extract was added with 1/100 volume of DNase I (Roche, cat. no. 4716728001) and 1/100 volume of MspI (NEB, cat. no. R0106L). The reaction was incubated at 37° C. for 1 hr. Afterward, the sample was mixed well with 1/10 volume of precipitation buffer A (5% (m/v) sodium deoxycholate), and the mixture was incubated at 37° C. for another 30 min. Finally, the sample was mixed with 1/25 volume of precipitation buffer B (1 M citric acid), vortexed for 10 s, and centrifuged at 5000 g for 5 min at 4° C. All abovementioned “volumes” refer to the total liquid volume at the step.

After centrifugation, the supernatant with capsids was filtered with a 0.45 μm filter (Millipore cat. no. SLHP033RS or cat. no. UFC30HV00) and buffer exchanged for at least 5 cycles with 100 kD MWCO centrifugal concentrator (Thermo Scientific, cat. no. 88503 or cat. no. 88533) to the final storage buffer (PBS with additional NaCl and 0.001% Pluronic F-68 (Thermo Scientific, cat. no. 24040032). In each cycle of buffer exchange, the concentrator tubes were centrifuged at 1000 g, 4° C. for 20 min or more to reduce the solution volume by at least 5 folds. The purified virus samples were titrated with qPCR at the same day, and the remaining samples were stored at 4° C. for further characterization.

For TEM and DLS analyses, sample purity was further improved by 3-5 cycles of buffer exchange with a 300 kD MWCO centrifugal concentrator (Pall, Cat. OD300C33). In some embodiments, the recovery rate at this step can be further optimized.

FIGS. 16A-16B depict data related to optimized AAV purification workflows. FIG. 16A depicts data related to the optimization of the storage conditions of XL.D1c-AAVDJ. Purified XL.D1c-AAVDJ was incubated in buffers with different ionic strengths (columns 1-6) or additives (columns 7-8) at 4 C. All buffers include the salt components indicated in the figure and an additional 0.001% Pluronic F-68. The remaining titers in the solutions were quantified with qPCR after 10 days of incubation. Titers of XL.D1c-AAVDJ are most robust at an ionic strength of ˜600 mM, and particles incubated with glycine amide (GlyNH2) shows improved stability of compared to particles incubated with NaCl at the same concentration. FIG. 16B depicts data related to the optimization of harvest times of XL.D1c-AAVDJ. XL.D1c-AAVDJ was produced following the protocol described in Example 7, and cell pellets were harvested and extracted at different time points after transfection. DNasel-protected titer in the extract was quantified with qPCR. The titer in the extract peaks at around 96 hours after transfection.

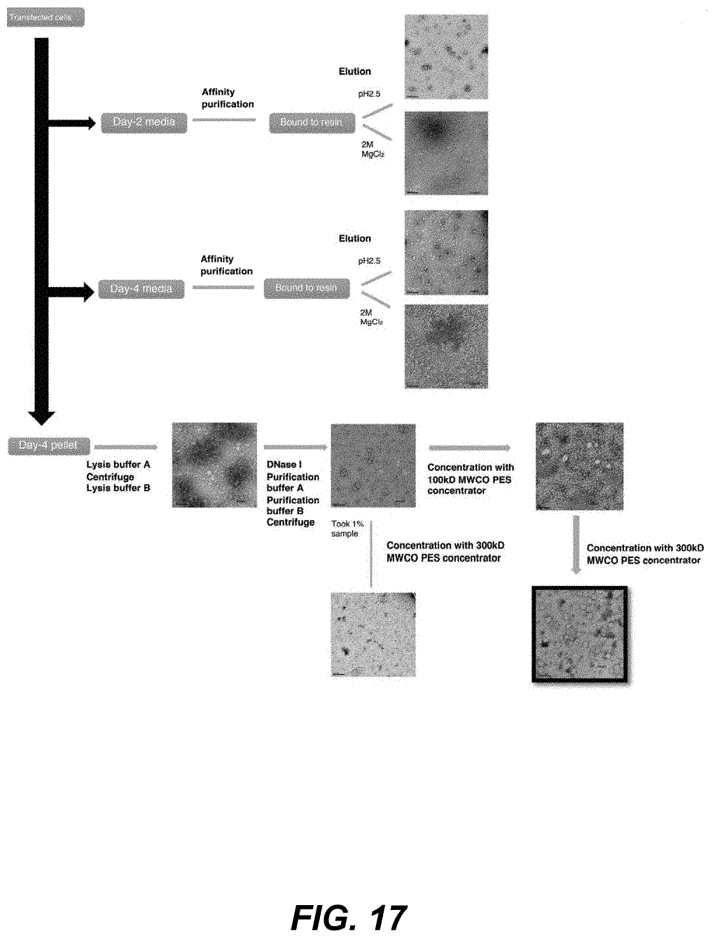

FIG. 17 depicts a non-limiting exemplary virus purification workflow. FIG. 17 shows electron micrographs of samples of XL.D1-AAV9 producer cells after different purification steps (scale bar: 100 nm).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A variant AAV capsid,
wherein the variant AAV capsid has a diameter of at least 30 nm.

2. The variant AAV capsid of claim 1, wherein the variant AAV capsid has a diameter of about 30 nm to about 35 nm, of about 30 nm to about 60 nm, of about 40 nm to about 60 nm, of about 45 nm to about 60 nm, of about 50 nm to about 65 nm, or of about 55 nm to about 60 nm.

3. The variant AAV capsid of claim 1, wherein the variant AAV capsid has a genetic cargo capacity of about 5.2 kb to about 8.5 kb, about 5.2 kb to about 5.5 kb, about 5.5 kb to about 6.0 kb, about 6.0 kb to about 6.5 kb, about 6.5 kb to about 7.0 kb, about 7.0 kb to about 7.5 kb, about 7.5 kb to about 8.0 kb, or about 8.0 kb to about 8.5 kb.

4. The variant AAV capsid of claim 2, wherein the genetic cargo capacity is: (i) the maximum length of a single-stranded DNA molecule that the variant AAV capsid is capable of protecting from DNAse I digestion; and/or (ii) the maximum length of a double-stranded DNA molecule that the variant AAV capsid is capable of protecting from DNAse I digestion.

5. The variant AAV capsid of claim 1, wherein the variant AAV capsid comprises a plurality of tandem multimers, wherein a tandem multimer comprises two or more AAV capsid proteins, wherein the tandem multimer comprises one or more linkers connecting the two or more AAV capsid proteins, and wherein said two or more AAV capsid proteins comprise two or more parental AAV capsid proteins, or derivatives thereof.

6. The variant AAV capsid of claim 5, wherein the tandem multimer comprises:
(a) a tandem dimer of a first capsid protein and a second capsid protein, wherein the tandem dimer comprises a first linker;
(b) a tandem trimer of a first capsid protein, a second capsid protein, and a third capsid protein, wherein the tandem trimer comprises a first linker and a second linker; or
(c) a tandem tetramer of a first capsid protein, a second capsid protein, a third capsid protein, and a fourth capsid protein, and wherein the tandem tetramer comprises a first linker, a second linker, and a third linker.

7. The variant AAV capsid of claim 6, wherein the first capsid protein, the second capsid protein, the third capsid protein, and/or the fourth capsid protein comprises a HI loop variant capsid protein, wherein a HI loop variant capsid protein comprises a removal of one or more amino acids in the capsid protein HI loop relative to a corresponding parental AAV capsid protein.

8. The variant AAV capsid of claim 7, wherein the HI loop variant capsid protein comprises the removal of 1, 2, 3, 4, 5, 6, 7, 8, 9, 10, 11, or 12 amino acids of the stretch of HI Loop amino acid residues between amino acid D657 and amino acid N668 (DPPTAFNKDKLN; SEQ ID NO: 127) of VP1 of AAV9, or the corresponding amino acids in the capsid protein of another AAV serotype.

9. The variant AAV capsid of claim 7, wherein the removal of one or more amino acids in the capsid protein HI loop further comprises an insertion of a flexible peptide linker in the HI loop, wherein the insertion of a flexible peptide linker replaces a contiguous stretch of from 2 amino acids to 18 amino acids of the parental AAV capsid protein.

10. The variant AAV capsid of claim 7, wherein the HI loop variant capsid protein comprises an amino acid sequence that is at least 80%, 85%, 90%, 95%, 98%, 99%, or 100% identical to SEQ ID NO: 114 or to SEQ ID NO: 115.

11. The variant AAV capsid of claim 5, wherein the tandem multimer comprises an amino acid sequence that is at least 80%, 85%, 90%, 95%, 98%, 99%, or 100% identical to SEQ ID NO: 124, SEQ ID NO: 125, SEQ ID NO: 116, SEQ ID NO: 118, SEQ ID NO: 119, SEQ ID NO: 120, SEQ ID NO: 121, and/or SEQ ID NO: 117.

12. The variant AAV capsid of claim 1, wherein the variant AAV capsid comprises a plurality of guided variant capsid proteins, wherein the guided variant capsid protein comprises an insertion of a guide peptide relative to a corresponding parental AAV capsid protein, wherein the insertion of a guide peptide is between any one of amino acid 1 to amino acid 240 of VP1 of AAV9, or the corresponding position in the capsid protein of another AAV serotype.

13. The variant AAV capsid of claim 12, wherein the guide peptide comprises a contiguous stretch of from about 2 amino acids to about 100 amino acids from the N-terminal region of a capsid protein of a larger virus species, wherein the capsid of the larger virus species comprises a triangulation (T) number of greater than 1, and wherein the insertion of a guide peptide is at a structurally analogous turn in the parental AAV capsid protein relative to the capsid protein of the larger virus species

14. The variant AAV capsid of claim 12, wherein the guide peptide comprises a contiguous stretch of at least about 10 amino acids of any one of the sequences of SEQ ID NOS: 89-102 or of a sequence comprising one mismatch or two mismatches relative to any one of the sequences of SEQ ID NOS: 89-102.

15. The variant AAV capsid of claim 12, wherein the guide peptide comprises a contiguous stretch of from about 2 amino acids to about 100 amino acids of any one of the sequences of SEQ ID NOS: 103-109.

16. The variant AAV capsid of claim 12, wherein the insertion of a guide peptide replaces:
(a) a contiguous stretch of from about 2 amino acids to about 200 amino acids of the parental AAV capsid protein;
(b) a contiguous stretch of from about 2 amino acids to about 200 amino acids of the parental AAV capsid protein following the VP1 start codon, VP2 start codon, and/or VP3 start codon;
(c) a contiguous stretch of from about 2 amino acids to about 200 amino acids of the parental AAV capsid protein following the VP2 start codon;
(d) a contiguous stretch of from about 2 amino acids to about 50 amino acids following the VP3 start codon; and/or
(e) amino acid A204 to amino acid G220 of VP1 of AAV9, or the corresponding position in the capsid protein of another AAV serotype.

17. The variant AAV capsid of claim 12, wherein the insertion of the guide peptide replaces amino acid A204 to amino acid G220 of VP1 of AAV9, or the corresponding position in the capsid protein of another AAV serotype.

18. The variant AAV capsid of claim 12, wherein the guided variant capsid protein comprises an amino acid sequence that is at least 80%, 85%, 90%, 95%, 98%, 99%, or 100% identical to SEQ ID NO: 123 and/or SEQ ID NO: 122.

19. A recombinant AAV (rAAV), the rAAV comprising:
a) the variant AAV capsid of claim 1; and
b) a heterologous nucleic acid, wherein the heterologous nucleic acid comprises a polynucleotide encoding a payload, and wherein the payload comprises a payload RNA agent and/or a payload protein.

20. A method of treating a disease or disorder in a subject, the method comprising: administering to the subject a therapeutically effective amount of the rAAV of claim 19.
